(19) United States
(12) Patent Application Publication
(10) Pub. No.: US 2026/0092275 A1
(43) Pub. Date: Apr. 2, 2026

(54) PRODUCTS AND COMPOSITIONS

(71) Applicant: Sirnaomics, Inc., Germantown, MD (US)

(72) Inventor: Dmitry SAMARSKY, Germantown, MD (US)

(21) Appl. No.: 19/082,089

(22) Filed: Mar. 17, 2025

Related U.S. Application Data
(63) Continuation of application No. PCT/US23/74474, filed on Sep. 18, 2023.
(60) Provisional application No. 63/407,462, filed on Sep. 16, 2022.

(51) Int. Cl. C12N 15/113; A61K 39/395; A61K 45/06

(52) U.S. Cl. CPC C12N 15/113; A61K 39/3955; A61K 45/06; C12N 2310/14; C12N 2310/313; C12N 2310/315; C12N 2310/321; C12N 2310/322; C12N 2310/351

(57) ABSTRACT

Nucleic acid products and compositions are provided, together with methods for their use, to modulate, in particular, interfere with or inhibit CFB and C5 gene expression.

### RELATED APPLICATIONS

This application is a continuation of International Application No. PCT/US23/74474, filed Sep. 18, 2023, which claims the benefit of and priority to U.S. Provisional Application No. 63/407,462, filed Sep. 16, 2022, the contents of each of which are incorporated herein by reference in their entireties.

### SEQUENCE LISTING

The instant application contains a Sequence Listing which has been submitted electronically in XML format and is hereby incorporated by reference in its entirety. Said XML document, created on Aug. 31, 2023, is named 4690_0079i_SL.xml and is 10,728 bytes in size.

### FIELD

Nucleic acid products, compositions and related methods of use are provided that modulate, in particular, interfere with or inhibit CFB and C5 gene expression in mammals and are useful to treat, prevent, or ameliorate CFB- and C5-associated disorders.

### BACKGROUND

The complement system is part of the innate immune system. Compared to the adaptive immune system, it is evolutionary older and conserved across most taxa. Its function includes decorating microbes of potentially pathogenic nature (a process referred to as opsonization) and target them for destruction, which is effected by a macromolecular assembly known as the membrane attachment complex (MAC). Certain components of the complement system, once activated, contribute to chemoattraction and activation of leukocytes.

Complement activation may be triggered by various factors, which all involve presence of microbes but may also involve components of the adaptive immune system such as Ig including IgM. Three main pathways of complement activation have been recognized and are referred to as classical pathway, alternative pathway and lectin pathway.

In functional terms, complement activation occurs inherently at a low level (spontaneous cleavage of C3 to yield C3a and C3b) and is reinforced in the presence of microbes via an enzymatic cascade converting inactive forms of enzymes (zymogenes) into their active counterparts. The term “convertase”, such as C3 convertase, is primarily a functional term and may refer to structurally distinct complexes. One type of C3 convertases is a complex of C3b and complement factor B (CFB, Factor B). Once formed, a C3 convertase can convert large amounts of C3 into its cleavage products C3a and C3b within short amount of time. The specific C3 convertase, which is a complex of C3b and Factor B has originally been described in the context of the alternative pathway, but may form also in the context of the other two pathways. Within the alternative pathway, Factor B is also a constituent of C5 convertase, a complex, which converts C5, a more downstream component of the pathway, into its active form. The formation of C5 by C5 convertase, cleaving C5 into C5b and C5a is the initiating event in the late steps of complement activation. Based on C5b, the membrane attack complex (MAC) is formed, which lyses a target membrane by building a pore out of C9 molecules.

### Disease

Paroxysmal Nocturnal Hemoglobinuria (PNH) is an acquired disorder of hematopoiesis characterized by a somatic mutation in the PIGA gene that prevents or impairs the synthesis of glycosylphosphatidylinosital (GPI) anchors. The deficiency on red blood cells (RBCs) of GPI-anchored proteins, including complement regulators CD55 and CD59 results in chronic intravascular hemolysis with recurrent exacerbations, anemia, smooth muscle cell dystonia, and high risk of thrombosis.

### Treatment

Eculizumab (C5 inhibitor, SOLIRIS) prevents complement mediated intravascular hemolysis and affords other clinical benefits.

However, because the RBCs of PNH patients on eculizumab are no longer lysed by complement yet exist with bound C3 fragments (C3-opsonized) they are removed (eliminated) by macrophages, likely via interaction with the complement receptor 3, producing a novel phenomenon called extravascular hemolysis.

Therefore, there is a need to provide further compounds and treatments having the potential of efficiently reducing the effects of PNH.

### SUMMARY

According to a first aspect, the disclosed embodiments are directed to a nucleic acid construct containing at least: [0014](a) a first nucleic acid portion that is at least partially complementary to at least a first portion of an RNA. which is transcribed from a CFB gene; [0015](b) a second nucleic acid portion that is at least partially complementary to at least a second portion of an RNA, which is transcribed from a C5 gene; [0016](c) a third nucleic acid portion that is at least partially complementary to the first nucleic acid portion of (a), so as to form a first nucleic acid duplex region therewith; [0017](d) a fourth nucleic acid portion that is at least partially complementary to the second nucleic acid portion of (b), so as to form a second nucleic acid duplex region therewith.

In particular, the inventors have found out that targeting C5 prevents intravascular hemolysis and targeting CFB prevents extravascular hemolysis at the same time. According to a second aspect, the disclosed embodiments are directed to a composition containing a nucleic acid construct according to the first aspect, and a physiologically acceptable excipient. According to a third aspect, the disclosed embodiments are directed to pharmaceutical composition containing a nucleic acid construct according to the first aspect.

According to a fourth aspect, the disclosed embodiments are directed to the nucleic acid construct according to the first aspect, for use in human or veterinary medicine or therapy.

According to a fifth aspect, the disclosed embodiments are directed to a nucleic acid construct according to the first aspect for use in a method of treating, ameliorating and/or preventing a disease or disorder.

According to a sixth aspect, the disclosed embodiments are directed to a method of treating a disease or disorder containing administration of a nucleic acid construct according to the first aspect, to an individual in need of treatment.

According to a seventh aspect, the disclosed embodiments are directed to a use of a nucleic acid construct according to the first aspect, for use in research as a gene function analysis tool.

According to an eighth aspect, the disclosed embodiments are directed to a use of a nucleic acid construct according to the first aspect in the manufacture of a medicament for a treatment of a disease or disorder.

Advantageous and/or exemplary features of constructs according to the disclosed embodiments are as follows:
1) they contain multiple (2 or more) at least partially double-stranded agents capable of triggering RNA interference, tied together into a single nanostructure predominantly through complementary (Watson-Crick) interactions;
2) optionally, other (e.g.) covalent bindings may be used to build the constructs and/or add various ligands (e.g., delivery/targeting moieties such as GalNAc and/or other carbohydrates, cholesterol, peptides, or small molecules, optionally attached via linkers);
3) the constructs of the disclosed embodiments predominantly comprise chemically modified nucleotides (e.g., 2′F, 2′OMe, LNO, PNA, MOE, BNA, PMO, phosphorothioate, phosphorodithioate, etc.), mostly (but not only) to increase resistance to nucleases;
4) the constructs contain “fragile” components (e.g., chemical linkers, unmodified nucleotides, etc.), which allow the constructs to disassemble upon exposure to certain biologic environments (e.g., exposure to extra- and/or intra-cellular fluids); particular examples could be (but not limited): a) cleavage of the oligo backbone by nucleases in the sites with non-modified nucleotides; b) cleavage of the chemical linkage due to the change of pH (e.g., in endosomes);
5) disassembly upon exposure to the certain biologic environments releases the active components (e.g., the at least partially double-stranded agents capable of triggering RNA interference) to modulate (up- or down-regulate, advantageously down-regulate) target gene expression in cells/organisms;
6) the constructs can be used to modulate, advantageously down-regulate or silence gene expression, to study gene function, or to treat various diseases associated with the target genes to be down-regulated.

### Effects Achieved by the Nucleic Acid Constructs

As can be seen from the this disclosure, including the examples below, the disclosed nucleic acid constructs, including CFB targeting antisense strands and C5 targeting antisense strands, are capable of reducing CFB and C5 gene expression at the same time in an effective manner. Furthermore, all antisense strands disclosed herein, which are being active against CFB in form of an mxRNA and all antisense strands being active against C5 in form of an mxRNA, as shown in the examples as well, are also active when being part of the muRNA nucleic acid constructs of the disclosed embodiments. This is because, without wishing to be bound by theory, it is believed that all antisense strands, no matter if they are in their mxRNA form or in their muRNA form are processed by the same RISC mechanism. For example, the antisense strands may be released from the muRNA constructs of the disclosed embodiments by dissembling in vivo.

As a consequence, the nucleic acid constructs according to the disclosed embodiments are capable of addressing the problem with Eculizumab set forth above, according to which RBCs are no longer lysed by complement yet exist with bound C3 fragments (C3-opsonized) they are removed (eliminated) by macrophages, likely via interaction with the complement receptor 3 producing a novel phenomenon called extravascular hemolysis. The nucleic acid constructs of the disclosed embodiments have the potential to be effective in treating PNH, as without being bound by theory, targeting C5 prevents intravascular hemolysis and targeting CFB prevents extravascular hemolysis.

Furthermore, it was surprisingly found that, in certain embodiments, the mentioned effects are achieved by using oligomeric compounds according to the disclosed embodiments for inhibiting the expression of CFB and C5 genes in the form of muRNA constructs having a reduced length of, e.g., 34 nucleosides compared to conventional siRNA molecules having greater lengths. This can, e.g., make a synthesis of muRNA molecules more cost and production efficient, because less units are needed.

For certain oligomeric compounds according to the disclosed embodiments, being in the form of muRNA constructs for inhibiting the expression of CFB and C5 genes, it was surprisingly found out that the aforementioned effects can be achieved by using short sense strands within the muRNA having a length of advantageously 14 nucleosides, which is shorter than the length of the sense strands in conventional siRNA molecules.

The effects and technical advantages achieved by using the novel oligomeric compounds for inhibiting CFB and C5 expression will become apparent in more detail in the detailed description and the examples.

### BRIEF DESCRIPTION OF THE DRAWINGS

FIG. 1 shows the effect of sequence structure optimization on the reduction in CFB gene expression. See Example 3. sss

FIG. 2 shows a study design including a timeline with the time points of applying the dose to the non-human primates (NHP) and time points for taking samples, as described in Example 4.

FIG. 3a shows a mean percent of remaining factor Bb (an established read-out for CFB down-regulation and complement pathway down-regulation) levels in the plasma for a single treatment oligomeric with the novel oligomeric constructs 106-13(4) (SEQ ID No. 1758) and 13(5) (SEQ ID No. 1757), as described in Example 4.

FIG. 3b shows a mean percent of remaining factor Bb levels in the plasma for a multiple treatment with the novel oligomeric constructs 106-13(4) (SEQ ID No. 1758) and 13(5) (SEQ ID No. 1757), as described in Example 4.

FIG. 4a shows a mean percent of remaining factor Bb levels in the plasma for groups treated with various doses of the novel oligomeric construct 106-13(4) (SEQ ID No. 1758) in comparison with the control group, as described in Example 4.

FIG. 4b shows a mean percent of remaining factor Bb levels in the plasma for groups treated with various doses of the novel oligomeric construct 13(5) (SEQ ID No. 1757) in comparison with the control group, as described in Example 4.

FIG. 5 shows an overview of a study protocol in mice with humanized liver, as described in Example 5.

FIG. 6 shows CFB knock-down in the mice at the mRNA level of two compounds (106-13(4) (SEQ ID No. 1758) and 13(5) (SEQ ID No. 1757) of the disclosed embodiments as compared to negative control after 2 and 6 weeks, as described in Example 5. sss

FIG. 7 shows amounts of CFB (“Factor B”) as well as of Factor Bb in plasma of the mice following administration of CFB-targeting compounds (106-13(4) (SEQ ID No. 1758) and 13(5) (SEQ ID No. 1757)), as compared to negative control after 2 and 6 weeks, as described in Example 5.

FIG. 8 shows single dose curves of certain C5 mxRNA compounds selected from Table 3e (SEQ ID Nos. 1959-2058) of the disclosed embodiments and their activity in inhibiting C5 gene expression (primary screening), as described in Example 7.

FIG. 9 shows dose curves of 25 C5 mxRNA compounds selected from Table 3e (SEQ ID Nos. 1959-2058) and their activity in inhibiting C5 gene expression (secondary screening), as described in Example 7.

FIG. 10 shows dose curves of C5 mxRNA lead compounds (C5-30 (SEQ ID No. 1988) and C5-37 (SEQ ID No. 1995)) selected from Table 3e (SEQ ID Nos. 1959-2058) for preparation in vivo and their dose curves, as described in Example 7.

FIG. 11 shows a study schedule and study information for a study described in Example 8, relating to C5 targeting mxRNA leads for candidate dose and duration response study in humanized liver-uPA-SCID mice (PXB) model.

FIG. 12 shows the effects of the C5 targeting mxRNA constructs, C5-30 (SEQ ID No. 1988) and C5-37 (SEQ ID NO. 1995) on dose and duration response in humanized liver-uPA-SCID mice (PXB) in the study described in Example 8.

FIG. 13a shows dose curves of C5 gene knockdown using muRNA constructs selected from Table 4b (SEQ ID Nos. 2067-2074) for preparation in vivo and their dose curves, as described in Example 9.

FIG. 13b shows dose curves of CFB gene knockdown using muRNA constructs selected from Table 4b (SEQ ID Nos. 2067-2074) for preparation in vivo and their dose curves, as described in Example 9.

FIG. 14 shows a schedule for a dose response study described in Example 10, evaluating human complement combination (C5 and CFB; muRNA) targeting leads for candidate in humanized liver-uPA-SCID mice model.

FIG. 15a shows results for CFB gene knockdown from dose response study (Example 10) evaluating human complement combination (C5 and CFB; muRNA) targeting leads (B106-C5-30 and B106-C5-37, SEQ ID Nos. 2067-2068) selected from Table 4b (SEQ ID Nos. 2067-2074) for candidate in humanized liver-uPA-SCID mice model.

FIG. 15b shows results for C5 gene knockdown from dose response study (Example 10) evaluating human complement combination (C5 and CFB; muRNA) targeting leads (B106-C5-30 and B106-C5-37, SEQ ID Nos. 2067-2068) selected from Table 4b (SEQ ID Nos. 2067-2074) for candidate in humanized liver-uPA-SCID mice model.

FIG. 16 shows the results of in vivo testing in a humanized mouse model of an advantageous construct of the disclosed embodiments, STP247G, construct B106-C5-30, SEQ ID Nos. 2067-2068. Shown are qPCR data at 2, 4, 8 and 12 weeks, obtained in accordance with Example 11 using a human C5 probe.

FIG. 17 shows the results of in vivo testing in a humanized mouse model of an advantageous construct of the disclosed embodiments, STP247G, construct B106-C5-30, SEQ ID Nos. 2067-2068. Shown are qPCR data at 2, 4, 8 and 12 weeks, obtained in accordance with Example 11 using a human CFB probe.

### DETAILED DESCRIPTION

Further embodiments (items) of the disclosed embodiments are described below by way of example only. These examples represent the best ways of putting the disclosed embodiments into practice that are currently known to the applicant although they are not the only ways in which this could be achieved.

It will be understood that the benefits and advantages described herein may relate to one embodiment or may relate to several embodiments. The embodiments are not limited to those that solve any or all of the stated problems or those that have any or all of the stated benefits and advantages. Embodiments labelled “advantageous” or “advantageously” are not intended to limit the scope of the claims but to show optional disclosed embodiments.

Features of different aspects and embodiments of the disclosed embodiments may be combined as appropriate, as would be apparent to a skilled person, and may be combined with any of the aspects of the disclosed embodiments.

### Definitions

The following definitions pertain to the disclosed embodiments throughout. In many instances, the definitions, in addition to the respective definition as such, provide non-exhaustive listings of possible implementations, which amount to certain advantageous embodiments.

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21st edition, 2005; and “Antisense Drug Technology, Principles, Strategies, and Applications” Edited by Stanley T. Crooke, CRC Press, Boca Raton, Florida; and Sambrook et al., “Molecular Cloning, A laboratory Manual,” 2nd Edition, Cold Spring Harbor Laboratory Press, 1989, which are hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

As used herein, “excipient” means any compound or mixture of compounds that is added to a composition as provided herein that is suitable for delivery of an oligomeric compound.

As used herein, “nucleoside” means a compound containing a nucleobase moiety and a sugar moiety. Nucleosides include, but are not limited to, naturally occurring nucleosides (as found in DNA and RNA) and modified nucleosides. Nucleosides may be linked to a phosphate moiety, phosphate-linked nucleosides also being referred to as “nucleotides”. The structural features and/or the lengths of oligomeric compounds or nucleic acid constructs disclosed herein is expressed in terms of “nucleosides” or “nucleotides”.

As used herein, “chemical modification” or “chemically modified” means a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

As used herein, “furanosyl” means a structure containing a 5-membered ring containing four carbon atoms and one oxygen atom.

As used herein, “naturally occurring sugar moiety” means a ribofuranosyl as found in naturally occurring RNA or a deoxyribofuranosyl as found in naturally occurring DNA. A “naturally occurring sugar moiety” as referred to herein is also termed as an “unmodified sugar moiety”. In particular, such a “naturally occurring sugar moiety” or an “unmodified sugar moiety” as referred to herein has a —H (DNA sugar moiety) or —OH (RNA sugar moiety) at the 2′-position of the sugar moiety, especially a —H (DNA sugar moiety) at the 2′-position of the sugar moiety.

As used herein, “sugar moiety” means a naturally occurring sugar moiety or a modified sugar moiety of a nucleoside. As used herein, “modified sugar moiety,” means a substituted sugar moiety or a sugar surrogate.

As used herein, “substituted sugar moiety” means a furanosyl that has been substituted. Substituted sugar moieties include, but are not limited to furanosyls containing substituents at the 2′-position, the 3-position, the 5′-position and/or the 4′-position. Certain substituted sugar moieties are bicyclic sugar moieties.

As used herein, “2′-substituted sugar moiety” means a furanosyl containing a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring).

As used herein, “MOE” means —OCH2CH2OCH3.

As used herein, “2′-F nucleoside” refers to a nucleoside containing a sugar containing fluorine at the 2′ position. Unless otherwise indicated, the fluorine in a 2′-F nucleoside is in the ribo position (replacing the OH of a natural ribose). Duplexes of uniformly modified 2′-fluorinated (ribo) oligonucleotides hybridized to RNA strands are not RNase H substrates while the analogs retain RNase H activity.

As used herein the term “sugar surrogate” means a structure that does not comprise a furanosyl and that is capable of replacing the naturally occurring sugar moiety of a nucleoside, such that the resulting nucleoside sub-units are capable of linking together and/or linking to other nucleosides to form an oligomeric compound, which is capable of hybridizing to a complementary oligomeric compound. Such structures include rings containing a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally containing additional substituents). Sugar surrogates also include more complex sugar replacements (e.g., the non-ring systems of peptide nucleic acid). Sugar surrogates include without limitation morpholinos, cyclohexenyls and cyclohexitols.

As used herein, “bicyclic sugar moiety” means a modified sugar moiety containing a 4 to 7 membered ring (including but not limited to a furanosyl) containing a bridge connecting two atoms of the 4 to 7 membered ring to form a second ring, resulting in a bicyclic structure. In certain embodiments, the 4 to 7 membered ring is a sugar ring. In certain embodiments, the 4 to 7 membered ring is a furanosyl. In certain such embodiments, the bridge connects the 2′-carbon and the 4′-carbon of the furanosyl.

As used herein, “nucleotide” means a nucleoside further containing a phosphate linking group. As used herein, “linked nucleosides” may or may not be linked by phosphate linkages and thus includes, but is not limited to “linked nucleotides.” As used herein, “linked nucleosides” are nucleosides that are connected in a continuous sequence (i.e., no additional nucleosides are present between those that are linked).

As used herein, “nucleobase” means a group of atoms that can be linked to a sugar moiety to create a nucleoside that is capable of incorporation into an oligonucleotide, and where the group of atoms is capable of bonding, more specifically hydrogen bonding, with a complementary naturally occurring nucleobase of another oligonucleotide or nucleic acid. Nucleobases may be naturally occurring or may be modified.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase,” means any nucleobase that is not a naturally occurring nucleobase.

As used herein, “modified nucleoside” means a nucleoside containing at least one chemical modification compared to naturally occurring RNA or DNA nucleosides. Modified nucleosides can comprise a modified sugar moiety and/or a modified nucleobase.

As used herein, “bicyclic nucleoside” or “BNA” means a nucleoside containing a bicyclic sugar moiety.

As used herein, “locked nucleic acid nucleoside” or “LNA” means a nucleoside containing a bicyclic sugar moiety containing a 4′-CH2-O-2′bridge.

As used herein, “2′-substituted nucleoside” means a nucleoside containing a substituent at the 2′-position of the sugar moiety other than H or OH. Unless otherwise indicated, a 2′-substituted nucleoside is not a bicyclic nucleoside.

As used herein, “deoxynucleoside” means a nucleoside containing 2′-H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

As used herein, “oligonucleotide” means a compound containing a plurality of linked nucleosides. In certain embodiments, an oligonucleotide contain one or more unmodified ribonucleosides (RNA) and/or unmodified deoxyribonucleosides (DNA) and/or one or more modified nucleosides.

As used herein, “modified oligonucleotide” means an oligonucleotide containing at least one modified nucleoside and/or at least one modified internucleoside linkage.

Advantageous modified internucleoside linkages are those, which confer increased stability as compared to the naturally occurring phosphodiesters. “Stability” refers in particular to stability against hydrolysis including enzyme-catalyzed hydrolysis, enzymes including exonucleases and endonucleases.

Advantageous positions for such modified internucleoside linkages include the termini and the hairpin loop of single-stranded oligomeric compounds of the disclosed embodiments. For example, the internucleoside linkages connecting first and second nucleoside and second and third nucleoside counting from the 5′ terminus, and/or the internucleoside linkages connecting first and second nucleoside and second and third nucleoside counting from the 3′ terminus are modified. In addition, a linkage connecting the terminal nucleoside of the 3′ terminus with a ligand, such as GalNAc, may be modified.

As discussed above, advantageous positions are in the hairpin loop of the single-stranded oligomeric compounds. In particular, all linkages, all but one linkages or the majority of linkages in the hairpin loop are modified. As used herein, “linkages in the hairpin loop” designates the linkages between nucleosides, which are not engaged in base pairing. For example, in a hairpin loop consisting of five nucleosides, there are four linkages between nucleosides, which are not engaged in base pairing. Advantageously, the term “linkages in the hairpin loop” also extends to the linkages connecting the stem to the loop, i.e., those linkages, which connect a base-paired nucleoside to a non-based paired nucleoside. Generally, there are two such positions in hairpins and mxRNAs in accordance with the disclosed embodiments.

Most advantageous is that modified internucleoside linkages are at both termini and in the hairpin loop.

As used herein, “linkage” or “linking group” means a group of atoms that link together two or more other groups of atoms.

As used herein “internucleoside linkage” means a covalent linkage between adjacent nucleosides in an oligonucleotide.

As used herein “naturally occurring internucleoside linkage” means a 3′ to 5′ phosphodiester linkage.

As used herein, “modified internucleoside linkage,” means any internucleoside linkage other than a naturally occurring internucleoside linkage. In particular, a “modified internucleoside linkage” as referred to herein can include a modified phosphorous linking group such as a phosphorothioate or phosphorodithioate internucleoside linkage.

As used herein, “terminal internucleoside linkage” means the linkage between the last two nucleosides of an oligonucleotide or defined region thereof.

As used herein, “phosphorus linking group” means a linking group containing a phosphorus atom and can include naturally occurring phosphorous linking groups as present in naturally occurring RNA or DNA, such as phosphodiester linking groups, or modified phosphorous linking groups that are not generally present in naturally occurring RNA or DNA, such as phosphorothioate or phosphorodithioate linking groups. Phosphorus linking groups can therefore include without limitation, phosphodiester, phosphorothioate, phosphorodithioate, phosphonate, methylphosphonate, phosphoramidate, phosphorothioamidate, thionoalkylphosphonate, phosphotriesters, thionoalkylphosphotriester and boranophosphate.

As used herein, “internucleoside phosphorus linking group” means a phosphorus linking group that directly links two nucleosides.

As used herein, “oligomeric compound” means a polymeric structure containing two or more substructures. In certain embodiments, an oligomeric compound contain an oligonucleotide, such as a modified oligonucleotide. In certain embodiments, an oligomeric compound further contain one or more conjugate groups and/or terminal groups and/or ligands. In certain embodiments, an oligomeric compound consists of an oligonucleotide. In certain embodiments, an oligomeric compound contain a backbone of one or more linked monomeric sugar moieties, where each linked monomeric sugar moiety is directly or indirectly attached to a heterocyclic base moiety. In certain embodiments, oligomeric compounds may also include monomeric sugar moieties that are not linked to a heterocyclic base moiety, thereby providing abasic sites. Oligomeric compounds may be defined in terms of a nucleobase sequence only, i.e., by specifying the sequence of A, G, C, U (or T). In such a case, the structure of the sugar-phosphate backbone is not particularly limited and may or may not comprise modified sugars and/or modified phosphates. On the other hand, oligomeric compounds may be more comprehensively defined, i.e., by specifying not only the nucleobase sequence, but also the structure of the backbone, in particular the modification status of the sugars (unmodified, 2′-OMe modified, 2′-F modified etc.) and/or of the phosphates. An mxRNA is one non-limiting example for an oligomeric compound.

As used herein, “nucleic acid construct” or “construct” refers to an assembly of two or more, such as four oligomeric compounds. The oligomeric compounds may be connected to each other by covalent bonds such phosphodiester bonds as they occur in naturally occurring nucleic acids or modified versions thereof as disclosed herein, or by non-covalent bonds such as hydrogen bonds, advantageously hydrogen bonds between nucleobases such as Watson-Crick base pairing. In certain embodiments, advantageous is that a construct contain four oligomeric compounds, two of which are connected covalently, thereby giving rise to two nucleic acid strands, which nucleic acid strands are bound to each other by hydrogen bonds. Complementarity between the strand may be throughout, but is not necessarily so. In particular, exemplary embodiments provide for an antisense strand targeting a first region of the mRNA to be connected covalently with a sense strand of another gene-targeting double stranded RNA molecule, and of the antisense strand of the mRNA-targeting double stranded RNA molecule to be connected covalently to a sense strand of the other mRNA-targeting double stranded RNA molecule. Since antisense and sense strands of the parent single-target-directed RNA molecules do not need to have the same length and advantageously do not have the same length with antisense portions being longer than sense portions, a advantageous construct of the disclosed embodiments contains a central region where the 3′ regions of the antisense portions of the parent single-target-directed RNA molecules face each other. In that region generally no or only partial base pairing will occur, while full complementarity is not excluded. Otherwise, where antisense and sense portions of the respective parent RNA molecules face each other; there is complementarity, advantageously full complementarity or 1 or 2 mismatches. A muRNA is non-limiting example for a nucleic acid construct.

The term “strand” has its art-established meaning and refers to a plurality of linked nucleosides, the linker not being particularly limited, but including phosphodiesters and variants thereof as disclosed herein. A strand may also be viewed as a plurality of linked nucleotides in which case the linker would be a covalent bond.

As used herein, “terminal group” means one or more atom attached to either, or both, the 3′ end or the 5′ end, also called “terminus” of an oligonucleotide. In certain embodiments, a terminal group contain one or more terminal group nucleosides, whereas a “terminal nucleoside” is only one nucleotide at the respective end (5′ end or 3′ end).

As used herein, “conjugate” or “conjugate group” means an atom or group of atoms bound to an oligonucleotide or oligomeric compound. In certain embodiments, a conjugate group links a ligand to a modified oligonucleotide or oligomeric compound. In general, conjugate groups can modify one or more properties of the compound to which they are attached, including, but not limited to pharmacodynamic, pharmacokinetic, binding, absorption, cellular distribution, cellular uptake, charge and/or clearance properties.

As used herein, “conjugate linker” or “linker” in the context of a conjugate group means a portion of a conjugate group containing any atom or group of atoms and which covalently link an oligonucleotide to another portion of the conjugate group. In certain embodiments, the point of attachment on the oligomeric compound is the 3′-oxygen atom of the 3′-hydroxyl group of the 3′ terminal nucleoside of the oligonucleotide. In certain embodiments, the point of attachment on the oligomeric compound is the 5′-oxygen atom of the 5′-hydroxyl group of the 5′ terminal nucleoside of the oligonucleotide. In certain embodiments, the bond for forming attachment to the oligomeric compound is a cleavable bond. In certain such embodiments, such cleavable bond constitutes all or part of a cleavable moiety.

In certain embodiments, conjugate groups comprise a cleavable moiety (e.g., a cleavable bond or cleavable nucleoside) and ligand portion that can comprise one or more ligands, such as a carbohydrate cluster portion, such as an N-Acetyl-Galactosamine, also referred to as “GalNAc”, cluster portion. In certain embodiments, the carbohydrate cluster portion is identified by the number and identity of the ligand. For example, in certain embodiments, the carbohydrate cluster portion contain 2 GalNAc groups. For example, in certain embodiments, the carbohydrate cluster portion contain 3 GalNAc groups and this is particularly advantageous. In certain embodiments, the carbohydrate cluster portion contain 4 GalNAc groups. Such ligand portions are attached to an oligomeric compound via a cleavable moiety, such as a cleavable bond or cleavable nucleoside. The ligands can be arranged in a linear or branched configuration, such as a biantennary or triantennary configurations. A advantageous carbohydrate cluster has the following formula:

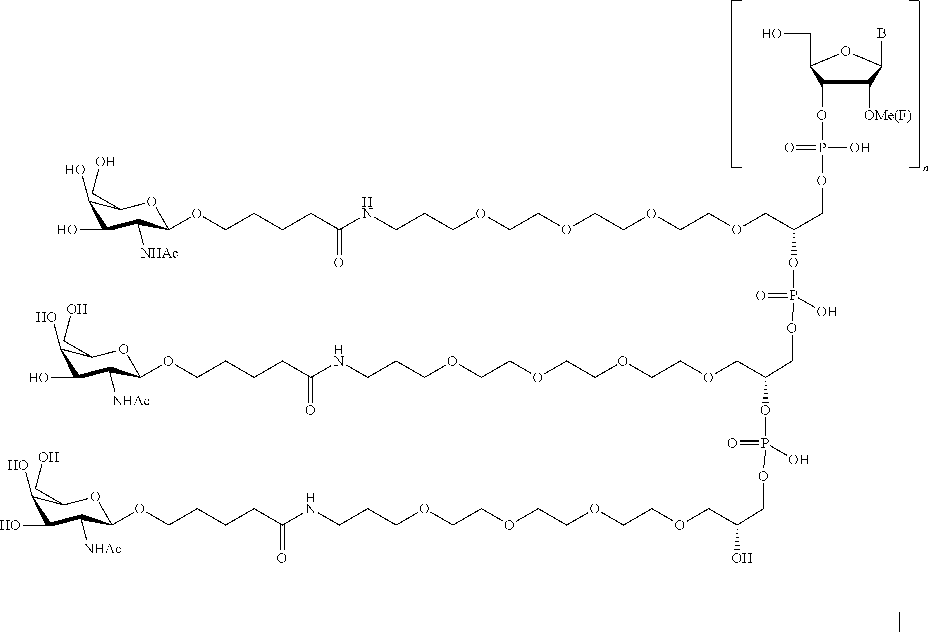

where in the structural formula one, two, or three phosphodiester linkages can also be substituted by phosphorothioate linkages.

As used herein, “cleavable moiety” means a bond or group that is capable of being cleaved under physiological conditions. In certain embodiments, a cleavable moiety is cleaved inside a cell or sub-cellular compartments, such as an endosome or lysosome. In certain embodiments, a cleavable moiety is cleaved by endogenous enzymes, such as nucleases. In certain embodiments, a cleavable moiety contain a group of atoms having one, two, three, four, or more than four cleavable bonds. In certain embodiments, a cleavable moiety is a phosphodiester linkage.

As used herein, “cleavable bond” means any chemical bond capable of being broken.

As used herein, “carbohydrate cluster” means a compound having one or more carbohydrate residues attached to a linker group.

As used herein, “modified carbohydrate” means any carbohydrate having one or more chemical modifications relative to naturally occurring carbohydrates.

As used herein, “carbohydrate derivative” means any compound, which may be synthesized using a carbohydrate as a starting material or intermediate.

As used herein, “carbohydrate” means a naturally occurring carbohydrate, a modified carbohydrate, or a carbohydrate derivative. A carbohydrate is a biomolecule including carbon (C), hydrogen (H) and oxygen (O) atoms. Carbohydrates can include monosaccharide, disaccharides, trisaccharides, tetrasaccharides, oligosaccharides or polysaccharides, such as one or more galactose moieties, one or more lactose moieties, one or more N-Acetyl-Galactosamine moieties, and/or one or more mannose moieties. A particularly advantageous carbohydrate is N-Acetyl-Galactosamine.

As used herein, “strand” means an oligomeric compound containing linked nucleosides.

As used herein, “single strand” or “single-stranded” means an oligomeric compound containing linked nucleosides that are connected in a continuous sequence without a break there between. Such single strands may include regions of sufficient self-complementarity so as to be capable of forming a stable self-duplex in a hairpin structure.

As used herein, “hairpin” means a single stranded oligomeric compound that includes a duplex formed by base pairing between sequences in the strand that are self-complementary and opposite in directionality.

As used herein, “hairpin loop” means an unpaired loop of linked nucleosides in a hairpin that is created as a result of hybridization of the self-complementary sequences. The resulting structure looks like a loop or a U-shape.

In particular, short hairpin RNA, also denoted as shRNA, contain a duplex region and a loop connecting the regions forming the duplex. The end of the duplex region, which does not carry the loop, may be blunt-ended or carry (a) 3′ and/or (a) 5′ overhang(s). Advantageously the constructs are blunt-ended constructs. The term “shRNA” is more generic than “mxRNA”, as defined below, and may include compounds in which the loop is not or not exclusively formed out of an antisense strand. In particular, shRNA includes an antisense strand, also called guide strand, being complementary to a region of a target RNA, and a sense strand, i.e., a passenger strand, being substantially complementary to the antisense strand. More particularly, the antisense strand and the sense strand within the shRNA are directly linked, e.g., by a phosphate or a phosphorothioate, or linked by a third portion of linked nucleosides forming the loop, which means that the 3′ end of the antisense strand is linked to the 5′ end of the sense strand via covalent bonding over several other groups. Such direct linkage does not include a gap or nick.

As used herein, “directionality” means the end-to-end chemical orientation of an oligonucleotide based on the chemical convention of numbering of carbon atoms in the sugar moiety meaning that there will be a 5′-end defined by the 5′ carbon of the sugar moiety, and a 3′-end defined by the 3′ carbon of the sugar moiety. In a duplex or double stranded oligonucleotide, the respective strands run in opposite 5′ to 3′ directions to permit base pairing between them.

As used herein, “duplex”, or also abbreviated as “dup”, means two or more complementary strand regions, or strands, of an oligonucleotide or oligonucleotides, hybridized together by way of non-covalent, sequence-specific interaction there between. Most commonly, the hybridization in the duplex will be between nucleobases adenine (A) and thymine (T), and/or (A) adenine and uracil (U), and/or guanine (G) and cytosine (C). The duplex may be part of a single stranded structure, where self-complementarity leads to hybridization, or as a result of hybridization between respective strands in a double stranded construct.

As used herein, “double strand” or “double stranded” means a pair of oligomeric compounds that are hybridized to one another. In certain embodiments, a double-stranded oligomeric compound contain a first and a second oligomeric compound.

As used herein, “expression” means the process by which a gene ultimately results in a protein. Expression includes, but is not limited to, transcription, post-transcriptional modification (e.g., splicing, polyadenylation, addition of 5′-cap), and translation.

As used herein, “transcription” or “transcribed” refers to the first of several steps of DNA based gene expression in which a target sequence of DNA is copied into RNA (especially mRNA) by the enzyme RNA polymerase. During transcription, a DNA sequence is read by an RNA polymerase, which produces a complementary, antiparallel RNA sequence called a primary transcript.

As used herein, “target sequence” means a sequence to which an oligomeric compound is intended to hybridize to result in a desired activity with respect to gene expression. Oligonucleotides have sufficient complementarity to their target sequences to allow hybridization under physiological conditions.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In both DNA and RNA, guanine (G) is complementary to cytosine (C). In certain embodiments, complementary nucleobase means a nucleobase of an oligomeric compound that is capable of base pairing with a nucleobase of its target sequence. For example, if a nucleobase at a certain position of an oligomeric compound is capable of hydrogen bonding with a nucleobase at a certain position of a target sequence, then the position of hydrogen bonding between the oligomeric compound and the target sequence is considered complementary at that nucleobase pair. Nucleobases containing certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another.

As used herein, “complementary” in reference to oligomeric compounds (e.g., linked nucleosides, oligonucleotides) means the capacity of such oligomeric compounds or regions thereof to hybridize to a target sequence, or to a region of the oligomeric compound itself, through nucleobase complementarity.

Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or regions are 80%>complementary. In certain embodiments, complementary oligomeric compounds or regions are 90%>complementary. In certain embodiments, complementary oligomeric compounds or regions are at least 95% complementary. In certain embodiments, complementary oligomeric compounds or regions are 100% complementary.

As used herein, “self-complementarity” in reference to oligomeric compounds means a compound that may fold back on itself, creating a duplex as a result of nucleobase hybridization of internal complementary strand regions. Depending on how close together and/or how long the strand regions are, then the compound may form hairpin loops, junctions, bulges or internal loops.

As used herein, “mismatch” means a nucleobase of an oligomeric compound that is not capable of pairing with a nucleobase at a corresponding position of a target sequence, or at a corresponding position of the oligomeric compound itself when the oligomeric compound hybridizes as a result of self-complementarity, when the oligomeric compound and the target sequence and/or self-complementary regions of the oligomeric compound, are aligned.

As used herein, “hybridization” means the pairing of complementary oligomeric compounds (e.g., an oligomeric compound and its target sequence). While not limited to a particular mechanism, the most common mechanism of pairing involves hydrogen bonding, which may be Watson-Crick, Hoogsteen or reversed Hoogsteen hydrogen bonding, between complementary nucleobases.

As used herein, “specifically hybridizes” means the ability of an oligomeric compound to hybridize to one nucleic acid site with greater affinity than it hybridizes to another nucleic acid site.

As used herein, “fully complementary” in reference to an oligomeric compound or region thereof means that each nucleobase of the oligomeric compound or region thereof is capable of pairing with a nucleobase of a complementary nucleic acid target sequence or a self-complementary region of the oligomeric compound. Thus, a fully complementary oligomeric compound or region thereof contain no mismatches or unhybridized nucleobases with respect to its target sequence or a self-complementary region of the oligomeric compound.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein, “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression.

As used herein, “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

As used herein, “differently modified” mean chemical modifications or chemical substituents that are different from one another, including absence of modifications. Thus, for example, a MOE nucleoside and an unmodified naturally occurring RNA nucleoside are “differently modified,” even though the naturally occurring nucleoside is unmodified. Likewise, DNA and RNA oligonucleotides are “differently modified,” even though both are naturally occurring unmodified nucleosides. Nucleosides that are the same but for containing different nucleobases are not differently modified. For example, a nucleoside containing a 2′-OMe modified sugar moiety and an unmodified adenine nucleobase and a nucleoside containing a 2′-OMe modified sugar moiety and an unmodified thymine nucleobase are not differently modified.

As used herein, “the same type of modifications” refers to modifications that are the same as one another, including absence of modifications. Thus, for example, two unmodified RNA nucleosides have “the same type of modification,” even though the RNA nucleosides are unmodified. Such nucleosides having the same type modification may comprise different nucleobases.

As used herein, “region” or “regions”, or “portion” or “portions”, mean a plurality of linked nucleosides that have a function or character as defined herein, in particular with reference to the claims and definitions as provided herein. Typically, such regions or portions comprise at least 10, at least 11, at least 12 or at least 13 linked nucleosides. For example, such regions can comprise 13 to 20 linked nucleosides, such as 13 to 16 or 18 to 20 linked nucleosides. Typically a first region as defined herein consists essentially of 18 to 20 nucleosides and a second region as defined herein consists essentially of 13 to 16 linked nucleosides.

As used herein, “pharmaceutically acceptable carrier or diluent” means any substance suitable for use in administering to an animal. In certain embodiments, a pharmaceutically acceptable carrier or diluent is sterile saline. In certain embodiments, such sterile saline is pharmaceutical grade saline.

As used herein, “substituent” and “substituent group,” means an atom or group that replaces the atom or group of a named parent compound. For example, a substituent of a modified nucleoside is any atom or group that differs from the atom or group found in a naturally occurring nucleoside (e.g., a modified 2′-substituent is any atom or group at the 2′-position of a nucleoside other than H or OH). Substituent groups can be protected or unprotected. In certain embodiments, compounds of the present disclosure have substituents at one or at more than one position of the parent compound. Substituents may also be further substituted with other substituent groups and may be attached directly or via a linking group such as oxygen or an alkyl or hydrocarbyl group to a parent compound.

Such substituents can be present as the modification on the sugar moiety, in particular a substituent present at the 2′-position of the sugar moiety. Unless otherwise indicated, groups amenable for use as substituents include without limitation, one or more of halo, hydroxyl, alkyl, alkenyl, alkynyl, acyl, carboxyl, alkoxy, alkoxyalkylene and amino substituents. Certain substituents as described herein can represent modifications directly attached to a ring of a sugar moiety (such as a halo, such as fluoro, directly attached to a sugar ring), or a modification indirectly linked to a ring of a sugar moiety by way of an oxygen linking atom that itself is directly linked to the sugar moiety (such as an alkoxyalkylene, such as methoxyethylene, linked to an oxygen atom, overall providing an MOE substituent as described herein attached to the 2′-position of the sugar moiety).

As used herein, “alkyl,” as used herein, means a saturated straight or branched monovalent C1-6 hydrocarbon radical, with methyl being a most advantageous alkyl as a substituent at the 2′-position of the sugar moiety. The alkyl group typically attaches to an oxygen linking atom at the 2′poisition of the sugar, therefore, overall providing a —Oalkyl substituent, such as an —OCH3 substituent, on a sugar moiety of an oligomeric compound according to the disclosed embodiments. This will be well understood be a person skilled in the art.

As used herein, “alkylene” means a saturated straight or branched divalent hydrocarbon radical of the general formula —CnH2n- where n is 1-6. Methylene or ethylene are advantageous alkylenes.

As used herein, “alkenyl” means a straight or branched unsaturated monovalent C2-6 hydrocarbon radical, with ethenyl or propenyl being most advantageous alkenyls as a substituent at the 2′-position of the sugar moiety. As will be well understood in the art, the degree of unsaturation that is present in an alkenyl radical is the presence of at least one carbon to carbon double bond. The alkenyl group typically attaches to an oxygen linking atom at the 2′-position of the sugar, therefore, overall providing a —Oalkenyl substituent, such as an —OCH2CH═CH2 substituent, on a sugar moiety of an oligomeric compound according to the disclosed embodiments. This will be well understood be a person skilled in the art.

As used herein, “alkynyl” means a straight or branched unsaturated C2-6 hydrocarbon radical, with ethynyl being a most advantageous alkynyl as a substituent at the 2′-position of the sugar moiety. As will be well understood in the art, the degree of unsaturation that is present in an alkynyl radical is the presence of at least one carbon to carbon triple bond. The alkynyl group typically attaches to an oxygen linking atom at the 2′-position of the sugar, therefore, overall providing a —Oalkynyl substituent on a sugar moiety of an oligomeric compound according to the disclosed embodiments. This will be well understood be a person skilled in the art.

As used herein, “carboxyl” is a radical having a general formula —CO2H.

As used herein, “acyl” means a radical formed by removal of a hydroxyl group from a carboxyl radical as defined herein and has the general Formula —C(O)—X where X is typically C1-6 alkyl.

As used herein, “alkoxy” means a radical formed between an alkyl group, such as a C1-6 alkyl group, and an oxygen atom where the oxygen atom is used to attach the alkoxy group either to a parent molecule (such as at the 2′-position of a sugar moiety), or to another group such as an alkylene group as defined herein. Examples of alkoxy groups include without limitation, methoxy, ethoxy, propoxy, isopropoxy, n-butoxy, sec-butoxy and tert-butoxy. Alkoxy groups as used herein may optionally include further substituent groups.

As used herein, alkoxyalkylene means an alkoxy group as defined herein that is attached to an alkylene group also as defined herein, and where the oxygen atom of the alkoxy group attaches to the alkylene group and the alkylene attaches to a parent molecule. The alkylene group typically attaches to an oxygen linking atom at the 2′-position of the sugar, therefore, overall providing a —Oalkylenealkoxy substituent, such as an —OCH2CH2OCH3 substituent, on a sugar moiety of an oligomeric compound according to the disclosed embodiments. This will be well understood by a person skilled in the art and is generally referred to as an MOE substituent as defined herein and as known in the art.

As used herein, “amino” includes primary, secondary and tertiary amino groups.

As used herein, “halo” and “halogen,” mean an atom selected from fluorine, chlorine, bromine and iodine.

As used herein, the term “mxRNA” is in particular understood as defined in WO 2020/044186 A2, which is incorporated by reference herein in its entirety. In particular, an mxRNA is a hairpin-shaped RNA molecule consisting of an antisense portion (also referred to as the guide strand) and a sense portion (also referred to the passenger strand). The mxRNA contain duplex region and a hairpin loop, where the mxRNA has an approximate length of about 34 nucleotides. The duplex region contain a region in which parts of the antisense portion and substantially the entire sense portion, typically 14 or 15 nucleotides of each strand, are base-paired. The hairpin loop connects both regions, i.e., antisense region and sense region, of that duplex via, e.g., a phosphate or a phosphorothioate linker, i.e., covalently, while the antisense portion typically has a length of about 18 to 20 nucleotides and, therefore, forms the antisense duplex region and the loop. The loop, of which the antisense portion is part, furthermore connects the sense, forming the second strand of the loop, and the antisense portion.

As used herein, the term “factor Bb” denotes the corresponding and commonly known protein, which binds to C3b within the C3 convertase within complement activation. Factor Bb is an active subunit of CFB and can be produced by a cleavage of CFB into factors Ba and Bb due to factor D, for example in the alternative pathway of the complement activation, after CFB is bound to C3b. The factor Bb level can, such as in the examples of the disclosed embodiments, be used as an indicator for the success of a silencing of CFB expression.

As used herein, the term “complement component C5” or just “C5” denotes the corresponding and commonly known protein, which decomposes into C5a and C5b, where C5b forms part of the membrane attack complex at the late stage of the complement activation. C5 is a protein that is in humans encoded by the C5 gene. Complement component C5 is the fifth component of complement, which plays an important role in inflammatory and cell killing processes. This protein is composed of alpha and beta polypeptide chains that are linked by a disulfide bridge. An activation peptide, C5a, which is an anaphylatoxin that possesses potent spasmogenic and chemotactic activity, is derived from the alpha polypeptide via cleavage with a C5-convertase. The C5b macromolecular cleavage product can form a complex with the C6 complement component, and this complex is the basis for formation of the membrane attack complex, which includes additional complement components.

As used herein, the term “muRNA” or “multi RNA” includes nucleic acid constructs containing more than one, typically two, RNA sequences, i.e., first and second nucleic acid portions, targeting different regions of the mRNA; or one region of the mRNA and an mRNA region of another target molecule. The targeting RNA sequences are also referred to as “antisense” or “guide” strands, while the respective passenger strands, i.e., third and fourth nucleic acid portions being complementary to the first and second portion, respectively, are also included in the nucleic acid construct. In particular, such muRNA are designed such that subsequent to in vivo administration, they are disassembled and the first and second nucleic acid portions are released. A particular example for such muRNA is shown below, where (1) is the first nucleic acid portion, (2) is the third nucleic acid portion being complementary to (1), (3) is the second nucleic acid portion being complementary to the fourth nucleic acid portion, while (5) is a labile linker while (6) is a ligand, which will both be explained below.

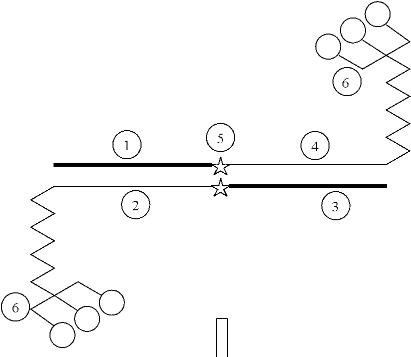

It will also be understood that oligomeric compounds as described herein may have one or more non-hybridizing nucleosides at one or both ends of one or both strands (overhangs) and/or one or more internal non-hybridizing nucleosides (mismatches) provided there is sufficient complementarity to maintain hybridization under physiologically relevant conditions. Alternatively, oligomeric compounds as described herein may be blunt ended at least one end.

The term “containing” is used herein to mean including the method steps or elements identified, but that such steps or elements do not comprise an exclusive list and as such, there may be present additional steps or elements.

Further, to the extent that the term “includes” is used in either the detailed description or the claims, such term is intended to be inclusive in a manner similar to the term “containing” as “containing” is interpreted when employed as a transitional word in a claim.

The disclosed embodiments relate to the following aspects and embodiments

muRNA Nucleic Acid Constructs

According to a first aspect, the disclosed embodiments are directed to a nucleic acid construct containing at least:
(a) a first nucleic acid portion that is at least partially complementary to at least a first portion of an RNA, which is transcribed from a CFB gene;
(b) a second nucleic acid portion that is at least partially complementary to at least a second portion of an RNA, which is transcribed from a C5 gene;
(c) a third nucleic acid portion that is at least partially complementary to the first nucleic acid portion of (a), so as to form a first nucleic acid duplex region therewith;
(d) a fourth nucleic acid portion that is at least partially complementary to the second nucleic acid portion of (b), so as to form a second nucleic acid duplex region therewith.

The construct may be designed such that subsequent to in vivo administration the construct disassembles to yield at least first and second discrete nucleic acid targeting molecules that respectively target the RNA portions transcribed from the target genes of (a) and (b);

whereby (i) the first nucleic acid targeting molecule is capable of modulating expression of the target gene of (a), and contain, or is derived from, at least the first nucleic acid portion of (a), and (ii) the second nucleic acid targeting molecule is capable of modulating expression of the target gene of (b), and contain, or is derived from, the second nucleic acid portion of (b).

The construct may be designed to disassemble such that the first and second discrete nucleic acid targeting molecules are respectively processed by independent RNAi-induced silencing complexes.

### Sequence Features, Labile Functionality and Structural Features of the RNA Molecules

The construct according to the first aspect and its aforementioned embodiments may at least comprise one labile functionality such that subsequent to in vivo administration the construct is cleaved so as to yield the at least first and second discrete nucleic acid targeting molecules.

The labile functionality may comprise one or more unmodified nucleotides. In particular the one or more unmodified nucleotides of the labile functionality represent one or more cleavage positions within the construct whereby subsequent to in vivo administration the construct is cleaved at the one or more cleavage positions so as to yield the at least first and second discrete nucleic acid targeting molecules. Especially, the cleavage positions may be respectively located within the construct so that subsequent to cleavage the first discrete nucleic acid targeting molecule contain, or is derived from, the first nucleic acid duplex region, and the second discrete nucleic acid targeting molecule contain, or is derived from, the second nucleic acid duplex region. Advantageously, the first discrete nucleic acid targeting molecule contain or consists of the first nucleic acid portion of (a) and the third nucleic acid portion of (c), and/or the second discrete nucleic acid targeting molecule contain or consists of the second nucleic acid portion of (b) and the fourth nucleic acid portion of (d).

In certain embodiments
(a) the first nucleic acid portion has a nucleobase sequence selected from Table 1a (SEQ ID Nos. 1-252);
(b) the second nucleic acid portion has a nucleobase sequence selected from Table 1c (SEQ ID Nos. 505-754);
(c) the third nucleic acid portion has a nucleobase sequence selected from Table 1b (SEQ ID Nos. 253-504); and/or
(d) the fourth nucleic acid portion has a nucleobase sequence selected from Table 1d (SEQ ID NOs: 755-1004).
where the third and fourth nucleobase sequences, to the extent they have a length of 14 nucleobases, may be shorter by one, two or three nucleobases, where advantageously the 5-terminal nucleobase(s) is/are absent. The compounds, which have been shown to be active as single molecules in the CFB inhibition and the C5 inhibition according to the examples disclosed herein are also plausibly active when used within a degradable nucleic acid construct according to the disclosed embodiments. This is because, without wishing to be bound by a particular theory, that the CFB and C5 antisense strands, after decomposition of the nucleic acid construct, are proceed by the same RISC mechanism as if they would be used in the form of single-stranded mxRNA molecules, which are demonstrated to be highly active in the respective CFB and C5 knock down herein (see examples).

In certain such embodiments, the first nucleic acid portion of (a) may be directly or indirectly linked to the fourth nucleic acid portion of (d) as a primary structure.

In certain embodiments, the first and the fourth nucleic acid portions have the nucleobase sequences of SEQ ID NOs: 13 and 784, 13 and 791, 106 and 784, 106 and 791 and where the sequences of SEQ ID NOs 784 and 791 may be shorter by one, two, three or four nucleobases, where advantageously the 5-terminal nucleobase(s) is/are absent.

In certain embodiments, the second nucleic acid portion of (b) may be directly or indirectly linked to the third nucleic acid portion of (c) as a primary structure.

In certain embodiments, the second and third nucleic acid portions have the nucleobase sequences of SEQ ID NOs: 534 and 265, 534 and 358, 541 and 265, 541 and 358, advantageously 534 and 358 and/or 541 and 265; and where the sequences of SEQ ID NOs: 265 and 358: may be shorter by one, two, three or four nucleobases, where advantageously the 5-terminal nucleobase(s) is/are absent.

In certain embodiments, the construct may further comprise 1 to 8 additional nucleic acid portions that are respectively at least partially complementary to an additional 1 to 8 portions of RNA transcribed from one or more target genes, which target genes may be the same or different to each other, and/or the same or different to the target genes defined in (a) and/or (b), and where each of the 1 to 8 additional nucleic acid portions respectively form additional duplex regions with respective passenger nucleic acid portions that are respectively at least partially complementary therewith. In particular, the second nucleic acid portion of (b), and the 1 to 8 additional nucleic acid portions, may be directly or indirectly linked to selected passenger nucleic acid portions as respective primary structures.

In certain embodiments the direct or indirect linking may represent either (i) an internucleotide bond, (ii) an internucleotide nick, or (iii) a nucleic acid linker portion of 1, 2, 3, 4, 5, 6, 7, 8, 9, or 10 nucleotides, the nucleic acid linker advantageously being single stranded. Advantageously, the linking may be direct, thereby giving rise to (a) contiguous strand(s).

In certain embodiments, there may exist some complementarity between the first nucleic acid portion of (a) and the second nucleic acid portion of (b), or the third nucleic acid portion of (c) and the fourth nucleic acid portion of (d). Advantageously the complementarity
(i) may be 1, 2, 3, 4, 5, 6, 7, 8, 9, or 10, advantageously 2, 3, 4 or 5 base pairs; and/or
(ii) may be between the first nucleic acid portion of (a) and the second nucleic acid portion of (b).

In certain embodiments, the internucleotide bond may involve at least one of the one or more unmodified nucleotides, where advantageously cleavage may occur at the 3′ position of (at least one of) the unmodified nucleotide(s).

In certain embodiments, the first nucleic acid portion of (a), and/or the second nucleic acid portion of (b), and/or the third nucleic acid portion of (c), and/or the fourth nucleic acid portion of (d), may be respectively 7 to 25 nucleotides in length. Optionally, the first nucleic acid portion of (a) and/or the second nucleic acid portion of (b) may have a length of 18 to 21, more advantageously 18 to 20, and yet more advantageously 19 nucleotides. In advantageous embodiments, the first nucleic acid portion of (a) and the second nucleic acid portion of (b) have a length of 19 nucleotides. It may be further advantageous that the third nucleic acid portion of (c), and/or the fourth nucleic acid portion of (d) have a length of 11 to 20, more advantageously 13 to 16, and yet more advantageously 14 or 15, most advantageously 14 nucleotides.

In certain embodiments, the first nucleic portion of (a) and the second nucleic acid portion of (b) may have a length of 19 nucleotides and the third nucleic acid portion of (c) as well as the fourth nucleic acid portion of (b) may have a length of 14 nucleotides.

In certain embodiments, the unmodified nucleotide(s) is/are at any of position 18 to 25, more advantageously at any of positions 18 to 21, and/or the 3′ terminal position of the first nucleic acid portion of (a) and/or of the third nucleic acid portion of (c).

In certain embodiments, where the unmodified nucleotide is at position 19.

In certain embodiments, the first nucleic portion of (a) and the second nucleic acid portion of (b) may have a length of 19 nucleotides and the third nucleic acid portion of (c) as well as the fourth nucleic acid portion of (b) may have a length of 14 nucleotides and the unmodified nucleoside is at position 19 of the first nucleic acid portion of (a) and the second nucleic acid portion of (b).

In certain embodiments, the nucleic acid linker portion may be 1 to 8 nucleotides in length, advantageously 2 to 7 or 3 to 6 nucleotides in length, more advantageously about 4 or 5 and most advantageously 4 nucleotides in length.

In certain embodiments, one, more of all of the duplex regions independently may have a length of 10 to 19, more advantageously 13 to 19, and yet more advantageously 13, 14 or 15 base pairs, most advantageously 14 base pairs, where optionally there is one mismatch within the duplex region.

In certain embodiments, the nucleic acid construct may be blunt ended.

In certain embodiments,
the first nucleic acid portion of (a); and/or
the second nucleic acid portion of (b); and/or
the third nucleic acid portion of (c); and/or
the fourth nucleic acid portion of (d); and/or
to the extent present, the 1 to 8 additional nucleic acid portions as defined previously herein; and/or
to the extent present, the passenger nucleic acid portions as defined previously herein;
may have an overhang.

In certain embodiments, the target RNA may be an mRNA or another RNA molecule.

The construct of any one of the preceding claims, where
(a) the first nucleic acid portion is selected from the first 19 nucleotides of the sequences shown in Table 3a, or in Table 3b (SEQ ID Nos. 1505-1758), or is represented by the nucleic acid sequence: 5′[phos] mU #fU #mG fA mA fU mG fA mA fA mC fG mA fC mU #fU #mC #fU #rC (SEQ ID NO: 2075); or
5′[phos] mU #fU #mG fC mC fA mC fA mG fA mC fU mC fA mG fA #mG #mA #rG (SEQ ID NO: 2076);
(b) the second nucleic acid portion is selected from Table 3c or is represented by the nucleic acid sequence 5′[phos] mG #fA #mU fA mG fU mU fG mU fA mA fA mC fA mG #fU #fU #fC #rC (SEQ ID NO: 2077); or
5′[phos] mU #fU #mA fC mA fA mC fA mG fA mA fU mA fU mG #fG #mU #fA #rU (SEQ ID NO: 2078);
(c) the fourth nucleic acid portion is selected from Table 3d or is represented by the nucleic acid sequence: fC #mU #fG mU fU mU fA mC fA mA fC mU mA #mU #mC #[3XGalNAc](SEQ ID NO: 2079); or
fC #mA #fU mA fU mU fC mU fG mU fU mG mU #mA #mA #[3XGalNAc](SEQ ID NO: 2090 ####); and/or
(d) the third nucleic acid portion is selected from the sequences consisting of the last 15 nucleotides of the sequences shown in Table 3a (SEQ ID Nos. 1505-1756), the last 14 nucleotides of the first entry of Table 3b (SEQ ID No. 1757), or the last 11 nucleotides of the second entry of Table 3b (SEQ ID No. 1758), or is represented by the nucleic acid sequence: fC mU fG mA fG mU fC mU fG mU fG mG mC #mA #mA #[3XGalNAc](SEQ ID NO: 2080); or
fC mU fG mA fG mU fC mU fG mU fG mG mC #mA #mA #(SEQ ID NO: 2081).

In certain embodiment, the construct contain two strands, where the nucleobase sequence of the first strand is shown in Construct ID NO: B106-C5-30, B106-C5-37, B13-C5-30, or B13-C5-37 of Table 4a, advantageously Table 4b, and the nucleobase sequence of the second strand is shown in corresponding Construct ID NO: B106-C5-30, B106-C5-37, B13-C5-30, or B13-C5-37 of Table 4a, advantageously Table 4b. In particular this means that the constructs can comprise or consist of both strands of B106-C5-30, B13-C5-30, B106-C5-37 or B106-C30 shown in Table 4a, advantageously in Table 4b.

Advantageously, the first strand is shown below:

(SEQ ID NO: 2082)
5′[phos]mU# fU# mG fA mA fU mG fA mA fA mC
fG mA fC mU# fU# mC# fU# rC fC# mU# fG mU
fU mU fA mC fA nA fC mU mA# mU# mC#
[3XGalNAC];
(SEQ ID NO: 2083)
5′[phos]mU# fU# mG fA mA fU mG fA mA fA mC
fG mA fC mU# fU# mC# fU# rC fC# mA# fU mA
fU mU fC mU fG mU fU mG mU# mA# mA#
[3XGalNAc];
(SEQ ID NO: 2084)
5′[phos]mU# fU# mG fC mC fA mC fA mG fA mC
fU mC fA mG fA# mG# mA# rG fC# mU# fG mU
fU mU fA mC fA mA fC mU mA# mU# mC#
[3XGalNAc];
or
(SEQ ID NO: 2085)
5′[phos]mU# fU# mG fC mC fA mC fA mG fA mC
fU mC fA mG fA# mG# mA# rG fC# mA# fU mA
fU mU fC mU fG mU fU mG mU# mA# mA#
[3XGalNAc],
and/or
where the second strand is shown below:
(SEQ ID NO: 2086)
5′[phos]mG# fA# mU fA mG fU mU fG mU fA mA
fA mC fA mG# fU# fU# fC# rC fA# mG# fU mC
fG mU fU mU fC mA fU mU mC# mA# mA#
[3XGalNAc];
(SEQ ID NO: 2087)
5′[phos]mU# fU# mA fC mA fA mC fA mG fA mA
fU mA fU mG# fG# mU# fA# rU fA# mG# fU mC
fG mU fU mU fC mA fU mU mC# mA# mA#
[3XGalNAc];
(SEQ ID NO: 2088)
5′[phos]mG# fA# mU fA mG fU mU fG mU fA mA
fA mC fA mG# fU# fU# fC# rC fC mU fG mA
fG mU fC mU fG mU fG mG mC# mA# mA#
[3XGalNAc];
or
(SEQ ID NO: 2089)
5′[phos]mU# fU# mA fC mA fA mC fA mG fA mA
fU mA fU mG# fG# mU# fA# rU fC mU fG mA
fG mU fC mU fG mU fG mG mC# mA# mA#
[3XGalNAc],

where Phos being phosphate; [mN], N being any nucleoside, designates 2′-OMe; [fN], N being any nucleoside, designates: 2′-F; [rA], N being any nucleoside, designates: 2′-OH; [#] designates a phosphorothioate connecting two adjacent nucleosides; and [3XGalNAc] designates the following ligand, where the strand to which the ligand is bound is shown in square brackets:

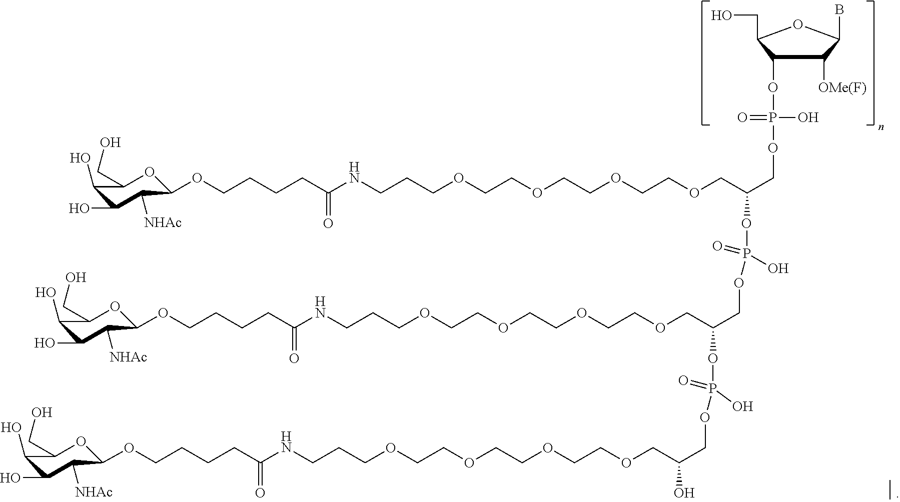

The combination of the first mentioned first strand and the first mentioned second strand is particularly advantageous and also referred to as “STP247G” herein.

In certain embodiments, the construct is selected from Construct ID NO: B106-C5-30, B106-C5-37, B13-C5-30 and B13-C5-37 (SEQ ID Nos. 2067-2074, in the order presented), advantageously, where the construct is Construct ID NO: B106-C5-30, which is STP247G (SEQ ID Nos. 2067-2068).

### Ligands

The nucleic acid construct according to the second aspect and the aforementioned embodiments may further comprise one or more ligands.

In certain embodiments, the first nucleic acid portion of (a), and/or the second nucleic acid portion of (b), and/or the third nucleic acid portion of (c), and/or the fourth nucleic acid portion of (d), and/or, to the extent present, the 1 to 8 additional nucleic acid portions as defined previously herein, and/or the passenger nucleic acid portions as defined previously herein, respectively may have a 5′ to 3′ directionality thereby defining 5′ and 3′ regions thereof.

In certain embodiments, one or more ligands are conjugated at the 3′ region, advantageously the 3′ end, of any of (i) the third nucleic acid portion of (c), and/or (ii) the fourth nucleic acid portion of (d), and/or, to the extent present, the (iii) passenger nucleic acid portions as defined previously herein.

In certain embodiments, one or more ligands may be conjugated at one or more regions intermediate of the 5′ and 3′ regions of any of the nucleic acid portions, advantageously of the third nucleic acid portion of (c), and/or the fourth nucleic acid portion of (d), and/or the passenger nucleic acid portions as defined previously herein.

In certain embodiments, one or more ligands may be conjugated at the 5′ region, advantageously the 5′ end, of any of the nucleic acid portions.

In certain embodiments, the one or more ligands may be any cell directing moiety, such as lipids, carbohydrates, aptamers, vitamins and/or peptides that bind cellular membrane or a specific target on cellular surface. In a advantageous embodiment, the one or more carbohydrates can be a monosaccharide, disaccharide, trisaccharide, tetrasaccharides, oligosaccharide or polysaccharide. In a more advantageous embodiment, the one or more carbohydrates may comprise one or more hexose moieties. Especially, the one or more hexose moieties may be one or more galactose moieties, one or more lactose moieties, one or more N-Acetyl-Galactosamine moieties, and/or one or more mannose moieties. The hexose moiety may be comprise two or three N-Acetyl-Galactosamine moieties. In particular, the hexose moiety may comprise three N-Acetyl-Galactosamine moieties.

In certain embodiments, the one or more ligands may be attached in a linear configuration, or in a branched configuration. Advantageously, where the one or more ligands may be attached as a biantennary or triantennary configuration, or as a configuration based on single ligands at different positions.

Advantageously, the ligand may have the following structure:

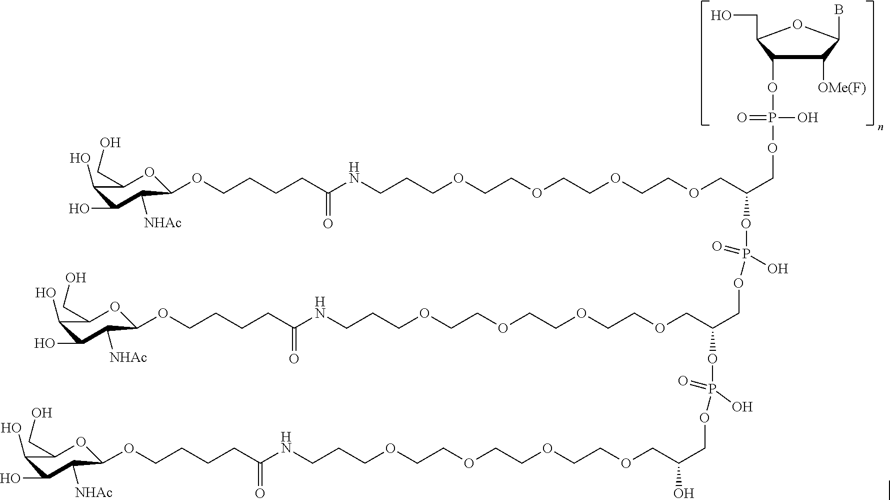

### Internucleoside Linkages

The nucleotide construct according to the second aspect of the disclosed embodiments or its aforementioned embodiments may comprise one or more phosphorothioate or phosphorodithioate internucleotide linkages.

In certain embodiments, the nucleic acid construct may comprise 1 to 15 phosphorothioate or phosphorodithioate internucleotide linkages.

In certain embodiments, the nucleic acid construct may comprise one or more phosphorothioate or phosphorodithioate internucleotide linkages at one or more of the 5′ and/or 3′ regions of the first nucleic acid portion of (a), and/or the second nucleic acid portion of (b), and/or the third nucleic acid portion of (c), and/or the fourth nucleic acid portion of (d), and/or the 1 to 8 additional nucleic acid portions as defined previously herein, and/or the passenger nucleic acid portions as defined in previously herein.

In certain embodiments, the nucleic acid construct may comprise phosphorothioate or phosphorodithioate internucleotide linkages between at least two adjacent nucleotides of the nucleic acid linker portion as defined in previously herein.

In certain embodiments, the nucleic acid construct may comprise a phosphorothioate or phosphorodithioate internucleotide linkage between each adjacent nucleotide that is present in the nucleic acid linker portion.

In certain embodiments, the nucleic acid construct may contain a phosphorothioate or phosphorodithioate internucleotide linkage linking:
the first nucleic acid portion of (a) to the nucleic acid linker portion as defined in previously herein; and/or
the second nucleic acid portion of (b) to the nucleic acid linker portion as defined previously herein; and/or
the third nucleic acid portion of (c) to the nucleic acid linker portion as defined previously herein and/or
the fourth nucleic acid portion of (d) to the nucleic acid linker portion as defined previously herein; and/or
the 1 to 8 additional nucleic acid portions as defined previously herein to the nucleic acid linker portion as further defined previously herein; and/or
the passenger nucleic acid portions as defined previously herein to the nucleic acid linker portion as further defined previously herein.

### Modifications

In the nucleic acid construct according to the second aspect of the disclosed embodiments and its aforementioned embodiments, at least one nucleotide of at least one of the following may be modified:
the first nucleic acid portion of (a); and/or
the second nucleic acid portion of (b); and/or
the third nucleic acid portion of (c); and/or
the fourth nucleic acid portion of (d); and/or
to the extent present, the 1 to 8 additional nucleic acid portions as defined previously herein; and/or
to the extent present, the passenger nucleic acid portions as defined previously herein; and/or
to the extent present, the nucleic acid linker portion as further defined previously herein.

In a advantageous embodiment, one or more of the odd numbered nucleotides starting from the 5′ region of one of the following may be modified, and/or where one or more of the even numbered nucleotides starting from the 5′ region of one of the following are modified, where typically the modification of the even numbered nucleotides is a second modification that is different from the modification of odd numbered nucleotides:
the first nucleic acid portion of (a); and/or
the second nucleic acid portion of (b); and/or
the third nucleic acid portion of (c); and/or
the fourth nucleic acid portion of (d); and/or
to the extent present, the 1 to 8 additional nucleic acid portions as defined previously herein; and/or
to the extent present, the passenger nucleic acid portions as defined previously herein.

In certain embodiments, one or more of the odd numbered nucleotides starting from the 3′ region of the third nucleic acid portion of (c) may be modified by a modification that is different from the modification of odd numbered nucleotides starting from the 5′ region of the first nucleic acid portion of (a); and/or
one or more of the odd numbered nucleotides starting from the 3′ region of the fourth nucleic acid portion of (d) may be modified by a modification that is different from the modification of odd numbered nucleotides starting from the 5′ region of the second nucleic acid portion of (b); and/or
one or more of the odd numbered nucleotides starting from the 3′ region of the passenger nucleic acid portions as defined previously herein, to the extent present, may be modified by a modification that is different from the modification of odd numbered nucleotides starting from the 5′ region of the 1 to 8 additional nucleic acid portions as defined previously herein; and/or
where one or more of the nucleotides of a nucleic acid linker portion as further defined previously herein, to the extent present, may be modified by a modification that (i) is different from the modification of an adjacent nucleotide of the 3′ region of the first nucleic acid portion of (a); and/or (ii) is different from the modification of an adjacent nucleotide of the 3′ region of the second nucleic acid portion of (b); and/or is different from the modification of an adjacent nucleotide of the 3′ region of the 1 to 8 additional nucleic acid portions, to the extent present, as defined previously herein.

In certain embodiments, one or more of the even numbered nucleotides starting from the 3′ region of: (i) the third nucleic acid portion of (c), and/or (ii) the fourth nucleic acid portion of (d), and/or (iii) the passenger nucleic acid portions as defined previously herein, to the extent present, may be modified by a modification that is different from the modification of odd numbered nucleotides starting from the 3′ region of these respective portions.

In certain embodiments, at least one or more of the modified even numbered nucleotides of (i) the first nucleic acid portion of (a), and/or (ii) the second nucleic acid portion of (b), and/or (iii), to the extent present, the 1 to 8 additional nucleic acid portions as defined previously herein, may be adjacent to at least one or more differently modified odd numbered nucleotides of these respective portions.

In certain embodiments, at least one or more of the modified even numbered nucleotides of (i) the third nucleic acid portion of (c), and/or (ii) the fourth nucleic acid portion of (d), and/or (iii), to the extent present, the passenger nucleic acid portions as defined previously herein, may be adjacent to at least one or more differently modified odd numbered nucleotides of these respective portions.

In certain embodiments, a plurality of adjacent nucleotides of (i) the first nucleic acid portion of (a), and/or (ii) the second nucleic acid portion of (b), and/or (iii), to the extent present, the 1 to 8 additional nucleic acid portions as defined previously herein, may be modified by a common modification.

In certain embodiments, a plurality of adjacent nucleotides of (i) the third nucleic acid portion of (c), and/or (ii) the fourth nucleic acid portion of (d), and/or (iii), to the extent present, the passenger nucleic acid portions as defined previously herein, may be modified by a common modification.

In certain embodiments, the plurality of adjacent commonly modified nucleotides may be 2 to 4 adjacent nucleotides, advantageously 3 or 4 adjacent nucleotides.

In certain embodiments, the plurality of adjacent commonly modified nucleotides may be located in the 5′ region of (i) the third nucleic acid portion of (c), and/or (ii) the fourth nucleic acid portion of (d), and/or (iii), to the extent present, the passenger nucleic acid portions previously herein.

In certain embodiments, a plurality of adjacent commonly modified nucleotides may be located in the nucleic acid linker portion as further defined previously herein.

In certain embodiments, the one or more of the modified nucleotides of first nucleic acid portion of (a) may not have a common modification present in the corresponding nucleotide of the third nucleic acid portion of (c) of the first duplex region; and/or one or more of the modified nucleotides of second nucleic acid portion of (b) may not have a common modification present in the corresponding nucleotide of the fourth nucleic acid portion of (d) of the second duplex region; and/or one or more of the modified nucleotides of the 1 to 8 additional nucleic acid portions, to the extent present, as defined previously herein, may not have a common modification present in the corresponding nucleotide of the corresponding passenger nucleic acid portions of the respective duplex regions.

In certain embodiments, the one or more of the modified nucleotides of the first nucleic acid portion of (a) may be shifted by at least one nucleotide relative to a commonly modified nucleotide of the third nucleic acid portion of (c); and/or one or more of the modified nucleotides of the second nucleic acid portion of (b) may be shifted by at least one nucleotide relative to a commonly modified nucleotide of the fourth nucleic acid portion of (d); and/or one or more of the modified nucleotides of the 1 to 8 additional nucleic acid portions, to the extent present, as defined previously herein may be shifted by at least one nucleotide relative to a commonly modified nucleotide of the passenger nucleic acid portions, to the extent present, as defined previously herein.

In certain embodiments, the modification and/or modifications may be each and individually sugar, phosphate, or base modifications.

In certain embodiments, the modification may be selected from nucleotides with 2′ modified sugars; conformationally restricted nucleotides (CRN) sugar such as locked nucleic acid (LNA), (S)-constrained ethyl bicyclic nucleic acid, and constrained ethyl (cEt), tricyclo-DNA; morpholino, unlocked nucleic acid (UNA), glycol nucleic acid (GNA), D-hexitol nucleic acid (HNA), and cyclohexene nucleic acid (CeNA). In advantageous embodiments, where the 2′ modified sugar may be selected from 2′-O-alkyl modified sugar, 2′-O-methyl modified sugar, 2′-O-methoxyethyl modified sugar, 2′-O-allyl modified sugar, 2′-C-allyl modified sugar, 2′-deoxy modified sugar such as 2′-deoxy ribose, 2′-F modified sugar, 2′-arabino-fluoro modified sugar, 2′-O-benzyl modified sugar, 2′-amino modified sugar, and 2′-O-methyl-4-pyridine modified sugar.

In certain embodiments, the base modification may be any one of an abasic nucleotide and a non-natural base containing nucleotide.

In certain embodiments, at least one modification may be a 2′-O-methyl modification in a ribose moiety.

In certain embodiments, at least one modification may be a 2′-F modification in a ribose moiety.

In certain embodiments, the nucleotides at any of positions 2 and 14 downstream from the first nucleotide of the 5′ region of (i) the first nucleic acid portion of (a); and/or (ii) the second nucleic acid portion of (b); and/or (iii), to the extent present, the 1 to 8 additional nucleic acid portions as defined previously herein; may not contain 2′-O-methyl modifications in ribose moieties.

In certain embodiments, one, two or all three nucleotides of (i) the third nucleic acid portion of (c); and/or (ii) the fourth nucleic acid portion of (d); and/or (iii), to the extent present, the passenger nucleic acid portions as defined previously herein; that respectively correspond in position to any of the nucleotides at any of positions 11 to 13 downstream from the first nucleotide of the 5′ region of (i) the first nucleic acid portion of (a); and/or (ii) the second nucleic acid portion of (b); and/or (iii) the 1 to 8 additional nucleic acid portions, to the extent present, as defined previously herein; may not contain 2′-O-methyl modifications in ribose moieties.

In certain embodiments, the nucleotides at any of positions 2 and 14 downstream from the first of (i) the first nucleic acid portion of (a); and/or (ii) the second nucleic acid portion of (b); and/or (iii), to the extent present, the 1 to 8 additional nucleic acid portions as defined previously herein; may contain 2′-F modifications in ribose moieties.

In certain embodiments, one, two or all three nucleotides of (i) the third nucleic acid portion of (c); and or (ii) the fourth nucleic acid portion of (d); and/or (iii), to the extent present, the passenger nucleic acid portions as defined previously herein; that respectively correspond in position to any of the nucleotides at any of positions 11 to 13 downstream from the first nucleotide of the 5′ region of (i) the first nucleic acid portion of (a); and/or (ii) the second nucleic acid portion of (b); and/or (iii), to the extent present, the 1 to 8 additional nucleic acid portions as defined previously herein; may contain 2′-F modifications in ribose moieties.

In certain embodiments, all remaining nucleotides may contain either 2′-O-methyl modifications or 2′-F modifications in ribose moieties, advantageously with the exception of the unmodified nucleotide(s) in accordance with the labile linkage defined herein. Advantageously, the remaining nucleotides may contain 2′-O-methyl modifications in ribose moieties.

In certain embodiments, the one or more, advantageously one, unmodified nucleotide represents any of the nucleotides of the nucleic acid linker portion as further defined previously herein, advantageously the nucleotide of the nucleic acid linker portion as further defined previously herein that is adjacent to (i) the third nucleic acid portion of (c); and or (ii) the fourth nucleic acid portion of (d); and/or (iii), to the extent present, the passenger nucleic acid portions as defined previously herein.

In certain embodiments,
(a) the first nucleic acid portion may be selected from Table 3a;
(b) the second nucleic acid portion may be selected from Table 3a;
(c) the third nucleic acid portion may be selected from Table 3b; and/or
(d) the fourth nucleic acid portion may be selected from Table 3b.

In advantageous embodiments, the first nucleic acid portion and the second nucleic acid portion may be selected from Table 3a, where the first and second nucleic acid portions are different; and the third and fourth nucleic acid portions may be selected from Table 3b.

In certain embodiments, the 3′ terminal positions of the first and the third nucleic acid portions may be replaced with an unmodified nucleotide.

In certain embodiments, the nucleic acid construct may comprise at least one vinylphosphonate modification, such as at least one vinylphosphonate modification in the 5′ region of (i) the first nucleic acid portion of (a); and/or (ii) the second nucleic acid portion of (b); and/or (iii), to the extent present, the 1 to 8 additional nucleic acid portions as defined previously herein.

In certain embodiments, one or more nucleotides of
the first nucleic acid portion of (a); and/or
the second nucleic acid portion of (b); and/or
the third nucleic acid portion of (c); and/or
the fourth nucleic acid portion of (d); and/or
to the extent present, the 1 to 8 additional nucleic acid portions as defined previously herein; and/or
to the extent present, the passenger nucleic acid portions as defined previously herein;
may be an inverted an inverted nucleotide and may be attached to the adjacent nucleotide via the 3′ carbon of the nucleotide and the 3′ carbon of the adjacent nucleotide, and/or may be an inverted nucleotide and may be attached to the adjacent nucleotide via the 5′ carbon of the nucleotide and the 5′ carbon of the adjacent nucleotide.

In certain embodiments, the inverted nucleotide may be attached to the adjacent nucleotide via a phosphate group by way of a phosphodiester linkage; or may be attached to the adjacent nucleotide via a phosphorothioate group; or may be attached to the adjacent nucleotide via a phosphorodithioate group.

In certain embodiments, the modifications among strands within the constructs include alternating modification pattern, advantageously with odd-numbered nucleotides being fluoro-substituted and even-numbered nucleotides being —OME substituted.

Compositions and Pharmaceutical Compositions Including muRNA Oligomeric Constructs

According to a second aspect, the disclosed embodiments is directed to a composition containing a nucleic acid construct according to the first aspect, and a physiologically acceptable excipient.

According to a third aspect, the disclosed embodiments is directed to a pharmaceutical composition containing a nucleic acid construct according to the first aspect.

The pharmaceutical composition may further comprise a pharmaceutically acceptable excipient, diluent, antioxidant, and/or preservative.

The oligomeric compound according to the first aspect and/or the construct according to the second aspect may be the only pharmaceutically active agent(s).

Alternatively, the pharmaceutical composition furthermore contain one or more further pharmaceutically active agents. The further pharmaceutically active agent(s) is/are (an) agent(s), which modulate(s) the innate and/or the adaptive immune system, for example a further oligomeric compound, which is directed to an immune system target, and/or compounds targeting other components of the immune system, such as components of the proximal complement pathway, in particular Lectin pathway: MASP-2 targeting compounds and/or C3-targeting compounds and/or compounds selected from the group consisting of Sutimlimab, Narsoplimab, Pegcetacoplan AMY-102, IONIS-FB-LRx-LPNO23, Lapalizumab, Mini-FH/AMY-201 MicroCept, and GLG561 or combinations thereof.

Diseases to be Treated by the muRNA Oligomeric Compounds and Further Uses

According to a fourth aspect, the disclosed embodiments are directed to the nucleic acid construct according to first aspect of the disclosed embodiments, for use in human or veterinary medicine or therapy.

According to a fifth aspect, the disclosed embodiments are directed to the nucleic acid construct according to the first aspect of the disclosed embodiments, for use in a method of treating, ameliorating and/or preventing a disease or disorder.

The disease or disorder is a disease or disorder associated with CFB and/or C5 or a disease or disorder requiring reduction of CFB and/or C5 expression.

In particular, the disease or disorder is selected from the group consisting of hematological diseases, such as, Paroxysmal Nocturnal Hemoglobinuria (PNH); nephrological diseases, such as Atypical Hemoytic-Uremic Syndrome (aHUS), C3 glomerulonephritis, dense deposit dissease, Immune Complex Membranoproliferative Glomerulonephritis, IgA nephropathy; neurological diseases, such as Generalized Myasthenia Gravis (GMG), Relapsing Neuromyelitisi Optica (NMO), Amyotrophic Lateral Sclerosis (ALS), ophthalmological diseases, such as Age-related Macular Degeneration, Geographic Atrophy; oncological diseases; rheumatological diseases; and transplant-related diseases.

The disease or disorder is advantageously PNH. Due to the inventors' finding that the nucleic acid constructs disclosed herein targeting CFB and C5 and producing their knock down, these nucleic acid constructs have the potential of being effective in PNH therapy. This is because it is assumed, without being bound by any theory, that the C5-targeting part of the nucleic acid constructs prevent intravascular hemolysis and the CFB-targeting part of the nucleic acid constructs prevent extravascular hemolysis.

According to a sixth aspect, the disclosed embodiments are directed to a method of treating a disease or disorder containing administration the nucleic acid construct according to the first aspect of the disclosed embodiments, to an individual in need of treatment.

The nucleic acid construct may be administered subcutaneously or intravenously to the individual.

According to a seventh aspect, the disclosed embodiments are directed to a use of a nucleic acid construct according to the first aspect, for use in research as a gene function analysis tool.

According to an eighth aspect, the disclosed embodiments are directed to a use of the nucleic acid construct according to the first aspect in the manufacture of a medicament for a treatment of a disease or disorder.

### Constructs and Sequences of the Novel Oligomeric Compounds

The following Tables show nucleobase sequences of antisense and sense strands of oligomeric compounds of the disclosed embodiments as well as of nucleobase sequences of single-stranded oligomeric compounds of the disclosed embodiments, and definitions of modified oligomeric compounds of the disclosed embodiments (the notation including nucleobase sequence, sugar modifications, and, where applicable, modified phosphates).

The notation used is common in the art and as the following meaning:
A represents adenine;
U represents uracil;
C represents cytosine;
G represents guanine.
5Phos represents a 5′ terminal phosphate group, which is advantageous but not indispensable;
m represents a methyl modification at the 2′ position of the sugar of the underlying nucleoside;
f represents a fluoro modification at the 2′ position of the sugar of the underlying nucleoside;
r indicates an unmodified (2′-OH) ribonucleotide;
[Ps] or #represents a phosphorothioate inter-nucleoside linkage;
i represents an inverted inter-nucleoside linkage, which can be either 3′-3′, or 5′-5′;
3× GalNAc represents a trivalent GalNAc.

Tables 1a and 1 b below show nucleobase sequences of antisense and sense strands of 252 oligomeric compounds in accordance with the examples.

TABLE 1a
Nucleobase sequences of the CFB antisense strands
of 252 constructs

| SEQ | Experimental | |
|---|---|---|
| ID NO. | Label | 19 mer Antisense |
| 1 | CFB01 | UAGAAAACCCAAAUCCUCA |
| 2 | CFB02 | GCUGUCUGAUCCAUCUAGC |
| 3 | CFB03 | AACCAUGCCACAGAGACUC |
| 4 | CFB04 | GAUCCAUCUAGCACCAGGU |
| 5 | CFB05 | AAAACCCAAAUCCUCAUCU |
| 6 | CFB06 | UCCAUCUAGCACCAGGUAG |
| 7 | CFB07 | GAAAACCCAAAUCCUCAUC |
| 8 | CFB08 | UGUCUGAUCCAUCUAGCAC |
| 9 | CFB09 | AACCCAAAUCCUCAUCUUG |
| 10 | CFB10 | AUCCAUCUAGCACCAGGUA |
| 11 | CFB11 | AAACCCAAAUCCUCAUCUU |
| 12 | CFB12 | CCAUGCCACAGAGACUCAG |
| 13 | CFB13 | AUGCCACAGAGACUCAGAG |
| 14 | CFB14 | GAUGAUGACAUGGCGGGUG |
| 15 | CFB15 | UUCCAUAUCCUUGACUUUG |
| 16 | CFB16 | UACACCAACUUGAAUGAAA |
| 17 | CFB17 | UGCCACAGAGACUCAGAGA |
| 18 | CFB18 | ACCAUGCCACAGAGACUCA |
| 19 | CFB19 | CCAUCUAGCACCAGGUAGA |
| 20 | CFB20 | UUUCCAUAUCCUUGACUUU |
| 21 | CFB21 | UGAUCCAUCUAGCACCAGG |
| 22 | CFB22 | GCCACAGAGACUCAGAGAC |
| 23 | CFB23 | CUGAUCCAUCUAGCACCAG |
| 24 | CFB24 | GACCUCCUUCCGAGUCAGC |
| 25 | CFB25 | GUCUGAUCCAUCUAGCACC |
| 26 | CFB26 | GUCUUGGCAGGAAGGCUCC |
| 27 | CFB27 | AUCUAGCACCAGGUAGAUG |
| 28 | CFB28 | AAAGUACUCAGACACCACA |
| 29 | CFB29 | CUGUCUGAUCCAUCUAGCA |
| 30 | CFB30 | CAUCUAGCACCAGGUAGAU |
| 31 | CFB31 | CCAAAUCCUCAUCUUGGAG |
| 32 | CFB32 | CAUAGUCAUAAAAUUCAGG |
| 33 | CFB33 | GAGGAUGAUGACAUGGCGG |
| 34 | CFB34 | ACAAUCUGUGUUCUGGCAC |
| 35 | CFB35 | UUGAGCUUGAUCAGGGCAA |
| 36 | CFB36 | CAGUGGAAAGAGAUCUCAU |
| 37 | CFB37 | UAGAUGUUCAUGGAGCCUG |
| 38 | CFB38 | GGCAAGUGGUAGUUGGAGG |
| 39 | CFB39 | UCACACCAUAACUUGCCAC |
| 40 | CFB40 | GAAAGAGAUCUCAUCACUC |
| 41 | CFB41 | UUCAACUUGUGGUCUUCAU |
| 42 | CFB42 | GAAACGACUUCUCUUGUGA |
| 43 | CFB43 | GGUAUGUGGCAUAUGUCAC |
| 44 | CFB44 | UGUCUUUCUUGGAAGCCAA |
| 45 | CFB45 | ACAUCCAGAUAAUCCUCCC |
| 46 | CFB46 | CUUGACUUUGUCAUAGCCU |
| 47 | CFB47 | GAAACUCCAGACCUAGACC |
| 48 | CFB48 | CAUAACUUGCCACCUUCUC |
| 49 | CFB49 | UCAUAGUCAUAAAAUUCAG |
| 50 | CFB50 | UUGGCUCCUGUGAAGUUGC |
| 51 | CFB51 | CAAAGUACUCAGACACCAC |
| 52 | CFB52 | UGCUCAUUGUCUUUCUUGG |
| 53 | CFB53 | AUAAAAUUCAGGAAUUCCU |
| 54 | CFB54 | UGAGAUCUUGGCCUGCCAU |
| 55 | CFB55 | UGAGCAUCUCUCUCACAGC |
| 56 | CFB56 | UAACCGUCAUAGCAGUGGA |
| 57 | CFB57 | CAGAGCUUUGAUAUCCUGU |
| 58 | CFB58 | AACAAUGUGCUGCUGUCAG |
| 59 | CFB59 | GGGUACGGGUAGAAGCCAG |
| 60 | CFB60 | CCAGACCUAGACCUGGUCA |
| 61 | CFB61 | CUUCUCUUGUGAACUAUCA |
| 62 | CFB62 | AUUCAGGAAUUCCUGCUUC |
| 63 | CFB63 | UCCAGGUUUUCCAUAUCCU |
| 64 | CFB64 | CCAACUUGAAUGAAACGAC |
| 65 | CFB65 | UGUGCUGCUGUCAGCACAA |
| 66 | CFB66 | AGACCUCCUUCCGAGUCAG |
| 67 | CFB67 | UCAAUUAAGUUGACUAGAC |
| 68 | CFB68 | CUGACACGUUCGCCGCUGG |
| 69 | CFB69 | UCAUUGUCUUUCUUGGAAG |
| 70 | CFB70 | ACCAACUUGAAUGAAACGA |
| 71 | CFB71 | GCACAAAGUACUCAGACAC |
| 72 | CFB72 | CUGCAGUGGUAGGUGACGC |
| 73 | CFB73 | GUCAUGAGGAUGAUGACAU |
| 74 | CFB74 | CUUCAACUUGUGGUCUUCA |
| 75 | CFB75 | GGUAGUUGGAGGAAGCCUC |
| 76 | CFB76 | UCAUGCUGUACACUGCCUG |
| 77 | CFB77 | UUGUCUUUCUUGGAAGCCA |
| 78 | CFB78 | UCGACUCCUUCUAUGGUCU |
| 79 | CFB79 | AGACAUCCAGAUAAUCCUC |
| 80 | CFB80 | CUGAGAUCUUGGCCUGCCA |
| 81 | CFB81 | GACGCUGUCUUCAAGGCGG |
| 82 | CFB82 | AAGACAGGAAAGCUUCGGC |
| 83 | CFB83 | UUUGAACACAUGUUGCUCA |
| 84 | CFB84 | CAUAAAAUUCAGGAAUUCC |
| 85 | CFB85 | ACCCAAAUCCUCAUCUUGG |
| 86 | CFB86 | AAAGAGAUCUCAUCACUCA |
| 87 | CFB87 | CAAAGCAUUGAUGUUCACU |
| 88 | CFB88 | CAGGAAUUCCUGCUUCUUU |
| 89 | CFB89 | CAUGAAGGAGUCUUGGCAG |
| 90 | CFB90 | AAAGCUUCGGCCACCUCUU |
| 91 | CFB91 | UUGACUUUGUCAUAGCCUG |
| 92 | CFB92 | GUCCAAGCUGAAACUCCAG |
| 93 | CFB93 | CCCAAAUCCUCAUCUUGGA |
| 94 | CFB94 | GCAGCUGUUUUAAUUCAAU |
| 95 | CFB95 | AAACGACUUCUCUUGUGAA |
| 96 | CFB96 | CCCGGAACAUCCAAGCGGG |
| 97 | CFB97 | CCAAACACAUAGACAUCCA |
| 98 | CFB98 | CUUCACACCAUAACUUGCC |
| 99 | CFB99 | UUCUCUUGUGAACUAUCAA |
| 100 | CFB100 | AAUUCAGGAAUUCCUGCUU |
| 101 | CFB101 | AGACACUUUGACCCAAAUU |
| 102 | CFB102 | ACACAAACAGAGCUUUGAU |
| 103 | CFB103 | ACAAACAGAGCUUUGAUAU |
| 104 | CFB104 | CUUGGAGUUUCUCCUUCAG |
| 105 | CFB105 | UUCACACCAUAACUUGCCA |
| 106 | CFB106 | UUGAAUGAAACGACUUCUC |
| 107 | CFB107 | CCAGGUUUUCCAUAUCCUU |
| 108 | CFB108 | AGCAUCUCUCUCACAGCUG |
| 109 | CFB109 | GACAUCCAGAUAAUCCUCC |
| 110 | CFB110 | UCGAGUUGUUCCCUCGGUG |
| 111 | CFB111 | AACACAUGUUGCUCAUUGU |
| 112 | CFB112 | UUCUCAAUUAAGUUGACUA |
| 113 | CFB113 | GGAAGCCAAAGCAUUGAUG |
| 114 | CFB114 | AGCAGUGGAAAGAGAUCUC |
| 115 | CFB115 | UACACUGCCUGGAGGGCCU |
| 116 | CFB116 | CUAGACCUGGUCACAUUCC |
| 117 | CFB117 | UCAGACACAAACAGAGCUU |
| 118 | CFB118 | GGAGUUUCUCCUUCAGCCA |
| 119 | CFB119 | AAUGUGCUGCUGUCAGCAC |
| 120 | CFB120 | ACAGAGCUUUGAUAUCCUG |
| 121 | CFB121 | UGAUAUCCUGUGCAGGGAG |
| 122 | CFB122 | CAGGGCAACGUCAUAGUCA |
| 123 | CFB123 | CAGACCUAGACCUGGUCAC |
| 124 | CFB124 | AAGUACUCAGACACCACAG |
| 125 | CFB125 | GAAGGCUCCGUCCCGCUCC |
| 126 | CFB126 | AGGGCAACGUCAUAGUCAU |
| 127 | CFB127 | GCUGUUUUAAUUCAAUCCC |
| 128 | CFB128 | AAGAGAUCUCAUCACUCAC |
| 129 | CFB129 | UGGUCUUCAUAAUUGAUUU |
| 130 | CFB130 | CCAUAUCUUGGCUUCACAC |
| 131 | CFB131 | ACACCAACUUGAAUGAAAC |
| 132 | CFB132 | CAGCUGUUUUAAUUCAAUC |
| 133 | CFB133 | AUAACUUGCCACCUUCUCA |
| 134 | CFB134 | GUGAGCAGGUACCUGCUUU |
| 135 | CFB135 | CUUGAUGUAGACCUCCUUC |
| 136 | CFB136 | UGGCAAGUGGUAGUUGGAG |
| 137 | CFB137 | AGGAAGCCUCAAAGCUCGA |
| 138 | CFB138 | CAAUGACAGUAAUUGGGUC |
| 139 | CFB139 | CUUUGAACACAUGUUGCUC |
| 140 | CFB140 | CAAAUCCUCAUCUUGGAGU |
| 141 | CFB141 | UGGAGUUUCUCCUUCAGCC |
| 142 | CFB142 | CCAUAACUUGCCACCUUCU |
| 143 | CFB143 | AGCUGUUUUAAUUCAAUCC |
| 144 | CFB144 | AAAGCUCGAGUUGUUCCCU |
| 145 | CFB145 | GGGCAACGUCAUAGUCAUA |
| 146 | CFB146 | CUUCCAGGUUUUCCAUAUC |
| 147 | CFB147 | UUCCAGGUUUUCCAUAUCC |
| 148 | CFB148 | CCCAUGUUGUGCAAUCCAU |
| 149 | CFB149 | CCAUAUCCUUGACUUUGAA |
| 150 | CFB150 | UUGACUUUGAACACAUGUU |
| 151 | CFB151 | CCUCAUCUUGGAGUUUCUC |
| 152 | CFB152 | ACAUGUUGCUCAUUGUCUU |
| 153 | CFB153 | CACCAACUUGAAUGAAACG |
| 154 | CFB154 | CACAGAUCGCUGUCUGCCC |
| 155 | CFB155 | CUCACAGCUGCCUUUCUUA |
| 156 | CFB156 | GGGCCGCCAGAAUCACCUC |
| 157 | CFB157 | UCCAAGCUGAAACUCCAGA |
| 158 | CFB158 | CUUGAUCAGGGCAACGUCA |
| 159 | CFB159 | UGUUCCCAAACCAUGCCAC |
| 160 | CFB160 | UACCUGCUUUUGCCGCUUC |
| 161 | CFB161 | GUUGCUCAUUGUCUUUCUU |
| 162 | CFB162 | ACACGUUCGCCGCUGGGAG |
| 163 | CFB163 | CCAUUCUUGAUGUAGACCU |
| 164 | CFB164 | CUUGAGCUUGAUCAGGGCA |
| 165 | CFB165 | CAUUCUUGAUGUAGACCUC |
| 166 | CFB166 | AUGAAGGAGUCUUGGCAGG |
| 167 | CFB167 | CUUGGCUUCACACCAUAAC |
| 168 | CFB168 | UAUCUUGGCUUCACACCAU |
| 169 | CFB169 | UCUCACAGCUGCCUUUCUU |
| 170 | CFB170 | CCCAAUGCUGUCUGAUCCA |
| 171 | CFB171 | GGAGUGGUGGUCACACCUC |
| 172 | CFB172 | CAUAGGGACUCACUCCUCC |
| 173 | CFB173 | CCUGACUUCAACUUGUGGU |
| 174 | CFB174 | CUUCUCAAUUAAGUUGACU |
| 175 | CFB175 | GAGUUUCUCCUUCAGCCAG |
| 176 | CFB176 | GAGCUUUGAUAUCCUGUGC |
| 177 | CFB177 | AUGUCCUUGACUUUGUCAU |
| 178 | CFB178 | GCAGGUACGUGUCUGCACA |
| 179 | CFB179 | GAAACAAUGUGCUGCUGUC |
| 180 | CFB180 | GAUAUCCUGUGCAGGGAGC |
| 181 | CFB181 | AGACUCAGAGACUGGCUUU |
| 182 | CFB182 | UCAAUGACAGUAAUUGGGU |
| 183 | CFB183 | AGAGCCACCUUCCUGACAC |
| 184 | CFB184 | CCUUGACUUUGAACACAUG |
| 185 | CFB185 | AAUGAAACGACUUCUCUUG |
| 186 | CFB186 | GGAAGACAGGAAAGCUUCG |
| 187 | CFB187 | AGCUUUGAUAUCCUGUGCA |
| 188 | CFB188 | UUCUUGAGCUUGAUCAGGG |
| 189 | CFB189 | UGGAUUGCUCUGCACUCUG |
| 190 | CFB190 | GCAUAUUGAGCAUCUCUCU |
| 191 | CFB191 | UAUCCUUGACUUUGAACAC |
| 192 | CFB192 | UGCAGACAUCCACUACUCC |
| 193 | CFB193 | UAGACCUCCUUCCGAGUCA |
| 194 | CFB194 | CACCUUCUCAAUUAAGUUG |
| 195 | CFB195 | GAGAAGUCGGAAGGAGCCG |
| 196 | CFB196 | CUGCACAGGGUACGGGUAG |
| 197 | CFB197 | AAUGACAGUAAUUGGGUCC |
| 198 | CFB198 | UGUUAGUCCCUGACUUCAA |
| 199 | CFB199 | CAUAUCCUUGACUUUGAAC |
| 200 | CFB200 | GGUACGUGUCUGCACAGGG |
| 201 | CFB201 | UGUCAGCACAAAGUACUCA |
| 202 | CFB202 | GUGGUCUUCAUAAUUGAUU |
| 203 | CFB203 | ACAGAGACUCAGAGACUGG |
| 204 | CFB204 | AUAGACAUCCAGAUAAUCC |
| 205 | CFB205 | CCUCCUUCCGAGUCAGCUU |
| 206 | CFB206 | UCAUGGAGCCUGAAGGGUC |
| 207 | CFB207 | GUGGCAUAUGUCACUAGAC |
| 208 | CFB208 | AAAGCAUUGAUGUUCACUU |
| 209 | CFB209 | ACUCACUCCUCCAGUACAA |
| 210 | CFB210 | AGAUGUCCUUGACUUUGUC |
| 211 | CFB211 | CUGUUUUAAUUCAAUCCCA |
| 212 | CFB212 | GUAGAUGUUCAUGGAGCCU |
| 213 | CFB213 | CAUAGCAGUGGAAAGAGAU |
| 214 | CFB214 | CCAUUCACUUGGCAGGUGC |
| 215 | CFB215 | CAUUGAUGUUCACUUGGUU |
| 216 | CFB216 | UCAGCCAGGGCAGCACUUG |
| 217 | CFB217 | GCUCAGUGUCCAAGCUGAA |
| 218 | CFB218 | UCAGAGACUGGCUUUCAUC |
| 219 | CFB219 | CAUCCAGAUAAUCCUCCCU |
| 220 | CFB220 | CCUUCUCAAUUAAGUUGAC |
| 221 | CFB221 | UCCAGACCUAGACCUGGUC |
| 222 | CFB222 | GCAACGUCAUAGUCAUAAA |
| 223 | CFB223 | CUUGAAUGAAACGACUUCU |
| 224 | CFB224 | UCCUCCUCAGACACAAACA |
| 225 | CFB225 | GAAGCCAAAGCAUUGAUGU |
| 226 | CFB226 | AUGAAACGACUUCUCUUGU |
| 227 | CFB227 | CAACUUGUGGUCUUCAUAA |
| 228 | CFB228 | ACCAGGUAGAUGUUCAUGG |
| 229 | CFB229 | UCUGUGUUCUGGCACCUGC |
| 230 | CFB230 | UAACUUGCCACCUUCUCAA |
| 231 | CFB231 | UGCCAUGGUUGCUUGUGGU |
| 232 | CFB232 | AGACAAAUGGGCCUGAUAG |
| 233 | CFB233 | UAAGUUGACUAGACACUUU |
| 234 | CFB234 | ACAUGGCGGGUGCGGUUCC |
| 235 | CFB235 | CCCGGAUCUCAUCAAUGAC |
| 236 | CFB236 | GGAGGAAGCCUCAAAGCUC |
| 237 | CFB237 | GACUUUGAACACAUGUUGC |
| 238 | CFB238 | UCUCCUCCUCAGACACAAA |
| 239 | CFB239 | CAGGAAGGCUCCGUCCCGC |
| 240 | CFB240 | CCGCCAGAAUCACCUCUGC |
| 241 | CFB241 | UGGAAAGAGAUCUCAUCAC |
| 242 | CFB242 | CAAGUCCCGGAUCUCAUCA |
| 243 | CFB243 | UGAGCUUGAUCAGGGCAAC |
| 244 | CFB244 | UGUUGCUCAUUGUCUUUCU |
| 245 | CFB245 | UUGCUUGUGGUAAUCGGUA |
| 246 | CFB246 | CUCAUCACUCACAUUGUAG |
| 247 | CFB247 | UCAGUGUCCAAGCUGAAAC |
| 248 | CFB248 | CCCAUUCUUGAUGUAGACC |
| 249 | CFB249 | AAACAAUGUGCUGCUGUCA |
| 250 | CFB250 | UGACUUCAACUUGUGGUCU |
| 251 | 13(5) as | UUGCCACAGAGACUCAGAG |
| 252 | 106-13(4) as | UUGAAUGAAACGACUUCUC |

The first nucleobase on the terminal 5′ position (the sequences in the table are presented from a 5′(Ieft) to a 3′(right direction) can be freely selected from U, A, G and C instead of the nucleobase disclosed in the table.

TABLE 1b
Nucleobase sequences of the CFB sense strands of
252 constructs

| SEQ | Experimental | 15 mer, 14 mer or |
|---|---|---|
| ID NO. | Label | 11 mer Sense |
| 253 | CFB01 | GAUUUGGGUUUUCUA |
| 254 | CFB02 | GAUGGAUCAGACAGC |
| 255 | CFB03 | CUCUGUGGCAUGGUU |
| 256 | CFB04 | GGUGCUAGAUGGAUC |
| 257 | CFB05 | GAGGAUUUGGGUUUU |
| 258 | CFB06 | CUGGUGCUAGAUGGA |
| 259 | CFB07 | AGGAUUUGGGUUUUC |
| 260 | CFB08 | UAGAUGGAUCAGACA |
| 261 | CFB09 | AUGAGGAUUUGGGUU |
| 262 | CFB10 | UGGUGCUAGAUGGAU |
| 263 | CFB11 | UGAGGAUUUGGGUUU |
| 264 | CFB12 | GUCUCUGUGGCAUGG |
| 265 | CFB13 | GAGUCUCUGUGGCAU |
| 266 | CFB14 | CGCCAUGUCAUCAUC |
| 267 | CFB15 | GUCAAGGAUAUGGAA |
| 268 | CFB16 | AUUCAAGUUGGUGUA |
| 269 | CFB17 | UGAGUCUCUGUGGCA |
| 270 | CFB18 | UCUCUGUGGCAUGGU |
| 271 | CFB19 | CCUGGUGCUAGAUGG |
| 272 | CFB20 | UCAAGGAUAUGGAAA |
| 273 | CFB21 | GUGCUAGAUGGAUCA |
| 274 | CFB22 | CUGAGUCUCUGUGGC |
| 275 | CFB23 | UGCUAGAUGGAUCAG |
| 276 | CFB24 | ACUCGGAAGGAGGUC |
| 277 | CFB25 | CUAGAUGGAUCAGAC |
| 278 | CFB26 | CCUUCCUGCCAAGAC |
| 279 | CFB27 | UACCUGGUGCUAGAU |
| 280 | CFB28 | GUGUCUGAGUACUUU |
| 281 | CFB29 | AGAUGGAUCAGACAG |
| 282 | CFB30 | ACCUGGUGCUAGAUG |
| 283 | CFB31 | AAGAUGAGGAUUUGG |
| 284 | CFB32 | AAUUUUAUGACUAUG |
| 285 | CFB33 | CAUGUCAUCAUCCUC |
| 286 | CFB34 | CAGAACACAGAUUGU |
| 287 | CFB35 | CCUGAUCAAGCUCAA |
| 288 | CFB36 | GAUCUCUUUCCACUG |
| 289 | CFB37 | CUCCAUGAACAUCUA |
| 290 | CFB38 | CAACUACCACUUGCC |
| 291 | CFB39 | CAAGUUAUGGUGUGA |
| 292 | CFB40 | GAUGAGAUCUCUUUC |
| 293 | CFB41 | AGACCACAAGUUGAA |
| 294 | CFB42 | AAGAGAAGUCGUUUC |
| 295 | CFB43 | CAUAUGCCACAUACC |
| 296 | CFB44 | CUUCCAAGAAAGACA |
| 297 | CFB45 | GGAUUAUCUGGAUGU |
| 298 | CFB46 | UAUGACAAAGUCAAG |
| 299 | CFB47 | UAGGUCUGGAGUUUC |
| 300 | CFB48 | AGGUGGCAAGUUAUG |
| 301 | CFB49 | AUUUUAUGACUAUGA |
| 302 | CFB50 | CUUCACAGGAGCCAA |
| 303 | CFB51 | UGUCUGAGUACUUUG |
| 304 | CFB52 | GAAAGACAAUGAGCA |
| 305 | CFB53 | AUUCCUGAAUUUUAU |
| 306 | CFB54 | CAGGCCAAGAUCUCA |
| 307 | CFB55 | UGAGAGAGAUGCUCA |
| 308 | CFB56 | CUGCUAUGACGGUUA |
| 309 | CFB57 | GAUAUCAAAGCUCUG |
| 310 | CFB58 | CAGCAGCACAUUGUU |
| 311 | CFB59 | CUUCUACCCGUACCC |
| 312 | CFB60 | CAGGUCUAGGUCUGG |
| 313 | CFB61 | AGUUCACAAGAGAAG |
| 314 | CFB62 | CAGGAAUUCCUGAAU |
| 315 | CFB63 | UAUGGAAAACCUGGA |
| 316 | CFB64 | UUUCAUUCAAGUUGG |
| 317 | CFB65 | GCUGACAGCAGCACA |
| 318 | CFB66 | CUCGGAAGGAGGUCU |
| 319 | CFB67 | AGUCAACUUAAUUGA |
| 320 | CFB68 | CGGCGAACGUGUCAG |
| 321 | CFB69 | CAAGAAAGACAAUGA |
| 322 | CFB70 | UUCAUUCAAGUUGGU |
| 323 | CFB71 | CUGAGUACUUUGUGC |
| 324 | CFB72 | CACCUACCACUGCAG |
| 325 | CFB73 | CAUCAUCCUCAUGAC |
| 326 | CFB74 | GACCACAAGUUGAAG |
| 327 | CFB75 | CUUCCUCCAACUACC |
| 328 | CFB76 | CAGUGUACAGCAUGA |
| 329 | CFB77 | UUCCAAGAAAGACAA |
| 330 | CFB78 | CAUAGAAGGAGUCGA |
| 331 | CFB79 | AUUAUCUGGAUGUCU |
| 332 | CFB80 | AGGCCAAGAUCUCAG |
| 333 | CFB81 | CUUGAAGACAGCGUC |
| 334 | CFB82 | AAGCUUUCCUGUCUU |
| 335 | CFB83 | CAACAUGUGUUCAAA |
| 336 | CFB84 | UUCCUGAAUUUUAUG |
| 337 | CFB85 | GAUGAGGAUUUGGGU |
| 338 | CFB86 | UGAUGAGAUCUCUUU |
| 339 | CFB87 | AACAUCAAUGCUUUG |
| 340 | CFB88 | AAGCAGGAAUUCCUG |
| 341 | CFB89 | CAAGACUCCUUCAUG |
| 342 | CFB90 | GGUGGCCGAAGCUUU |
| 343 | CFB91 | CUAUGACAAAGUCAA |
| 344 | CFB92 | AGUUUCAGCUUGGAC |
| 345 | CFB93 | AGAUGAGGAUUUGGG |
| 346 | CFB94 | AAUUAAAACAGCUGC |
| 347 | CFB95 | CAAGAGAAGUCGUUU |
| 348 | CFB96 | CUUGGAUGUUCCGGG |
| 349 | CFB97 | UGUCUAUGUGUUUGG |
| 350 | CFB98 | AGUUAUGGUGUGAAG |
| 351 | CFB99 | UAGUUCACAAGAGAA |
| 352 | CFB100 | AGGAAUUCCUGAAUU |
| 353 | CFB101 | UGGGUCAAAGUGUCU |
| 354 | CFB102 | AAGCUCUGUUUGUGU |
| 355 | CFB103 | CAAAGCUCUGUUUGU |
| 356 | CFB104 | AGGAGAAACUCCAAG |
| 357 | CFB105 | AAGUUAUGGUGUGAA |
| 358 | CFB106 | AGUCGUUUCAUUCAA |
| 359 | CFB107 | AUAUGGAAAACCUGG |
| 360 | CFB108 | UGUGAGAGAGAUGCU |
| 361 | CFB109 | GAUUAUCUGGAUGUC |
| 362 | CFB110 | GAGGGAACAACUCGA |
| 363 | CFB111 | UGAGCAACAUGUGUU |
| 364 | CFB112 | CAACUUAAUUGAGAA |
| 365 | CFB113 | AAUGCUUUGGCUUCC |
| 366 | CFB114 | UCUCUUUCCACUGCU |
| 367 | CFB115 | CCUCCAGGCAGUGUA |
| 368 | CFB116 | UGUGACCAGGUCUAG |
| 369 | CFB117 | UCUGUUUGUGUCUGA |
| 370 | CFB118 | UGAAGGAGAAACUCC |
| 371 | CFB119 | UGACAGCAGCACAUU |
| 372 | CFB120 | AUAUCAAAGCUCUGU |
| 373 | CFB121 | CUGCACAGGAUAUCA |
| 374 | CFB122 | UAUGACGUUGCCCUG |
| 375 | CFB123 | CCAGGUCUAGGUCUG |
| 376 | CFB124 | GGUGUCUGAGUACUU |
| 377 | CFB125 | CGGGACGGAGCCUUC |
| 378 | CFB126 | CUAUGACGUUGCCCU |
| 379 | CFB127 | UUGAAUUAAAACAGC |
| 380 | CFB128 | GUGAUGAGAUCUCUU |
| 381 | CFB129 | CAAUUAUGAAGACCA |
| 382 | CFB130 | GAAGCCAAGAUAUGG |
| 383 | CFB131 | CAUUCAAGUUGGUGU |
| 384 | CFB132 | GAAUUAAAACAGCUG |
| 385 | CFB133 | AAGGUGGCAAGUUAU |
| 386 | CFB134 | CAGGUACCUGCUCAC |
| 387 | CFB135 | GAGGUCUACAUCAAG |
| 388 | CFB136 | AACUACCACUUGCCA |
| 389 | CFB137 | GCUUUGAGGCUUCCU |
| 390 | CFB138 | CAAUUACUGUCAUUG |
| 391 | CFB139 | AACAUGUGUUCAAAG |
| 392 | CFB140 | CAAGAUGAGGAUUUG |
| 393 | CFB141 | GAAGGAGAAACUCCA |
| 394 | CFB142 | GGUGGCAAGUUAUGG |
| 395 | CFB143 | UGAAUUAAAACAGCU |
| 396 | CFB144 | AACAACUCGAGCUUU |
| 397 | CFB145 | ACUAUGACGUUGCCC |
| 398 | CFB146 | UGGAAAACCUGGAAG |
| 399 | CFB147 | AUGGAAAACCUGGAA |
| 400 | CFB148 | AUUGCACAACAUGGG |
| 401 | CFB149 | AAGUCAAGGAUAUGG |
| 402 | CFB150 | UGUGUUCAAAGUCAA |
| 403 | CFB151 | AACUCCAAGAUGAGG |
| 404 | CFB152 | CAAUGAGCAACAUGU |
| 405 | CFB153 | UCAUUCAAGUUGGUG |
| 406 | CFB154 | AGACAGCGAUCUGUG |
| 407 | CFB155 | AAAGGCAGCUGUGAG |
| 408 | CFB156 | UGAUUCUGGCGGCCC |
| 409 | CFB157 | GAGUUUCAGCUUGGA |
| 410 | CFB158 | GUUGCCCUGAUCAAG |
| 411 | CFB159 | CAUGGUUUGGGAACA |
| 412 | CFB160 | CGGCAAAAGCAGGUA |
| 413 | CFB161 | AAGACAAUGAGCAAC |
| 414 | CFB162 | CAGCGGCGAACGUGU |
| 415 | CFB163 | CUACAUCAAGAAUGG |
| 416 | CFB164 | CUGAUCAAGCUCAAG |
| 417 | CFB165 | UCUACAUCAAGAAUG |
| 418 | CFB166 | CCAAGACUCCUUCAU |
| 419 | CFB167 | UGGUGUGAAGCCAAG |
| 420 | CFB168 | UGUGAAGCCAAGAUA |
| 421 | CFB169 | AAGGCAGCUGUGAGA |
| 422 | CFB170 | UCAGACAGCAUUGGG |
| 423 | CFB171 | UGUGACCACCACUCC |
| 424 | CFB172 | GAGUGAGUCCCUAUG |
| 425 | CFB173 | CAAGUUGAAGUCAGG |
| 426 | CFB174 | AACUUAAUUGAGAAG |
| 427 | CFB175 | CUGAAGGAGAAACUC |
| 428 | CFB176 | AGGAUAUCAAAGCUC |
| 429 | CFB177 | CAAAGUCAAGGACAU |
| 430 | CFB178 | CAGACACGUACCUGC |
| 431 | CFB179 | GCAGCACAUUGUUUC |
| 432 | CFB180 | CCUGCACAGGAUAUC |
| 433 | CFB181 | CCAGUCUCUGAGUCU |
| 434 | CFB182 | AAUUACUGUCAUUGA |
| 435 | CFB183 | CAGGAAGGUGGCUCU |
| 436 | CFB184 | UGUUCAAAGUCAAGG |
| 437 | CFB185 | AGAAGUCGUUUCAUU |
| 438 | CFB186 | GCUUUCCUGUCUUCC |
| 439 | CFB187 | CAGGAUAUCAAAGCU |
| 440 | CFB188 | GAUCAAGCUCAAGAA |
| 441 | CFB189 | GUGCAGAGCAAUCCA |
| 442 | CFB190 | AGAUGCUCAAUAUGC |
| 443 | CFB191 | UCAAAGUCAAGGAUA |
| 444 | CFB192 | UAGUGGAUGUCUGCA |
| 445 | CFB193 | UCGGAAGGAGGUCUA |
| 446 | CFB194 | UUAAUUGAGAAGGUG |
| 447 | CFB195 | UCCUUCCGACUUCUC |
| 448 | CFB196 | CCGUACCCUGUGCAG |
| 449 | CFB197 | CCAAUUACUGUCAUU |
| 450 | CFB198 | AGUCAGGGACUAACA |
| 451 | CFB199 | AAAGUCAAGGAUAUG |
| 452 | CFB200 | GUGCAGACACGUACC |
| 453 | CFB201 | UACUUUGUGCUGACA |
| 454 | CFB202 | AAUUAUGAAGACCAC |
| 455 | CFB203 | UCUCUGAGUCUCUGU |
| 456 | CFB204 | UAUCUGGAUGUCUAU |
| 457 | CFB205 | UGACUCGGAAGGAGG |
| 458 | CFB206 | CUUCAGGCUCCAUGA |
| 459 | CFB207 | AGUGACAUAUGCCAC |
| 460 | CFB208 | GAACAUCAAUGCUUU |
| 461 | CFB209 | ACUGGAGGAGUGAGU |
| 462 | CFB210 | AAGUCAAGGACAUCU |
| 463 | CFB211 | AUUGAAUUAAAACAG |
| 464 | CFB212 | UCCAUGAACAUCUAC |
| 465 | CFB213 | CUUUCCACUGCUAUG |
| 466 | CFB214 | CUGCCAAGUGAAUGG |
| 467 | CFB215 | AAGUGAACAUCAAUG |
| 468 | CFB216 | UGCUGCCCUGGCUGA |
| 469 | CFB217 | GCUUGGACACUGAGC |
| 470 | CFB218 | AAAGCCAGUCUCUGA |
| 471 | CFB219 | AGGAUUAUCUGGAUG |
| 472 | CFB220 | ACUUAAUUGAGAAGG |
| 473 | CFB221 | AGGUCUAGGUCUGGA |
| 474 | CFB222 | UGACUAUGACGUUGC |
| 475 | CFB223 | GUCGUUUCAUUCAAG |
| 476 | CFB224 | UGUGUCUGAGGAGGA |
| 477 | CFB225 | CAAUGCUUUGGCUUC |
| 478 | CFB226 | GAGAAGUCGUUUCAU |
| 479 | CFB227 | GAAGACCACAAGUUG |
| 480 | CFB228 | GAACAUCUACCUGGU |
| 481 | CFB229 | GUGCCAGAACACAGA |
| 482 | CFB230 | GAAGGUGGCAAGUUA |
| 483 | CFB231 | CAAGCAACCAUGGCA |
| 484 | CFB232 | CAGGCCCAUUUGUCU |
| 485 | CFB233 | UGUCUAGUCAACUUA |
| 486 | CFB234 | CCGCACCCGCCAUGU |
| 487 | CFB235 | UUGAUGAGAUCCGGG |
| 488 | CFB236 | UUUGAGGCUUCCUCC |
| 489 | CFB237 | CAUGUGUUCAAAGUC |
| 490 | CFB238 | UGUCUGAGGAGGAGA |
| 491 | CFB239 | GACGGAGCCUUCCUG |
| 492 | CFB240 | AGGUGAUUCUGGCGG |
| 493 | CFB241 | UGAGAUCUCUUUCCA |
| 494 | CFB242 | GAGAUCCGGGACUUG |
| 495 | CFB243 | CCCUGAUCAAGCUCA |
| 496 | CFB244 | AGACAAUGAGCAACA |
| 497 | CFB245 | GAUUACCACAAGCAA |
| 498 | CFB246 | AAUGUGAGUGAUGAG |
| 499 | CFB247 | CAGCUUGGACACUGA |
| 500 | CFB248 | UACAUCAAGAAUGGG |
| 501 | CFB249 | AGCAGCACAUUGUUU |
| 502 | CFB250 | CACAAGUUGAAGUCA |
| 503 | 13(5) s | AGUCUCUGUGGCAA |
| 504 | 106-13(4) s | GUUUCAUUCAA |
| 505 | 24151 | ACACAGUUUGGCCUGGAGA |
| 506 | 24152 | GGAAUCUUGAAGUCAGGAA |
| 507 | 24153 | CUGGGCUUGUAGCUGGCAC |
| 508 | 24154 | UCAAGUAAUUAUAGUGAGU |
| 509 | 24155 | AAACAGGUUUGUCUGUAUG |

TABLE 1c
Nucleobse sequences of the C5
antisense strands of 250 constructs

| SEQ ID NO. | Antisense ID | 19mer Antisense |
|---|---|---|
| 510 | 24156 | GCAGACAUUUUAACACAGA |
| 511 | 24157 | ACCUGGAGCUGGUUGCCAC |
| 512 | 24158 | GAUAAAAUCAAGUAAUUAU |
| 513 | 24159 | GACACAGUUUGGCCUGGAG |
| 514 | 24160 | CGGAAUCUUGAAGUCAGGA |
| 515 | 24161 | AGACAUUUUAACACAGAAC |
| 516 | 24162 | UGAAGUCAGGAAAAGAGAU |
| 517 | 24163 | CACAGUUUGGCCUGGAGAA |
| 518 | 24164 | AAUUAUAGUGAGUUAUUUU |
| 519 | 24165 | UCCAAGUCAGAUGUCUCUU |
| 520 | 24166 | UCAGGAAAAGAGAUAAUUC |
| 521 | 24167 | GGCAAGACAUAUUCUUUAA |
| 522 | 24168 | GAAGGCCAAUUUCCAGAGG |
| 523 | 24169 | CAAGUAAUUAUAGUGAGUU |
| 524 | 24170 | AUAAAAUCAAGUAAUUAUA |
| 525 | 24171 | AUCAAGUAAUUAUAGUGAG |
| 526 | 24172 | GCCAAUUUCCAGAGGAAGC |
| 527 | 24173 | AGUAAUUAUAGUGAGUUAU |
| 528 | 24174 | UAAAGGUACUUGUUGUUUA |
| 529 | 24175 | GACUGCUGUUUCAGAAUCA |
| 530 | 24176 | ACUGCUGUUUCAGAAUCAA |
| 531 | 24177 | AUAUAAAGGUACUUGUUGU |
| 532 | 24178 | UGUAAACAGUUCCUUUCAA |
| 533 | 24179 | GGUAACUUUGGCUGAGAGA |
| 534 | 24180 | UAUAGUUGUAAACAGUUCC |
| 535 | 24181 | ACAUAUUCUUUAACUUCAA |
| 536 | 24182 | AAGCAGUCCUUUUACACUC |
| 537 | 24183 | UAGUGAGUUAUUUUGUCAA |
| 538 | 24184 | AGGAAGACAUCUUUGAACA |
| 539 | 24185 | GCAGUCCUUUUACACUCAA |
| 540 | 24186 | AGUUAUUUUGUCAAUAUAU |
| 541 | 24187 | GUACAACAGAAUAUGGUAU |
| 542 | 24188 | GUUAUUUUGUCAAUAUAUG |
| 543 | 24189 | CAGGCUUCAGGAAAAGAGG |
| 544 | 24190 | AGGAAAAGAGAUAAUUCCA |
| 545 | 24191 | UGUUACAGCAAUAUAAAGG |
| 546 | 24192 | UAUAAGCAUAUGCAAUCUC |
| 547 | 24193 | CAUAUUCUUUAACUUCAAA |
| 548 | 24194 | AAGACAUCUUUGAACACCU |
| 549 | 24195 | CCAGGAAGACAUCUUUGAA |
| 550 | 24196 | UACAGCAAUAUAAAGGUAC |
| 551 | 24197 | CAUUGUCAUAGGUUAUUGG |
| 552 | 24198 | UGAGUUAUUUUGUCAAUAU |
| 553 | 24199 | AGUGAGUUAUUUUGUCAAU |
| 554 | 24200 | GUGAGUUAUUUUGUCAAUA |
| 555 | 24201 | GAAUUUUCCUUGAAAGAUC |
| 556 | 24202 | ACUGUUACAGCAAUAUAAA |
| 557 | 24203 | AAAUCCAUUGUCAUAGGUU |
| 558 | 24204 | AAUCCAUUGUCAUAGGUUA |
| 559 | 24205 | GAGAAAUCCAUUGUCAUAG |
| 560 | 24206 | AAGACAUAUUCUUUAACUU |
| 561 | 24207 | AAGUGCAGAUUCCCUCCAC |
| 562 | 24208 | AUCCAUUGUCAUAGGUUAU |
| 563 | 24209 | AGACAUCUUUGAACACCUU |
| 564 | 24210 | CCAUUGUCAUAGGUUAUUG |
| 565 | 24211 | UGAAGAGAAAUCCAUUGUC |
| 566 | 24212 | GACAUAUUCUUUAACUUCA |
| 567 | 24213 | CAGUCCUUUUACACUCAAA |
| 568 | 24214 | AAUUUUCCUUGAAAGAUCC |
| 569 | 24215 | UGAAAUUGUAUUUUAUCUG |
| 570 | 24216 | AGUAAUUUCAAAAUUCUUA |
| 571 | 24217 | CAAAAUUCUUAAAGUUCUU |
| 572 | 24218 | UGAAUUUUGGUUCUGCUCU |
| 573 | 24219 | UGUCAUUUUAUAAUUAUGU |
| 574 | 24220 | CCAAAUCCUGUACUGACAA |
| 575 | 24221 | GGAUAACUUUUAAUAGAGA |
| 576 | 24222 | UUUAAGUCUUCUCUUAUUC |
| 577 | 24223 | GAUAAUUCCAAUAUGAUCA |
| 578 | 24224 | GGAUAAAUGAACAUGGCCU |
| 579 | 24225 | CAAGGUUCAUCAUUUUCUU |
| 580 | 24226 | UGGAAGUGCUAUAAAACAU |
| 581 | 24227 | CCAAGUACUCUUAAAGCAA |
| 582 | 24228 | UCCAAUGAUUUCCUGUUUC |
| 583 | 24229 | UAUGGUAUAUUCAUUUCCA |
| 584 | 24230 | GAACAAGAUGAACUUCCCA |
| 585 | 24231 | UGAACUUCAGGAAUUUUAG |
| 586 | 24232 | AAGUCUUCUCUUAUUCCAA |
| 587 | 24233 | GAAUGUUUAUACUUUGAUA |
| 588 | 24234 | CCGGAAUCGUACACAAAGG |
| 589 | 24235 | CAUACCUCUGCUCUUCUGA |
| 590 | 24236 | GAUCAAUUUCUUCUACCAU |
| 591 | 24237 | CAACAUUGUGUUUUGCAUU |
| 592 | 24238 | UAACUUUAUAAGCAUAUGC |
| 593 | 24239 | CAGGAUAACUUUUAAUAGA |
| 594 | 24240 | UUUUAUUGGUUGAUACUGU |
| 595 | 24241 | UGCAACUGUUUUCUUCUGG |
| 596 | 24242 | UGCUUUGAUACAACUUCCA |
| 597 | 24243 | CCAAAGCUUCUCUCUUCAA |
| 598 | 24244 | GGGAACUCCUUUCGUCUGC |
| 599 | 24245 | UAUGACAGUUCUUUGACUG |
| 600 | 24246 | UUGCAGAAUAACAUGUCCA |
| 601 | 24247 | CAGAAGUCCUAUAGUUGUA |
| 602 | 24248 | GAUAACUUUUAAUAGAGAU |
| 603 | 24249 | ACUAAGAUUUCUUUUCCAA |
| 604 | 24250 | GAUAAAUGAACAUGGCCUG |
| 605 | 24251 | GAUGAACAUGUUGUGUCUC |
| 606 | 24252 | UGAUCAUCUUUUAAGUCUU |
| 607 | 24253 | GAGCAAUUCCAUUUAUCAA |
| 608 | 24254 | UGUGAAUUUUCCUUGAAAG |
| 609 | 24255 | UCAAAAUUCUUAAAGUUCU |
| 610 | 24256 | UAAACUCCAGCACCGUCAC |
| 611 | 24257 | UUGAUAUUGGAAGUGCUAU |
| 612 | 24258 | GUGCAUUCAGUGUUACUGG |
| 613 | 24259 | AAUGUUUAUACUUUGAUAA |
| 614 | 24260 | AAAUUGUAUUUUAUCUGGA |
| 615 | 24261 | UAAGUCUUCUCUUAUUCCA |
| 616 | 24262 | AGAAGUCCUAUAGUUGUAA |
| 617 | 24263 | CUUGCUUUUAUAGUAAUUU |
| 618 | 24264 | UCAACAUUGUGUUUUGCAU |
| 619 | 24265 | AGGCAGUUGUUUCUACCAU |
| 620 | 24266 | UAUGAUCAAUUUCUUCUAC |
| 621 | 24267 | GUAAUUUCAAAAUUCUUAA |
| 622 | 24268 | AUCAACAUUGUGUUUUGCA |
| 623 | 24269 | CAAAGUAUUCCCAAAAGGC |
| 624 | 24270 | AAAACAUGGUACACUGUUU |
| 625 | 24271 | ACACAGAACUGCAUCCCAG |
| 626 | 24272 | UCCAAGUACUCUUAAAGCA |
| 627 | 24273 | UGUAGUAUGACAGUUCUUU |
| 628 | 24274 | CAGAAUAGCUUUCCCUUUU |
| 629 | 24275 | GCUUUUAUAGUAAUUUCAA |
| 630 | 24276 | GAAGCUACUCCAUCAUCAA |
| 631 | 24277 | UGCCACUAAUUCUAAGUAA |
| 632 | 24278 | GUAGACUCUAUGACUGUUA |
| 633 | 24279 | CUAAGAUUUCUUUUCCAAA |
| 634 | 24280 | CAAUAUUUAACCAGACUGA |
| 635 | 24281 | UAGUAAUUUCAAAAUUCUU |
| 636 | 24282 | GGAAAAGAGAUAAUUCCAA |
| 637 | 24283 | AAAAUGCUUGACACGAUGA |
| 638 | 24284 | UAUAUUCAUUUCCAGGAAG |
| 639 | 24285 | UAAUAAAAGCAAGUGCCAC |
| 640 | 24286 | UACUAUGCGUUUGUAAUCA |
| 641 | 24287 | GAAUUGAAAUACUCUUUCC |
| 642 | 24288 | UCAGGAUAACUUUUAAUAG |
| 643 | 24289 | UACAACUUCCAAAUACACA |
| 644 | 24290 | AAUCAUUCUCUAAUAAAAG |
| 645 | 24291 | GAAUAGCUUUCCCUUUUGA |
| 646 | 24292 | GGAAAUUCUUGUCUGUCAU |
| 647 | 24293 | UUGAAUUUUGGUUCUGCUC |
| 648 | 24294 | AGUGAGCUUUACAAAUAAG |
| 649 | 24295 | AAACAUGGUACACUGUUUA |
| 650 | 24296 | UAGACUCUAUGACUGUUAC |
| 651 | 24297 | UUUUCUUGGGAGUCAUCUG |
| 652 | 24298 | GUAGGCAAGGAGAUGUCCA |
| 653 | 24299 | AAAGCCACCUCCAUACCUC |
| 654 | 24300 | UAAGAUUUCUUUUCCAAAC |
| 655 | 24301 | AAAGCUGCAAACUUCCUCA |
| 656 | 24302 | UCUAAUAAAAGCAAGUGCC |
| 657 | 24303 | CAAUUGUUUGUGCAUUCAG |
| 658 | 24304 | ACCAAAGCUUCUCUCUUCA |
| 659 | 24305 | GCAACUGUUUUCUUCUGGG |
| 660 | 24306 | AUGCUUUGAUACAACUUCC |
| 661 | 24307 | AAUUCAUCUAAAUUAGCUA |
| 662 | 24308 | UGUGACGAUGUAAUAGACC |
| 663 | 24309 | GGUACACUGUUUAUCUGGU |
| 664 | 24310 | GGUUGCCUUGUACUUGACA |
| 665 | 24311 | CCAAGGAAAUUCUUGUCUG |
| 666 | 24312 | UUAACCAGACUGAAUCAGA |
| 667 | 24313 | UAGCAGCUAUUUCUUCUAU |
| 668 | 24314 | CCAGUUUUGUAGAUAUCCA |
| 669 | 24315 | AAUGAUUUCCUGUUUCCAG |
| 670 | 24316 | AGACAAGAUCUCACCUACA |
| 671 | 24317 | GUGGAUUACCUUUAACCAA |
| 672 | 24318 | UGAUACUGUGAAUUUUCCU |
| 673 | 24319 | AAUACUGGGACAACGCUCA |
| 674 | 24320 | CAUAAUUAAGUACUGUCUU |
| 675 | 24321 | CAAACUUCCUCAGAGGUAC |
| 676 | 24322 | AAGGCCAUGUUAUUUCAGA |
| 677 | 24323 | UGCAAACUGUAUGCAGCUG |
| 678 | 24324 | AGAAUAGCUUUCCCUUUUG |
| 679 | 24325 | UACUGUGAAUUUUCCUUGA |
| 680 | 24326 | CCAACUUCAAAGAGUUCAA |
| 681 | 24327 | CUGAAUUUUGAUAUUGGAA |
| 682 | 24328 | AAAGGCUGUAAGAUAUAAG |
| 683 | 24329 | UUGCUUUUAUAGUAAUUUC |
| 684 | 24330 | GUCCAAGUACUCUUAAAGC |
| 685 | 24331 | ACACUAAUUCUGCUGUCUG |
| 686 | 24332 | CUGGCUUGCUUACUGGUAA |
| 687 | 24333 | GUUGCCUUGUACUUGACAA |
| 688 | 24334 | CAGCAUUUCUGUAGGACAU |
| 689 | 24335 | UUAUACUUUGAUAAGAUGC |
| 690 | 24336 | UUAAGUACUGUCUUCCUUU |
| 691 | 24337 | CUGCAAACUUCCUCAGAGG |
| 692 | 24338 | AAGACAGUUUCUCUUUUGG |
| 693 | 24339 | AUUCAGUUUGUAGGGAGAG |
| 694 | 24340 | UGGAAUGUUUAUACUUUGA |
| 695 | 24341 | UAACAGGGUCUUCAUGUGU |
| 696 | 24342 | AGAGAUUGUUGCAUCAAAU |
| 697 | 24343 | GAGUCAUCUGCAUUUGCAU |
| 698 | 24344 | GAAUGUUUAUAUUUAGCAG |
| 699 | 24345 | GGAACAAGAUGAACUUCCC |
| 700 | 24346 | GGUAUUUCUGCCUCUUCAG |
| 701 | 24347 | CAGAACUGCAUCCCAGAAG |
| 702 | 24348 | UUCCGGUGUCCAAUAACCU |
| 703 | 24349 | CAUCAAUUGUUUGUGCAUU |
| 704 | 24350 | AAUACUCUUUCCAAGGGCU |
| 705 | 24351 | UCAAAUGCUUCAGUGUAUC |
| 706 | 24352 | AAGCAAGUGCCACUAAUUC |
| 707 | 24353 | UUAAUAGAGAUUGUUGCAU |
| 708 | 24354 | AUAGAGAUUGUUGCAUCAA |
| 709 | 24355 | GAACUCCUUUCGUCUGCUA |
| 710 | 24356 | GUCAUUUUAUAAUUAUGUA |
| 711 | 24357 | AAGAAUUGAAAUACUCUUU |
| 712 | 24358 | AAAGCCUUUCUAAUUCCAA |
| 713 | 24359 | UCAAUAUUUAACCAGACUG |
| 714 | 24360 | UGGUAUAUUCAUUUCCAGG |
| 715 | 24361 | UGAAACAAUUGAACGAAAC |
| 716 | 24362 | AAUAUCUUGCUUUUAUAGU |
| 717 | 24363 | AGAGAUUGUCAGAUCCAAU |
| 718 | 24364 | AGCAAUUCCAUUUAUCAAC |
| 719 | 24365 | UUAUCAGGAUAACUUUUAA |
| 720 | 24366 | CGAAUUUCUGGCUUGCUUA |
| 721 | 24367 | UCAAGUUCAGACUGGUGAG |
| 722 | 24368 | AGAGGUACUGGUUUUGUGA |
| 723 | 24369 | GGUAUUUUCUUUCAAGCAA |
| 724 | 24370 | AGGUACUGGUUUUGUGAAC |
| 725 | 24371 | CAGAUUCCCUCCACAGCAG |
| 726 | 24372 | UAGUCAGCAUUUCUGUAGG |
| 727 | 24373 | AUUAGCACGGAGCUGGCUU |
| 728 | 24374 | UAAGAGACACAGUUUGGCC |
| 729 | 24375 | GAACAUGUUGUGUCUCUAG |
| 730 | 24376 | CAUAAGUGCAAACUGUAUG |
| 731 | 24377 | CAAAUUCAGUUUGUAGGGA |
| 732 | 24378 | ACAAGCAGUCCUUUUACAC |
| 733 | 24379 | AAGUAUUCCCAAAAGGCCC |
| 734 | 24380 | CAAUCACAGUAAAGGCUGU |
| 735 | 24381 | AAUAGCUUUCCCUUUUGAC |
| 736 | 24382 | UAUUGGUUGAUACUGUGAA |
| 737 | 24383 | CAGAAAUGGCCAAUGUAAA |
| 738 | 24384 | UUGUAAUCAGAGUUUCCGU |
| 739 | 24385 | ACUCAGGCUUUAAUGAUCA |
| 740 | 24386 | CAGCUAUUUCUUCUAUCUU |
| 741 | 24387 | AUAUCUUGCUUUUAUAGUA |
| 742 | 24388 | ACAAGGUUCAUCAUUUUCU |
| 743 | 24389 | AUUGUGUUUUGCAUUGCUG |
| 744 | 24390 | GCCACAAUAAAGAAUUACA |
| 745 | 24391 | GGUGGAUUACCUUUAACCA |
| 746 | 24392 | CUUCUCUCUUCAAAGCUGA |
| 747 | 24393 | UGGGAUGCUUCAAUAUCCU |
| 748 | 24394 | AAAUCUUCUAAACUGUAGU |
| 749 | 24395 | AAAUUCAGUUUGUAGGGAG |
| 750 | 24396 | CUGUCAUUUUAUAAUUAUG |
| 751 | 24397 | AAUGCUUUGAUACAACUUC |
| 752 | 24398 | UCCUCUUUAUAUUUAGCCU |
| 753 | 24399 | GCAAAUUCAUCUAAAUUAG |
| 754 | 24400 | UUCCAAAUACACAUAAGAA |

The first nucleobase on the terminal 5′ position (the sequences in the table are presented from a 5′(Ieft) to a 3′(right direction) can be freely selected from U, A, G and C instead of the nucleobase disclosed in the table.

TABLE 1d
Nucleobase sequences of the C5
sense strands of 250 constructs

| SEQ ID NO. | Sense ID | 14mer Sense |
|---|---|---|
| 755 | 14151 | AGGCCAAACUGUGU |
| 756 | 14152 | GACUUCAAGAUUCC |
| 757 | 14153 | AGCUACAAGCCCAG |
| 758 | 14154 | CUAUAAUUACUUGA |
| 759 | 14155 | AGACAAACCUGUUU |
| 760 | 14156 | GUUAAAAUGUCUGC |
| 761 | 14157 | AACCAGCUCCAGGU |
| 762 | 14158 | UACUUGAUUUUAUC |
| 763 | 14159 | GGCCAAACUGUGUC |
| 764 | 14160 | ACUUCAAGAUUCCG |
| 765 | 14161 | GUGUUAAAAUGUCU |
| 766 | 14162 | UUUUCCUGACUUCA |
| 767 | 14163 | CAGGCCAAACUGUG |
| 768 | 14164 | AACUCACUAUAAUU |
| 769 | 14165 | ACAUCUGACUUGGA |
| 770 | 14166 | AUCUCUUUUCCUGA |
| 771 | 14167 | GAAUAUGUCUUGCC |
| 772 | 14168 | GGAAAUUGGCCUUC |
| 773 | 14169 | ACUAUAAUUACUUG |
| 774 | 14170 | UUACUUGAUUUUAU |
| 775 | 14171 | UAUAAUUACUUGAU |
| 776 | 14172 | CUCUGGAAAUUGGC |
| 777 | 14173 | UCACUAUAAUUACU |
| 778 | 14174 | AACAAGUACCUUUA |
| 779 | 14175 | CUGAAACAGCAGUC |
| 780 | 14176 | UCUGAAACAGCAGU |
| 781 | 14177 | AAGUACCUUUAUAU |
| 782 | 14178 | AGGAACUGUUUACA |
| 783 | 14179 | CAGCCAAAGUUACC |
| 784 | 14180 | UGUUUACAACUAUA |
| 785 | 14181 | GUUAAAGAAUAUGU |
| 786 | 14182 | UAAAAGGACUGCUU |
| 787 | 14183 | AAAAUAACUCACUA |
| 788 | 14184 | AAAGAUGUCUUCCU |
| 789 | 14185 | UGUAAAAGGACUGC |
| 790 | 14186 | UUGACAAAAUAACU |
| 791 | 14187 | AUAUUCUGUUGUAC |
| 792 | 14188 | AUUGACAAAAUAAC |
| 793 | 14189 | UUUCCUGAAGCCUG |
| 794 | 14190 | UUAUCUCUUUUCCU |
| 795 | 14191 | AUAUUGCUGUAACA |
| 796 | 14192 | UGCAUAUGCUUAUA |
| 797 | 14193 | AGUUAAAGAAUAUG |
| 798 | 14194 | UUCAAAGAUGUCUU |
| 799 | 14195 | AGAUGUCUUCCUGG |
| 800 | 14196 | UUUAUAUUGCUGUA |
| 801 | 14197 | AACCUAUGACAAUG |
| 802 | 14198 | GACAAAAUAACUCA |
| 803 | 14199 | CAAAAUAACUCACU |
| 804 | 14200 | ACAAAAUAACUCAC |
| 805 | 14201 | UUCAAGGAAAAUUC |
| 806 | 14202 | AUUGCUGUAACAGU |
| 807 | 14203 | AUGACAAUGGAUUU |
| 808 | 14204 | UAUGACAAUGGAUU |
| 809 | 14205 | ACAAUGGAUUUCUC |
| 810 | 14206 | AAAGAAUAUGUCUU |
| 811 | 14207 | GGGAAUCUGCACUU |
| 812 | 14208 | CUAUGACAAUGGAU |
| 813 | 14209 | GUUCAAAGAUGUCU |
| 814 | 14210 | ACCUAUGACAAUGG |
| 815 | 14211 | UGGAUUUCUCUUCA |
| 816 | 14212 | UUAAAGAAUAUGUC |
| 817 | 14213 | GUGUAAAAGGACUG |
| 818 | 14214 | UUUCAAGGAAAAUU |
| 819 | 14215 | AAAAUACAAUUUCA |
| 820 | 14216 | AUUUUGAAAUUACU |
| 821 | 14217 | CUUUAAGAAUUUUG |
| 822 | 14218 | AGAACCAAAAUUCA |
| 823 | 14219 | AUUAUAAAAUGACA |
| 824 | 14220 | AGUACAGGAUUUGG |
| 825 | 14221 | AUUAAAAGUUAUCC |
| 826 | 14222 | AGAGAAGACUUAAA |
| 827 | 14223 | AUAUUGGAAUUAUC |
| 828 | 14224 | AUGUUCAUUUAUCC |
| 829 | 14225 | AAUGAUGAACCUUG |
| 830 | 14226 | UUAUAGCACUUCCA |
| 831 | 14227 | UUAAGAGUACUUGG |
| 832 | 14228 | AGGAAAUCAUUGGA |
| 833 | 14229 | AUGAAUAUACCAUA |
| 834 | 14230 | AGUUCAUCUUGUUC |
| 835 | 14231 | AUUCCUGAAGUUCA |
| 836 | 14232 | AUAAGAGAAGACUU |
| 837 | 14233 | AAGUAUAAACAUUC |
| 838 | 14234 | GUGUACGAUUCCGG |
| 839 | 14235 | AGAGCAGAGGUAUG |
| 840 | 14236 | AGAAGAAAUUGAUC |
| 841 | 14237 | AAAACACAAUGUUG |
| 842 | 14238 | UGCUUAUAAAGUUA |
| 843 | 14239 | UAAAAGUUAUCCUG |
| 844 | 14240 | AUCAACCAAUAAAA |
| 845 | 14241 | AGAAAACAGUUGCA |
| 846 | 14242 | GUUGUAUCAAAGCA |
| 847 | 14243 | GAGAGAAGCUUUGG |
| 848 | 14244 | CGAAAGGAGUUCCC |
| 849 | 14245 | AAAGAACUGUCAUA |
| 850 | 14246 | AUGUUAUUCUGCAA |
| 851 | 14247 | CUAUAGGACUUCUG |
| 852 | 14248 | UAUUAAAAGUUAUC |
| 853 | 14249 | AAAGAAAUCUUAGU |
| 854 | 14250 | CAUGUUCAUUUAUC |
| 855 | 14251 | ACAACAUGUUCAUC |
| 856 | 14252 | UUAAAAGAUGAUCA |
| 857 | 14253 | AAAUGGAAUUGCUC |
| 858 | 14254 | AAGGAAAAUUCACA |
| 859 | 14255 | UUUAAGAAUUUUGA |
| 860 | 14256 | GGUGCUGGAGUUUA |
| 861 | 14257 | ACUUCCAAUAUCAA |
| 862 | 14258 | AACACUGAAUGCAC |
| 863 | 14259 | AAAGUAUAAACAUU |
| 864 | 14260 | AUAAAAUACAAUUU |
| 865 | 14261 | UAAGAGAAGACUUA |
| 866 | 14262 | ACUAUAGGACUUCU |
| 867 | 14263 | ACUAUAAAAGCAAG |
| 868 | 14264 | AAACACAAUGUUGA |
| 869 | 14265 | AGAAACAACUGCCU |
| 870 | 14266 | AGAAAUUGAUCAUA |
| 871 | 14267 | AAUUUUGAAAUUAC |
| 872 | 14268 | AACACAAUGUUGAU |
| 873 | 14269 | UUGGGAAUACUUUG |
| 874 | 14270 | GUGUACCAUGUUUU |
| 875 | 14271 | AUGCAGUUCUGUGU |
| 876 | 14272 | UAAGAGUACUUGGA |
| 877 | 14273 | ACUGUCAUACUACA |
| 878 | 14274 | GGAAAGCUAUUCUG |
| 879 | 14275 | AUUACUAUAAAAGC |
| 880 | 14276 | GAUGGAGUAGCUUC |
| 881 | 14277 | UAGAAUUAGUGGCA |
| 882 | 14278 | GUCAUAGAGUCUAC |
| 883 | 14279 | AAAAGAAAUCUUAG |
| 884 | 14280 | CUGGUUAAAUAUUG |
| 885 | 14281 | UUUUGAAAUUACUA |
| 886 | 14282 | AUUAUCUCUUUUCC |
| 887 | 14283 | GUGUCAAGCAUUUU |
| 888 | 14284 | UGGAAAUGAAUAUA |
| 889 | 14285 | ACUUGCUUUUAUUA |
| 890 | 14286 | ACAAACGCAUAGUA |
| 891 | 14287 | GAGUAUUUCAAUUC |
| 892 | 14288 | AAAAGUUAUCCUGA |
| 893 | 14289 | AUUUGGAAGUUGUA |
| 894 | 14290 | AUUAGAGAAUGAUU |
| 895 | 14291 | AGGGAAAGCUAUUC |
| 896 | 14292 | AGACAAGAAUUUCC |
| 897 | 14293 | GAACCAAAAUUCAA |
| 898 | 14294 | UUGUAAAGCUCACU |
| 899 | 14295 | AGUGUACCAUGUUU |
| 900 | 14296 | AGUCAUAGAGUCUA |
| 901 | 14297 | GACUCCCAAGAAAA |
| 902 | 14298 | AUCUCCUUGCCUAC |
| 903 | 14299 | AUGGAGGUGGCUUU |
| 904 | 14300 | GAAAAGAAAUCUUA |
| 905 | 14301 | AAGUUUGCAGCUUU |
| 906 | 14302 | UUGCUUUUAUUAGA |
| 907 | 14303 | UGCACAAACAAUUG |
| 908 | 14304 | AGAGAAGCUUUGGU |
| 909 | 14305 | AAGAAAACAGUUGC |
| 910 | 14306 | UUGUAUCAAAGCAU |
| 911 | 14307 | AAUUUAGAUGAAUU |
| 912 | 14308 | AUUACAUCGUCACA |
| 913 | 14309 | AUAAACAGUGUACC |
| 914 | 14310 | AGUACAAGGCAACC |
| 915 | 14311 | AAGAAUUUCCUUGG |
| 916 | 14312 | UUCAGUCUGGUUAA |
| 917 | 14313 | AGAAAUAGCUGCUA |
| 918 | 14314 | AUCUACAAAACUGG |
| 919 | 14315 | AACAGGAAAUCAUU |
| 920 | 14316 | GUGAGAUCUUGUCU |
| 921 | 14317 | UAAAGGUAAUCCAC |
| 922 | 14318 | AAUUCACAGUAUCA |
| 923 | 14319 | GUUGUCCCAGUAUU |
| 924 | 14320 | AGUACUUAAUUAUG |
| 925 | 14321 | UCUGAGGAAGUUUG |
| 926 | 14322 | AAUAACAUGGCCUU |
| 927 | 14323 | GCAUACAGUUUGCA |
| 928 | 14324 | GGGAAAGCUAUUCU |
| 929 | 14325 | GAAAAUUCACAGUA |
| 930 | 14326 | CUCUUUGAAGUUGG |
| 931 | 14327 | AUAUCAAAAUUCAG |
| 932 | 14328 | AUCUUACAGCCUUU |
| 933 | 14329 | UACUAUAAAAGCAA |
| 934 | 14330 | AAGAGUACUUGGAC |
| 935 | 14331 | AGCAGAAUUAGUGU |
| 936 | 14332 | AGUAAGCAAGCCAG |
| 937 | 14333 | AAGUACAAGGCAAC |
| 938 | 14334 | CUACAGAAAUGCUG |
| 939 | 14335 | UUAUCAAAGUAUAA |
| 940 | 14336 | AAGACAGUACUUAA |
| 941 | 14337 | GAGGAAGUUUGCAG |
| 942 | 14338 | AGAGAAACUGUCUU |
| 943 | 14339 | CCUACAAACUGAAU |
| 944 | 14340 | GUAUAAACAUUCCA |
| 945 | 14341 | UGAAGACCCUGUUA |
| 946 | 14342 | AUGCAACAAUCUCU |
| 947 | 14343 | AAUGCAGAUGACUC |
| 948 | 14344 | AAAUAUAAACAUUC |
| 949 | 14345 | GUUCAUCUUGUUCC |
| 950 | 14346 | GAGGCAGAAAUACC |
| 951 | 14347 | GGGAUGCAGUUCUG |
| 952 | 14348 | AUUGGACACCGGAA |
| 953 | 14349 | ACAAACAAUUGAUG |
| 954 | 14350 | UUGGAAAGAGUAUU |
| 955 | 14351 | ACUGAAGCAUUUGA |
| 956 | 14352 | AGUGGCACUUGCUU |
| 957 | 14353 | ACAAUCUCUAUUAA |
| 958 | 14354 | GCAACAAUCUCUAU |
| 959 | 14355 | GACGAAAGGAGUUC |
| 960 | 14356 | AAUUAUAAAAUGAC |
| 961 | 14357 | GUAUUUCAAUUCUU |
| 962 | 14358 | AUUAGAAAGGCUUU |
| 963 | 14359 | UGGUUAAAUAUUGA |
| 964 | 14360 | AAAUGAAUAUACCA |
| 965 | 14361 | GUUCAAUUGUUUCA |
| 966 | 14362 | AAAAGCAAGAUAUU |
| 967 | 14363 | AUCUGACAAUCUCU |
| 968 | 14364 | UAAAUGGAAUUGCU |
| 969 | 14365 | AGUUAUCCUGAUAA |
| 970 | 14366 | AAGCCAGAAAUUCG |
| 971 | 14367 | CAGUCUGAACUUGA |
| 972 | 14368 | AAACCAGUACCUCU |
| 973 | 14369 | UGAAAGAAAAUACC |
| 974 | 14370 | CAAAACCAGUACCU |
| 975 | 14371 | GUGGAGGGAAUCUG |
| 976 | 14372 | AGAAAUGCUGACUA |
| 977 | 14373 | AGCUCCGUGCUAAU |
| 978 | 14374 | AACUGUGUCUCUUA |
| 979 | 14375 | GACACAACAUGUUC |
| 980 | 14376 | AGUUUGCACUUAUG |
| 981 | 14377 | ACAAACUGAAUUUG |
| 982 | 14378 | AAAGGACUGCUUGU |
| 983 | 14379 | UUUUGGGAAUACUU |
| 984 | 14380 | CUUUACUGUGAUUG |
| 985 | 14381 | AAGGGAAAGCUAUU |
| 986 | 14382 | AGUAUCAACCAAUA |
| 987 | 14383 | AUUGGCCAUUUCUG |
| 988 | 14384 | AACUCUGAUUACAA |
| 989 | 14385 | AUUAAAGCCUGAGU |
| 990 | 14386 | AGAAGAAAUAGCUG |
| 991 | 14387 | UAAAAGCAAGAUAU |
| 992 | 14388 | AUGAUGAACCUUGU |
| 993 | 14389 | AUGCAAAACACAAU |
| 994 | 14390 | UUCUUUAUUGUGGC |
| 995 | 14391 | AAAGGUAAUCCACC |
| 996 | 14392 | UUUGAAGAGAGAAG |
| 997 | 14393 | AUUGAAGCAUCCCA |
| 998 | 14394 | AGUUUAGAAGAUUU |
| 999 | 14395 | UACAAACUGAAUUU |
| 1000 | 14396 | UUAUAAAAUGACAG |
| 1001 | 14397 | UGUAUCAAAGCAUU |
| 1002 | 14398 | AAAUAUAAAGAGGA |
| 1003 | 14399 | UUAGAUGAAUUUGC |
| 1004 | 14400 | AUGUGUAUUUGGAA |

Tables 2a-2b below show the nucleobase sequences composing the 252 hairpin constructs of the disclosed embodiments as selected in accordance with the examples. The nucleobase sequences are a direct fusion of the antisense sequences of Table 1a with the corresponding sense sequences of Table 1b.

TABLE 2a
Nucleobase sequences of 252 CFB constructs in which the sense and the
antisense sequences of Tables 1a and 1b are combined.

| SEQ ID NO. | Sense ID | Antisense ID | 33mer CFB Hairpin sequence |
|---|---|---|---|
| 1005 | 14151 | 24151 | ACACAGUUUGGCCUGGAGAAGGCCAAACUGUGU |
| 1006 | 14152 | 24152 | GGAAUCUUGAAGUCAGGAAGACUUCAAGAUUCC |
| 1007 | 14153 | 24153 | CUGGGCUUGUAGCUGGCACAGCUACAAGCCCAG |
| 1008 | 14154 | 24154 | UCAAGUAAUUAUAGUGAGUCUAUAAUUACUUGA |
| 1009 | 14155 | 24155 | AAACAGGUUUGUCUGUAUGAGACAAACCUGUUU |
| 1010 | 14156 | 24156 | GCAGACAUUUUAACACAGAGUUAAAAUGUCUGC |
| 1011 | 14157 | 24157 | ACCUGGAGCUGGUUGCCACAACCAGCUCCAGGU |
| 1012 | 14158 | 24158 | GAUAAAAUCAAGUAAUUAUUACUUGAUUUUAUC |
| 1013 | 14159 | 24159 | GACACAGUUUGGCCUGGAGGGCCAAACUGUGUC |
| 1014 | 14160 | 24160 | CGGAAUCUUGAAGUCAGGAACUUCAAGAUUCCG |
| 1015 | 14161 | 24161 | AGACAUUUUAACACAGAACGUGUUAAAAUGUCU |
| 1016 | 14162 | 24162 | UGAAGUCAGGAAAAGAGAUUUUUCCUGACUUCA |
| 1017 | 14163 | 24163 | CACAGUUUGGCCUGGAGAACAGGCCAAACUGUG |
| 1018 | 14164 | 24164 | AAUUAUAGUGAGUUAUUUUAACUCACUAUAAUU |
| 1019 | 14165 | 24165 | UCCAAGUCAGAUGUCUCUUACAUCUGACUUGGA |
| 1020 | 14166 | 24166 | UCAGGAAAAGAGAUAAUUCAUCUCUUUUCCUGA |
| 1021 | 14167 | 24167 | GGCAAGACAUAUUCUUUAAGAAUAUGUCUUGCC |
| 1022 | 14168 | 24168 | GAAGGCCAAUUUCCAGAGGGGAAAUUGGCCUUC |
| 1023 | 14169 | 24169 | CAAGUAAUUAUAGUGAGUUACUAUAAUUACUUG |
| 1024 | 14170 | 24170 | AUAAAAUCAAGUAAUUAUAUUACUUGAUUUUAU |
| 1025 | 14171 | 24171 | AUCAAGUAAUUAUAGUGAGUAUAAUUACUUGAU |
| 1026 | 14172 | 24172 | GCCAAUUUCCAGAGGAAGCCUCUGGAAAUUGGC |
| 1027 | 14173 | 24173 | AGUAAUUAUAGUGAGUUAUUCACUAUAAUUACU |
| 1028 | 14174 | 24174 | UAAAGGUACUUGUUGUUUAAACAAGUACCUUUA |
| 1029 | 14175 | 24175 | GACUGCUGUUUCAGAAUCACUGAAACAGCAGUC |
| 1030 | 14176 | 24176 | ACUGCUGUUUCAGAAUCAAUCUGAAACAGCAGU |
| 1031 | 14177 | 24177 | AUAUAAAGGUACUUGUUGUAAGUACCUUUAUAU |
| 1032 | 14178 | 24178 | UGUAAACAGUUCCUUUCAAAGGAACUGUUUACA |
| 1033 | 14179 | 24179 | GGUAACUUUGGCUGAGAGACAGCCAAAGUUACC |
| 1034 | 14180 | 24180 | UAUAGUUGUAAACAGUUCCUGUUUACAACUAUA |
| 1035 | 14181 | 24181 | ACAUAUUCUUUAACUUCAAGUUAAAGAAUAUGU |
| 1036 | 14182 | 24182 | AAGCAGUCCUUUUACACUCUAAAAGGACUGCUU |
| 1037 | 14183 | 24183 | UAGUGAGUUAUUUUGUCAAAAAAUAACUCACUA |
| 1038 | 14184 | 24184 | AGGAAGACAUCUUUGAACAAAAGAUGUCUUCCU |
| 1039 | 14185 | 24185 | GCAGUCCUUUUACACUCAAUGUAAAAGGACUGC |
| 1040 | 14186 | 24186 | AGUUAUUUUGUCAAUAUAUUUGACAAAAUAACU |
| 1041 | 14187 | 24187 | GUACAACAGAAUAUGGUAUAUAUUCUGUUGUAC |
| 1042 | 14188 | 24188 | GUUAUUUUGUCAAUAUAUGAUUGACAAAAUAAC |
| 1043 | 14189 | 24189 | CAGGCUUCAGGAAAAGAGGUUUCCUGAAGCCUG |
| 1044 | 14190 | 24190 | AGGAAAAGAGAUAAUUCCAUUAUCUCUUUUCCU |
| 1045 | 14191 | 24191 | UGUUACAGCAAUAUAAAGGAUAUUGCUGUAACA |
| 1046 | 14192 | 24192 | UAUAAGCAUAUGCAAUCUCUGCAUAUGCUUAUA |
| 1047 | 14193 | 24193 | CAUAUUCUUUAACUUCAAAAGUUAAAGAAUAUG |
| 1048 | 14194 | 24194 | AAGACAUCUUUGAACACCUUUCAAAGAUGUCUU |
| 1049 | 14195 | 24195 | CCAGGAAGACAUCUUUGAAAGAUGUCUUCCUGG |
| 1050 | 14196 | 24196 | UACAGCAAUAUAAAGGUACUUUAUAUUGCUGUA |
| 1051 | 14197 | 24197 | CAUUGUCAUAGGUUAUUGGAACCUAUGACAAUG |
| 1052 | 14198 | 24198 | UGAGUUAUUUUGUCAAUAUGACAAAAUAACUCA |
| 1053 | 14199 | 24199 | AGUGAGUUAUUUUGUCAAUCAAAAUAACUCACU |
| 1054 | 14200 | 24200 | GUGAGUUAUUUUGUCAAUAACAAAAUAACUCAC |
| 1055 | 14201 | 24201 | GAAUUUUCCUUGAAAGAUCUUCAAGGAAAAUUC |
| 1056 | 14202 | 24202 | ACUGUUACAGCAAUAUAAAAUUGCUGUAACAGU |
| 1057 | 14203 | 24203 | AAAUCCAUUGUCAUAGGUUAUGACAAUGGAUUU |
| 1058 | 14204 | 24204 | AAUCCAUUGUCAUAGGUUAUAUGACAAUGGAUU |
| 1059 | 14205 | 24205 | GAGAAAUCCAUUGUCAUAGACAAUGGAUUUCUC |
| 1060 | 14206 | 24206 | AAGACAUAUUCUUUAACUUAAAGAAUAUGUCUU |
| 1061 | 14207 | 24207 | AAGUGCAGAUUCCCUCCACGGGAAUCUGCACUU |
| 1062 | 14208 | 24208 | AUCCAUUGUCAUAGGUUAUCUAUGACAAUGGAU |
| 1063 | 14209 | 24209 | AGACAUCUUUGAACACCUUGUUCAAAGAUGUCU |
| 1064 | 14210 | 24210 | CCAUUGUCAUAGGUUAUUGACCUAUGACAAUGG |
| 1065 | 14211 | 24211 | UGAAGAGAAAUCCAUUGUCUGGAUUUCUCUUCA |
| 1066 | 14212 | 24212 | GACAUAUUCUUUAACUUCAUUAAAGAAUAUGUC |
| 1067 | 14213 | 24213 | CAGUCCUUUUACACUCAAAGUGUAAAAGGACUG |
| 1068 | 14214 | 24214 | AAUUUUCCUUGAAAGAUCCUUUCAAGGAAAAUU |
| 1069 | 14215 | 24215 | UGAAAUUGUAUUUUAUCUGAAAAUACAAUUUCA |
| 1070 | 14216 | 24216 | AGUAAUUUCAAAAUUCUUAAUUUUGAAAUUACU |
| 1071 | 14217 | 24217 | CAAAAUUCUUAAAGUUCUUCUUUAAGAAUUUUG |
| 1072 | 14218 | 24218 | UGAAUUUUGGUUCUGCUCUAGAACCAAAAUUCA |
| 1073 | 14219 | 24219 | UGUCAUUUUAUAAUUAUGUAUUAUAAAAUGACA |
| 1074 | 14220 | 24220 | CCAAAUCCUGUACUGACAAAGUACAGGAUUUGG |
| 1075 | 14221 | 24221 | GGAUAACUUUUAAUAGAGAAUUAAAAGUUAUCC |
| 1076 | 14222 | 24222 | UUUAAGUCUUCUCUUAUUCAGAGAAGACUUAAA |
| 1077 | 14223 | 24223 | GAUAAUUCCAAUAUGAUCAAUAUUGGAAUUAUC |
| 1078 | 14224 | 24224 | GGAUAAAUGAACAUGGCCUAUGUUCAUUUAUCC |
| 1079 | 14225 | 24225 | CAAGGUUCAUCAUUUUCUUAAUGAUGAACCUUG |
| 1080 | 14226 | 24226 | UGGAAGUGCUAUAAAACAUUUAUAGCACUUCCA |
| 1081 | 14227 | 24227 | CCAAGUACUCUUAAAGCAAUUAAGAGUACUUGG |
| 1082 | 14228 | 24228 | UCCAAUGAUUUCCUGUUUCAGGAAAUCAUUGGA |
| 1083 | 14229 | 24229 | UAUGGUAUAUUCAUUUCCAAUGAAUAUACCAUA |
| 1084 | 14230 | 24230 | GAACAAGAUGAACUUCCCAAGUUCAUCUUGUUC |
| 1085 | 14231 | 24231 | UGAACUUCAGGAAUUUUAGAUUCCUGAAGUUCA |
| 1086 | 14232 | 24232 | AAGUCUUCUCUUAUUCCAAAUAAGAGAAGACUU |
| 1087 | 14233 | 24233 | GAAUGUUUAUACUUUGAUAAAGUAUAAACAUUC |
| 1088 | 14234 | 24234 | CCGGAAUCGUACACAAAGGGUGUACGAUUCCGG |
| 1089 | 14235 | 24235 | CAUACCUCUGCUCUUCUGAAGAGCAGAGGUAUG |
| 1090 | 14236 | 24236 | GAUCAAUUUCUUCUACCAUAGAAGAAAUUGAUC |
| 1091 | 14237 | 24237 | CAACAUUGUGUUUUGCAUUAAAACACAAUGUUG |
| 1092 | 14238 | 24238 | UAACUUUAUAAGCAUAUGCUGCUUAUAAAGUUA |
| 1093 | 14239 | 24239 | CAGGAUAACUUUUAAUAGAUAAAAGUUAUCCUG |
| 1094 | 14240 | 24240 | UUUUAUUGGUUGAUACUGUAUCAACCAAUAAAA |
| 1095 | 14241 | 24241 | UGCAACUGUUUUCUUCUGGAGAAAACAGUUGCA |
| 1096 | 14242 | 24242 | UGCUUUGAUACAACUUCCAGUUGUAUCAAAGCA |
| 1097 | 14243 | 24243 | CCAAAGCUUCUCUCUUCAAGAGAGAAGCUUUGG |
| 1098 | 14244 | 24244 | GGGAACUCCUUUCGUCUGCCGAAAGGAGUUCCC |
| 1099 | 14245 | 24245 | UAUGACAGUUCUUUGACUGAAAGAACUGUCAUA |
| 1100 | 14246 | 24246 | UUGCAGAAUAACAUGUCCAAUGUUAUUCUGCAA |
| 1101 | 14247 | 24247 | CAGAAGUCCUAUAGUUGUACUAUAGGACUUCUG |
| 1102 | 14248 | 24248 | GAUAACUUUUAAUAGAGAUUAUUAAAAGUUAUC |
| 1103 | 14249 | 24249 | ACUAAGAUUUCUUUUCCAAAAAGAAAUCUUAGU |
| 1104 | 14250 | 24250 | GAUAAAUGAACAUGGCCUGCAUGUUCAUUUAUC |
| 1105 | 14251 | 24251 | GAUGAACAUGUUGUGUCUCACAACAUGUUCAUC |
| 1106 | 14252 | 24252 | UGAUCAUCUUUUAAGUCUUUUAAAAGAUGAUCA |
| 1107 | 14253 | 24253 | GAGCAAUUCCAUUUAUCAAAAAUGGAAUUGCUC |
| 1108 | 14254 | 24254 | UGUGAAUUUUCCUUGAAAGAAGGAAAAUUCACA |
| 1109 | 14255 | 24255 | UCAAAAUUCUUAAAGUUCUUUUAAGAAUUUUGA |
| 1110 | 14256 | 24256 | UAAACUCCAGCACCGUCACGGUGCUGGAGUUUA |
| 1111 | 14257 | 24257 | UUGAUAUUGGAAGUGCUAUACUUCCAAUAUCAA |
| 1112 | 14258 | 24258 | GUGCAUUCAGUGUUACUGGAACACUGAAUGCAC |
| 1113 | 14259 | 24259 | AAUGUUUAUACUUUGAUAAAAAGUAUAAACAUU |
| 1114 | 14260 | 24260 | AAAUUGUAUUUUAUCUGGAAUAAAAUACAAUUU |
| 1115 | 14261 | 24261 | UAAGUCUUCUCUUAUUCCAUAAGAGAAGACUUA |
| 1116 | 14262 | 24262 | AGAAGUCCUAUAGUUGUAAACUAUAGGACUUCU |
| 1117 | 14263 | 24263 | CUUGCUUUUAUAGUAAUUUACUAUAAAAGCAAG |
| 1118 | 14264 | 24264 | UCAACAUUGUGUUUUGCAUAAACACAAUGUUGA |
| 1119 | 14265 | 24265 | AGGCAGUUGUUUCUACCAUAGAAACAACUGCCU |
| 1120 | 14266 | 24266 | UAUGAUCAAUUUCUUCUACAGAAAUUGAUCAUA |
| 1121 | 14267 | 24267 | GUAAUUUCAAAAUUCUUAAAAUUUUGAAAUUAC |
| 1122 | 14268 | 24268 | AUCAACAUUGUGUUUUGCAAACACAAUGUUGAU |
| 1123 | 14269 | 24269 | CAAAGUAUUCCCAAAAGGCUUGGGAAUACUUUG |
| 1124 | 14270 | 24270 | AAAACAUGGUACACUGUUUGUGUACCAUGUUUU |
| 1125 | 14271 | 24271 | ACACAGAACUGCAUCCCAGAUGCAGUUCUGUGU |
| 1126 | 14272 | 24272 | UCCAAGUACUCUUAAAGCAUAAGAGUACUUGGA |
| 1127 | 14273 | 24273 | UGUAGUAUGACAGUUCUUUACUGUCAUACUACA |
| 1128 | 14274 | 24274 | CAGAAUAGCUUUCCCUUUUGGAAAGCUAUUCUG |
| 1129 | 14275 | 24275 | GCUUUUAUAGUAAUUUCAAAUUACUAUAAAAGC |
| 1130 | 14276 | 24276 | GAAGCUACUCCAUCAUCAAGAUGGAGUAGCUUC |
| 1131 | 14277 | 24277 | UGCCACUAAUUCUAAGUAAUAGAAUUAGUGGCA |
| 1132 | 14278 | 24278 | GUAGACUCUAUGACUGUUAGUCAUAGAGUCUAC |
| 1133 | 14279 | 24279 | CUAAGAUUUCUUUUCCAAAAAAAGAAAUCUUAG |
| 1134 | 14280 | 24280 | CAAUAUUUAACCAGACUGACUGGUUAAAUAUUG |
| 1135 | 14281 | 24281 | UAGUAAUUUCAAAAUUCUUUUUUGAAAUUACUA |
| 1136 | 14282 | 24282 | GGAAAAGAGAUAAUUCCAAAUUAUCUCUUUUCC |
| 1137 | 14283 | 24283 | AAAAUGCUUGACACGAUGAGUGUCAAGCAUUUU |
| 1138 | 14284 | 24284 | UAUAUUCAUUUCCAGGAAGUGGAAAUGAAUAUA |
| 1139 | 14285 | 24285 | UAAUAAAAGCAAGUGCCACACUUGCUUUUAUUA |
| 1140 | 14286 | 24286 | UACUAUGCGUUUGUAAUCAACAAACGCAUAGUA |
| 1141 | 14287 | 24287 | GAAUUGAAAUACUCUUUCCGAGUAUUUCAAUUC |
| 1142 | 14288 | 24288 | UCAGGAUAACUUUUAAUAGAAAAGUUAUCCUGA |
| 1143 | 14289 | 24289 | UACAACUUCCAAAUACACAAUUUGGAAGUUGUA |
| 1144 | 14290 | 24290 | AAUCAUUCUCUAAUAAAAGAUUAGAGAAUGAUU |
| 1145 | 14291 | 24291 | GAAUAGCUUUCCCUUUUGAAGGGAAAGCUAUUC |
| 1146 | 14292 | 24292 | GGAAAUUCUUGUCUGUCAUAGACAAGAAUUUCC |
| 1147 | 14293 | 24293 | UUGAAUUUUGGUUCUGCUCGAACCAAAAUUCAA |
| 1148 | 14294 | 24294 | AGUGAGCUUUACAAAUAAGUUGUAAAGCUCACU |
| 1149 | 14295 | 24295 | AAACAUGGUACACUGUUUAAGUGUACCAUGUUU |
| 1150 | 14296 | 24296 | UAGACUCUAUGACUGUUACAGUCAUAGAGUCUA |
| 1151 | 14297 | 24297 | UUUUCUUGGGAGUCAUCUGGACUCCCAAGAAAA |
| 1152 | 14298 | 24298 | GUAGGCAAGGAGAUGUCCAAUCUCCUUGCCUAC |
| 1153 | 14299 | 24299 | AAAGCCACCUCCAUACCUCAUGGAGGUGGCUUU |
| 1154 | 14300 | 24300 | UAAGAUUUCUUUUCCAAACGAAAAGAAAUCUUA |
| 1155 | 14301 | 24301 | AAAGCUGCAAACUUCCUCAAAGUUUGCAGCUUU |
| 1156 | 14302 | 24302 | UCUAAUAAAAGCAAGUGCCUUGCUUUUAUUAGA |
| 1157 | 14303 | 24303 | CAAUUGUUUGUGCAUUCAGUGCACAAACAAUUG |
| 1158 | 14304 | 24304 | ACCAAAGCUUCUCUCUUCAAGAGAAGCUUUGGU |
| 1159 | 14305 | 24305 | GCAACUGUUUUCUUCUGGGAAGAAAACAGUUGC |
| 1160 | 14306 | 24306 | AUGCUUUGAUACAACUUCCUUGUAUCAAAGCAU |
| 1161 | 14307 | 24307 | AAUUCAUCUAAAUUAGCUAAAUUUAGAUGAAUU |
| 1162 | 14308 | 24308 | UGUGACGAUGUAAUAGACCAUUACAUCGUCACA |
| 1163 | 14309 | 24309 | GGUACACUGUUUAUCUGGUAUAAACAGUGUACC |
| 1164 | 14310 | 24310 | GGUUGCCUUGUACUUGACAAGUACAAGGCAACC |
| 1165 | 14311 | 24311 | CCAAGGAAAUUCUUGUCUGAAGAAUUUCCUUGG |
| 1166 | 14312 | 24312 | UUAACCAGACUGAAUCAGAUUCAGUCUGGUUAA |
| 1167 | 14313 | 24313 | UAGCAGCUAUUUCUUCUAUAGAAAUAGCUGCUA |
| 1168 | 14314 | 24314 | CCAGUUUUGUAGAUAUCCAAUCUACAAAACUGG |
| 1169 | 14315 | 24315 | AAUGAUUUCCUGUUUCCAGAACAGGAAAUCAUU |
| 1170 | 14316 | 24316 | AGACAAGAUCUCACCUACAGUGAGAUCUUGUCU |
| 1171 | 14317 | 24317 | GUGGAUUACCUUUAACCAAUAAAGGUAAUCCAC |
| 1172 | 14318 | 24318 | UGAUACUGUGAAUUUUCCUAAUUCACAGUAUCA |
| 1173 | 14319 | 24319 | AAUACUGGGACAACGCUCAGUUGUCCCAGUAUU |
| 1174 | 14320 | 24320 | CAUAAUUAAGUACUGUCUUAGUACUUAAUUAUG |
| 1175 | 14321 | 24321 | CAAACUUCCUCAGAGGUACUCUGAGGAAGUUUG |
| 1176 | 14322 | 24322 | AAGGCCAUGUUAUUUCAGAAAUAACAUGGCCUU |
| 1177 | 14323 | 24323 | UGCAAACUGUAUGCAGCUGGCAUACAGUUUGCA |
| 1178 | 14324 | 24324 | AGAAUAGCUUUCCCUUUUGGGGAAAGCUAUUCU |
| 1179 | 14325 | 24325 | UACUGUGAAUUUUCCUUGAGAAAAUUCACAGUA |
| 1180 | 14326 | 24326 | CCAACUUCAAAGAGUUCAACUCUUUGAAGUUGG |
| 1181 | 14327 | 24327 | CUGAAUUUUGAUAUUGGAAAUAUCAAAAUUCAG |
| 1182 | 14328 | 24328 | AAAGGCUGUAAGAUAUAAGAUCUUACAGCCUUU |
| 1183 | 14329 | 24329 | UUGCUUUUAUAGUAAUUUCUACUAUAAAAGCAA |
| 1184 | 14330 | 24330 | GUCCAAGUACUCUUAAAGCAAGAGUACUUGGAC |
| 1185 | 14331 | 24331 | ACACUAAUUCUGCUGUCUGAGCAGAAUUAGUGU |
| 1186 | 14332 | 24332 | CUGGCUUGCUUACUGGUAAAGUAAGCAAGCCAG |
| 1187 | 14333 | 24333 | GUUGCCUUGUACUUGACAAAAGUACAAGGCAAC |
| 1188 | 14334 | 24334 | CAGCAUUUCUGUAGGACAUCUACAGAAAUGCUG |
| 1189 | 14335 | 24335 | UUAUACUUUGAUAAGAUGCUUAUCAAAGUAUAA |
| 1190 | 14336 | 24336 | UUAAGUACUGUCUUCCUUUAAGACAGUACUUAA |
| 1191 | 14337 | 24337 | CUGCAAACUUCCUCAGAGGGAGGAAGUUUGCAG |
| 1192 | 14338 | 24338 | AAGACAGUUUCUCUUUUGGAGAGAAACUGUCUU |
| 1193 | 14339 | 24339 | AUUCAGUUUGUAGGGAGAGCCUACAAACUGAAU |
| 1194 | 14340 | 24340 | UGGAAUGUUUAUACUUUGAGUAUAAACAUUCCA |
| 1195 | 14341 | 24341 | UAACAGGGUCUUCAUGUGUUGAAGACCCUGUUA |
| 1196 | 14342 | 24342 | AGAGAUUGUUGCAUCAAAUAUGCAACAAUCUCU |
| 1197 | 14343 | 24343 | GAGUCAUCUGCAUUUGCAUAAUGCAGAUGACUC |
| 1198 | 14344 | 24344 | GAAUGUUUAUAUUUAGCAGAAAUAUAAACAUUC |
| 1199 | 14345 | 24345 | GGAACAAGAUGAACUUCCCGUUCAUCUUGUUCC |
| 1200 | 14346 | 24346 | GGUAUUUCUGCCUCUUCAGGAGGCAGAAAUACC |
| 1201 | 14347 | 24347 | CAGAACUGCAUCCCAGAAGGGGAUGCAGUUCUG |
| 1202 | 14348 | 24348 | UUCCGGUGUCCAAUAACCUAUUGGACACCGGAA |
| 1203 | 14349 | 24349 | CAUCAAUUGUUUGUGCAUUACAAACAAUUGAUG |
| 1204 | 14350 | 24350 | AAUACUCUUUCCAAGGGCUUUGGAAAGAGUAUU |
| 1205 | 14351 | 24351 | UCAAAUGCUUCAGUGUAUCACUGAAGCAUUUGA |
| 1206 | 14352 | 24352 | AAGCAAGUGCCACUAAUUCAGUGGCACUUGCUU |
| 1207 | 14353 | 24353 | UUAAUAGAGAUUGUUGCAUACAAUCUCUAUUAA |
| 1208 | 14354 | 24354 | AUAGAGAUUGUUGCAUCAAGCAACAAUCUCUAU |
| 1209 | 14355 | 24355 | GAACUCCUUUCGUCUGCUAGACGAAAGGAGUUC |
| 1210 | 14356 | 24356 | GUCAUUUUAUAAUUAUGUAAAUUAUAAAAUGAC |
| 1211 | 14357 | 24357 | AAGAAUUGAAAUACUCUUUGUAUUUCAAUUCUU |
| 1212 | 14358 | 24358 | AAAGCCUUUCUAAUUCCAAAUUAGAAAGGCUUU |
| 1213 | 14359 | 24359 | UCAAUAUUUAACCAGACUGUGGUUAAAUAUUGA |
| 1214 | 14360 | 24360 | UGGUAUAUUCAUUUCCAGGAAAUGAAUAUACCA |
| 1215 | 14361 | 24361 | UGAAACAAUUGAACGAAACGUUCAAUUGUUUCA |
| 1216 | 14362 | 24362 | AAUAUCUUGCUUUUAUAGUAAAAGCAAGAUAUU |
| 1217 | 14363 | 24363 | AGAGAUUGUCAGAUCCAAUAUCUGACAAUCUCU |
| 1218 | 14364 | 24364 | AGCAAUUCCAUUUAUCAACUAAAUGGAAUUGCU |
| 1219 | 14365 | 24365 | UUAUCAGGAUAACUUUUAAAGUUAUCCUGAUAA |
| 1220 | 14366 | 24366 | CGAAUUUCUGGCUUGCUUAAAGCCAGAAAUUCG |
| 1221 | 14367 | 24367 | UCAAGUUCAGACUGGUGAGCAGUCUGAACUUGA |
| 1222 | 14368 | 24368 | AGAGGUACUGGUUUUGUGAAAACCAGUACCUCU |
| 1223 | 14369 | 24369 | GGUAUUUUCUUUCAAGCAAUGAAAGAAAAUACC |
| 1224 | 14370 | 24370 | AGGUACUGGUUUUGUGAACCAAAACCAGUACCU |
| 1225 | 14371 | 24371 | CAGAUUCCCUCCACAGCAGGUGGAGGGAAUCUG |
| 1226 | 14372 | 24372 | UAGUCAGCAUUUCUGUAGGAGAAAUGCUGACUA |
| 1227 | 14373 | 24373 | AUUAGCACGGAGCUGGCUUAGCUCCGUGCUAAU |
| 1228 | 14374 | 24374 | UAAGAGACACAGUUUGGCCAACUGUGUCUCUUA |
| 1229 | 14375 | 24375 | GAACAUGUUGUGUCUCUAGGACACAACAUGUUC |
| 1230 | 14376 | 24376 | CAUAAGUGCAAACUGUAUGAGUUUGCACUUAUG |
| 1231 | 14377 | 24377 | CAAAUUCAGUUUGUAGGGAACAAACUGAAUUUG |
| 1232 | 14378 | 24378 | ACAAGCAGUCCUUUUACACAAAGGACUGCUUGU |
| 1233 | 14379 | 24379 | AAGUAUUCCCAAAAGGCCCUUUUGGGAAUACUU |
| 1234 | 14380 | 24380 | CAAUCACAGUAAAGGCUGUCUUUACUGUGAUUG |
| 1235 | 14381 | 24381 | AAUAGCUUUCCCUUUUGACAAGGGAAAGCUAUU |
| 1236 | 14382 | 24382 | UAUUGGUUGAUACUGUGAAAGUAUCAACCAAUA |
| 1237 | 14383 | 24383 | CAGAAAUGGCCAAUGUAAAAUUGGCCAUUUCUG |
| 1238 | 14384 | 24384 | UUGUAAUCAGAGUUUCCGUAACUCUGAUUACAA |
| 1239 | 14385 | 24385 | ACUCAGGCUUUAAUGAUCAAUUAAAGCCUGAGU |
| 1240 | 14386 | 24386 | CAGCUAUUUCUUCUAUCUUAGAAGAAAUAGCUG |
| 1241 | 14387 | 24387 | AUAUCUUGCUUUUAUAGUAUAAAAGCAAGAUAU |
| 1242 | 14388 | 24388 | ACAAGGUUCAUCAUUUUCUAUGAUGAACCUUGU |
| 1243 | 14389 | 24389 | AUUGUGUUUUGCAUUGCUGAUGCAAAACACAAU |
| 1244 | 14390 | 24390 | GCCACAAUAAAGAAUUACAUUCUUUAUUGUGGC |
| 1245 | 14391 | 24391 | GGUGGAUUACCUUUAACCAAAAGGUAAUCCACC |
| 1246 | 14392 | 24392 | CUUCUCUCUUCAAAGCUGAUUUGAAGAGAGAAG |
| 1247 | 14393 | 24393 | UGGGAUGCUUCAAUAUCCUAUUGAAGCAUCCCA |
| 1248 | 14394 | 24394 | AAAUCUUCUAAACUGUAGUAGUUUAGAAGAUUU |
| 1249 | 14395 | 24395 | AAAUUCAGUUUGUAGGGAGUACAAACUGAAUUU |
| 1250 | 14396 | 24396 | CUGUCAUUUUAUAAUUAUGUUAUAAAAUGACAG |
| 1251 | 14397 | 24397 | AAUGCUUUGAUACAACUUCUGUAUCAAAGCAUU |
| 1252 | 14398 | 24398 | UCCUCUUUAUAUUUAGCCUAAAUAUAAAGAGGA |
| 1253 | 14399 | 24399 | GCAAAUUCAUCUAAAUUAGUUAGAUGAAUUUGC |
| 1254 | 14400 | 24400 | UUCCAAAUACACAUAAGAAAUGUGUAUUUGGAA |

TABLE 2b
Nucleobase sequences of 250 C5 constructs in which the sense
and the antisense sequences of Tables 1c and 1d are combined.

| SEQ ID | Sense | Antisense | |
|---|---|---|---|
| NO. | ID | ID | 33 mer C5 Hairpin sequences |
| 1255 | 14151 | 24151 | ACACAGUUUGGCCUGGAGAAGGCCAAACUGUGU |
| 1256 | 14152 | 24152 | GGAAUCUUGAAGUCAGGAAGACUUCAAGAUUCC |
| 1257 | 14153 | 24153 | CUGGGCUUGUAGCUGGCACAGCUACAAGCCCAG |
| 1258 | 14154 | 24154 | UCAAGUAAUUAUAGUGAGUCUAUAAUUACUUGA |
| 1259 | 14155 | 24155 | AAACAGGUUUGUCUGUAUGAGACAAACCUGUUU |
| 1260 | 14156 | 24156 | GCAGACAUUUUAACACAGAGUUAAAAUGUCUGC |
| 1261 | 14157 | 24157 | ACCUGGAGCUGGUUGCCACAACCAGCUCCAGGU |
| 1262 | 14158 | 24158 | GAUAAAAUCAAGUAAUUAUUACUUGAUUUUAUC |
| 1263 | 14159 | 24159 | GACACAGUUUGGCCUGGAGGGCCAAACUGUGUC |
| 1264 | 14160 | 24160 | CGGAAUCUUGAAGUCAGGAACUUCAAGAUUCCG |
| 1265 | 14161 | 24161 | AGACAUUUUAACACAGAACGUGUUAAAAUGUCU |
| 1266 | 14162 | 24162 | UGAAGUCAGGAAAAGAGAUUUUUCCUGACUUCA |
| 1267 | 14163 | 24163 | CACAGUUUGGCCUGGAGAACAGGCCAAACUGUG |
| 1268 | 14164 | 24164 | AAUUAUAGUGAGUUAUUUUAACUCACUAUAAUU |
| 1269 | 14165 | 24165 | UCCAAGUCAGAUGUCUCUUACAUCUGACUUGGA |
| 1270 | 14166 | 24166 | UCAGGAAAAGAGAUAAUUCAUCUCUUUUCCUGA |
| 1271 | 14167 | 24167 | GGCAAGACAUAUUCUUUAAGAAUAUGUCUUGCC |
| 1272 | 14168 | 24168 | GAAGGCCAAUUUCCAGAGGGGAAAUUGGCCUUC |
| 1273 | 14169 | 24169 | CAAGUAAUUAUAGUGAGUUACUAUAAUUACUUG |
| 1274 | 14170 | 24170 | AUAAAAUCAAGUAAUUAUAUUACUUGAUUUUAU |
| 1275 | 14171 | 24171 | AUCAAGUAAUUAUAGUGAGUAUAAUUACUUGAU |
| 1276 | 14172 | 24172 | GCCAAUUUCCAGAGGAAGCCUCUGGAAAUUGGC |
| 1277 | 14173 | 24173 | AGUAAUUAUAGUGAGUUAUUCACUAUAAUUACU |
| 1278 | 14174 | 24174 | UAAAGGUACUUGUUGUUUAAACAAGUACCUUUA |
| 1279 | 14175 | 24175 | GACUGCUGUUUCAGAAUCACUGAAACAGCAGUC |
| 1280 | 14176 | 24176 | ACUGCUGUUUCAGAAUCAAUCUGAAACAGCAGU |
| 1281 | 14177 | 24177 | AUAUAAAGGUACUUGUUGUAAGUACCUUUAUAU |
| 1282 | 14178 | 24178 | UGUAAACAGUUCCUUUCAAAGGAACUGUUUACA |
| 1283 | 14179 | 24179 | GGUAACUUUGGCUGAGAGACAGCCAAAGUUACC |
| 1284 | 14180 | 24180 | UAUAGUUGUAAACAGUUCCUGUUUACAACUAUA |
| 1285 | 14181 | 24181 | ACAUAUUCUUUAACUUCAAGUUAAAGAAUAUGU |
| 1286 | 14182 | 24182 | AAGCAGUCCUUUUACACUCUAAAAGGACUGCUU |
| 1287 | 14183 | 24183 | UAGUGAGUUAUUUUGUCAAAAAAUAACUCACUA |
| 1288 | 14184 | 24184 | AGGAAGACAUCUUUGAACAAAAGAUGUCUUCCU |
| 1289 | 14185 | 24185 | GCAGUCCUUUUACACUCAAUGUAAAAGGACUGC |
| 1290 | 14186 | 24186 | AGUUAUUUUGUCAAUAUAUUUGACAAAAUAACU |
| 1291 | 14187 | 24187 | GUACAACAGAAUAUGGUAUAUAUUCUGUUGUAC |
| 1292 | 14188 | 24188 | GUUAUUUUGUCAAUAUAUGAUUGACAAAAUAAC |
| 1293 | 14189 | 24189 | CAGGCUUCAGGAAAAGAGGUUUCCUGAAGCCUG |
| 1294 | 14190 | 24190 | AGGAAAAGAGAUAAUUCCAUUAUCUCUUUUCCU |
| 1295 | 14191 | 24191 | UGUUACAGCAAUAUAAAGGAUAUUGCUGUAACA |
| 1296 | 14192 | 24192 | UAUAAGCAUAUGCAAUCUCUGCAUAUGCUUAUA |
| 1297 | 14193 | 24193 | CAUAUUCUUUAACUUCAAAAGUUAAAGAAUAUG |
| 1298 | 14194 | 24194 | AAGACAUCUUUGAACACCUUUCAAAGAUGUCUU |
| 1299 | 14195 | 24195 | CCAGGAAGACAUCUUUGAAAGAUGUCUUCCUGG |
| 1300 | 14196 | 24196 | UACAGCAAUAUAAAGGUACUUUAUAUUGCUGUA |
| 1301 | 14197 | 24197 | CAUUGUCAUAGGUUAUUGGAACCUAUGACAAUG |
| 1302 | 14198 | 24198 | UGAGUUAUUUUGUCAAUAUGACAAAAUAACUCA |
| 1303 | 14199 | 24199 | AGUGAGUUAUUUUGUCAAUCAAAAUAACUCACU |
| 1304 | 14200 | 24200 | GUGAGUUAUUUUGUCAAUAACAAAAUAACUCAC |
| 1305 | 14201 | 24201 | GAAUUUUCCUUGAAAGAUCUUCAAGGAAAAUUC |
| 1306 | 14202 | 24202 | ACUGUUACAGCAAUAUAAAAUUGCUGUAACAGU |
| 1307 | 14203 | 24203 | AAAUCCAUUGUCAUAGGUUAUGACAAUGGAUUU |
| 1308 | 14204 | 24204 | AAUCCAUUGUCAUAGGUUAUAUGACAAUGGAUU |
| 1309 | 14205 | 24205 | GAGAAAUCCAUUGUCAUAGACAAUGGAUUUCUC |
| 1310 | 14206 | 24206 | AAGACAUAUUCUUUAACUUAAAGAAUAUGUCUU |
| 1311 | 14207 | 24207 | AAGUGCAGAUUCCCUCCACGGGAAUCUGCACUU |
| 1312 | 14208 | 24208 | AUCCAUUGUCAUAGGUUAUCUAUGACAAUGGAU |
| 1313 | 14209 | 24209 | AGACAUCUUUGAACACCUUGUUCAAAGAUGUCU |
| 1314 | 14210 | 24210 | CCAUUGUCAUAGGUUAUUGACCUAUGACAAUGG |
| 1315 | 14211 | 24211 | UGAAGAGAAAUCCAUUGUCUGGAUUUCUCUUCA |
| 1316 | 14212 | 24212 | GACAUAUUCUUUAACUUCAUUAAAGAAUAUGUC |
| 1317 | 14213 | 24213 | CAGUCCUUUUACACUCAAAGUGUAAAAGGACUG |
| 1318 | 14214 | 24214 | AAUUUUCCUUGAAAGAUCCUUUCAAGGAAAAUU |
| 1319 | 14215 | 24215 | UGAAAUUGUAUUUUAUCUGAAAAUACAAUUUCA |
| 1320 | 14216 | 24216 | AGUAAUUUCAAAAUUCUUAAUUUUGAAAUUACU |
| 1321 | 14217 | 24217 | CAAAAUUCUUAAAGUUCUUCUUUAAGAAUUUUG |
| 1322 | 14218 | 24218 | UGAAUUUUGGUUCUGCUCUAGAACCAAAAUUCA |
| 1323 | 14219 | 24219 | UGUCAUUUUAUAAUUAUGUAUUAUAAAAUGACA |
| 1324 | 14220 | 24220 | CCAAAUCCUGUACUGACAAAGUACAGGAUUUGG |
| 1325 | 14221 | 24221 | GGAUAACUUUUAAUAGAGAAUUAAAAGUUAUCC |
| 1326 | 14222 | 24222 | UUUAAGUCUUCUCUUAUUCAGAGAAGACUUAAA |
| 1327 | 14223 | 24223 | GAUAAUUCCAAUAUGAUCAAUAUUGGAAUUAUC |
| 1328 | 14224 | 24224 | GGAUAAAUGAACAUGGCCUAUGUUCAUUUAUCC |
| 1329 | 14225 | 24225 | CAAGGUUCAUCAUUUUCUUAAUGAUGAACCUUG |
| 1330 | 14226 | 24226 | UGGAAGUGCUAUAAAACAUUUAUAGCACUUCCA |
| 1331 | 14227 | 24227 | CCAAGUACUCUUAAAGCAAUUAAGAGUACUUGG |
| 1332 | 14228 | 24228 | UCCAAUGAUUUCCUGUUUCAGGAAAUCAUUGGA |
| 1333 | 14229 | 24229 | UAUGGUAUAUUCAUUUCCAAUGAAUAUACCAUA |
| 1334 | 14230 | 24230 | GAACAAGAUGAACUUCCCAAGUUCAUCUUGUUC |
| 1335 | 14231 | 24231 | UGAACUUCAGGAAUUUUAGAUUCCUGAAGUUCA |
| 1336 | 14232 | 24232 | AAGUCUUCUCUUAUUCCAAAUAAGAGAAGACUU |
| 1337 | 14233 | 24233 | GAAUGUUUAUACUUUGAUAAAGUAUAAACAUUC |
| 1338 | 14234 | 24234 | CCGGAAUCGUACACAAAGGGUGUACGAUUCCGG |
| 1339 | 14235 | 24235 | CAUACCUCUGCUCUUCUGAAGAGCAGAGGUAUG |
| 1340 | 14236 | 24236 | GAUCAAUUUCUUCUACCAUAGAAGAAAUUGAUC |
| 1341 | 14237 | 24237 | CAACAUUGUGUUUUGCAUUAAAACACAAUGUUG |
| 1342 | 14238 | 24238 | UAACUUUAUAAGCAUAUGCUGCUUAUAAAGUUA |
| 1343 | 14239 | 24239 | CAGGAUAACUUUUAAUAGAUAAAAGUUAUCCUG |
| 1344 | 14240 | 24240 | UUUUAUUGGUUGAUACUGUAUCAACCAAUAAAA |
| 1345 | 14241 | 24241 | UGCAACUGUUUUCUUCUGGAGAAAACAGUUGCA |
| 1346 | 14242 | 24242 | UGCUUUGAUACAACUUCCAGUUGUAUCAAAGCA |
| 1347 | 14243 | 24243 | CCAAAGCUUCUCUCUUCAAGAGAGAAGCUUUGG |
| 1348 | 14244 | 24244 | GGGAACUCCUUUCGUCUGCCGAAAGGAGUUCCC |
| 1349 | 14245 | 24245 | UAUGACAGUUCUUUGACUGAAAGAACUGUCAUA |
| 1350 | 14246 | 24246 | UUGCAGAAUAACAUGUCCAAUGUUAUUCUGCAA |
| 1351 | 14247 | 24247 | CAGAAGUCCUAUAGUUGUACUAUAGGACUUCUG |
| 1352 | 14248 | 24248 | GAUAACUUUUAAUAGAGAUUAUUAAAAGUUAUC |
| 1353 | 14249 | 24249 | ACUAAGAUUUCUUUUCCAAAAAGAAAUCUUAGU |
| 1354 | 14250 | 24250 | GAUAAAUGAACAUGGCCUGCAUGUUCAUUUAUC |
| 1355 | 14251 | 24251 | GAUGAACAUGUUGUGUCUCACAACAUGUUCAUC |
| 1356 | 14252 | 24252 | UGAUCAUCUUUUAAGUCUUUUAAAAGAUGAUCA |
| 1357 | 14253 | 24253 | GAGCAAUUCCAUUUAUCAAAAAUGGAAUUGCUC |
| 1358 | 14254 | 24254 | UGUGAAUUUUCCUUGAAAGAAGGAAAAUUCACA |
| 1359 | 14255 | 24255 | UCAAAAUUCUUAAAGUUCUUUUAAGAAUUUUGA |
| 1360 | 14256 | 24256 | UAAACUCCAGCACCGUCACGGUGCUGGAGUUUA |
| 1361 | 14257 | 24257 | UUGAUAUUGGAAGUGCUAUACUUCCAAUAUCAA |
| 1362 | 14258 | 24258 | GUGCAUUCAGUGUUACUGGAACACUGAAUGCAC |
| 1363 | 14259 | 24259 | AAUGUUUAUACUUUGAUAAAAAGUAUAAACAUU |
| 1364 | 14260 | 24260 | AAAUUGUAUUUUAUCUGGAAUAAAAUACAAUUU |
| 1365 | 14261 | 24261 | UAAGUCUUCUCUUAUUCCAUAAGAGAAGACUUA |
| 1366 | 14262 | 24262 | AGAAGUCCUAUAGUUGUAAACUAUAGGACUUCU |
| 1367 | 14263 | 24263 | CUUGCUUUUAUAGUAAUUUACUAUAAAAGCAAG |
| 1368 | 14264 | 24264 | UCAACAUUGUGUUUUGCAUAAACACAAUGUUGA |
| 1369 | 14265 | 24265 | AGGCAGUUGUUUCUACCAUAGAAACAACUGCCU |
| 1370 | 14266 | 24266 | UAUGAUCAAUUUCUUCUACAGAAAUUGAUCAUA |
| 1371 | 14267 | 24267 | GUAAUUUCAAAAUUCUUAAAAUUUUGAAAUUAC |
| 1372 | 14268 | 24268 | AUCAACAUUGUGUUUUGCAAACACAAUGUUGAU |
| 1373 | 14269 | 24269 | CAAAGUAUUCCCAAAAGGCUUGGGAAUACUUUG |
| 1374 | 14270 | 24270 | AAAACAUGGUACACUGUUUGUGUACCAUGUUUU |
| 1375 | 14271 | 24271 | ACACAGAACUGCAUCCCAGAUGCAGUUCUGUGU |
| 1376 | 14272 | 24272 | UCCAAGUACUCUUAAAGCAUAAGAGUACUUGGA |
| 1377 | 14273 | 24273 | UGUAGUAUGACAGUUCUUUACUGUCAUACUACA |
| 1378 | 14274 | 24274 | CAGAAUAGCUUUCCCUUUUGGAAAGCUAUUCUG |
| 1379 | 14275 | 24275 | GCUUUUAUAGUAAUUUCAAAUUACUAUAAAAGC |
| 1380 | 14276 | 24276 | GAAGCUACUCCAUCAUCAAGAUGGAGUAGCUUC |
| 1381 | 14277 | 24277 | UGCCACUAAUUCUAAGUAAUAGAAUUAGUGGCA |
| 1382 | 14278 | 24278 | GUAGACUCUAUGACUGUUAGUCAUAGAGUCUAC |
| 1383 | 14279 | 24279 | CUAAGAUUUCUUUUCCAAAAAAAGAAAUCUUAG |
| 1384 | 14280 | 24280 | CAAUAUUUAACCAGACUGACUGGUUAAAUAUUG |
| 1385 | 14281 | 24281 | UAGUAAUUUCAAAAUUCUUUUUUGAAAUUACUA |
| 1386 | 14282 | 24282 | GGAAAAGAGAUAAUUCCAAAUUAUCUCUUUUCC |
| 1387 | 14283 | 24283 | AAAAUGCUUGACACGAUGAGUGUCAAGCAUUUU |
| 1388 | 14284 | 24284 | UAUAUUCAUUUCCAGGAAGUGGAAAUGAAUAUA |
| 1389 | 14285 | 24285 | UAAUAAAAGCAAGUGCCACACUUGCUUUUAUUA |
| 1390 | 14286 | 24286 | UACUAUGCGUUUGUAAUCAACAAACGCAUAGUA |
| 1391 | 14287 | 24287 | GAAUUGAAAUACUCUUUCCGAGUAUUUCAAUUC |
| 1392 | 14288 | 24288 | UCAGGAUAACUUUUAAUAGAAAAGUUAUCCUGA |
| 1393 | 14289 | 24289 | UACAACUUCCAAAUACACAAUUUGGAAGUUGUA |
| 1394 | 14290 | 24290 | AAUCAUUCUCUAAUAAAAGAUUAGAGAAUGAUU |
| 1395 | 14291 | 24291 | GAAUAGCUUUCCCUUUUGAAGGGAAAGCUAUUC |
| 1396 | 14292 | 24292 | GGAAAUUCUUGUCUGUCAUAGACAAGAAUUUCC |
| 1397 | 14293 | 24293 | UUGAAUUUUGGUUCUGCUCGAACCAAAAUUCAA |
| 1398 | 14294 | 24294 | AGUGAGCUUUACAAAUAAGUUGUAAAGCUCACU |
| 1399 | 14295 | 24295 | AAACAUGGUACACUGUUUAAGUGUACCAUGUUU |
| 1400 | 14296 | 24296 | UAGACUCUAUGACUGUUACAGUCAUAGAGUCUA |
| 1401 | 14297 | 24297 | UUUUCUUGGGAGUCAUCUGGACUCCCAAGAAAA |
| 1402 | 14298 | 24298 | GUAGGCAAGGAGAUGUCCAAUCUCCUUGCCUAC |
| 1403 | 14299 | 24299 | AAAGCCACCUCCAUACCUCAUGGAGGUGGCUUU |
| 1404 | 14300 | 24300 | UAAGAUUUCUUUUCCAAACGAAAAGAAAUCUUA |
| 1405 | 14301 | 24301 | AAAGCUGCAAACUUCCUCAAAGUUUGCAGCUUU |
| 1406 | 14302 | 24302 | UCUAAUAAAAGCAAGUGCCUUGCUUUUAUUAGA |
| 1407 | 14303 | 24303 | CAAUUGUUUGUGCAUUCAGUGCACAAACAAUUG |
| 1408 | 14304 | 24304 | ACCAAAGCUUCUCUCUUCAAGAGAAGCUUUGGU |
| 1409 | 14305 | 24305 | GCAACUGUUUUCUUCUGGGAAGAAAACAGUUGC |
| 1410 | 14306 | 24306 | AUGCUUUGAUACAACUUCCUUGUAUCAAAGCAU |
| 1411 | 14307 | 24307 | AAUUCAUCUAAAUUAGCUAAAUUUAGAUGAAUU |
| 1412 | 14308 | 24308 | UGUGACGAUGUAAUAGACCAUUACAUCGUCACA |
| 1413 | 14309 | 24309 | GGUACACUGUUUAUCUGGUAUAAACAGUGUACC |
| 1414 | 14310 | 24310 | GGUUGCCUUGUACUUGACAAGUACAAGGCAACC |
| 1415 | 14311 | 24311 | CCAAGGAAAUUCUUGUCUGAAGAAUUUCCUUGG |
| 1416 | 14312 | 24312 | UUAACCAGACUGAAUCAGAUUCAGUCUGGUUAA |
| 1417 | 14313 | 24313 | UAGCAGCUAUUUCUUCUAUAGAAAUAGCUGCUA |
| 1418 | 14314 | 24314 | CCAGUUUUGUAGAUAUCCAAUCUACAAAACUGG |
| 1419 | 14315 | 24315 | AAUGAUUUCCUGUUUCCAGAACAGGAAAUCAUU |
| 1420 | 14316 | 24316 | AGACAAGAUCUCACCUACAGUGAGAUCUUGUCU |
| 1421 | 14317 | 24317 | GUGGAUUACCUUUAACCAAUAAAGGUAAUCCAC |
| 1422 | 14318 | 24318 | UGAUACUGUGAAUUUUCCUAAUUCACAGUAUCA |
| 1423 | 14319 | 24319 | AAUACUGGGACAACGCUCAGUUGUCCCAGUAUU |
| 1424 | 14320 | 24320 | CAUAAUUAAGUACUGUCUUAGUACUUAAUUAUG |
| 1425 | 14321 | 24321 | CAAACUUCCUCAGAGGUACUCUGAGGAAGUUUG |
| 1426 | 14322 | 24322 | AAGGCCAUGUUAUUUCAGAAAUAACAUGGCCUU |
| 1427 | 14323 | 24323 | UGCAAACUGUAUGCAGCUGGCAUACAGUUUGCA |
| 1428 | 14324 | 24324 | AGAAUAGCUUUCCCUUUUGGGGAAAGCUAUUCU |
| 1429 | 14325 | 24325 | UACUGUGAAUUUUCCUUGAGAAAAUUCACAGUA |
| 1430 | 14326 | 24326 | CCAACUUCAAAGAGUUCAACUCUUUGAAGUUGG |
| 1431 | 14327 | 24327 | CUGAAUUUUGAUAUUGGAAAUAUCAAAAUUCAG |
| 1432 | 14328 | 24328 | AAAGGCUGUAAGAUAUAAGAUCUUACAGCCUUU |
| 1433 | 14329 | 24329 | UUGCUUUUAUAGUAAUUUCUACUAUAAAAGCAA |
| 1434 | 14330 | 24330 | GUCCAAGUACUCUUAAAGCAAGAGUACUUGGAC |
| 1435 | 14331 | 24331 | ACACUAAUUCUGCUGUCUGAGCAGAAUUAGUGU |
| 1436 | 14332 | 24332 | CUGGCUUGCUUACUGGUAAAGUAAGCAAGCCAG |
| 1437 | 14333 | 24333 | GUUGCCUUGUACUUGACAAAAGUACAAGGCAAC |
| 1438 | 14334 | 24334 | CAGCAUUUCUGUAGGACAUCUACAGAAAUGCUG |
| 1439 | 14335 | 24335 | UUAUACUUUGAUAAGAUGCUUAUCAAAGUAUAA |
| 1440 | 14336 | 24336 | UUAAGUACUGUCUUCCUUUAAGACAGUACUUAA |
| 1441 | 14337 | 24337 | CUGCAAACUUCCUCAGAGGGAGGAAGUUUGCAG |
| 1442 | 14338 | 24338 | AAGACAGUUUCUCUUUUGGAGAGAAACUGUCUU |
| 1443 | 14339 | 24339 | AUUCAGUUUGUAGGGAGAGCCUACAAACUGAAU |
| 1444 | 14340 | 24340 | UGGAAUGUUUAUACUUUGAGUAUAAACAUUCCA |
| 1445 | 14341 | 24341 | UAACAGGGUCUUCAUGUGUUGAAGACCCUGUUA |
| 1446 | 14342 | 24342 | AGAGAUUGUUGCAUCAAAUAUGCAACAAUCUCU |
| 1447 | 14343 | 24343 | GAGUCAUCUGCAUUUGCAUAAUGCAGAUGACUC |
| 1448 | 14344 | 24344 | GAAUGUUUAUAUUUAGCAGAAAUAUAAACAUUC |
| 1449 | 14345 | 24345 | GGAACAAGAUGAACUUCCCGUUCAUCUUGUUCC |
| 1450 | 14346 | 24346 | GGUAUUUCUGCCUCUUCAGGAGGCAGAAAUACC |
| 1451 | 14347 | 24347 | CAGAACUGCAUCCCAGAAGGGGAUGCAGUUCUG |
| 1452 | 14348 | 24348 | UUCCGGUGUCCAAUAACCUAUUGGACACCGGAA |
| 1453 | 14349 | 24349 | CAUCAAUUGUUUGUGCAUUACAAACAAUUGAUG |
| 1454 | 14350 | 24350 | AAUACUCUUUCCAAGGGCUUUGGAAAGAGUAUU |
| 1455 | 14351 | 24351 | UCAAAUGCUUCAGUGUAUCACUGAAGCAUUUGA |
| 1456 | 14352 | 24352 | AAGCAAGUGCCACUAAUUCAGUGGCACUUGCUU |
| 1457 | 14353 | 24353 | UUAAUAGAGAUUGUUGCAUACAAUCUCUAUUAA |
| 1458 | 14354 | 24354 | AUAGAGAUUGUUGCAUCAAGCAACAAUCUCUAU |
| 1459 | 14355 | 24355 | GAACUCCUUUCGUCUGCUAGACGAAAGGAGUUC |
| 1460 | 14356 | 24356 | GUCAUUUUAUAAUUAUGUAAAUUAUAAAAUGAC |
| 1461 | 14357 | 24357 | AAGAAUUGAAAUACUCUUUGUAUUUCAAUUCUU |
| 1462 | 14358 | 24358 | AAAGCCUUUCUAAUUCCAAAUUAGAAAGGCUUU |
| 1463 | 14359 | 24359 | UCAAUAUUUAACCAGACUGUGGUUAAAUAUUGA |
| 1464 | 14360 | 24360 | UGGUAUAUUCAUUUCCAGGAAAUGAAUAUACCA |
| 1465 | 14361 | 24361 | UGAAACAAUUGAACGAAACGUUCAAUUGUUUCA |
| 1466 | 14362 | 24362 | AAUAUCUUGCUUUUAUAGUAAAAGCAAGAUAUU |
| 1467 | 14363 | 24363 | AGAGAUUGUCAGAUCCAAUAUCUGACAAUCUCU |
| 1468 | 14364 | 24364 | AGCAAUUCCAUUUAUCAACUAAAUGGAAUUGCU |
| 1469 | 14365 | 24365 | UUAUCAGGAUAACUUUUAAAGUUAUCCUGAUAA |
| 1470 | 14366 | 24366 | CGAAUUUCUGGCUUGCUUAAAGCCAGAAAUUCG |
| 1471 | 14367 | 24367 | UCAAGUUCAGACUGGUGAGCAGUCUGAACUUGA |
| 1472 | 14368 | 24368 | AGAGGUACUGGUUUUGUGAAAACCAGUACCUCU |
| 1473 | 14369 | 24369 | GGUAUUUUCUUUCAAGCAAUGAAAGAAAAUACC |
| 1474 | 14370 | 24370 | AGGUACUGGUUUUGUGAACCAAAACCAGUACCU |
| 1475 | 14371 | 24371 | CAGAUUCCCUCCACAGCAGGUGGAGGGAAUCUG |
| 1476 | 14372 | 24372 | UAGUCAGCAUUUCUGUAGGAGAAAUGCUGACUA |
| 1477 | 14373 | 24373 | AUUAGCACGGAGCUGGCUUAGCUCCGUGCUAAU |
| 1478 | 14374 | 24374 | UAAGAGACACAGUUUGGCCAACUGUGUCUCUUA |
| 1479 | 14375 | 24375 | GAACAUGUUGUGUCUCUAGGACACAACAUGUUC |
| 1480 | 14376 | 24376 | CAUAAGUGCAAACUGUAUGAGUUUGCACUUAUG |
| 1481 | 14377 | 24377 | CAAAUUCAGUUUGUAGGGAACAAACUGAAUUUG |
| 1482 | 14378 | 24378 | ACAAGCAGUCCUUUUACACAAAGGACUGCUUGU |
| 1483 | 14379 | 24379 | AAGUAUUCCCAAAAGGCCCUUUUGGGAAUACUU |
| 1484 | 14380 | 24380 | CAAUCACAGUAAAGGCUGUCUUUACUGUGAUUG |
| 1485 | 14381 | 24381 | AAUAGCUUUCCCUUUUGACAAGGGAAAGCUAUU |
| 1486 | 14382 | 24382 | UAUUGGUUGAUACUGUGAAAGUAUCAACCAAUA |
| 1487 | 14383 | 24383 | CAGAAAUGGCCAAUGUAAAAUUGGCCAUUUCUG |
| 1488 | 14384 | 24384 | UUGUAAUCAGAGUUUCCGUAACUCUGAUUACAA |
| 1489 | 14385 | 24385 | ACUCAGGCUUUAAUGAUCAAUUAAAGCCUGAGU |
| 1490 | 14386 | 24386 | CAGCUAUUUCUUCUAUCUUAGAAGAAAUAGCUG |
| 1491 | 14387 | 24387 | AUAUCUUGCUUUUAUAGUAUAAAAGCAAGAUAU |
| 1492 | 14388 | 24388 | ACAAGGUUCAUCAUUUUCUAUGAUGAACCUUGU |
| 1493 | 14389 | 24389 | AUUGUGUUUUGCAUUGCUGAUGCAAAACACAAU |
| 1494 | 14390 | 24390 | GCCACAAUAAAGAAUUACAUUCUUUAUUGUGGC |
| 1495 | 14391 | 24391 | GGUGGAUUACCUUUAACCAAAAGGUAAUCCACC |
| 1496 | 14392 | 24392 | CUUCUCUCUUCAAAGCUGAUUUGAAGAGAGAAG |
| 1497 | 14393 | 24393 | UGGGAUGCUUCAAUAUCCUAUUGAAGCAUCCCA |
| 1498 | 14394 | 24394 | AAAUCUUCUAAACUGUAGUAGUUUAGAAGAUUU |
| 1499 | 14395 | 24395 | AAAUUCAGUUUGUAGGGAGUACAAACUGAAUUU |
| 1500 | 14396 | 24396 | CUGUCAUUUUAUAAUUAUGUUAUAAAAUGACAG |
| 1501 | 14397 | 24397 | AAUGCUUUGAUACAACUUCUGUAUCAAAGCAUU |
| 1502 | 14398 | 24398 | UCCUCUUUAUAUUUAGCCUAAAUAUAAAGAGGA |
| 1503 | 14399 | 24399 | GCAAAUUCAUCUAAAUUAGUUAGAUGAAUUUGC |
| 1504 | 14400 | 24400 | UUCCAAAUACACAUAAGAAAUGUGUAUUUGGAA |

TABLE 3a
Modified CFB hairpin constructs

| SEQ ID | Construct | |
|---|---|---|
| No. | ID NO: | |
| 1505 | 1 | PmU.fA.mG.mA.mA.mA.mA.mC.mC.mC.mA.mA.mA.fU.mC |
| | | .mC.mU.mC.mA.mG.mA.fU.fU.fU.mG.mG.mG.mU.mU.mU |
| | | .mU.mC.mU.mA |
| 1506 | 2 | PmU.fC.mU.mG.mU.mC.mU.mG.mA.mU.mC.mC.mA.fU.mC |
| | | .mU.mA.mG.mC.mG.mA.fU.fG.fG.mA.mU.mC.mA.mG.mA |
| | | .mC.mA.mG.mA |
| 1507 | 3 | PmU.fA.mC.mC.mA.mU.mG.mC.mC.mA.mC.mA.mG.fA.mG |
| | | .mA.mC.mU.mC.mC.mU.fC.fU.fG.mU.mG.mG.mC.mA.mU |
| | | .mG.mG.mU.mA |
| 1508 | 4 | PmU.fA.mU.mC.mC.mA.mU.mC.mU.mA.mG.mC.mA.fC.mC |
| | | .mA.mG.mG.mU.mG.mG.fU.fG.fC.mU.mA.mG.mA.mU.mG |
| | | .mG.mA.mU.mA |
| 1509 | 5 | PmU.fA.mA.mA.mC.mC.mC.mA.mA.mA.mU.mC.mC.fU.mC |
| | | .mA.mU.mC.mU.mG.mA.fG.fG.fA.mU.mU.mU.mG.mG.mG |
| | | .mU.mU.mU.mA |
| 1510 | 6 | PmU.fC.mC.mA.mU.mC.mU.mA.mG.mC.mA.mC.mC.fA.mG |
| | | .mG.mU.mA.mG.mC.mU.fG.fG.fU.mG.mC.mU.mA.mG.mA |
| | | .mU.mG.mG.mA |
| 1511 | 7 | PmU.f.A.mA.m.A.mA.mC.mC.mC.mA.mA.mA.mU.mC.fC. |
| | | mU.mC.mA.mU.mC.mA.mG.fG.fA.fU.mU.mU.mG.mG.mG. |
| | | mU.mU.mU.mU.mA |
| 1512 | 8 | PmU.fG.mU.mC.mU.mG.mA.mU.mC.mC.mA.mU.mC.fU.mA |
| | | .mG.mC.mA.mC.mU.mA.fG.fA.fU.mG.mG.mA.mU.mC.mA |
| | | .mG.mA.mC.mA |
| 1513 | 9 | PmU.fA.mC.mC.mC.mA.m.A.mA.mU.mC.mC.mU.mC.fA.m |
| | | U.mC.mU.mU.mG.mA.mU.fG.fA.fG.mG.mA.mU.mU.mU.m |
| | | G.mG.mG.mU.mA |
| 1514 | 10 | PmU.fU.mC.mC.mA.mU.mC.mU.mA.mG.mC.mA.mC.fC.mA |
| | | .mG.mG.mU.mA.mU.mG.fG.fU.fG.mC.mU.mA.mG.mA.mU |
| | | .mG.mG.mA.mA |
| 1515 | 11 | PmU.f.A.m.A.mC.mC.mC.mA.mA.mA.mU.mC.mC.mU.fC. |
| | | mA.mU.mC.mU.mU.mU.mG.fA.fG.fG.mA.mU.mU.mU.mG. |
| | | mG.mG.mU.mU.mA |
| 1516 | 12 | PmU.fC.mA.mU.mG.mC.mC.mA.mC.mA.mG.mA.mG.fA.mC |
| | | .mU.mC.mA.mG.mG.mU.fC.fU.fC.mU.mG.mU.mG.mG.mC |
| | | .mA.mU.mG.mA |
| 1517 | 13 | PmU.fU.mG.mC.mC.mA.mC.mA.mG.mA.mG.mA.mC.fU.mC |
| | | .mA.mG.mA.mG.mG.mA.fG.fU.fC.mU.mC.mU.mG.mU.mG |
| | | .mG.mC.mA.mA |
| 1518 | 14 | PmU.fA.mU.mG.mA.mU.mG.mA.mC.mA.mU.mG.mG.fC.mG |
| | | .mG.mG.mU.mG.mC.mG.fC.fC.fA.mU.mG.mU.mC.mA.mU |
| | | .mC.mA.mU.mA |
| 1519 | 15 | PmU.fU.mC.mC.mA.mU.mA.mU.mC.mC.mU.mU.mG.fA.mC |
| | | .mU.mU.mU.mG.mG.mU.fC.fA.fA.mG.mG.mA.mU.mA.mU |
| | | .mG.mG.mA.mA |
| 1520 | 16 | PmU.fA.mC.mA.mC.mC.mA.mA.mC.mU.mU.mG.mA.fA.mU |
| | | .mG.mA.mA.mA.mA.mU.fU.fC.fA.mA.mG.mU.mU.mG.mG |
| | | .mU.mG.mU.mA |
| 1521 | 17 | PmU.fG.mC.mC.mA.mC.mA.mG.mA.mG.mA.mC.mU.fC.mA |
| | | .mG.mA.mG.mA.mU.mG.fA.fG.fU.mC.mU.mC.mU.mG.mU |
| | | .mG.mG.mC.mA |
| 1522 | 18 | PmU.fC.mC.mA.mU.mG.mC.mC.mA.mC.mA.mG.mA.fG.mA |
| | | .mC.mU.mC.mA.mU.mC.fU.fC.fU.mG.mU.mG.mG.mC.mA |
| | | .mU.mG.mG.mA |
| 1523 | 19 | PmU.fC.mA.mU.mC.mU.mA.mG.mC.mA.mC.mC.mA.fG.mG |
| | | .mU.mA.mG.mA.mC.mC.fU.fG.fG.mU.mG.mC.mU.mA.mG |
| | | .mA.mU.mG.mA |
| 1524 | 20 | PmU.fU.mU.mC.mC.mA.mU.mA.mU.mC.mC.mU.mU.fG.mA |
| | | .mC.mU.mU.mU.mU.mC.fA.fA.fG.mG.mA.mU.mA.mU.mG |
| | | .mG.mA.mA.mA |
| 1525 | 21 | PmU.fG.mA.mU.mC.mC.mA.mU.mC.mU.mA.mG.mC.fA.mC |
| | | .mC.mA.mG.mG.mG.mU.fG.fC.fU.mA.mG.mA.mU.mG.mG |
| | | .mA.mU.mC.mA |
| 1526 | 22 | PmU.fC.mC.mA.mC.mA.mG.mA.mG.mA.mC.mU.mC.fA.mG |
| | | .mA.mG.mA.mC.mC.mU.fG.fA.fG.mU.mC.mU.mC.mU.mG |
| | | .mU.mG.mG.mA |
| 1527 | 23 | PmU.fU.mG.mA.mU.mC.mC.mA.mU.mC.mU.mA.mG.fC.mA |
| | | .mC.mC.mA.mG.mU.mG.fC.fU.fA.mG.mA.mU.mG.mG.mA |
| | | .mU.mC.mA.mA |
| 1528 | 24 | PmU.fA.mC.mC.mU.mC.mC.mU.mU.mC.mC.mG.mA.fG.mU |
| | | .mC.mA.mG.mC.mA.mC.fU.fC.fG.mG.mA.mA.mG.mG.mA |
| | | .mG.mG.mU.mA |
| 1529 | 25 | PmU.fU.mC.mU.mG.mA.mU.mC.mC.mA.mU.mC.mU.fA.mG |
| | | .mC.mA.mC.mC.mC.mU.fA.fG.fA.mU.mG.mG.mA.mU.mC |
| | | .mA.mG.mA.mA |
| 1530 | 26 | PmU.fU.mC.mU.mU.mG.mG.mC.mA.mG.mG.mA.mA.fG.mG |
| | | .mC.mU.mC.mC.mC.mC.fU.fU.fC.mC.mU.mG.mC.mC.mA |
| | | .mA.mG.mA.mA |
| 1531 | 27 | PmU.fU.mC.mU.mA.mG.mC.mA.mC.mC.mA.mG.mG.fU.mA |
| | | .mG.mA.mU.mG.mU.mA.fC.fC.fU.mG.mG.mU.mG.mC.mU |
| | | .mA.mG.mA.mA |
| 1532 | 28 | PmU.fA.m.A.mG.mU.mA.mC.mU.mC.mA.mG.mA.mC.fA.m |
| | | C.mC.mA.mC.mA.mG.mU.fG.fU.fC.mU.mG.mA.mG.mU.m |
| | | A.mC.mU.mU.mA |
| 1533 | 29 | PmU.fU.mG.mU.mC.mU.mG.mA.mU.mC.mC.mA.mU.fC.mU |
| | | .mA.mG.mC.mA.mA.mG.fA.fU.fG.mG.mA.mU.mC.mA.mG |
| | | .mA.mC.mA.mA |
| 1534 | 30 | PmU.fA.mU.mC.mU.mA.mG.mC.mA.mC.mC.mA.mG.fG.mU |
| | | .mA.mG.mA.mU.mA.mC.fC.fU.fG.mG.mU.mG.mC.mU.mA |
| | | .mG.mA.mU.mA |
| 1535 | 31 | PmU.fC.mA.mA.mA.mU.mC.mC.mU.mC.mA.mU.mC.fU.mU |
| | | .mG.mG.mA.mG.mA.m.A.fG.fA.fU.mG.mA.mG.mG.mA.m |
| | | U.mU.mU.mG.mA |
| 1536 | 32 | PmU.fA.mU.mA.mG.mU.mC.mA.mU.mA.mA.mA.mA.fU.mU |
| | | .mC.mA.mG.mG.mA.mA.fU.fU.fU.mU.mA.mU.mG.mA.mC |
| | | .mU.mA.mU.mA |
| 1537 | 33 | PmU.fA.mG.mG.mA.mU.mG.mA.mU.mG.mA.mC.mA.fU.mG |
| | | .mG.mC.mG.mG.mC.mA.fU.fG.fU.mC.mA.mU.mC.mA.mU |
| | | .mC.mC.mU.mA |
| 1538 | 34 | PmU.fC.mA.mA.mU.mC.mU.mG.mU.mG.mU.mU.mC.fU.mG |
| | | .mG.mC.mA.mC.mC.mA.fG.fA.fA.mC.mA.mC.mA.mG.mA |
| | | .mU.mU.mG.mA |
| 1539 | 35 | PmU.fU.mG.mA.mG.mC.mU.mU.mG.mA.mU.mC.mA.fG.mG |
| | | .mG.mC.mA.mA.mC.mC.fU.fG.fA.mU.mC.mA.mA.mG.mC |
| | | .mU.mC.mA.mA |
| 1540 | 36 | PmU.fA.mG.mU.mG.mG.mA.mA.mA.mG.mA.mG.m.A.fU.m |
| | | C.mU.mC.mA.mU.mG.mA.fU.fC.fU.mC.mU.mU.mU.mC.m |
| | | C.mA.mC.mU.mA |
| 1541 | 37 | PmU.fA.mG.mA.mU.mG.mU.mU.mC.mA.mU.mG.mG.fA.mG |
| | | .mC.mC.mU.mG.mC.mU.fC.fC.fA.mU.mG.mA.mA.mC.mA |
| | | .mU.mC.mU.mA |
| 1542 | 38 | PmU.fG.mC.mA.mA.mG.mU.mG.mG.mU.mA.mG.mU.fU.mG |
| | | .mG.mA.mG.mG.mC.mA.fA.fC.fU.mA.mC.mC.mA.mC.mU |
| | | .mU.mG.mC.mA |
| 1543 | 39 | PmU.fC.mA.mC.mA.mC.mC.mA.mU.mA.mA.mC.mU.fU.mG |
| | | .mC.mC.mA.mC.mC.mA.fA.fG.fU.mU.mA.mU.mG.mG.mU |
| | | .mG.mU.mG.mA |
| 1544 | 40 | PmU.fA.mA.mA.mG.mA.mG.mA.mU.mC.mU.mC.mA.fU.mC |
| | | .mA.mC.mU.mC.mG.mA.fU.fG.fA.mG.mA.mU.mC.mU.mC |
| | | .mU.mU.mU.mA |
| 1545 | 41 | PmU.fU.mC.mA.mA.mC.mU.mU.mG.mU.mG.mG.mU.fC.mU |
| | | .mU.mC.mA.mU.mA.mG.fA.fC.fC.mA.mC.mA.mA.mG.mU |
| | | .mU.mG.mA.mA |
| 1546 | 42 | PmU.f.A.m.A.m.A.mC.mG.m.A.mC.mU.mU.mC.mU.mC.f |
| | | U.mU.mG.mU.mG.mA.mA.mA.fG.fA.fG.mA.mA.mG.mU.m |
| | | C.mG.mU.mU.mU.mA |
| 1547 | 43 | PmU.fG.mU.mA.mU.mG.mU.mG.mG.mC.mA.mU.mA.fU.mG |
| | | .mU.mC.mA.mC.mC.mA.fU.fA.fU.mG.mC.mC.mA.mC.mA |
| | | .mU.mA.mC.mA |
| 1548 | 44 | PmU.fG.mU.mC.mU.mU.mU.mC.mU.mU.mG.mG.mA.fA.mG |
| | | .mC.mC.mA.mA.mC.mU.fU.fC.fC.mA.mA.mG.mA.mA.mA |
| | | .mG.mA.mC.mA |
| 1549 | 45 | PmU.fC.mA.mU.mC.mC.mA.mG.mA.mU.mA.mA.mU.fC.mC |
| | | .mU.mC.mC.mC.mG.mG.fA.fU.fU.mA.mU.mC.mU.mG.mG |
| | | .mA.mU.mG.mA |
| 1550 | 46 | PmU.fU.mU.mG.mA.mC.mU.mU.mU.mG.mU.mC.mA.fU.mA |
| | | .mG.mC.mC.mU.mU.mA.fU.fG.fA.mC.mA.mA.mA.mG.mU |
| | | .mC.mA.mA.mA |
| 1551 | 47 | PmU.fA.m.A.mA.mC.mU.mC.mC.mA.mG.mA.mC.mC.fU.m |
| | | A.mG.mA.mC.mC.mU.mA.fG.fG.fU.mC.mU.mG.mG.mA.m |
| | | G.mU.mU.mU.mA |
| 1552 | 48 | PmU.fA.mU.mA.mA.mC.mU.mU.mG.mC.mC.m.A.mC.fC.m |
| | | U.mU.mC.mU.mC.mA.mG.fG.fU.fG.mG.mC.mA.mA.mG.m |
| | | U.mU.mA.mU.mA |
| 1553 | 49 | PmU.fC.mA.mU.mA.mG.mU.mC.mA.mU.mA.mA.mA.fA.mU |
| | | .mU.mC.mA.mG.mA.mU.fU.fU.fU.mA.mU.mG.mA.mC.mU |
| | | .mA.mU.mG.mA |
| 1554 | 50 | PmU.fU.mG.mG.mC.mU.mC.mC.mU.mG.mU.mG.mA.fA.mG |
| | | .mU.mU.mG.mC.mC.mU.fU.fC.fA.mC.mA.mG.mG.mA.mG |
| | | .mC.mC.mA.mA |
| 1555 | 51 | PmU.fA.m.A.mA.mG.mU.mA.mC.mU.mC.mA.mG.m.A.fC. |
| | | mA.mC.mC.mA.mC.mU.mG.fU.fC.fU.mG.mA.mG.mU.mA. |
| | | mC.mU.mU.mU.mA |
| 1556 | 52 | PmU.fG.mC.mU.mC.mA.mU.mU.mG.mU.mC.mU.mU.fU.mC |
| | | .mU.mU.mG.mG.mG.mA.fA.fA.fG.mA.mC.mA.mA.mU.mG |
| | | .mA.mG.mC.mA |
| 1557 | 53 | PmU.fU.mA.mA.m.A.mA.mU.mU.mC.mA.mG.mG.mA.fA.m |
| | | U.mU.mC.mC.mU.mA.mU.fU.fC.fC.mU.mG.mA.mA.mU.m |
| | | U.mU.mU.mA.mA |
| 1558 | 54 | PmU.fG.mA.mG.mA.mU.mC.mU.mU.mG.mG.mC.mC.fU.mG |
| | | .mC.mC.mA.mU.mC.mA.fG.fG.fC.mC.mA.mA.mG.mA.mU |
| | | .mC.mU.mC.mA |
| 1559 | 55 | PmU.fG.mA.mG.mC.mA.mU.mC.mU.mC.mU.mC.mU.fC.mA |
| | | .mC.mA.mG.mC.mU.mG.fA.fG.fA.mG.mA.mG.mA.mU.mG |
| | | .mC.mU.mC.mA |
| 1560 | 56 | PmU.fA.m.A.mC.mC.mG.mU.mC.mA.mU.mA.mG.mC.fA.m |
| | | G.mU.mG.mG.mA.mC.mU.fG.fC.fU.mA.mU.mG.mA.mC.m |
| | | G.mG.mU.mU.mA |
| 1561 | 57 | PmU.fA.mG.mA.mG.mC.mU.mU.mU.mG.mA.mU.mA.fU.mC |
| | | .mC.mU.mG.mU.mG.mA.fU.fA.fU.mC.mA.mA.mA.mG.mC |
| | | .mU.mC.mU.mA |
| 1562 | 58 | PmU.fA.mC.mA.mA.mU.mG.mU.mG.mC.mU.mG.mC.fU.mG |
| | | .mU.mC.mA.mG.mC.mA.fG.fC.fA.mG.mC.mA.mC.mA.mU |
| | | .mU.mG.mU.mA |
| 1563 | 59 | PmU.fG.mG.mU.mA.mC.mG.mG.mG.mU.mA.mG.mA.fA.mG |
| | | .mC.mC.mA.mG.mC.mU.fU.fC.fU.mA.mC.mC.mC.mG.mU |
| | | .mA.mC.mC.mA |
| 1564 | 60 | PmU.fC.mA.mG.mA.mC.mC.mU.mA.mG.mA.mC.mC.fU.mG |
| | | .mG.mU.mC.mA.mC.mA.fG.fG.fU.mC.mU.mA.mG.mG.mU |
| | | .mC.mU.mG.mA |
| 1565 | 61 | PmU.fU.mU.mC.mU.mC.mU.mU.mG.mU.mG.mA.mA.fC.mU |
| | | .mA.mU.mC.mA.mA.mG.fU.fU.fC.mA.mC.mA.mA.mG.mA |
| | | .mG.mA.mA.mA |
| 1566 | 62 | PmU.fU.mU.mC.mA.mG.mG.mA.m.A.mU.mU.mC.mC.fU.m |
| | | G.mC.mU.mU.mC.mC.m.A.fG.fG.fA.mA.mU.mU.mC.mC. |
| | | mU.mG.mA.mA.mA |
| 1567 | 63 | PmU.fC.mC.mA.mG.mG.mU.mU.mU.mU.mC.mC.mA.fU.mA |
| | | .mU.mC.mC.mU.mU.mA.fU.fG.fG.mA.mA.mA.mA.mC.mC |
| | | .mU.mG.mG.mA |
| 1568 | 64 | PmU.fC.mA.mA.mC.mU.mU.mG.mA.mA.mU.mG.mA.fA.mA |
| | | .mC.mG.mA.mC.mU.mU.fU.fC.fA.mU.mU.mC.mA.mA.mG |
| | | .mU.mU.mG.mA |
| 1569 | 65 | PmU.fG.mU.mG.mC.mU.mG.mC.mU.mG.mU.mC.mA.fG.mC |
| | | .mA.mC.mA.mA.mG.mC.fU.fG.fA.mC.mA.mG.mC.mA.mG |
| | | .mC.mA.mC.mA |
| 1570 | 66 | PmU.fG.m.A.mC.mC.mU.mC.mC.mU.mU.mC.mC.mG.fA.m |
| | | G.mU.mC.mA.mG.mC.mU.fC.fG.fG.mA.mA.mG.mG.mA.m |
| | | G.mG.mU.mC.mA |
| 1571 | 67 | PmU.fC.mA.mA.mU.mU.mA.mA.mG.mU.mU.mG.mA.fC.mU |
| | | .mA.mG.mA.mC.mA.mG.fU.fC.fA.mA.mC.mU.mU.mA.mA |
| | | .mU.mU.mG.mA |
| 1572 | 68 | PmU.fU.mG.mA.mC.mA.mC.mG.mU.mU.mC.mG.mC.fC.mG |
| | | .mC.mU.mG.mG.mC.mG.fG.fC.fG.mA.mA.mC.mG.mU.mG |
| | | .mU.mC.mA.mA |
| 1573 | 69 | PmU.fC.mA.mU.mU.mG.mU.mC.mU.mU.mU.mC.mU.fU.mG |
| | | .mG.mA.mA.mG.mC.mA.fA.fG.fA.m.A.mA.mG.mA.mC.m |
| | | A.mA.mU.mG.mA |
| 1574 | 70 | PmU.fC.mC.mA.mA.mC.mU.mU.mG.mA.mA.mU.mG.fA.mA |
| | | .mA.mC.mG.mA.mU.mU.fC.fA.fU.mU.mC.mA.mA.mG.mU |
| | | .mU.mG.mG.mA |
| 1575 | 71 | PmU.fC.mA.mC.mA.mA.mA.mG.mU.mA.mC.mU.mC.fA.mG |
| | | .mA.mC.mA.mC.mC.mU.fG.fA.fG.mU.mA.mC.mU.mU.mU |
| | | .mG.mU.mG.mA |
| 1576 | 72 | PmU.fU.mG.mC.mA.mG.mU.mG.mG.mU.mA.mG.mG.fU.mG |
| | | .mA.mC.mG.mC.mC.mA.fC.fC.fU.mA.mC.mC.mA.mC.mU |
| | | .mG.mC.mA.mA |
| 1577 | 73 | PmU.fU.mC.mA.mU.mG.mA.mG.mG.mA.mU.mG.mA.fU.mG |
| | | .mA.mC.mA.mU.mC.mA.fU.fC.fA.mU.mC.mC.mU.mC.mA |
| | | .mU.mG.mA.mA |
| 1578 | 74 | PmU.fU.mU.mC.mA.mA.mC.mU.mU.mG.mU.mG.mG.fU.mC |
| | | .mU.mU.mC.mA.mG.mA.fC.fC.fA.mC.mA.mA.mG.mU.mU |
| | | .mG.mA.mA.mA |
| 1579 | 75 | PmU.fG.mU.mA.mG.mU.mU.mG.mG.mA.mG.mG.mA.fA.mG |
| | | .mC.mC.mU.mC.mC.mU.fU.fC.fC.mU.mC.mC.mA.mA.mC |
| | | .mU.mA.mC.mA |
| 1580 | 76 | PmU.fC.mA.mU.mG.mC.mU.mG.mU.mA.mC.mA.mC.fU.mG |
| | | .mC.mC.mU.mG.mC.mA.fG.fU.fG.mU.mA.mC.mA.mG.mC |
| | | .mA.mU.mG.mA |
| 1581 | 77 | PmU.fU.mG.mU.mC.mU.mU.mU.mC.mU.mU.mG.mG.fA.mA |
| | | .mG.mC.mC.mA.mU.mU.fC.fC.fA.mA.mG.mA.mA.mA.mG |
| | | .mA.mC.mA.mA |
| 1582 | 78 | PmU.fC.mG.mA.mC.mU.mC.mC.mU.mU.mC.mU.mA.fU.mG |
| | | .mG.mU.mC.mU.mC.mA.fU.fA.fG.mA.mA.mG.mG.mA.mG |
| | | .mU.mC.mG.mA |
| 1583 | 79 | PmU.fG.mA.mC.mA.mU.mC.mC.mA.mG.mA.mU.mA.fA.mU |
| | | .mC.mC.mU.mC.mA.mU.fU.fA.fU.mC.mU.mG.mG.mA.mU |
| | | .mG.mU.mC.mA |
| 1584 | 80 | PmU.fU.mG.mA.mG.mA.mU.mC.mU.mU.mG.mG.mC.fC.mU |
| | | .mG.mC.mC.mA.mA.mG.fG.fC.fC.mA.mA.mG.mA.mU.mC |
| | | .mU.mC.mA.mA |
| 1585 | 81 | PmU.fA.mC.mG.mC.mU.mG.mU.mC.mU.mU.mC.mA.fA.mG |
| | | .mG.mC.mG.mG.mC.mU.fU.fG.fA.mA.mG.mA.mC.mA.mG |
| | | .mC.mG.mU.mA |
| 1586 | 82 | PmU.fA.mG.mA.mC.mA.mG.mG.mA.m.A.mA.mG.mC.fU.m |
| | | U.mC.mG.mG.mC.mA.mA.fG.fC.fU.mU.mU.mC.mC.mU.m |
| | | G.mU.mC.mU.mA |
| 1587 | 83 | PmU.fU.mU.mG.mA.mA.mC.mA.mC.mA.mU.mG.mU.fU.mG |
| | | .mC.mU.mC.mA.mC.m.A.fA.fC.fA.mU.mG.mU.mG.mU.m |
| | | U.mC.mA.mA.mA |
| 1588 | 84 | PmU.fA.mU.mA.m.A.mA.mA.mU.mU.mC.mA.mG.mG.fA.m |
| | | .A.mU.mU.mC.mC.mU.mU.fC.fC.fU.mG.mA.mA.mU.mU. |
| | | mU.mU.mA.mU.mA |
| 1589 | 85 | PmU.fC.mC.mC.mA.mA.mA.mU.mC.mC.mU.mC.mA.fU.mC |
| | | .mU.mU.mG.mG.mG.mA.fU.fG.fA.mG.mG.mA.mU.mU.mU |
| | | .mG.mG.mG.mA |
| 1590 | 86 | PmU.fA.mA.mG.mA.mG.mA.mU.mC.mU.mC.mA.mU.fC.mA |
| | | .mC.mU.mC.mA.mU.mG.fA.fU.fG.mA.mG.mA.mU.mC.mU |
| | | .mC.mU.mU.mA |
| 1591 | 87 | PmU.fA.mA.mA.mG.mC.mA.mU.mU.mG.mA.mU.mG.fU.mU |
| | | .mC.mA.mC.mU.mA.mA.fC.fA.fU.mC.mA.mA.mU.mG.mC |
| | | .mU.mU.mU.mA |
| 1592 | 88 | PmU.fA.mG.mG.mA.mA.mU.mU.mC.mC.mU.mG.mC.fU.mU |
| | | .mC.mU.mU.mU.mA.mA.fG.fC.fA.mG.mG.mA.mA.mU.mU |
| | | .mC.mC.mU.mA |
| 1593 | 89 | PmU.fA.mU.mG.mA.mA.mG.mG.mA.mG.mU.mC.mU.fU.mG |
| | | .mG.mC.mA.mG.mC.mA.fA.fG.fA.mC.mU.mC.mC.mU.mU |
| | | .mC.mA.mU.mA |
| 1594 | 90 | PmU.fA.mA.mG.mC.mU.mU.mC.mG.mG.mC.mC.mA.fC.mC |
| | | .mU.mC.mU.mU.mG.mG.fU.fG.fG.mC.mC.mG.mA.mA.mG |
| | | .mC.mU.mU.mA |
| 1595 | 91 | PmU.fU.mG.mA.mC.mU.mU.mU.mG.mU.mC.mA.mU.fA.mG |
| | | .mC.mC.mU.mG.mC.mU.fA.fU.fG.mA.mC.mA.mA.mA.mG |
| | | .mU.mC.mA.mA |
| 1596 | 92 | PmU.fU.mC.mC.mA.mA.mG.mC.mU.mG.mA.mA.m.A.fC.m |
| | | U.mC.mC.mA.mG.mA.mG.fU.fU.fU.mC.mA.mG.mC.mU.m |
| | | U.mG.mG.mA.mA |
| 1597 | 93 | PmU.fC.mC.mA.mA.mA.mU.mC.mC.mU.mC.mA.mU.fC.mU |
| | | .mU.mG.mG.mA.mA.mG.fA.fU.fG.mA.mG.mG.mA.mU.mU |
| | | .mU.mG.mG.mA |
| 1598 | 94 | PmU.fC.mA.mG.mC.mU.mG.mU.mU.mU.mU.mA.mA.fU.mU |
| | | .mC.mA.mA.mU.mA.mA.fU.fU.fA.mA.mA.mA.mC.mA.mG |
| | | .mC.mU.mG.mA |
| 1599 | 95 | PmU.fA.mA.mC.mG.mA.mC.mU.mU.mC.mU.mC.mU.fU.mG |
| | | .mU.mG.mA.mA.mC.m.A.fA.fG.fA.mG.mA.mA.mG.mU.m |
| | | C.mG.mU.mU.mA |
| 1600 | 96 | PmU.fC.mC.mG.mG.mA.mA.mC.mA.mU.mC.mC.mA.fA.mG |
| | | .mC.mG.mG.mG.mC.mU.fU.fG.fG.mA.mU.mG.mU.mU.mC |
| | | .mC.mG.mG.mA |
| 1601 | 97 | PmU.fC.mA.mA.mA.mC.mA.mC.mA.mU.mA.mG.mA.fC.mA |
| | | .mU.mC.mC.mA.mU.mG.fU.fC.fU.mA.mU.mG.mU.mG.mU |
| | | .mU.mU.mG.mA |
| 1602 | 98 | PmU.fU.mU.mC.mA.mC.mA.mC.mC.mA.mU.mA.mA.fC.mU |
| | | .mU.mG.mC.mC.mA.mG.fU.fU.fA.mU.mG.mG.mU.mG.mU |
| | | .mG.mA.mA.mA |
| 1603 | 99 | PmU.fU.mC.mU.mC.mU.mU.mG.mU.mG.mA.mA.mC.fU.mA |
| | | .mU.mC.mA.mA.mU.mA.fG.fU.fU.mC.mA.mC.mA.mA.mG |
| | | .mA.mG.mA.mA |
| 1604 | 100 | PmU.fA.mU.mU.mC.mA.mG.mG.mA.mA.mU.mU.mC.fC.mU |
| | | .mG.mC.mU.mU.mA.mG.fG.fA.fA.mU.mU.mC.mC.mU.mG |
| | | .mA.mA.mU.mA |
| 1605 | 101 | PmU.fG.mA.mC.mA.mC.mU.mU.mU.mG.mA.mC.mC.fC.mA |
| | | .mA.mA.mU.mU.mU.mG.fG.fG.fU.mC.mA.mA.mA.mG.mU |
| | | .mG.mU.mC.mA |
| 1606 | 102 | PmU.fC.mA.mC.mA.mA.mA.mC.mA.mG.mA.mG.mC.fU.mU |
| | | .mU.mG.mA.mU.mA.m.A.fG.fC.fU.mC.mU.mG.mU.mU.m |
| | | U.mG.mU.mG.mA |
| 1607 | 103 | PmU.fC.mA.mA.mA.mC.mA.mG.mA.mG.mC.mU.mU.fU.mG |
| | | .mA.mU.mA.mU.mC.m.A.fA.fA.fG.mC.mU.mC.mU.mG.m |
| | | U.mU.mU.mG.mA |
| 1608 | 104 | PmU.fU.mU.mG.mG.mA.mG.mU.mU.mU.mC.mU.mC.fC.mU |
| | | .mU.mC.mA.mG.mA.mG.fG.fA.fG.mA.mA.mA.mC.mU.mC |
| | | .mC.mA.mA.mA |
| 1609 | 105 | PmU.fU.mC.mA.mC.mA.mC.mC.mA.mU.mA.mA.mC.fU.mU |
| | | .mG.mC.mC.mA.mA.mA.fG.fU.fU.mA.mU.mG.mG.mU.mG |
| | | .mU.mG.mA.mA |
| 1610 | 106 | PmU.fU.mG.mA.mA.mU.mG.mA.mA.mA.mC.mG.mA.fC.mU |
| | | .mU.mC.mU.mC.mA.mG.fU.fC.fG.mU.mU.mU.mC.mA.mU |
| | | .mU.mC.mA.mA |
| 1611 | 107 | PmU.fC.mA.mG.mG.mU.mU.mU.mU.mC.mC.mA.mU.fA.mU |
| | | .mC.mC.mU.mU.mA.mU.fA.fU.fG.mG.mA.mA.mA.mA.mC |
| | | .mC.mU.mG.mA |
| 1612 | 108 | PmU.fG.mC.mA.mU.mC.mU.mC.mU.mC.mU.mC.mA.fC.mA |
| | | .mG.mC.mU.mG.mU.mG.fU.fG.fA.mG.mA.mG.mA.mG.mA |
| | | .mU.mG.mC.mA |
| 1613 | 109 | PmU.fA.mC.mA.mU.mC.mC.mA.mG.mA.mU.mA.m.A.fU.m |
| | | C.mC.mU.mC.mC.mG.mA.fU.fU.fA.mU.mC.mU.mG.mG.m |
| | | A.mU.mG.mU.mA |
| 1614 | 110 | PmU.fC.mG.mA.mG.mU.mU.mG.mU.mU.mC.mC.mC.fU.mC |
| | | .mG.mG.mU.mG.mG.mA.fG.fG.fG.m.A.mA.mC.mA.mA.m |
| | | C.mU.mC.mG.mA |
| 1615 | 111 | PmU.f.A.mC.mA.mC.mA.mU.mG.mU.mU.mG.mC.mU.fC.m |
| | | A.mU.mU.mG.mU.mU.mG.fA.fG.fC.mA.mA.mC.mA.mU.m |
| | | G.mU.mG.mU.mA |
| 1616 | 112 | PmU.fU.mC.mU.mC.mA.mA.mU.mU.mA.mA.mG.mU.fU.mG |
| | | .mA.mC.mU.mA.mC.mA.fA.fC.fU.mU.mA.mA.mU.mU.mG |
| | | .mA.mG.mA.mA |
| 1617 | 113 | PmU.fG.mA.mA.mG.mC.mC.mA.mA.mA.mG.mC.mA.fU.mU |
| | | .mG.mA.mU.mG.mA.mA.fU.fG.fC.mU.mU.mU.mG.mG.mC |
| | | .mU.mU.mC.mA |
| 1618 | 114 | PmU.fG.mC.mA.mG.mU.mG.mG.mA.mA.mA.mG.mA.fG.mA |
| | | .mU.mC.mU.mC.mU.mC.fU.fC.fU.mU.mU.mC.mC.mA.mC |
| | | .mU.mG.mC.mA |
| 1619 | 115 | PmU.f.A.mC.mA.mC.mU.mG.mC.mC.mU.mG.mG.mA.fG.m |
| | | G.mG.mC.mC.mU.mC.mC.fU.fC.fC.mA.mG.mG.mC.mA.m |
| | | G.mU.mG.mU.mA |
| 1620 | 116 | PmU.fU.mA.mG.mA.mC.mC.mU.mG.mG.mU.mC.mA.fC.mA |
| | | .mU.mU.mC.mC.mU.mG.fU.fG.fA.mC.mC.mA.mG.mG.mU |
| | | .mC.mU.mA.mA |
| 1621 | 117 | PmU.fC.mA.mG.mA.mC.mA.mC.mA.m.A.mA.mC.mA.fG.m |
| | | A.mG.mC.mU.mU.mU.mC.fU.fG.fU.mU.mU.mG.mU.mG.m |
| | | U.mC.mU.mG.mA |
| 1622 | 118 | PmU.fG.mA.mG.mU.mU.mU.mC.mU.mC.mC.mU.mU.fC.mA |
| | | .mG.mC.mC.mA.mU.mG.fA.fA.fG.mG.mA.mG.mA.mA.mA |
| | | .mC.mU.mC.mA |
| 1623 | 119 | PmU.fA.mU.mG.mU.mG.mC.mU.mG.mC.mU.mG.mU.fC.mA |
| | | .mG.mC.mA.mC.mU.mG.fA.fC.fA.mG.mC.mA.mG.mC.mA |
| | | .mC.mA.mU.mA |
| 1624 | 120 | PmU.fC.mA.mG.mA.mG.mC.mU.mU.mU.mG.mA.mU.fA.mU |
| | | .mC.mC.mU.mG.mA.mU.fA.fU.fC.mA.mA.mA.mG.mC.mU |
| | | .mC.mU.mG.mA |
| 1625 | 121 | PmU.fG.mA.mU.mA.mU.mC.mC.mU.mG.mU.mG.mC.fA.mG |
| | | .mG.mG.mA.mG.mC.mU.fG.fC.fA.mC.mA.mG.mG.mA.mU |
| | | .mA.mU.mC.mA |
| 1626 | 122 | PmU.fA.mG.mG.mG.mC.mA.mA.mC.mG.mU.mC.mA.fU.mA |
| | | .mG.mU.mC.mA.mU.mA.fU.fG.fA.mC.mG.mU.mU.mG.mC |
| | | .mC.mC.mU.mA |
| 1627 | 123 | PmU.fA.mG.mA.mC.mC.mU.mA.mG.mA.mC.mC.mU.fG.mG |
| | | .mU.mC.mA.mC.mC.mC.fA.fG.fG.mU.mC.mU.mA.mG.mG |
| | | .mU.mC.mU.mA |
| 1628 | 124 | PmU.fA.mG.mU.mA.mC.mU.mC.mA.mG.mA.mC.mA.fC.mC |
| | | .mA.mC.mA.mG.mG.mG.fU.fG.fU.mC.mU.mG.mA.mG.mU |
| | | .mA.mC.mU.mA |
| 1629 | 125 | PmU.fA.mA.mG.mG.mC.mU.mC.mC.mG.mU.mC.mC.fC.mG |
| | | .mC.mU.mC.mC.mC.mG.fG.fG.fA.mC.mG.mG.mA.mG.mC |
| | | .mC.mU.mU.mA |
| 1630 | 126 | PmU.fG.mG.mG.mC.mA.mA.mC.mG.mU.mC.mA.mU.fA.mG |
| | | .mU.mC.mA.mU.mC.mU.fA.fU.fG.mA.mC.mG.mU.mU.mG |
| | | .mC.mC.mC.mA |
| 1631 | 127 | PmU.fC.mU.mG.mU.mU.mU.mU.mA.mA.mU.mU.mC.fA.mA |
| | | .mU.mC.mC.mC.mU.mU.fG.fA.fA.mU.mU.mA.mA.mA.mA |
| | | .mC.mA.mG.mA |
| 1632 | 128 | PmU.fA.mG.mA.mG.mA.mU.mC.mU.mC.mA.mU.mC.fA.mC |
| | | .mU.mC.mA.mC.mG.mU.fG.fA.fU.mG.mA.mG.mA.mU.mC |
| | | .mU.mC.mU.mA |
| 1633 | 129 | PmU.fG.mG.mU.mC.mU.mU.mC.mA.mU.mA.mA.mU.fU.mG |
| | | .mA.mU.mU.mU.mC.mA.fA.fU.fU.mA.mU.mG.mA.mA.mG |
| | | .mA.mC.mC.mA |
| 1634 | 130 | PmU.fC.mA.mU.mA.mU.mC.mU.mU.mG.mG.mC.mU.fU.mC |
| | | .mA.mC.mA.mC.mG.m.A.fA.fG.fC.mC.mA.mA.mG.mA.m |
| | | U.mA.mU.mG.mA |
| 1635 | 131 | PmU.fC.mA.mC.mC.mA.mA.mC.mU.mU.mG.mA.mA.fU.mG |
| | | .mA.mA.mA.mC.mC.mA.fU.fU.fC.mA.mA.mG.mU.mU.mG |
| | | .mG.mU.mG.mA |
| 1636 | 132 | PmU.fA.mG.mC.mU.mG.mU.mU.mU.mU.mA.mA.mU.fU.mC |
| | | .mA.mA.mU.mC.mG.m.A.fA.fU.fU.mA.mA.mA.mA.mC.m |
| | | A.mG.mC.mU.mA |
| 1637 | 133 | PmU.fU.mA.mA.mC.mU.mU.mG.mC.mC.mA.mC.mC.fU.mU |
| | | .mC.mU.mC.mA.mA.mA.fG.fG.fU.mG.mG.mC.mA.mA.mG |
| | | .mU.mU.mA.mA |
| 1638 | 134 | PmU.fU.mG.mA.mG.mC.mA.mG.mG.mU.mA.mC.mC.fU.mG |
| | | .mC.mU.mU.mU.mC.mA.fG.fG.fU.mA.mC.mC.mU.mG.mC |
| | | .mU.mC.mA.mA |
| 1639 | 135 | PmU.fU.mU.mG.mA.mU.mG.mU.mA.mG.m.A.mC.mC.fU.m |
| | | C.mC.mU.mU.mC.mG.mA.fG.fG.fU.mC.mU.mA.mC.mA.m |
| | | U.mC.mA.mA.mA |
| 1640 | 136 | PmU.fG.mG.mC.mA.mA.mG.mU.mG.mG.mU.mA.mG.fU.mU |
| | | .mG.mG.mA.mG.mA.m.A.fC.fU.fA.mC.mC.mA.mC.mU.m |
| | | U.mG.mC.mC.mA |
| 1641 | 137 | PmU.fG.mG.mA.mA.mG.mC.mC.mU.mC.mA.mA.mA.fG.mC |
| | | .mU.mC.mG.mA.mG.mC.fU.fU.fU.mG.mA.mG.mG.mC.mU |
| | | .mU.mC.mC.mA |
| 1642 | 138 | PmU.fA.mA.mU.mG.mA.mC.mA.mG.mU.mA.mA.mU.fU.mG |
| | | .mG.mG.mU.mC.mC.mA.fA.fU.fU.mA.mC.mU.mG.mU.mC |
| | | .mA.mU.mU.mA |
| 1643 | 139 | PmU.fU.mU.mU.mG.mA.mA.mC.mA.mC.mA.mU.mG.fU.mU |
| | | .mG.mC.mU.mC.mA.mA.fC.fA.fU.mG.mU.mG.mU.mU.mC |
| | | .mA.mA.mA.mA |
| 1644 | 140 | PmU.fA.m.A.m.A.mU.mC.mC.mU.mC.mA.mU.mC.mU.fU. |
| | | mG.mG.mA.mG.mU.mC.mA.f.A.fG.fA.mU.mG.mA.mG.mG |
| | | .mA.mU.mU.mU.mA |
| 1645 | 141 | PmU.fG.mG.mA.mG.mU.mU.mU.mC.mU.mC.mC.mU.fU.mC |
| | | .mA.mG.mC.mC.mG.mA.fA.fG.fG.mA.mG.mA.mA.mA.mC |
| | | .mU.mC.mC.mA |
| 1646 | 142 | PmU.fC.mA.mU.mA.mA.mC.mU.mU.mG.mC.mC.mA.fC.mC |
| | | .mU.mU.mC.mU.mG.mG.fU.fG.fG.mC.mA.mA.mG.mU.mU |
| | | .mA.mU.mG.mA |
| 1647 | 143 | PmU.fG.mC.mU.mG.mU.mU.mU.mU.mA.mA.mU.mU.fC.mA |
| | | .mA.mU.mC.mC.mU.mG.fA.fA.fU.mU.mA.mA.mA.mA.mC |
| | | .mA.mG.mC.mA |
| 1648 | 144 | PmU.fA.mA.mG.mC.mU.mC.mG.mA.mG.mU.mU.mG.fU.mU |
| | | .mC.mC.mC.mU.mA.m.A.fC.fA.fA.mC.mU.mC.mG.mA.m |
| | | G.mC.mU.mU.mA |
| 1649 | 145 | PmU.fG.mG.mC.mA.mA.mC.mG.mU.mC.mA.mU.mA.fG.mU |
| | | .mC.mA.mU.mA.mA.mC.fU.fA.fU.mG.mA.mC.mG.mU.mU |
| | | .mG.mC.mC.mA |
| 1650 | 146 | PmU.fU.mU.mC.mC.mA.mG.mG.mU.mU.mU.mU.mC.fC.mA |
| | | .mU.mA.mU.mC.mU.mG.fG.fA.fA.mA.mA.mC.mC.mU.mG |
| | | .mG.mA.mA.mA |
| 1651 | 147 | PmU.fU.mC.mC.mA.mG.mG.mU.mU.mU.mU.mC.mC.fA.mU |
| | | .mA.mU.mC.mC.mA.mU.fG.fG.fA.m.A.mA.mA.mC.mC.m |
| | | U.mG.mG.mA.mA |
| 1652 | 148 | PmU.fC.mC.mA.mU.mG.mU.mU.mG.mU.mG.mC.mA.fA.mU |
| | | .mC.mC.mA.mU.mA.mU.fU.fG.fC.mA.mC.mA.mA.mC.mA |
| | | .mU.mG.mG.mA |
| 1653 | 149 | PmU.fC.mA.mU.mA.mU.mC.mC.mU.mU.mG.mA.mC.fU.mU |
| | | .mU.mG.mA.mA.mA.mA.fG.fU.fC.mA.mA.mG.mG.mA.mU |
| | | .mA.mU.mG.mA |
| 1654 | 150 | PmU.fU.mG.mA.mC.mU.mU.mU.mG.mA.mA.mC.mA.fC.mA |
| | | .mU.mG.mU.mU.mU.mG.fU.fG.fU.mU.mC.mA.mA.mA.mG |
| | | .mU.mC.mA.mA |
| 1655 | 151 | PmU.fC.mU.mC.mA.mU.mC.mU.mU.mG.mG.mA.mG.fU.mU |
| | | .mU.mC.mU.mC.mA.m.A.fC.fU.fC.mC.mA.mA.mG.mA.m |
| | | U.mG.mA.mG.mA |
| 1656 | 152 | PmU.fC.mA.mU.mG.mU.mU.mG.mC.mU.mC.mA.mU.fU.mG |
| | | .mU.mC.mU.mU.mC.mA.fA.fU.fG.mA.mG.mC.mA.mA.mC |
| | | .mA.mU.mG.mA |
| 1657 | 153 | PmU.fA.mC.mC.mA.mA.mC.mU.mU.mG.mA.mA.mU.fG.mA |
| | | .mA.mA.mC.mG.mU.mC.fA.fU.fU.mC.mA.mA.mG.mU.mU |
| | | .mG.mG.mU.mA |
| 1658 | 154 | PmU.fA.mC.mA.mG.mA.mU.mC.mG.mC.mU.mG.mU.fC.mU |
| | | .mG.mC.mC.mC.mA.mG.fA.fC.fA.mG.mC.mG.mA.mU.mC |
| | | .mU.mG.mU.mA |
| 1659 | 155 | PmU.fU.mC.mA.mC.mA.mG.mC.mU.mG.mC.mC.mU.fU.mU |
| | | .mC.mU.mU.mA.mA.mA.fA.fG.fG.mC.mA.mG.mC.mU.mG |
| | | .mU.mG.mA.mA |
| 1660 | 156 | PmU.fG.mG.mC.mC.mG.mC.mC.mA.mG.mA.mA.mU.fC.mA |
| | | .mC.mC.mU.mC.mU.mG.fA.fU.fU.mC.mU.mG.mG.mC.mG |
| | | .mG.mC.mC.mA |
| 1661 | 157 | PmU.fC.mC.mA.mA.mG.mC.mU.mG.mA.mA.mA.mC.fU.mC |
| | | .mC.mA.mG.mA.mG.mA.fG.fU.fU.mU.mC.mA.mG.mC.mU |
| | | .mU.mG.mG.mA |
| 1662 | 158 | PmU.fU.mU.mG.mA.mU.mC.mA.mG.mG.mG.mC.mA.fA.mC |
| | | .mG.mU.mC.mA.mG.mU.fU.fG.fC.mC.mC.mU.mG.mA.mU |
| | | .mC.mA.mA.mA |
| 1663 | 159 | PmU.fG.mU.mU.mC.mC.mC.mA.mA.mA.mC.mC.mA.fU.mG |
| | | .mC.mC.mA.mC.mC.mA.fU.fG.fG.mU.mU.mU.mG.mG.mG |
| | | .mA.mA.mC.mA |
| 1664 | 160 | PmU.fA.mC.mC.mU.mG.mC.mU.mU.mU.mU.mG.mC.fC.mG |
| | | .mC.mU.mU.mC.mC.mG.fG.fC.fA.mA.mA.mA.mG.mC.mA |
| | | .mG.mG.mU.mA |
| 1665 | 161 | PmU.fU.mU.mG.mC.mU.mC.mA.mU.mU.mG.mU.mC.fU.mU |
| | | .mU.mC.mU.mU.mA.mA.fG.fA.fC.mA.mA.mU.mG.mA.mG |
| | | .mC.mA.mA.mA |
| 1666 | 162 | PmU.fC.mA.mC.mG.mU.mU.mC.mG.mC.mC.mG.mC.fU.mG |
| | | .mG.mG.mA.mG.mC.mA.fG.fC.fG.mG.mC.mG.mA.mA.mC |
| | | .mG.mU.mG.mA |
| 1667 | 163 | PmU.fC.mA.mU.mU.mC.mU.mU.mG.mA.mU.mG.mU.fA.mG |
| | | .mA.mC.mC.mU.mC.mU.fA.fC.fA.mU.mC.mA.mA.mG.mA |
| | | .mA.mU.mG.mA |
| 1668 | 164 | PmU.fU.mU.mG.mA.mG.mC.mU.mU.mG.mA.mU.mC.fA.mG |
| | | .mG.mG.mC.mA.mC.mU.fG.fA.fU.mC.mA.mA.mG.mC.mU |
| | | .mC.mA.mA.mA |
| 1669 | 165 | PmU.fA.mU.mU.mC.mU.mU.mG.mA.mU.mG.mU.mA.fG.mA |
| | | .mC.mC.mU.mC.mU.mC.fU.fA.fC.mA.mU.mC.mA.mA.mG |
| | | .mA.mA.mU.mA |
| 1670 | 166 | PmU.fU.mG.mA.mA.mG.mG.mA.mG.mU.mC.mU.mU.fG.mG |
| | | .mC.mA.mG.mG.mC.mC.fA.fA.fG.mA.mC.mU.mC.mC.mU |
| | | .mU.mC.mA.mA |
| 1671 | 167 | PmU.fU.mU.mG.mG.mC.mU.mU.mC.mA.mC.mA.mC.fC.mA |
| | | .mU.mA.mA.mC.mU.mG.fG.fU.fG.mU.mG.mA.mA.mG.mC |
| | | .mC.mA.mA.mA |
| 1672 | 168 | PmU.fA.mU.mC.mU.mU.mG.mG.mC.mU.mU.mC.mA.fC.mA |
| | | .mC.mC.mA.mU.mU.mG.fU.fG.fA.mA.mG.mC.mC.mA.mA |
| | | .mG.mA.mU.mA |
| 1673 | 169 | PmU.fC.mU.mC.mA.mC.mA.mG.mC.mU.mG.mC.mC.fU.mU |
| | | .mU.mC.mU.mU.mA.mA.fG.fG.fC.mA.mG.mC.mU.mG.mU |
| | | .mG.mA.mG.mA |
| 1674 | 170 | PmU.fC.mC.mA.mA.mU.mG.mC.mU.mG.mU.mC.mU.fG.mA |
| | | .mU.mC.mC.mA.mU.mC.fA.fG.fA.mC.mA.mG.mC.mA.mU |
| | | .mU.mG.mG.mA |
| 1675 | 171 | PmU.fG.mA.mG.mU.mG.mG.mU.mG.mG.mU.mC.mA.fC.mA |
| | | .mC.mC.mU.mC.mU.mG.fU.fG.fA.mC.mC.mA.mC.mC.mA |
| | | .mC.mU.mC.mA |
| 1676 | 172 | PmU.fA.mU.mA.mG.mG.mG.mA.mC.mU.mC.mA.mC.fU.mC |
| | | .mC.mU.mC.mC.mG.mA.fG.fU.fG.mA.mG.mU.mC.mC.mC |
| | | .mU.mA.mU.mA |
| 1677 | 173 | PmU.fC.mU.mG.mA.mC.mU.mU.mC.mA.mA.mC.mU.fU.mG |
| | | .mU.mG.mG.mU.mC.mA.fA.fG.fU.mU.mG.mA.mA.mG.mU |
| | | .mC.mA.mG.mA |
| 1678 | 174 | PmU.fU.mU.mC.mU.mC.mA.mA.mU.mU.mA.mA.mG.fU.mU |
| | | .mG.mA.mC.mU.mA.mA.fC.fU.fU.mA.mA.mU.mU.mG.mA |
| | | .mG.mA.mA.mA |
| 1679 | 175 | PmU.fA.mG.mU.mU.mU.mC.mU.mC.mC.mU.mU.mC.fA.mG |
| | | .mC.mC.mA.mG.mC.mU.fG.fA.fA.mG.mG.mA.mG.mA.mA |
| | | .mA.mC.mU.mA |
| 1680 | 176 | PmU.fA.mG.mC.mU.mU.mU.mG.mA.mU.mA.mU.mC.fC.mU |
| | | .mG.mU.mG.mC.mA.mG.fG.fA.fU.mA.mU.mC.mA.mA.mA |
| | | .mG.mC.mU.mA |
| 1681 | 177 | PmU.fU.mG.mU.mC.mC.mU.mU.mG.mA.mC.mU.mU.fU.mG |
| | | .mU.mC.mA.mU.mC.mA.fA.fA.fG.mU.mC.mA.mA.mG.mG |
| | | .mA.mC.mA.mA |
| 1682 | 178 | PmU.fC.mA.mG.mG.mU.mA.mC.mG.mU.mG.mU.mC.fU.mG |
| | | .mC.mA.mC.mA.mC.mA.fG.fA.fC.mA.mC.mG.mU.mA.mC |
| | | .mC.mU.mG.mA |
| 1683 | 179 | PmU.f.A.mA.mA.mC.mA.mA.mU.mG.mU.mG.mC.mU.fG.m |
| | | C.mU.mG.mU.mC.mG.mC.fA.fG.fC.mA.mC.mA.mU.mU.m |
| | | G.mU.mU.mU.mA |
| 1684 | 180 | PmU.fA.mU.mA.mU.mC.mC.mU.mG.mU.mG.mC.mA.fG.mG |
| | | .mG.mA.mG.mC.mC.mC.fU.fG.fC.mA.mC.mA.mG.mG.mA |
| | | .mU.mA.mU.mA |
| 1685 | 181 | PmU.fG.mA.mC.mU.mC.mA.mG.mA.mG.mA.mC.mU.fG.mG |
| | | .mC.mU.mU.mU.mC.mC.fA.fG.fU.mC.mU.mC.mU.mG.mA |
| | | .mG.mU.mC.mA |
| 1686 | 182 | PmU.fC.mA.mA.mU.mG.mA.mC.mA.mG.mU.mA.mA.fU.mU |
| | | .mG.mG.mG.mU.mA.mA.fU.fU.fA.mC.mU.mG.mU.mC.mA |
| | | .mU.mU.mG.mA |
| 1687 | 183 | PmU.fG.mA.mG.mC.mC.mA.mC.mC.mU.mU.mC.mC.fU.mG |
| | | .mA.mC.mA.mC.mC.mA.fG.fG.fA.mA.mG.mG.mU.mG.mG |
| | | .mC.mU.mC.mA |
| 1688 | 184 | PmU.fC.mU.mU.mG.mA.mC.mU.mU.mU.mG.mA.mA.fC.mA |
| | | .mC.mA.mU.mG.mU.mG.fU.fU.fC.mA.mA.mA.mG.mU.mC |
| | | .mA.mA.mG.mA |
| 1689 | 185 | PmU.fA.mU.mG.mA.m.A.mA.mC.mG.mA.mC.mU.mU.fC.m |
| | | U.mC.mU.mU.mG.mA.mG.fA.fA.fG.mU.mC.mG.mU.mU.m |
| | | U.mC.mA.mU.mA |
| 1690 | 186 | PmU.fG.mA.mA.mG.mA.mC.mA.mG.mG.mA.mA.mA.fG.mC |
| | | .mU.mU.mC.mG.mG.mC.fU.fU.fU.mC.mC.mU.mG.mU.mC |
| | | .mU.mU.mC.mA |
| 1691 | 187 | PmU.fG.mC.mU.mU.mU.mG.mA.mU.mA.mU.mC.mC.fU.mG |
| | | .mU.mG.mC.mA.mC.mA.fG.fG.fA.mU.mA.mU.mC.mA.mA |
| | | .mA.mG.mC.mA |
| 1692 | 188 | PmU.fU.mC.mU.mU.mG.mA.mG.mC.mU.mU.mG.mA.fU.mC |
| | | .mA.mG.mG.mG.mG.mA.fU.fC.fA.mA.mG.mC.mU.mC.mA |
| | | .mA.mG.mA.mA |
| 1693 | 189 | PmU.fG.mG.mA.mU.mU.mG.mC.mU.mC.mU.mG.mC.fA.mC |
| | | .mU.mC.mU.mG.mG.mU.fG.fC.fA.mG.mA.mG.mC.mA.mA |
| | | .mU.mC.mC.mA |
| 1694 | 190 | PmU.fC.mA.mU.mA.mU.mU.mG.mA.mG.mC.mA.mU.fC.mU |
| | | .mC.mU.mC.mU.mA.mG.fA.fU.fG.mC.mU.mC.mA.mA.mU |
| | | .mA.mU.mG.mA |
| 1695 | 191 | PmU.fA.mU.mC.mC.mU.mU.mG.mA.mC.mU.mU.mU.fG.mA |
| | | .mA.mC.mA.mC.mU.mC.fA.fA.fA.mG.mU.mC.mA.mA.mG |
| | | .mG.mA.mU.mA |
| 1696 | 192 | PmU.fG.mC.mA.mG.mA.mC.mA.mU.mC.mC.mA.mC.fU.mA |
| | | .mC.mU.mC.mC.mU.mA.fG.fU.fG.mG.mA.mU.mG.mU.mC |
| | | .mU.mG.mC.mA |
| 1697 | 193 | PmU.fA.mG.mA.mC.mC.mU.mC.mC.mU.mU.mC.mC.fG.mA |
| | | .mG.mU.mC.mA.mU.mC.fG.fG.fA.mA.mG.mG.mA.mG.mG |
| | | .mU.mC.mU.mA |
| 1698 | 194 | PmU.fA.mC.mC.mU.mU.mC.mU.mC.mA.mA.mU.mU.fA.mA |
| | | .mG.mU.mU.mG.mU.mU.fA.fA.fU.mU.mG.mA.mG.mA.mA |
| | | .mG.mG.mU.mA |
| 1699 | 195 | PmU.fA.mG.mA.mA.mG.mU.mC.mG.mG.mA.mA.mG.fG.mA |
| | | .mG.mC.mC.mG.mU.mC.fC.fU.fU.mC.mC.mG.mA.mC.mU |
| | | .mU.mC.mU.mA |
| 1700 | 196 | PmU.fU.mG.mC.mA.mC.mA.mG.mG.mG.mU.mA.mC.fG.mG |
| | | .mG.mU.mA.mG.mC.mC.fG.fU.fA.mC.mC.mC.mU.mG.mU |
| | | .mG.mC.mA.mA |
| 1701 | 197 | PmU.fA.mU.mG.mA.mC.mA.mG.mU.m.A.mA.mU.mU.fG.m |
| | | G.mG.mU.mC.mC.mC.mC.fA.fA.fU.mU.mA.mC.mU.mG.m |
| | | U.mC.mA.mU.mA |
| 1702 | 198 | PmU.fG.mU.mU.mA.mG.mU.mC.mC.mC.mU.mG.mA.fC.mU |
| | | .mU.mC.mA.mA.mA.mG.fU.fC.fA.mG.mG.mG.mA.mC.mU |
| | | .mA.mA.mC.mA |
| 1703 | 199 | PmU.fA.mU.mA.mU.mC.mC.mU.mU.mG.mA.mC.mU.fU.mU |
| | | .mG.mA.mA.mC.mA.mA.fA.fG.fU.mC.mA.mA.mG.mG.mA |
| | | .mU.mA.mU.mA |
| 1704 | 200 | PmU.fG.mU.mA.mC.mG.mU.mG.mU.mC.mU.mG.mC.fA.mC |
| | | .mA.mG.mG.mG.mG.mU.fG.fC.fA.mG.mA.mC.mA.mC.mG |
| | | .mU.mA.mC.mA |
| 1705 | 201 | PmU.fG.mU.mC.mA.mG.mC.mA.mC.mA.mA.mA.mG.fU.mA |
| | | .mC.mU.mC.mA.mU.mA.fC.fU.fU.mU.mG.mU.mG.mC.mU |
| | | .mG.mA.mC.mA |
| 1706 | 202 | PmU.fU.mG.mG.mU.mC.mU.mU.mC.mA.mU.mA.mA.fU.mU |
| | | .mG.mA.mU.mU.mA.mA.fU.fU.fA.mU.mG.mA.mA.mG.mA |
| | | .mC.mC.mA.mA |
| 1707 | 203 | PmU.fC.mA.mG.mA.mG.mA.mC.mU.mC.mA.mG.mA.fG.mA |
| | | .mC.mU.mG.mG.mU.mC.fU.fC.fU.mG.mA.mG.mU.mC.mU |
| | | .mC.mU.mG.mA |
| 1708 | 204 | PmU.fU.mA.mG.mA.mC.mA.mU.mC.mC.mA.mG.mA.fU.mA |
| | | .mA.mU.mC.mC.mU.mA.fU.fC.fU.mG.mG.mA.mU.mG.mU |
| | | .mC.mU.mA.mA |
| 1709 | 205 | PmU.fC.mU.mC.mC.mU.mU.mC.mC.mG.mA.mG.mU.fC.mA |
| | | .mG.mC.mU.mU.mU.mG.fA.fC.fU.mC.mG.mG.mA.mA.mG |
| | | .mG.mA.mG.mA |
| 1710 | 206 | PmU.fC.mA.mU.mG.mG.mA.mG.mC.mC.mU.mG.mA.fA.mG |
| | | .mG.mG.mU.mC.mC.mU.fU.fC.fA.mG.mG.mC.mU.mC.mC |
| | | .mA.mU.mG.mA |
| 1711 | 207 | PmU.fU.mG.mG.mC.mA.mU.mA.mU.mG.mU.mC.mA.fC.mU |
| | | .mA.mG.mA.mC.mA.mG.fU.fG.fA.mC.mA.mU.mA.mU.mG |
| | | .mC.mC.mA.mA |
| 1712 | 208 | PmU.fA.mA.mG.mC.mA.mU.mU.mG.mA.mU.mG.mU.fU.mC |
| | | .mA.mC.mU.mU.mG.mA.fA.fC.fA.mU.mC.mA.mA.mU.mG |
| | | .mC.mU.mU.mA |
| 1713 | 209 | PmU.fC.mU.mC.mA.mC.mU.mC.mC.mU.mC.mC.mA.fG.mU |
| | | .mA.mC.mA.mA.mA.mC.fU.fG.fG.mA.mG.mG.mA.mG.mU |
| | | .mG.mA.mG.mA |
| 1714 | 210 | PmU.fG.mA.mU.mG.mU.mC.mC.mU.mU.mG.mA.mC.fU.mU |
| | | .mU.mG.mU.mC.mA.mA.fG.fU.fC.mA.mA.mG.mG.mA.mC |
| | | .mA.mU.mC.mA |
| 1715 | 211 | PmU.fU.mG.mU.mU.mU.mU.mA.mA.mU.mU.mC.mA.fA.mU |
| | | .mC.mC.mC.mA.mA.mU.fU.fG.fA.mA.mU.mU.mA.mA.mA |
| | | .mA.mC.mA.mA |
| 1716 | 212 | PmU.fU.mA.mG.mA.mU.mG.mU.mU.mC.mA.mU.mG.fG.mA |
| | | .mG.mC.mC.mU.mU.mC.fC.fA.fU.mG.mA.mA.mC.mA.mU |
| | | .mC.mU.mA.mA |
| 1717 | 213 | PmU.fA.mU.mA.mG.mC.mA.mG.mU.mG.mG.mA.mA.fA.mG |
| | | .mA.mG.mA.mU.mC.mU.fU.fU.fC.mC.mA.mC.mU.mG.mC |
| | | .mU.mA.mU.mA |
| 1718 | 214 | PmU.fC.mA.mU.mU.mC.mA.mC.mU.mU.mG.mG.mC.fA.mG |
| | | .mG.mU.mG.mC.mC.mU.fG.fC.fC.mA.mA.mG.mU.mG.mA |
| | | .mA.mU.mG.mA |
| 1719 | 215 | PmU.fA.mU.mU.mG.mA.mU.mG.mU.mU.mC.mA.mC.fU.mU |
| | | .mG.mG.mU.mU.mA.mA.fG.fU.fG.mA.mA.mC.mA.mU.mC |
| | | .mA.mA.mU.mA |
| 1720 | 216 | PmU.fC.mA.mG.mC.mC.mA.mG.mG.mG.mC.mA.mG.fC.mA |
| | | .mC.mU.mU.mG.mU.mG.fC.fU.fG.mC.mC.mC.mU.mG.mG |
| | | .mC.mU.mG.mA |
| 1721 | 217 | PmU.fC.mU.mC.mA.mG.mU.mG.mU.mC.mC.mA.mA.fG.mC |
| | | .mU.mG.mA.mA.mG.mC.fU.fU.fG.mG.mA.mC.mA.mC.mU |
| | | .mG.mA.mG.mA |
| 1722 | 218 | PmU.fC.mA.mG.mA.mG.mA.mC.mU.mG.mG.mC.mU.fU.mU |
| | | .mC.mA.mU.mC.mA.mA.fA.fG.fC.mC.mA.mG.mU.mC.mU |
| | | .mC.mU.mG.mA |
| 1723 | 219 | PmU.fA.mU.mC.mC.mA.mG.mA.mU.mA.mA.mU.mC.fC.mU |
| | | .mC.mC.mC.mU.mA.mG.fG.fA.fU.mU.mA.mU.mC.mU.mG |
| | | .mG.mA.mU.mA |
| 1724 | 220 | PmU.fC.mU.mU.mC.mU.mC.mA.mA.mU.mU.mA.mA.fG.mU |
| | | .mU.mG.mA.mC.mA.mC.fU.fU.fA.mA.mU.mU.mG.mA.mG |
| | | .mA.mA.mG.mA |
| 1725 | 221 | PmU.fC.mC.mA.mG.mA.mC.mC.mU.mA.mG.mA.mC.fC.mU |
| | | .mG.mG.mU.mC.mA.mG.fG.fU.fC.mU.mA.mG.mG.mU.mC |
| | | .mU.mG.mG.mA |
| 1726 | 222 | PmU.fC.mA.mA.mC.mG.mU.mC.mA.mU.mA.mG.mU.fC.mA |
| | | .mU.mA.mA.mA.mU.mG.fA.fC.fU.mA.mU.mG.mA.mC.mG |
| | | .mU.mU.mG.mA |
| 1727 | 223 | PmU.fU.mU.mG.mA.mA.mU.mG.mA.mA.mA.mC.mG.fA.mC |
| | | .mU.mU.mC.mU.mG.mU.fC.fG.fU.mU.mU.mC.mA.mU.mU |
| | | .mC.mA.mA.mA |
| 1728 | 224 | PmU.fC.mC.mU.mC.mC.mU.mC.mA.mG.mA.mC.mA.fC.mA |
| | | .mA.mA.mC.mA.mU.mG.fU.fG.fU.mC.mU.mG.mA.mG.mG |
| | | .mA.mG.mG.mA |
| 1729 | 225 | PmU.fA.mA.mG.mC.mC.mA.mA.mA.mG.mC.mA.mU.fU.mG |
| | | .mA.mU.mG.mU.mC.mA.fA.fU.fG.mC.mU.mU.mU.mG.mG |
| | | .mC.mU.mU.mA |
| 1730 | 226 | PmU.fU.mG.mA.mA.mA.mC.mG.mA.mC.mU.mU.mC.fU.mC |
| | | .mU.mU.mG.mU.mG.mA.fG.fA.fA.mG.mU.mC.mG.mU.mU |
| | | .mU.mC.mA.mA |
| 1731 | 227 | PmU.fA.m.A.mC.mU.mU.mG.mU.mG.mG.mU.mC.mU.fU.m |
| | | C.mA.mU.mA.mA.mG.mA.fA.fG.fA.mC.mC.mA.mC.mA.m |
| | | A.mG.mU.mU.mA |
| 1732 | 228 | PmU.fC.mC.mA.mG.mG.mU.mA.mG.mA.mU.mG.mU.fU.mC |
| | | .mA.mU.mG.mG.mG.mA.fA.fC.fA.mU.mC.mU.mA.mC.mC |
| | | .mU.mG.mG.mA |
| 1733 | 229 | PmU.fC.mU.mG.mU.mG.mU.mU.mC.mU.mG.mG.mC.fA.mC |
| | | .mC.mU.mG.mC.mG.mU.fG.fC.fC.mA.mG.mA.mA.mC.mA |
| | | .mC.mA.mG.mA |
| 1734 | 230 | PmU.fA.mA.mC.mU.mU.mG.mC.mC.mA.mC.mC.mU.fU.mC |
| | | .mU.mC.mA.mA.mG.mA.fA.fG.fG.mU.mG.mG.mC.mA.mA |
| | | .mG.mU.mU.mA |
| 1735 | 231 | PmU.fG.mC.mC.mA.mU.mG.mG.mU.mU.mG.mC.mU.fU.mG |
| | | .mU.mG.mG.mU.mC.mA.fA.fG.fC.mA.mA.mC.mC.mA.mU |
| | | .mG.mG.mC.mA |
| 1736 | 232 | PmU.fG.mA.mC.mA.mA.mA.mU.mG.mG.mG.mC.mC.fU.mG |
| | | .mA.mU.mA.mG.mC.mA.fG.fG.fC.mC.mC.mA.mU.mU.mU |
| | | .mG.mU.mC.mA |
| 1737 | 233 | PmU.fA.mA.mG.mU.mU.mG.mA.mC.mU.mA.mG.mA.fC.mA |
| | | .mC.mU.mU.mU.mU.mG.fU.fC.fU.mA.mG.mU.mC.mA.mA |
| | | .mC.mU.mU.mA |
| 1738 | 234 | PmU.fC.mA.mU.mG.mG.mC.mG.mG.mG.mU.mG.mC.fG.mG |
| | | .mU.mU.mC.mC.mC.mC.fG.fC.fA.mC.mC.mC.mG.mC.mC |
| | | .mA.mU.mG.mA |
| 1739 | 235 | PmU.fC.mC.mG.mG.mA.mU.mC.mU.mC.mA.mU.mC.fA.mA |
| | | .mU.mG.mA.mC.mU.mU.fG.fA.fU.mG.mA.mG.mA.mU.mC |
| | | .mC.mG.mG.mA |
| 1740 | 236 | PmU.fG.mA.mG.mG.mA.mA.mG.mC.mC.mU.mC.mA.fA.mA |
| | | .mG.mC.mU.mC.mU.mU.fU.fG.fA.mG.mG.mC.mU.mU.mC |
| | | .mC.mU.mC.mA |
| 1741 | 237 | PmU.fA.mC.mU.mU.mU.mG.mA.mA.mC.mA.mC.mA.fU.mG |
| | | .mU.mU.mG.mC.mC.mA.fU.fG.fU.mG.mU.mU.mC.mA.mA |
| | | .mA.mG.mU.mA |
| 1742 | 238 | PmU.fC.mU.mC.mC.mU.mC.mC.mU.mC.mA.mG.mA.fC.mA |
| | | .mC.mA.mA.mA.mU.mG.fU.fC.fU.mG.mA.mG.mG.mA.mG |
| | | .mG.mA.mG.mA |
| 1743 | 239 | PmU.fA.mG.mG.mA.mA.mG.mG.mC.mU.mC.mC.mG.fU.mC |
| | | .mC.mC.mG.mC.mG.mA.fC.fG.fG.mA.mG.mC.mC.mU.mU |
| | | .mC.mC.mU.mA |
| 1744 | 240 | PmU.fC.mG.mC.mC.mA.mG.mA.mA.mU.mC.mA.mC.fC.mU |
| | | .mC.mU.mG.mC.mA.mG.fG.fU.fG.mA.mU.mU.mC.mU.mG |
| | | .mG.mC.mG.mA |
| 1745 | 241 | PmU.fG.mG.mA.mA.mA.mG.mA.mG.mA.mU.mC.mU.fC.mA |
| | | .mU.mC.mA.mC.mU.mG.fA.fG.fA.mU.mC.mU.mC.mU.mU |
| | | .mU.mC.mC.mA |
| 1746 | 242 | PmU.fA.mA.mG.mU.mC.mC.mC.mG.mG.mA.mU.mC.fU.mC |
| | | .mA.mU.mC.mA.mG.mA.fG.fA.fU.mC.mC.mG.mG.mG.mA |
| | | .mC.mU.mU.mA |
| 1747 | 243 | PmU.fG.mA.mG.mC.mU.mU.mG.mA.mU.mC.mA.mG.fG.mG |
| | | .mC.mA.mA.mC.mC.mC.fC.fU.fG.mA.mU.mC.mA.mA.mG |
| | | .mC.mU.mC.mA |
| 1748 | 244 | PmU.fG.mU.mU.mG.mC.mU.mC.mA.mU.mU.mG.mU.fC.mU |
| | | .mU.mU.mC.mU.mA.mG.fA.fC.fA.mA.mU.mG.mA.mG.mC |
| | | .mA.mA.mC.mA |
| 1749 | 245 | PmU.fU.mG.mC.mU.mU.mG.mU.mG.mG.mU.mA.mA.fU.mC |
| | | .mG.mG.mU.mA.mG.mA.fU.fU.fA.mC.mC.mA.mC.mA.mA |
| | | .mG.mC.mA.mA |
| 1750 | 246 | PmU.fU.mC.mA.mU.mC.mA.mC.mU.mC.mA.mC.mA.fU.mU |
| | | .mG.mU.mA.mG.mA.mA.fU.fG.fU.mG.mA.mG.mU.mG.mA |
| | | .mU.mG.mA.mA |
| 1751 | 247 | PmU.fC.mA.mG.mU.mG.mU.mC.mC.mA.mA.mG.mC.fU.mG |
| | | .mA.mA.mA.mC.mC.mA.fG.fC.fU.mU.mG.mG.mA.mC.mA |
| | | .mC.mU.mG.mA |
| 1752 | 248 | PmU.fC.mC.mA.mU.mU.mC.mU.mU.mG.mA.mU.mG.fU.mA |
| | | .mG.mA.mC.mC.mU.mA.fC.fA.fU.mC.mA.mA.mG.mA.mA |
| | | .mU.mG.mG.mA |
| 1753 | 249 | PmU.fA.m.A.mC.mA.mA.mU.mG.mU.mG.mC.mU.mG.fC.m |
| | | U.mG.mU.mC.mA.mA.mG.fC.fA.fG.mC.mA.mC.mA.mU.m |
| | | U.mG.mU.mU.mA |
| 1754 | 250 | PmU.fG.mA.mC.mU.mU.mC.mA.mA.mC.mU.mU.mG.fU.mG |
| | | .mG.mU.mC.mU.mC.mA.fC.fA.fA.mG.mU.mU.mG.mA.mA |
| | | .mG.mU.mC.mA |
| 1755 | 106-13(4) | mU.fU.mG.fA.mA.fU.mG.fA.mA.fA.mC.fG.mA.fC.mU. |
| | as + s | fU.mC.fU.mC.fG.mU.fU.mU.fC.mA.fU.mU.fC.mA.fA. |
| | | 3xGalNAc |
| 1756 | 13(5) | mU.fU.mG.mC.mC.mA.mC.mA.mG.mA.mG.mA.mC.fU.mC. |
| | as +s | mA.mG.mA.mG.mA.fG.fU.fC.mU.mC.mU.mG.mU.mG.mG. |
| | | mC.mA.mA.3xGaINAc |

The first nucleobase on the terminal 5′ position (the sequences in the table are presented from a 5′(Ieft) to a 3′(right direction) can be freely selected from U, A, G and C instead of the nucleobase disclosed in the table.

Note=each of the above constructs may or may not have a phosphate modification at the 5′ end group. Furthermore, and independently, each of the above constructs may or may not have a “3× GalNAc” coupled to the 3′ end group. Advantageously the constructs contain a 3× GalNAc ligand, in particular a toothbrush ligand as defined herein. Particularly advantageous are constructs, which in addition have a 5′ phosphate, even though this is not a strict requirement, given that in the absence thereof, mammalian cells will add such phosphate in case it is absent from the molecule as administered.

TABLE 3b
Modified CFB hairpin constructs including
internucleoside linkages

| SEQ | Construct | Modified CFB hairpin |
|---|---|---|
| ID No. | ID NO. | constructs |
| 1757 | 13(5) | 5′-usUfsgccacagagacUfscs |
| | as + s | asgsasgsaGfUfCfucuguggcs |
| | | asas(SO-GalNAc)(SO-GalNAc) |
| | | (SO-GalNAc)-3′ |
| 1758 | 106-13(4) | 5′-usUfsgAfaUfgAfaAfcGfa |
| | as + s | sCfsusUfscsUfscGfuUfuCfa |
| | | UfuCfsasAfs(SO-GalNAc)(SO- |
| | | GalNAc)(SO-GalNAc)-3′ |

The first nucleobase on the terminal 5′ position (the sequences in the table are presented from a 5′(left) to a 3′(right direction) can be freely selected from U, A, G and C instead of the nucleobase disclosed in the table.

TABLE 3c

| SEQ ID | Construct | Antisense | |
|---|---|---|---|
| No. | ID NO. | ID | Modified 19mer C5 Antisense constructs |
| 1759 | 251 | 24151 | [5Phos][mU][Ps][fC][Ps][mA][fC][mA][fG][mU][fU][mU][fG][mG] |
| | | | [fC][mC][fU][Ps][mG][Ps][fG][Ps][mA][Ps][fG][Ps][mA] |
| 1760 | 252 | 24152 | [5Phos][mU][Ps][fG][Ps][mA][fA][mU][fC][mU][fU][mG][fA][mA] |
| | | | [fG][mU][fC][Ps][mA][Ps][fG][Ps][mG][Ps][fA][Ps][mA] |
| 1761 | 253 | 24153 | [5Phos][mU][Ps][fU][Ps][mG][fG][mG][fC][mU][fU][mG][fU][mA] |
| | | | [fG][mC][fU][Ps][mG][Ps][fG][Ps][mC][Ps][fA][Ps][mC] |
| 1762 | 254 | 24154 | [5Phos][mU][Ps][fC][Ps][mA][fA][mG][fU][mA][fA][mU][fU][mA] |
| | | | [fU][mA][fG][Ps][mU][Ps][fG][Ps][mA][Ps][fG][Ps][mU] |
| 1763 | 255 | 24155 | [5Phos][mU][Ps][fA][Ps][mA][fC][mA][fG][mG][fU][mU][fU][mG] |
| | | | [fU][mC][fU][Ps][mG][Ps][fU][Ps][mA][Ps][fU][Ps][mG] |
| 1764 | 256 | 24156 | [5Phos][mU][Ps][fC][Ps][mA][fG][mA][fC][mA][fU][mU][fU][mU] |
| | | | [fA][mA][fC][Ps][mA][Ps][fC][Ps][mA][Ps][fG][Ps][mA] |
| 1765 | 257 | 24157 | [5Phos][mU][Ps][fC][Ps][mC][fU][mG][fG][mA][fG][mC][fU][mG] |
| | | | [fG][mU][fU][Ps][mG][Ps][fC][Ps][mC][Ps][fA][Ps][mC] |
| 1766 | 258 | 24158 | [5Phos][mU][Ps][fA][Ps][mU][fA][mA][fA][mA][fU][mC][fA][mA] |
| | | | [fG][mU][fA][Ps][mA][Ps][fU][Ps][mU][Ps][fA][Ps][mU] |
| 1767 | 259 | 24159 | [5Phos][mU][Ps][fA][Ps][mC][fA][mC][fA][mG][fU][mU][fU][mG] |
| | | | [fG][mC][fC][Ps][mU][Ps][fG][Ps][mG][Ps][fA][Ps][mG] |
| 1768 | 260 | 24160 | [5Phos][mU][Ps][fG][Ps][mG][fA][mA][fU][mC][fU][mU][fG][mA] |
| | | | [fA][mG][fU][Ps][mC][Ps][fA][Ps][mG][Ps][fG][Ps][mA] |
| 1769 | 261 | 24161 | [5Phos][mU][Ps][fG][Ps][mA][fC][mA][fU][mU][fU][mU][fA][mA] |
| | | | [fC][mA][f C][Ps][mA][Ps][fG][Ps][mA][Ps][fA][Ps][mC] |
| 1770 | 262 | 24162 | [5Phos][mU][Ps][fG][Ps][mA][fA][mG][fU][mC][fA][mG][fG][mA] |
| | | | [fA][mA][fA][Ps][mG][Ps][fA][Ps][mG][Ps][fA][Ps][mU] |
| 1771 | 263 | 24163 | [5Phos][mU][Ps][fA][Ps][mC][fA][mG][fU][mU][fU][mG][fG][mC] |
| | | | [fC][mU][fG][Ps][mG][Ps][fA][Ps][mG][Ps][fA][Ps][mA] |
| 1772 | 264 | 24164 | [5Phos][mU][Ps][fA][Ps][mU][fU][mA][fU][mA][fG][mU][fG][mA] |
| | | | [fG][mU][fU][Ps][mA][Ps][fU][Ps][mU][Ps][fU][Ps][mU] |
| 1773 | 265 | 24165 | [5Phos][mU][Ps][fC][Ps][mC][fA][mA][fG][mU][fC][mA][fG][mA] |
| | | | [fU][mG][fU][Ps][mC][Ps][fU][Ps][mC][Ps][fU][Ps][mU] |
| 1774 | 266 | 24166 | [5Phos][mU][Ps][fC][Ps][mA][fG][mG][fA][mA][fA][mA][fG][mA] |
| | | | [fG][mA][fU][Ps][mA][Ps][fA][Ps][mU][Ps][fU][Ps][mC] |
| 1775 | 267 | 24167 | [5Phos][mU][Ps][fG][Ps][mC][fA][mA][fG][mA][fC][mA][fU][mA] |
| | | | [fU][mU][fC][Ps][mU][Ps][fU][Ps][mU][Ps][fA][Ps][mA] |
| 1776 | 268 | 24168 | [5Phos][mU][Ps][fA][Ps][mA][fG][mG][fC][mC][fA][mA][fU][mU] |
| | | | [fU][mC][fC][Ps][mA][Ps][fG][Ps][mA][Ps][fG][Ps][mG] |
| 1777 | 269 | 24169 | [5Phos][mU][Ps][fA][Ps][mA][fG][mU][fA][mA][fU][mU][fA][mU] |
| | | | [fA][mG][fU][Ps][mG][Ps][fA][Ps][mG][Ps][fU][Ps][mU] |
| 1778 | 270 | 24170 | [5Phos][mU][Ps][fU][Ps][mA][fA][mA][fA][mU][fC][mA][fA][mG] |
| | | | [fU][mA][fA][Ps][mU][Ps][fU][Ps][mA][Ps][fU][Ps][mA] |
| 1779 | 271 | 24171 | [5Phos][mU][Ps][fU][Ps][mC][fA][mA][fG][mU][fA][mA][fU][mU] |
| | | | [fA][mU][fA][Ps][mG][Ps][fU][Ps][mG][Ps][fA][Ps][mG] |
| 1780 | 272 | 24172 | [5Phos][mU][Ps][fC][Ps][mC][fA][mA][fU][mU][fU][mC][fC][mA] |
| | | | [fG][mA][fG][Ps][mG][Ps][fA][Ps][mA][Ps][fG][Ps][mC] |
| 1781 | 273 | 24173 | [5Phos][mU][Ps][fG][Ps][mU][fA][mA][fU][mU][fA][mU][fA][mG] |
| | | | [fU][mG][fA][Ps][mG][Ps][fU][Ps][mU][Ps][fA][Ps][mU] |
| 1782 | 274 | 24174 | [5Phos][mU][Ps][fA][Ps][mA][fA][mG][fG][mU][fA][mC][fU][mU] |
| | | | [fG][mU][fU][Ps][mG][Ps][fU][Ps][mU][Ps][fU][Ps][mA] |
| 1783 | 275 | 24175 | [5Phos][mU][Ps][fA][Ps][mC][fU][mG][fC][mU][fG][mU][fU][mU] |
| | | | [fC][mA][fG][Ps][mA][Ps][fA][Ps][mU][Ps][fC][Ps][mA] |
| 1784 | 276 | 24176 | [5Phos][mU][Ps][fC][Ps][mU][fG][mC][fU][mG][fU][mU][fU][mC] |
| | | | [fA][mG][fA][Ps][mA][Ps][fU][Ps][mC][Ps][fA][Ps][mA] |
| 1785 | 277 | 24177 | [5Phos][mU][Ps][fU][Ps][mA][fU][mA][fA][mA][fG][mG][fU][mA] |
| | | | [fC][mU][fU][Ps][mG][Ps][fU][Ps][mU][Ps][fG][Ps][mU] |
| 1786 | 278 | 24178 | [5Phos][mU][Ps][fG][Ps][mU][fA][mA][fA][mC][fA][mG][fU][mU] |
| | | | [fC][mC][fU][Ps][mU][Ps][fU][Ps][mC][Ps][fA][Ps][mA] |
| 1787 | 279 | 24179 | [5Phos][mU][Ps][fG][Ps][mU][fA][mA][fC][mU][fU][mU][fG][mG] |
| | | | [fC][mU][fG][Ps][mA][Ps][fG][Ps][mA][Ps][fG][Ps][mA] |
| 1788 | 280 | 24180 | [5Phos][mU][Ps][fA][Ps][mU][fA][mG][fU][mU][fG][mU][fA][mA] |
| | | | [fA][mC][fA][Ps][mG][Ps][fU][Ps][mU][Ps][fC][Ps][mC] |
| 1789 | 281 | 24181 | [5Phos][mU][Ps][fC][Ps][mA][fU][mA][fU][mU][fC][mU][fU][mU] |
| | | | [fA][mA][fC][Ps][mU][Ps][fU][Ps][mC][Ps][f A][Ps][mA] |
| 1790 | 282 | 24182 | [5Phos][mU][Ps][fA][Ps][mG][fC][mA][fG][mU][fC][mC][fU][mU] |
| | | | [fU][mU][fA][Ps][mC][Ps][fA][Ps][mC][Ps][fU][Ps][mC] |
| 1791 | 283 | 24183 | [5Phos][mU][Ps][fA][Ps][mG][fU][mG][fA][mG][fU][mU][fA][mU] |
| | | | [fU][mU][fU][Ps][mG][Ps][fU][Ps][mC][Ps][fA][Ps][mA] |
| 1792 | 284 | 24184 | [5Phos][mU][Ps][fG][Ps][mG][fA][mA][fG][mA][fC][mA][fU][mC] |
| | | | [fU][mU][fU][Ps][mG][Ps][fA][Ps][mA][Ps][fC][Ps][mA] |
| 1793 | 285 | 24185 | [5Phos][mU][Ps][fC][Ps][mA][fG][mU][fC][mC][fU][mU][fU][mU] |
| | | | [fA][mC][fA][Ps][mC][Ps][fU][Ps][mC][Ps][fA][Ps][mA] |
| 1794 | 286 | 24186 | [5Phos][mU][Ps][fG][Ps][mU][fU][mA][fU][mU][fU][mU][fG][mU] |
| | | | [fC][mA][fA][Ps][mU][Ps][fA][Ps][mU][Ps][fA][Ps][mU] |
| 1795 | 287 | 24187 | [5Phos][mU][Ps][fU][Ps][mA][fC][mA][fA][mC][fA][mG][fA][mA] |
| | | | [fU][mA][fU][Ps][mG][Ps][fG][Ps][mU][Ps][fA][Ps][mU] |
| 1796 | 288 | 24188 | [5Phos][mU][Ps][fU][Ps][mU][fA][mU][fU][mU][fU][mG][fU][mC] |
| | | | [fA][mA][fU][Ps][mA][Ps][fU][Ps][mA][Ps][fU][Ps][mG] |
| 1797 | 289 | 24189 | [5Phos][mU][Ps][fA][Ps][mG][fG][mC][fU][mU][fC][mA][fG][mG] |
| | | | [fA][mA][fA][Ps][mA][Ps][fG][Ps][mA][Ps][fG][Ps][mG] |
| 1798 | 290 | 24190 | [5Phos][mU][Ps][fG][Ps][mG][fA][mA][fA][mA][fG][mA][fG][mA] |
| | | | [fU][mA][fA][Ps][mU][Ps][fU][Ps][mC][Ps][fC][Ps][mA] |
| 1799 | 291 | 24191 | [5Phos][mU][Ps][fG][Ps][mU][fU][mA][fC][mA][fG][mC][fA][mA] |
| | | | [fU][mA][fU][Ps][mA][Ps][fA][Ps][mA][Ps][fG][Ps][mG] |
| 1800 | 292 | 24192 | [5Phos][mU][Ps][fA][Ps][mU][fA][mA][fG][mC][fA][mU][fA][mU] |
| | | | [fG][mC][fA][Ps][mA][Ps][fU][Ps][mC][Ps][fU][Ps][mC] |
| 1801 | 293 | 24193 | [5Phos][mU][Ps][fA][Ps][mU][fA][mU][fU][mC][fU][mU][fU][mA] |
| | | | [fA][mC][fU][Ps][mU][Ps][fC][Ps][mA][Ps][fA][Ps][mA] |
| 1802 | 294 | 24194 | [5Phos][mU][Ps][fA][Ps][mG][fA][mC][fA][mU][fC][mU][fU][mU] |
| | | | [fG][mA][fA][Ps][mC][Ps][fA][Ps][mC][Ps][fC][Ps][mU] |
| 1803 | 295 | 24195 | [5Phos][mU][Ps][fC][Ps][mA][fG][mG][fA][mA][fG][mA][fC][mA] |
| | | | [fU][mC][fU][Ps][mU][Ps][fU][Ps][mG][Ps][fA][Ps][mA] |
| 1804 | 296 | 24196 | [5Phos][mU][Ps][fA][Ps][mC][fA][mG][fC][mA][fA][mU][fA][mU] |
| | | | [fA][mA][fA][Ps][mG][Ps][fG][Ps][mU][Ps][fA][Ps][mC] |
| 1805 | 297 | 24197 | [5Phos][mU][Ps][fA][Ps][mU][fU][mG][fU][mC][fA][mU][fA][mG] |
| | | | [fG][mU][fU][Ps][mA][Ps][fU][Ps][mU][Ps][fG][Ps][mG] |
| 1806 | 298 | 24198 | [5Phos][mU][Ps][fG][Ps][mA][fG][mU][fU][mA][fU][mU][fU][mU] |
| | | | [fG][mU][fC][Ps][mA][Ps][fA][Ps][mU][Ps][fA][Ps][mU] |
| 1807 | 299 | 24199 | [5Phos][mU][Ps][fG][Ps][mU][fG][mA][fG][mU][fU][mA][fU][mU] |
| | | | [fU][mU][fG][Ps][mU][Ps][fC][Ps][mA][Ps][fA][Ps][mU] |
| 1808 | 300 | 24200 | [5Phos][mU][Ps][fU][Ps][mG][fA][mG][fU][mU][fA][mU][fU][mU] |
| | | | [fU][mG][fU][Ps][mC][Ps][fA][Ps][mA][Ps][fU][Ps][mA] |
| 1809 | 301 | 24201 | [5Phos][mU][Ps][fA][Ps][mA][fU][mU][fU][mU][fC][mC][fU][mU] |
| | | | [fG][mA][fA][Ps][mA][Ps][fG][Ps][mA][Ps][fU][Ps][mC] |
| 1810 | 302 | 24202 | [5Phos][mU][Ps][fC][Ps][mU][fG][mU][fU][mA][fC][mA][fG][mC] |
| | | | [fA][mA][fU][Ps][mA][Ps][fU][Ps][mA][Ps][fA][Ps][mA] |
| 1811 | 303 | 24203 | [5Phos][mU][Ps][fA][Ps][mA][fU][mC][fC][mA][fU][mU][fG][mU] |
| | | | [fC][mA][fU][Ps][mA][Ps][fG][Ps][mG][Ps][fU][Ps][mU] |
| 1812 | 304 | 24204 | [5Phos][mU][Ps][fA][Ps][mU][fC][mC][fA][mU][fU][mG][fU][mC] |
| | | | [fA][mU][fA][Ps][mG][Ps][fG][Ps][mU][Ps][fU][Ps][mA] |
| 1813 | 305 | 24205 | [5Phos][mU][Ps][fA][Ps][mG][fA][mA][fA][mU][fC][mC][fA][mU] |
| | | | [fU][mG][fU][Ps][mC][Ps][fA][Ps][mU][Ps][fA][Ps][mG] |
| 1814 | 306 | 24206 | [5Phos][mU][Ps][fA][Ps][mG][fA][mC][fA][mU][fA][mU][fU][mC] |
| | | | [fU][mU][fU][Ps][mA][Ps][fA][Ps][mC][Ps][fU][Ps][mU] |
| 1815 | 307 | 24207 | [5Phos][mU][Ps][fA][Ps][mG][fU][mG][fC][mA][fG][mA][fU][mU] |
| | | | [fC][mC][fC][Ps][mU][Ps][fC][Ps][mC][Ps][fA][Ps][mC] |
| 1816 | 308 | 24208 | [5Phos][mU][Ps][fU][Ps][mC][fC][mA][fU][mU][fG][mU][fC][mA] |
| | | | [fU][mA][fG][Ps][mG][Ps][fU][Ps][mU][Ps][fA][Ps][mU] |
| 1817 | 309 | 24209 | [5Phos][mU][Ps][fG][Ps][mA][fC][mA][fU][mC][fU][mU][fU][mG] |
| | | | [fA][mA][fC][Ps][mA][Ps][fC][Ps][mC][Ps][fU][Ps][mU] |
| 1818 | 310 | 24210 | [5Phos][mU][Ps][fC][Ps][mA][fU][mU][fG][mU][fC][mA][fU][mA] |
| | | | [fG][mG][fU][Ps][mU][Ps][fA][Ps][mU][Ps][fU][Ps][mG] |
| 1819 | 311 | 24211 | [5Phos][mU][Ps][fG][Ps][mA][fA][mG][fA][mG][fA][mA][fA][mU] |
| | | | [fC][mC][fA][Ps][mU][Ps][fU][Ps][mG][Ps][fU][Ps][mC] |
| 1820 | 312 | 24212 | [5Phos][mU][Ps][fA][Ps][mC][fA][mU][fA][mU][fU][mC][fU][mU] |
| | | | [fU][mA][fA][Ps][mC][Ps][fU][Ps][mU][Ps][fC][Ps][mA] |
| 1821 | 313 | 24213 | [5Phos][mU][Ps][fA][Ps][mG][fU][mC][fC][mU][fU][mU][fU][mA] |
| | | | [fC][mA][fC][Ps][mU][Ps][fC][Ps][mA][Ps][fA][Ps][mA] |
| 1822 | 314 | 24214 | [5Phos][mU][Ps][fA][Ps][mU][fU][mU][fU][mC][fC][mU][fU][mG] |
| | | | [fA][mA][fA][Ps][mG][Ps][fA][Ps][mU][Ps][fC][Ps][mC] |
| 1823 | 315 | 24215 | [5Phos][mU][Ps][fG][Ps][mA][fA][mA][fU][mU][fG][mU][fA][mU] |
| | | | [fU][mU][fU][Ps][mA][Ps][fU][Ps][mC][Ps][fU][Ps][mG] |
| 1824 | 316 | 24216 | [5Phos][mU][Ps][fG][Ps][mU][fA][mA][fU][mU][fU][mC][fA][mA] |
| | | | [fA][mA][fU][Ps][mU][Ps][fC][Ps][mU][Ps][fU][Ps][mA] |
| 1825 | 317 | 24217 | [5Phos][mU][Ps][fA][Ps][mA][fA][mA][fU][mU][fC][mU][fU][mA] |
| | | | [fA][mA][fG][Ps][mU][Ps][fU][Ps][mC][Ps][fU][Ps][mU] |
| 1826 | 318 | 24218 | [5Phos][mU][Ps][fG][Ps][mA][fA][mU][fU][mU][fU][mG][fG][mU] |
| | | | [fU][mC][fU][Ps][mG][Ps][fC][Ps][mU][Ps][fC][Ps][mU] |
| 1827 | 319 | 24219 | [5Phos][mU][Ps][fG][Ps][mU][fC][mA][fU][mU][fU][mU][fA][mU] |
| | | | [fA][mA][fU][Ps][mU][Ps][fA][Ps][mU][Ps][fG][Ps][mU] |
| 1828 | 320 | 24220 | [5Phos][mU][Ps][fC][Ps][mA][fA][mA][fU][mC][fC][mU][fG][mU] |
| | | | [fA][mC][fU][Ps][mG][Ps][fA][Ps][mC][Ps][fA][Ps][mA] |
| 1829 | 321 | 24221 | [5Phos][mU][Ps][fG][Ps][mA][fU][mA][fA][mC][fU][mU][fU][mU] |
| | | | [fA][mA][fU][Ps][mA][Ps][fG][Ps][mA][Ps][fG][Ps][mA] |
| 1830 | 322 | 24222 | [5Phos][mU][Ps][fU][Ps][mU][fA][mA][fG][mU][fC][mU][fU][mC] |
| | | | [fU][mC][fU][Ps][mU][Ps][fA][Ps][mU][Ps][fU][Ps][mC] |
| 1831 | 323 | 24223 | [5Phos][mU][Ps][fA][Ps][mU][fA][mA][fU][mU][fC][mC][fA][mA] |
| | | | [fU][mA][fU][Ps][mG][Ps][fA][Ps][mU][Ps][fC][Ps][mA] |
| 1832 | 324 | 24224 | [5Phos][mU][Ps][fG][Ps][mA][fU][mA][fA][mA][fU][mG][fA][mA] |
| | | | [fC][mA][fU][Ps][mG][Ps][fG][Ps][mC][Ps][fC][Ps][mU] |
| 1833 | 325 | 24225 | [5Phos][mU][Ps][fA][Ps][mA][fG][mG][fU][mU][fC][mA][fU][mC] |
| | | | [fA][mU][fU][Ps][mU][Ps][fU][Ps][mC][Ps][fU][Ps][mU] |
| 1834 | 326 | 24226 | [5Phos][mU][Ps][fG][Ps][mG][fA][mA][fG][mU][fG][mC][fU][mA] |
| | | | [fU][mA][fA][Ps][mA][Ps][fA][Ps][mC][Ps][fA][Ps][mU] |
| 1835 | 327 | 24227 | [5Phos][mU][Ps][fC][Ps][mA][fA][mG][fU][mA][fC][mU][fC][mU] |
| | | | [fU][mA][fA][Ps][mA][Ps][fG][Ps][mC][Ps][fA][Ps][mA] |
| 1836 | 328 | 24228 | [5Phos][mU][Ps][fC][Ps][mC][fA][mA][fU][mG][fA][mU][fU][mU] |
| | | | [fC][mC][fU][Ps][mG][Ps][fU][Ps][mU][Ps][fU][Ps][mC] |
| 1837 | 329 | 24229 | [5Phos][mU][Ps][fA][Ps][mU][fG][mG][fU][mA][fU][mA][fU][mU] |
| | | | [fC][mA][fU][Ps][mU][Ps][fU][Ps][mC][Ps][fC][Ps][mA] |
| 1838 | 330 | 24230 | [5Phos][mU][Ps][fA][Ps][mA][fC][mA][fA][mG][fA][mU][fG][mA] |
| | | | [fA][mC][fU][Ps][mU][Ps][fC][Ps][mC][Ps][fC][Ps][mA] |
| 1839 | 331 | 24231 | [5Phos][mU][Ps][fG][Ps][mA][fA][mC][fU][mU][fC][mA][fG][mG] |
| | | | [fA][mA][fU][Ps][mU][Ps][fU][Ps][mU][Ps][fA][Ps][mG] |
| 1840 | 332 | 24232 | [5Phos][mU][Ps][fA][Ps][mG][fU][mC][fU][mU][fC][mU][fC][mU] |
| | | | [fU][mA][fU][Ps][mU][Ps][fC][Ps][mC][Ps][fA][Ps][mA] |
| 1841 | 333 | 24233 | [5Phos][mU][Ps][fA][Ps][mA][fU][mG][fU][mU][fU][mA][fU][mA] |
| | | | [fC][mU][fU][Ps][mU][Ps][fG][Ps][mA][Ps][fU][Ps][mA] |
| 1842 | 334 | 24234 | [5Phos][mU][Ps][fC][Ps][mG][fG][mA][fA][mU][fC][mG][fU][mA] |
| | | | [fC][mA][fC][Ps][mA][Ps][fA][Ps][mA][Ps][fG][Ps][mG] |
| 1843 | 335 | 24235 | [5Phos][mU][Ps][fA][Ps][mU][fA][mC][fC][mU][fC][mU][fG][mC] |
| | | | [fU][mC][fU][Ps][mU][Ps][fC][Ps][mU][Ps][fG][Ps][mA] |
| 1844 | 336 | 24236 | [5Phos][mU][Ps][fA][Ps][mU][fC][mA][fA][mU][fU][mU][fC][mU] |
| | | | [fU][mC][fU][Ps][mA][Ps][fC][Ps][mC][Ps][fA][Ps][mU] |
| 1845 | 337 | 24237 | [5Phos][mU][Ps][fA][Ps][mA][fC][mA][fU][mU][fG][mU][fG][mU] |
| | | | [fU][mU][fU][Ps][mG][Ps][fC][Ps][mA][Ps][fU][Ps][mU] |
| 1846 | 338 | 24238 | [5Phos][mU][Ps][fA][Ps][mA][fC][mU][fU][mU][fA][mU][fA][mA] |
| | | | [fG][mC][fA][Ps][mU][Ps][fA][Ps][mU][Ps][fG][Ps][mC] |
| 1847 | 339 | 24239 | [5Phos][mU][Ps][fA][Ps][mG][fG][mA][fU][mA][fA][mC][fU][mU] |
| | | | [fU][mU][fA][Ps][mA][Ps][fU][Ps][mA][Ps][fG][Ps][mA] |
| 1848 | 340 | 24240 | [5Phos][mU][Ps][fU][Ps][mU][fU][mA][fU][mU][fG][mG][fU][mU] |
| | | | [fG][mA][fU][Ps][mA][Ps][fC][Ps][mU][Ps][fG][Ps][mU] |
| 1849 | 341 | 24241 | [5Phos][mU][Ps][fG][Ps][mC][fA][mA][fC][mU][fG][mU][fU][mU] |
| | | | [fU][mC][fU][Ps][mU][Ps][fC][Ps][mU][Ps][fG][Ps][mG] |
| 1850 | 342 | 24242 | [5Phos][mU][Ps][fG][Ps][mC][fU][mU][fU][mG][fA][mU][fA][mC] |
| | | | [fA][mA][fC][Ps][mU][Ps][fU][Ps][mC][Ps][fC][Ps][mA] |
| 1851 | 343 | 24243 | [5Phos][mU][Ps][fC][Ps][mA][fA][mA][fG][mC][fU][mU][fC][mU] |
| | | | [fC][mU][fC][Ps][mU][Ps][fU][Ps][mC][Ps][fA][Ps][mA] |
| 1852 | 344 | 24244 | [5Phos][mU][Ps][fG][Ps][mG][fA][mA][fC][mU][fC][mC][fU][mU] |
| | | | [fU][mC][fG][Ps][mU][Ps][fC][Ps][mU][Ps][fG][Ps][mC] |
| 1853 | 345 | 24245 | [5Phos][mU][Ps][fA][Ps][mU][fG][mA][fC][mA][fG][mU][fU][mC] |
| | | | [fU][mU][fU][Ps][mG][Ps][fA][Ps][mC][Ps][fU][Ps][mG] |
| 1854 | 346 | 24246 | [5Phos][mU][Ps][fU][Ps][mG][fC][mA][fG][mA][fA][mU][fA][mA] |
| | | | [fC][mA][fU][Ps][mG][Ps][fU][Ps][mC][Ps][fC][Ps][mA] |
| 1855 | 347 | 24247 | [5Phos][mU][Ps][fA][Ps][mG][fA][mA][fG][mU][fC][mC][fU][mA] |
| | | | [fU][mA][f G][Ps][mU][Ps][fU][Ps][mG][Ps][fU][Ps][mA] |
| 1856 | 348 | 24248 | [5Phos][mU][Ps][fA][Ps][mU][fA][mA][fC][mU][fU][mU][fU][mA] |
| | | | [fA][mU][fA][Ps][mG][Ps][fA][Ps][mG][Ps][fA][Ps][mU] |
| 1857 | 349 | 24249 | [5Phos][mU][Ps][fC][Ps][mU][fA][mA][fG][mA][fU][mU][fU][mC] |
| | | | [fU][mU][fU][Ps][mU][Ps][fC][Ps][mC][Ps][fA][Ps][mA] |
| 1858 | 350 | 24250 | [5Phos][mU][Ps][fA][Ps][mU][fA][mA][fA][mU][fG][mA][fA][mC] |
| | | | [fA][mU][fG][Ps][mG][Ps][fC][Ps][mC][Ps][fU][Ps][mG] |

The first nucleobase on the terminal 5′ position (the sequences in the table are presented from a 5′(left) to a 3′(right direction) can be freely selected from U, A, G and C instead of the nucleobase disclosed in the table.

Note=Each of the above constructs may or may not have a phosphate modification at the 5′ end group. Furthermore, and independently, each of the above constructs may or may not have a “3× GalNAc” coupled to the 3′ end group. Advantageously the construct contains a 3× GalNAc ligand. Particularly advantageous are constructs, which in addition have a 5′ phosphate, even though this is not a strict requirement, given that in the absence thereof, mammalian cells will add such phosphate in case it is absent from the molecule as administered.

TABLE 3d
Modified C5 sense constructs

| SEQ | Construct | Sense | |
|---|---|---|---|
| ID No. | ID NO. | ID | Modified 15 mer C5 Sense constructs |
| 1859 | 351 | 14151 | [fC][Ps][mA][Ps][fG][mG][fC][mC][fA][mA][fA][mC][fU][mG][fU |
| | | | ][Ps][mG][Ps][fA][3XGalNAc] |
| 1860 | 352 | 14152 | [fU][Ps][mG][Ps][fA][mC][fU][mU][fC][mA][fA][mG][fA][mU][fU |
| | | | ][Ps][mC][Ps][fA][3XGalNAc] |
| 1861 | 353 | 14153 | [fC][Ps][mA][Ps][fG][mC][fU][mA][fC][mA][fA][mG][fC][mC][fC |
| | | | ][Ps][mA][Ps][fA][3XGalNAc] |
| 1862 | 354 | 14154 | [fA][Ps][mC][Ps][fU][mA][fU][mA][fA][mU][fU][mA][fC][mU][fU] |
| | | | [Ps][mG][Ps][fA][3XGalNAc] |
| 1863 | 355 | 14155 | [fC][Ps][mA][Ps][fG][mA][fC][mA][fA][mA][fC][mC][fU][mG][fU] |
| | | | [Ps][mU][Ps][fA][3XGalNAc] |
| 1864 | 356 | 14156 | [fU][Ps][mG][Ps][fU][mU][fA][mA][fA][mA][fU][mG][fU][mC][fU |
| | | | ][Ps][mG][Ps][fA][3XGalNAc] |
| 1865 | 357 | 14157 | [fC][Ps][mA][Ps][fA][mC][fC][mA][fG][mC][fU][mC][fC][mA][fG |
| | | | ][Ps][mG][Ps][fA][3XGalNAc] |
| 1866 | 358 | 14158 | [fU][Ps][mU][Ps][fA][mC][fU][mU][fG][mA][fU][mU][fU][mU][fA |
| | | | ][Ps][mU][Ps][fA][3XGalNAc] |
| 1867 | 359 | 14159 | [fA][Ps][mG][Ps][fG][mC][fC][mA][fA][mA][fC][mU][fG][mU][fG |
| | | | ][Ps][mU][Ps][fA][3XGalNAc] |
| 1868 | 360 | 14160 | [fG][Ps][mA][Ps][fC][mU][fU][mC][fA][mA][fG][mA][fU][mU][fC |
| | | | ][Ps][mC][Ps][fA][3XGalNAc] |
| 1869 | 361 | 14161 | [fU][Ps][mG][Ps][fU][mG][fU][mU][fA][mA][fA][mA][fU][mG][fU |
| | | | ][Ps][mC][Ps][fA][3XGalNAc] |
| 1870 | 362 | 14162 | [fC][Ps][mU][Ps][fU][mU][fU][mC][fC][mU][fG][mA][fC][mU][fU |
| | | | ][Ps][mC][Ps][fA][3XGalNAc] |
| 1871 | 363 | 14163 | [fC][Ps][mC][Ps][fA][mG][fG][mC][fC][mA][fA][mA][fC][mU][fG |
| | | | ][Ps][mU][Ps][fA][3XGalNAc] |
| 1872 | 364 | 14164 | [fU][Ps][mA][Ps][fA][mC][fU][mC][fA][mC][fU][mA][fU][mA][fA] |
| | | | [Ps][mU][Ps][fA][3XGalNAc] |
| 1873 | 365 | 14165 | [fG][Ps][mA][Ps][fC][mA][fU][mC][fU][mG][fA][mC][fU][mU][fG |
| | | | ][Ps][mG][Ps][fA][3XGalNAc] |
| 1874 | 366 | 14166 | [fU][Ps][mA][Ps][fU][mC][fU][mC][fU][mU][fU][mU][fC][mC][fU |
| | | | ][Ps][mG][Ps][fA][3XGalNAc] |
| 1875 | 367 | 14167 | [fA][Ps][mG][Ps][fA][mA][fU][mA][fU][mG][fU][mC][fU][mU][fG |
| | | | ][Ps][mC][Ps][fA][3XGalNAc] |
| 1876 | 368 | 14168 | [fU][Ps][mG][Ps][fG][mA][fA][mA][fU][mU][fG][mG][fC][mC][fU |
| | | | ][Ps][mU][Ps][fA][3XGalNAc] |
| 1877 | 369 | 14169 | [fC][Ps][mA][Ps][fC][mU][fA][mU][fA][mA][fU][mU][fA][mC][fU] |
| | | | [Ps][mU][Ps][fA][3XGalNAc] |
| 1878 | 370 | 14170 | [fA][Ps][mU][Ps][fU][mA][fC][mU][fU][mG][fA][mU][fU][mU][fU |
| | | | ][Ps][mA][Ps][fA][3XGalNAc] |
| 1879 | 371 | 14171 | [fC][Ps][mU][Ps][fA][mU][fA][mA][fU][mU][fA][mC][fU][mU][fG] |
| | | | [Ps][mA][Ps][fA][3XGalNAc] |
| 1880 | 372 | 14172 | [fC][Ps][mC][Ps][fU][mC][fU][mG][fG][mA][fA][mA][fU][mU][fG |
| | | | ][Ps][mG][Ps][fA][3XGalNAc] |
| 1881 | 373 | 14173 | [fC][Ps][mU][Ps][fC][mA][fC][mU][fA][mU][fA][mA][fU][mU][fA] |
| | | | [Ps][mC][Ps][fA][3XGalNAc] |
| 1882 | 374 | 14174 | [fC][Ps][mA][Ps][fA][mC][fA][mA][fG][mU][fA][mC][fC][mU][fU] |
| | | | [Ps][mU][Ps][fA][3XGalNAc] |
| 1883 | 375 | 14175 | [fU][Ps][mC][Ps][fU][mG][fA][mA][fA][mC][fA][mG][fC][mA][fG |
| | | | ][Ps][mU][Ps][fA][3XGalNAc] |
| 1884 | 376 | 14176 | [fU][Ps][mU][Ps][fC][mU][fG][mA][fA][mA][fC][mA][fG][mC][fA] |
| | | | [Ps][mG][Ps][fA][3XGalNAc] |
| 1885 | 377 | 14177 | [fC][Ps][mA][Ps][fA][mG][fU][mA][fC][mC][fU][mU][fU][mA][fU] |
| | | | [Ps][mA][Ps][fA][3XGalNAc] |
| 1886 | 378 | 14178 | [fA][Ps][mA][Ps][fG][mG][fA][mA][fC][mU][fG][mU][fU][mU][fA |
| | | | ][Ps][mC][Ps][fA][3XGalNAc] |
| 1887 | 379 | 14179 | [fU][Ps][mC][Ps][fA][mG][fC][mC][fA][mA][fA][mG][fU][mU][fA] |
| | | | [Ps][mC][Ps][fA][3XGalNAc] |
| 1888 | 380 | 14180 | [fC][Ps][mU][Ps][fG][mU][fU][mU][fA][mC][fA][mA][fC][mU][fA] |
| | | | [Ps][mU][Ps][fA][3XGalNAc] |
| 1889 | 381 | 14181 | [fA][Ps][mG][Ps][fU][mU][fA][mA][fA][mG][fA][mA][fU][mA][fU] |
| | | | [Ps][mG][Ps][fA][3XGalNAc] |
| 1890 | 382 | 14182 | [fG][Ps][mU][Ps][fA][mA][fA][mA][fG][mG][fA][mC][fU][mG][fC |
| | | | ][Ps][mU][Ps][fA][3XGalNAc] |
| 1891 | 383 | 14183 | [fC][Ps][mA][Ps][fA][mA][fA][mU][fA][mA][fC][mU][fC][mA][fC] |
| | | | [Ps][mU][Ps][fA][3XGalNAc] |
| 1892 | 384 | 14184 | [fC][Ps][mA][Ps][fA][mA][fG][mA][fU][mG][fU][mC][fU][mU][fC |
| | | | ][Ps][mC][Ps][fA][3XGalNAc] |
| 1893 | 385 | 14185 | [fG][Ps][mU][Ps][fG][mU][fA][mA][fA][mA][fG][mG][fA][mC][fU |
| | | | ][Ps][mG][Ps][fA][3XGalNAc] |
| 1894 | 386 | 14186 | [fA][Ps][mU][Ps][fU][mG][fA][mC][fA][mA][fA][mA][fU][mA][fA] |
| | | | [Ps][mC][Ps][fA][3XGalNAc] |
| 1895 | 387 | 14187 | [fC][Ps][mA][Ps][fU][mA][fU][mU][fC][mU][fG][mU][fU][mG][fU |
| | | | ][Ps][mA][Ps][fA][3XGalNAc] |
| 1896 | 388 | 14188 | [fU][Ps][mA][Ps][fU][mU][fG][mA][fC][mA][fA][mA][fA][mU][fA] |
| | | | [Ps][mA][Ps][fA][3XGalNAc] |
| 1897 | 389 | 14189 | [fU][Ps][mU][Ps][fU][mU][fC][mC][fU][mG][fA][A][fG][m][fC |
| | | | ][Ps][mU][Ps][fA][3XGalNAc] |
| 1898 | 390 | 14190 | [fA][Ps][mU][Ps][fU][mA][fU][mC][fU][mC][fU][mU][fU][mU][fC |
| | | | ][Ps][mC][Ps][fA][3XGalNAc] |
| 1899 | 391 | 14191 | [fU][Ps][mA][Ps][fU][mA][fU][mU][fG][mC][fU][mG][fU][mA][fA |
| | | | ][Ps][mC][Ps][fA][3XGalNAc] |
| 1900 | 392 | 14192 | [fU][Ps][mU][Ps][fG][mC][fA][mU][fA][mU][fG][mC][fU][mU][fA |
| | | | ][Ps][mU][Ps][fA][3XGalNAc] |
| 1901 | 393 | 14193 | [fA][Ps][mA][Ps][fG][mU][fU][mA][fA][mA][fG][mA][fA][mU][fA] |
| | | | [Ps][mU][Ps][fA][3XGalNAc] |
| 1902 | 394 | 14194 | [fG][Ps][mU][Ps][fU][mC][fA][mA][fA][mG][fA][mU][fG][mU][fC |
| | | | ][Ps][mU][Ps][fA][3XGalNAc] |
| 1903 | 395 | 14195 | [fA][Ps][mA][Ps][fG][mA][fU][mG][fU][mC][fU][mU][fC][mC][fU |
| | | | ][Ps][mG][Ps][fA][3XGalNAc] |
| 1904 | 396 | 14196 | [fC][Ps][mU][Ps][fU][mU][fA][mU][fA][mU][fU][mG][fC][mU][fG |
| | | | ][Ps][mU][Ps][fA][3XGalNAc] |
| 1905 | 397 | 14197 | [fU][Ps][mA][Ps][fA][mC][fC][mU][fA][mU][fG][mA][fC][mA][fA] |
| | | | [Ps][mU][Ps][fA][3XGalNAc] |
| 1906 | 398 | 14198 | [fU][Ps][mG][Ps][fA][mC][fA][mA][fA][mA][fU][mA][fA][mC][fU] |
| | | | [Ps][mC][Ps][fA][3XGalNAc] |
| 1907 | 399 | 14199 | [fA][Ps][mC][Ps][fA][mA][fA][mA][fU][mA][fA][mC][fU][mC][fA] |
| | | | [Ps][mC][Ps][fA][3XGalNAc] |
| 1908 | 400 | 14200 | [fG][Ps][mA][Ps][fC][mA][fA][mA][fA][mU][fA][mA][fC][mU][fC] |
| | | | [Ps][mA][Ps][fA][3XGalNAc] |
| 1909 | 401 | 14201 | [fU][Ps][mU][Ps][fU][mC][fA][mA][fG][mG][fA][mA][fA][mA][fU] |
| | | | [Ps][mU][Ps][fA][3XGalNAc] |
| 1910 | 402 | 14202 | [fU][Ps][mA][Ps][fU][mU][fG][mC][fU][mG][fU][mA][fA][mC][fA |
| | | | ][Ps][mG][Ps][fA][3XGalNAc] |
| 1911 | 403 | 14203 | [fU][Ps][mA][Ps][fU][mG][fA][mC][fA][mA][fU][mG][fG][mA][fU |
| | | | ][Ps][mU][Ps][fA][3XGalNAc] |
| 1912 | 404 | 14204 | [fC][Ps][mU][Ps][fA][mU][fG][mA][fC][mA][fA][mU][fG][mG][fA |
| | | | ][Ps][mU][Ps][fA][3XGalNAc] |
| 1913 | 405 | 14205 | [fG][Ps][mA][Ps][fC][mA][fA][mU][fG][mG][fA][mU][fU][mU][fC |
| | | | ][Ps][mU][Ps][fA][3XGalNAc] |
| 1914 | 406 | 14206 | [fU][Ps][mA][Ps][fA][mA][fG][mA][fA][mU][fA][mU][fG][mU][fC] |
| | | | [Ps][mU][Ps][fA][3XGalNAc] |
| 1915 | 407 | 14207 | [[A][Ps][mG][Ps][fG][mG][fA][mA][fU][mC][fU][mG][fC][mA][fC |
| | | | ][Ps][mU][Ps][fA][3XGalNAc] |
| 1916 | 408 | 14208 | [fC][Ps][mC][Ps][fU][mA][fU][mG][fA][mC][fA][mA][fU][mG][fG |
| | | | ][Ps][mA][Ps][fA][3XGalNAc] |
| 1917 | 409 | 14209 | [fU][Ps][mG][Ps][fU][mU][fC][mA][fA][mA][fG][mA][fU][mG][fU |
| | | | ][Ps][mC][Ps][fA][3XGalNAc] |
| 1918 | 410 | 14210 | [fA][Ps][mA][Ps][fC][mC][fU][mA][fU][mG][fA][mC][fA][mA][fU] |
| | | | [Ps][mG][Ps][fA][3XGalNAc] |
| 1919 | 411 | 14211 | [fA][Ps][mU][Ps][fG][mG][fA][mU][fU][mU][fC][mU][fC][mU][fU |
| | | | ][Ps][mC][Ps][fA][3XGalNAc] |
| 1920 | 412 | 14212 | [fG][Ps][mU][Ps][fU][mA][fA][mA][fG][mA][fA][mU][fA][mU][fG] |
| | | | [Ps][mU][Ps][fA][3XGalNAc] |
| 1921 | 413 | 14213 | [fA][Ps][mG][Ps][fU][mG][fU][mA][fA][mA][fA][mG][fG][mA][fC |
| | | | ][Ps][mU][Ps][fA][3XGalNAc] |
| 1922 | 414 | 14214 | [fC][Ps][mU][Ps][fU][mU][fC][mA][fA][mG][fG][mA][fA][mA][fA] |
| | | | [Ps][mU][Ps][fA][3XGalNAc] |
| 1923 | 415 | 14215 | [fU][Ps][mA][Ps][fA][mA][fA][mU][fA][mC][fA][mA][fU][mU][fU] |
| | | | [Ps][mC][Ps][fA][3XGalNAc] |
| 1924 | 416 | 14216 | [fA][Ps][mA][Ps][fU][mU][fU][mU][fG][mA][fA][mA][fU][mU][fA] |
| | | | [Ps][mC][Ps][fA][3XGalNAc] |
| 1925 | 417 | 14217 | [fA][Ps][mC][Ps][fU][mU][fU][mA][fA][mG][fA][mA][fU][mU][fU] |
| | | | [Ps][mU][Ps][fA][3XGalNAc] |
| 1926 | 418 | 14218 | [fC][Ps][mA][Ps][fG][mA][fA][mC][fC][mA][fA][mA][fA][mU][fU] |
| | | | [Ps][mC][Ps][fA][3XGalNAc] |
| 1927 | 419 | 14219 | [fA][Ps][mA][Ps][fU][mU][fA][mU][fA][mA][fA][mA][fU][mG][fA] |
| | | | [Ps][mC][Ps][fA][3XGalNAc] |
| 1928 | 420 | 14220 | [fC][Ps][mA][Ps][fG][mU][fA][mC][fA][mG][fG][mA][fU][mU][fU |
| | | | ][Ps][mG][Ps][fA][3XGalNAc] |
| 1929 | 421 | 14221 | [fU][Ps][mA][Ps][fU][mU][fA][mA][fA][mA][fG][mU][fU][mA][fU] |
| | | | [Ps][mC][Ps][fA][3XGalNAc] |
| 1930 | 422 | 14222 | [fA][Ps][mA][Ps][fG][mA][fG][mA][fA][mG][fA][mC][fU][mU][fA] |
| | | | [Ps][mA][Ps][fA][3XGalNAc] |
| 1931 | 423 | 14223 | [fC][Ps][mA][Ps][fU][mA][fU][mU][fG][mG][fA][mA][fU][mU][fA] |
| | | | [Ps][mU][Ps][fA][3XGalNAc] |
| 1932 | 424 | 14224 | [fC][Ps][mA][Ps][fU][mG][fU][mU][fC][mA][fU][mU][fU][mA][fU |
| | | | ][Ps][mC][Ps][fA][3XGalNAc] |
| 1933 | 425 | 14225 | [fA][Ps][mA][Ps][fA][mU][fG][mA][fU][mG][fA][mA][fC][mC][fU] |
| | | | [Ps][mU][Ps][fA][3XGalNAc] |
| 1934 | 426 | 14226 | [fU][Ps][mU][Ps][fU][mA][fU][mA][fG][mC][fA][mC][fU][mU][fC |
| | | | ][Ps][mC][Ps][fA][3XGalNAc] |
| 1935 | 427 | 14227 | [fU][Ps][mU][Ps][fU][mA][fA][mG][fA][mG][fU][mA][fC][mU][fU |
| | | | ][Ps][mG][Ps][fA][3XGalNAc] |
| 1936 | 428 | 14228 | [fC][Ps][mA][Ps][fG][mG][fA][mA][fA][mU][fC][mA][fU][mU][fG |
| | | | ][Ps][mG][Ps][fA][3XGalNAc] |
| 1937 | 429 | 14229 | [fA][Ps][mA][Ps][fU][mG][fA][mA][fU][mA][fU][mA][fC][mC][fA] |
| | | | [Ps][mU][Ps][fA][3XGalNAc] |
| 1938 | 430 | 14230 | [fA][Ps][mA][Ps][fG][mU][fU][mC][fA][mU][fC][mU][fU][mG][fU |
| | | | ][Ps][mU][Ps][fA][3XGalNAc] |
| 1939 | 431 | 14231 | [fA][Ps][mA][Ps][fU][mU][fC][mC][fU][mG][fA][mA][fG][mU][fU |
| | | | ][Ps][mC][Ps][fA][3XGalNAc] |
| 1940 | 432 | 14232 | [fA][Ps][mA][Ps][fU][mA][fA][mG][fA][mG][fA][mA][fG][mA][fC] |
| | | | [Ps][mU][Ps][fA][3XGalNAc] |
| 1941 | 433 | 14233 | [fA][Ps][mA][Ps][fA][mG][fU][mA][fU][mA][fA][mA][fC][mA][fU] |
| | | | [Ps][mU][Ps][fA][3XGalNAc] |
| 1942 | 434 | 14234 | [fU][Ps][mG][Ps][fU][mG][fU][mA][fC][mG][fA][mU][fU][mC][fC |
| | | | ][Ps][mG][Ps][fA][3XGalNAc] |
| 1943 | 435 | 14235 | [fA][Ps][mA][Ps][fG][mA][fG][mC][fA][mG][fA][mG][fG][mU][fA |
| | | | ][Ps][mU][Ps][fA][3XGalNAc] |
| 1944 | 436 | 14236 | [fU][Ps][mA][Ps][fG][mA][fA][mG][fA][mA][fA][mU][fU][mG][fA] |
| | | | [Ps][mU][Ps][fA][3XGalNAc] |
| 1945 | 437 | 14237 | [fC][Ps][mA][Ps][fA][mA][fA][mC][fA][mC][fA][mA][fU][mG][fU] |
| | | | [Ps][mU][Ps][fA][3XGalNAc] |
| 1946 | 438 | 14238 | [fA][Ps][mU][Ps][fG][mC][fU][mU][fA][mU][fA][mA][fA][mG][fU] |
| | | | [Ps][mU][Ps][fA][3XGalNAc] |
| 1947 | 439 | 14239 | [fU][Ps][mU][Ps][fA][mA][fA][mA][fG][mU][fU][mA][fU][mC][fC] |
| | | | [Ps][mU][Ps][fA][3XGalNAc] |
| 1948 | 440 | 14240 | [fU][Ps][mA][Ps][fU][mC][fA][mA][fC][mC][fA][mA][fU][mA][fA] |
| | | | [Ps][mA][Ps][fA][3XGalNAc] |
| 1949 | 441 | 14241 | [fA][Ps][mA][Ps][fG][mA][fA][mA][fA][mC][fA][mG][fU][mU][fG] |
| | | | [Ps][mC][Ps][fA][3XGalNAc] |
| 1950 | 442 | 14242 | [fA][Ps][mG][Ps][fU][mU][fG][mU][fA][mU][fC][mA][fA][mA][fG |
| | | | ][Ps][mC][Ps][fA][3XGalNAc] |
| 1951 | 443 | 14243 | [fA][Ps][mG][Ps][fA][mG][fA][mG][fA][mA][fG][mC][fU][mU][fU |
| | | | ][Ps][mG][Ps][fA][3XGalNAc] |
| 1952 | 444 | 14244 | [fA][Ps][mC][Ps][fG][mG][fA][mA][fA][mG][fG][mA][fG][mU][fC |
| | | | ][Ps][mC][Ps][fA][3XGalNAc] |
| 1953 | 445 | 14245 | [fC][Ps][mA][Ps][fA][mA][fG][mA][fA][mC][fU][mG][fU][mC][fA] |
| | | | [Ps][mU][Ps][fA][3XGalNAc] |
| 1954 | 446 | 14246 | [fC][Ps][mA][Ps][fU][mG][fU][mU][fA][mU][fU][mC][fU][mG][fC |
| | | | ][Ps][mA][Ps][fA][3XGalNAc] |
| 1955 | 447 | 14247 | [fA][Ps][mC][Ps][fU][mA][fU][mA][fG][mG][fA][mC][fU][mU][fC |
| | | | ][Ps][mU][Ps][fA][3XGalNAc] |
| 1956 | 448 | 14248 | [fC][Ps][mU][Ps][fA][mU][fU][mA][fA][mA][fA][mG][fU][mU][fA] |
| | | | [Ps][mU][Ps][fA][3XGalNAc] |
| 1957 | 449 | 14249 | [fA][Ps][mA][Ps][fA][mA][fG][mA][fA][mA][fU][mC][fU][mU][fA] |
| | | | [Ps][mG][Ps][fA][3XGalNAc] |
| 1958 | 450 | 14250 | [fC][Ps][mC][Ps][fA][mU][fG][mU][fU][mC][fA][mU][fU][mU][fA |
| | | | ][Ps][mU][Ps][fA][3XGalNAc] |

TABLE 3e
Modified C5 hairpin constructs

| | | Antisense | | |
|---|---|---|---|---|
| SEQ ID | Construct | ID + | Expt'l | |
| No. | ID NO. | Sense ID | ID | Modified 33 mer C5 Hairpin constructs |
| 1959 | 451 | 14151_ | C5-m-01 | [5Phos][mU][Ps][fC][Ps][mA][fC][mA][fG][mU][f |
| | | 24151 | | U][mU][fG][mG][fC][mC][fU][Ps][mG]Ps][G]P |
| | | | | s][mA][Ps][fG][Ps][mA][Ps][mA] |
| | | | | [fG][mG][fC][mC][fA][mA][fA][mC][fU][mG][fU][ |
| | | | | Ps][mG][Ps][fA][3xGalNac] |
| 1960 | 452 | 14152_ | C5-m-02 | [5Phos][mU][Ps][fG][Ps][mA][fA][mU][fC][mU][f |
| | | 24152 | | U][mG][fA][mA][fG][mU][fC][Ps][mA][Ps][fG][Ps |
| | | | | ][mG][Ps][fA][Ps][mA][Ps][mG] |
| | | | | [fA][mC][fU][mU][fC][mA][fA][mG][fA][mU][fU][P |
| | | | | s][mC][Ps][fA][3xGalNac] |
| 1961 | 453 | 14153_ | C5-m-03 | [5Phos][mU][Ps][fU][Ps][mG][fG][mG][fC][mU][f |
| | | 24153 | | U][mG][fU][mA][fG][mC][fU][Ps][mG][Ps][fG][P |
| | | | | s][mC][Ps][fA][Ps][mC][Ps][mA] |
| | | | | [fG][mC][fU][mA][fC][mA][fA][mG][fC][mC][fC][ |
| | | | | Ps][mA][Ps][[A][3xGalNac] |
| 1962 | 454 | 14154_ | C5-m-04 | [5Phos][mU][Ps][fC][Ps][mA][fA][mG][fU][mA][f |
| | | 24154 | | A][mU][fU][mA][fU][mA][fG][Ps][mU][Ps][fG][Ps |
| | | | | ][mA][Ps][fG][Ps][mU][Ps][mC] |
| | | | | [fU][mA][fU][mA][fA][mU][fU][mA][fC][mU][fU][P |
| | | | | s][mG][Ps][fA][3xGalNac] |
| 1963 | 455 | 14155_ | C5-m-05 | [5Phos][mU][Ps][fA][Ps][mA][fC][mA][fG][mG][f |
| | | 24155 | | U][mU][fU][mG][fU][mC][fU][Ps][mG][Ps][fU][Ps |
| | | | | ][mA][Ps][fU][Ps][mG][Ps][mA] |
| | | | | [fG][mA][fC][mA][fA][mA][fC][mC][fU][mG][fU][ |
| | | | | Ps][mU][Ps][fA][3xGalNac] |
| 1964 | 456 | 14156_ | C5-m-06 | [5Phos][mU][Ps][fC][Ps][mA][fG][mA][fC][mA][f |
| | | 24156 | | U][mU][fU][mU][fA][mA][fC][Ps][mA][Ps][fC][Ps] |
| | | | | [mA][Ps][fG][Ps][mA][Ps][mG] |
| | | | | [fU][mU][fA][mA][fA][mA][fU][mG][fU][mC][fU][P |
| | | | | s][mG][Ps][fA][3xGalNac] |
| 1965 | 457 | 14157_ | C5-m-07 | [5Phos][mU][Ps][fC][Ps][mC][fU][mG][fG][mA][f |
| | | 24157 | | G][mC][fU][mG][fG][mU][fU][Ps][mG][Ps][fC][P |
| | | | | s][mC][Ps][fA][Ps][mC][Ps][mA] |
| | | | | [fA][mC][fC][mA][fG][mC][fU][mC][fC][mA][fG][ |
| | | | | Ps][mG][Ps][fA][3xGalNac] |
| 1966 | 458 | 14158_ | C5-m-08 | [5Phos][mU][Ps][fA][Ps][mU][fA][mA][fA][mA][f |
| | | 24158 | | U][mC][fA][mA][fG][mU][fA][Ps][mA][Ps][fU][Ps] |
| | | | | [mU][Ps][fA][Ps][mU][Ps][mU] |
| | | | | [fA][mC][fU][mU][fG][mA][fU][mU][fU][mU][A][ |
| | | | | Ps][mU][Ps][fA][3xGalNac] |
| 1967 | 459 | 14159_ | C5-m-09 | [5Phos][mU][Ps][fA][Ps][mC][fA][mC][fA][mG][f |
| | | 24159 | | U][mU][fU][mG][fG][mC][fC][Ps][mU][Ps][fG][P |
| | | | | s][mG][Ps][fA][Ps][mG][Ps][mG] |
| | | | | [fG][mC][fC][mA][fA][mA][fC][mU][fG][mU][fG][ |
| | | | | Ps][mU][Ps][fA][3xGalNac] |
| 1968 | 460 | 14160_ | C5-m-10 | [5Phos][mU][Ps][fG][Ps][mG][fA][mA][fU][mC][f |
| | | 24160 | | U][mU][fG][mA][fA][mG][fU][Ps][mC][Ps][fA][Ps |
| | | | | ][mG][Ps][fG][Ps][mA][Ps][mA] |
| | | | | [fC][mU][fU][mC][fA][mA][fG][mA][fU][mU][fC][ |
| | | | | Ps][mC][Ps][fA]3xGalNac] |
| 1969 | 461 | 14161_ | C5-m-11 | [5Phos][mU][Ps][fG][Ps][mA][fC][mA][fU][mU][f |
| | | 24161 | | U][mU][fA][mA][fC][mA][fC][Ps][mA][Ps][fG][Ps] |
| | | | | [mA][Ps][fA][Ps][mC][Ps][mG] |
| | | | | [fU][mG][fU][mU][fA][mA][fA][mA][fU][mG][fU][ |
| | | | | Ps][mC][Ps][fA][3xGalNac] |
| 1970 | 462 | 14162_ | C5-m-12 | [5Phos][mU][Ps][fG][Ps][mA][fA][mG][fU][mC][f |
| | | 24162 | | A][mG][fG][mA][fA][mA][fA][Ps][mG][Ps][fA][Ps] |
| | | | | [mG][Ps][fA][Ps][mU][Ps][mU] |
| | | | | [fU][mU][fU][mC][fC][mU][fG][mA][fC][mU][fU][ |
| | | | | Ps][mC][Ps][fA][3xGalNac] |
| 1971 | 463 | 14163_ | C5-m-13 | [5Phos][mU][Ps][fA][Ps][mC][fA][mG][fU][mU][f |
| | | 24163 | | U][mG][fG][mC][fC][mU][fG][Ps][mG][Ps][fA][P |
| | | | | s][mG][Ps][fA][Ps][mA][Ps][mC] |
| | | | | [fA][mG][fG][mC][fC][mA][fA][mA][fC][mU][fG][ |
| | | | | Ps][mU][Ps][fA][3xGalNac] |
| 1972 | 464 | 14164_ | C5-m-14 | [5Phos][mU][Ps][fA][Ps][mU][fU][mA][fU][mA][f |
| | | 24164 | | G][mU][fG][mA][fG][mU][fU][Ps][mA][Ps][fU][Ps |
| | | | | ][mU][Ps][fU][Ps][mU][Ps][mA] |
| | | | | [fA][mC][fU][mC][fA][mC][fU][mA][fU][mA][fA][P |
| | | | | s][mU][Ps][[A][3xGalNac] |
| 1973 | 465 | 14165_ | C5-m-15 | [5Phos][mU][Ps][fC][Ps][mC][fA][mA][fG][mU][f |
| | | 24165 | | C][mA][fG][mA][fU][mG][fU][Ps][mC][Ps][fU][Ps |
| | | | | ][mC][Ps][fU][Ps][mU][Ps][mA] |
| | | | | [fC][mA][fU][mC][fU][mG][fA][mC][fU][mU][fG][ |
| | | | | Ps][mG][Ps][fA][3xGalNac] |
| 1974 | 466 | 14166_ | C5-m-16 | [5Phos][mU][Ps][fC][Ps][mA][fG][G][fA][mA][f |
| | | 24166 | | A][mA][fG][mA][fG][mA][fU][Ps][mA][Ps][fA][Ps] |
| | | | | [mU][Ps][fU][Ps][mC][Ps][mA] |
| | | | | [fU][mC][fU][mC][fU][mU][fU][mU][fC][mC][fU][ |
| | | | | Ps][mG][Ps][fA][3xGalNac] |
| 1975 | 467 | 14167_ | C5-m-17 | [5Phos][mU][Ps][fG][Ps][mC][fA][mA][fG][mA][f |
| | | 24167 | | C][mA][fU][mA][fU][mU][fC][Ps][mU][Ps][fU][Ps |
| | | | | ][mU][Ps][fA][Ps][mA][Ps][mG] |
| | | | | [fA][mA][fU][mA][fU][mG][fU][mC][fU][mU][fG][ |
| | | | | Ps][mC][Ps][fA][3xGalNac] |
| 1976 | 468 | 14168_ | C5-m-18 | [5Phos][mU][Ps][fA][Ps][mA][fG][mG][fC][mC][f |
| | | 24168 | | A][mA][fU][mU][fU][mC][fC][Ps][mA][Ps][fG][Ps |
| | | | | ][mA][Ps][fG][Ps][mG][Ps][mG] |
| | | | | [fG][mA][fA][mA][fU][mU][fG][mG][fC][mC][fU][ |
| | | | | Ps][mU][Ps][fA][3xGalNac] |
| 1977 | 469 | 14169_ | C5-m-19 | [5Phos][mU][Ps][fA][Ps][mA][fG][mU][fA][mA][f |
| | | 24169 | | U][mU][fA][mU][fA][mG][fU][Ps][mG][Ps][fA][Ps |
| | | | | ][mG][Ps][fU][Ps][mU][Ps][mA] |
| | | | | [fC][mU][fA][mU][fA][mA][fU][mU][fA][mC][fU][P |
| | | | | s][mU][Ps][fA][3xGalNac] |
| 1978 | 470 | 14170_ | C5-m-20 | [5Phos][mU][Ps][fU][Ps][mA][fA][mA][fA][mU][f |
| | | 24170 | | C][mA][fA][mG][fU][mA][fA][Ps][mU][Ps][fU][Ps] |
| | | | | [mA][Ps][fU][Ps][mA][Ps][mU] |
| | | | | [fU][mA][fC][mU][fU][mG][fA][mU][fU][mU][fU][ |
| | | | | Ps][mA][Ps][fA][3xGalNac] |
| 1979 | 471 | 14171_ | C5-m-21 | [5Phos][mU][Ps][fU][Ps][mC][fA][mA][fG][mU][f |
| | | 24171 | | A][mA][fU][mU][fA][mU][fA][Ps][mG][Ps][fU][Ps] |
| | | | | [mG][Ps][fA][Ps][mG][Ps][mU] |
| | | | | [fA][mU][fA][mA][fU][mU][fA][mC][fU][mU][fG][P |
| | | | | s][mA][Ps][fA]3xGalNac] |
| 1980 | 472 | 14172_ | C5-m-22 | [5Phos][mU][Ps][fC][Ps][mC][fA][mA][fU][mU][f |
| | | 24172 | | U][mC][fC][mA][fG][mA][fG][Ps][mG][Ps][fA][Ps |
| | | | | ][mA][Ps][fG][Ps][mC][Ps][mC] |
| | | | | [fU][mC][fU][mG][fG][mA][fA][mA][fU][mU][fG][ |
| | | | | Ps][mG][Ps][fA]\|3xGalNac] |
| 1981 | 473 | 14173_ | C5-m-23 | [5Phos][mU][Ps][fG][Ps][mU][fA][mA][fU][mU][f |
| | | 24173 | | A][mU][fA][mG][fU][mG][fA][Ps][mG][Ps][fU][Ps |
| | | | | ][mU][Ps][fA][Ps][mU][Ps][mU] |
| | | | | [fC][mA][fC][mU][fA][mU][fA][mA][fU][mU][fA][P |
| | | | | s][mC][Ps][fA]\|3xGalNac] |
| 1982 | 474 | 14174_ | C5-m-24 | [5Phos][mU][Ps][fA][Ps][mA][fA][mG][fG][mU][f |
| | | 24174 | | A][mC][fU][mU][fG][mU][fU][Ps][mG][Ps][fU][Ps |
| | | | | ][mU][Ps][fU][Ps][mA][Ps][mA] |
| | | | | [fA][mC][fA][mA][fG][mU][fA][mC][fC][mU][fU][P |
| | | | | s][mU][Ps][[A][3xGalNac] |
| 1983 | 475 | 14175_ | C5-m-25 | [5Phos][mU][Ps][fA][Ps][mC][fU][mG][fC][mU][f |
| | | 24175 | | G][mU][fU][mU][fC][mA][fG][Ps][mA][Ps][fA][Ps |
| | | | | ][mU][Ps][fC][Ps][mA][Ps][mC] |
| | | | | [fU][mG][fA][mA][fA][mC][fA][mG][fC][mA][fG][ |
| | | | | Ps][mU][Ps][fA]3xGalNac] |
| 1984 | 476 | 14176_ | C5-m-26 | [5Phos][mU][Ps][fC][Ps][mU][fG][mC][fU][mG][f |
| | | 24176 | | U][mU][fU][mC][fA][mG][fA][Ps][mA][Ps][fU][Ps |
| | | | | ][mC][Ps][fA][Ps][mA][Ps][mU] |
| | | | | [fC][mU][fG][mA][fA][mA][fC][mA][fG][mC][fA][P |
| | | | | s][mG][Ps][fA][3xGalNac] |
| 1985 | 477 | 14177_ | C5-m-27 | [5Phos][mU][Ps][fU][Ps][mA][fU][mA][fA][mA][f |
| | | 24177 | | G][mG][fU][mA][fC][mU][fU][Ps][mG][Ps][fU][Ps |
| | | | | ][mU][Ps][fG][Ps][mU][Ps][mA] |
| | | | | [fA][G][fU][mA][fC][mC][fU][mU][fU][mA][fU][ |
| | | | | Ps][mA][Ps][fA][3xGalNac] |
| 1986 | 478 | 14178_ | C5-m-28 | [5Phos][mU][Ps][fG][Ps][mU][fA][mA][fA][mC][f |
| | | 24178 | | A][mG][fU][mU][fC][mC][fU][Ps][mU][Ps][fU][Ps |
| | | | | ][mC][Ps][fA][Ps][mA][Ps][mA] |
| | | | | [fG][mG][fA][mA][fC][mU][fG][mU][fU][mU][fA][ |
| | | | | Ps][mC][Ps][fA][3xGalNac] |
| 1987 | 479 | 14179_ | C5-m-29 | [5Phos][mU][Ps][fG][Ps][mU][fA][mA][fC][mU][f |
| | | 24179 | | U][mU][fG][mG][fC][mU][fG][Ps][mA][Ps][fG][P |
| | | | | s][mA][Ps][fG][Ps][mA][Ps][mC] |
| | | | | [fA][mG][fC][mC][fA][mA][fA][mG][fU][mU][fA][P |
| | | | | s][mC][Ps][fA][3xGalNac] |
| 1988 | 480 | 14180_ | C5-m-30 | [5Phos][mU][Ps][fA][Ps][mU][fA][mG][fU][mU][f |
| | | 24180 | | G][mU][fA][mA][fA][mC][fA][Ps][mG][Ps][fU][Ps |
| | | | | ][mU][Ps][fC][Ps][mC][Ps][mU] |
| | | | | [fG][mU][fU][mU][fA][mC][fA][mA][fC][mU][fA][P |
| | | | | s][mU][Ps][fA][3xGalNac] |
| 1989 | 481 | 14181_ | C5-m-31 | [5Phos][mU][Ps][fC][Ps][mA][fU][mA][fU][mU][f |
| | | 24181 | | C][mU][fU][mU][fA][mA][fC][Ps][mU][Ps][fU][Ps |
| | | | | [mC][Ps][fA][Ps][mA][Ps][mG] |
| | | | | [fU][mU][fA][mA][fA][mG][fA][mA][fU][mA][fU][P |
| | | | | s][mG][Ps][fA][3xGalNac] |
| 1990 | 482 | 14182_ | C5-m-32 | [5Phos][mU][Ps][fA][Ps][mG][fC][mA][fG][mU][f |
| | | 24182 | | C][mC][fU][mU][fU][mU][fA][Ps][mC][Ps][fA][Ps |
| | | | | ][mC][Ps][fU][Ps][mC][Ps][mU] |
| | | | | [fA][mA][fA][mA][fG][mG][fA][mC][fU][mG][fC][ |
| | | | | Ps][mU][Ps][fA][3xGalNac] |
| 1991 | 483 | 14183_ | C5-m-33 | [5Phos][mU][Ps][fA][Ps][mG][fU][mG][fA][mG][f |
| | | 24183 | | U][mU][fA][mU][fU][mU][fU][Ps][mG][Ps][fU][Ps |
| | | | | ][mC][Ps][fA][Ps][mA][Ps][mA] |
| | | | | [fA][mA][fA][mU][fA][mA][fC][mU][fC][mA][fC][P |
| | | | | s][mU][Ps][fA][3xGalNac] |
| 1992 | 484 | 14184_ | C5-m-34 | [5Phos][mU][Ps][fG][Ps][mG][fA][mA][fG][mA][f |
| | | 24184 | | C][mA][fU][mC][fU][mU][fU][Ps][mG][Ps][fA][Ps |
| | | | | ][mA][Ps][fC][Ps][mA][Ps][mA] |
| | | | | [fA][mA][fG][mA][fU][mG][fU][mC][fU][mU][fC][ |
| | | | | Ps][mC][Ps][fA][3xGalNac] |
| 1993 | 485 | 14185_ | C5-m-35 | [5Phos][mU][Ps][fC][Ps][mA][fG][mU][fC][mC][f |
| | | 24185 | | U][mU][fU][mU][fA][mC][fA][Ps][mC][Ps][fU][Ps |
| | | | | ][mC][Ps][fA][Ps][mA][Ps][mU] |
| | | | | [fG][mU][fA][mA][fA][mA][fG][mG][fA][mC][fU][ |
| | | | | Ps][mG][Ps][fA][3xGalNac] |
| 1994 | 486 | 14186_ | C5-m-36 | [5Phos][mU][Ps][fG][Ps][mU][fU][mA][fU][mU][f |
| | | 24186 | | U][mU][fG][mU][fC][mA][fA][Ps][mU][Ps][fA][Ps |
| | | | | ][mU][Ps][fA][Ps][mU][Ps][mU] |
| | | | | [fU][mG][fA][mC][fA][mA][fA][mA][fU][mA][fA][P |
| | | | | s][mC][Ps][fA][3xGalNac] |
| 1995 | 487 | 14187_ | C5-m-37 | [5Phos][mU][Ps][fU][Ps][mA][fC][mA][fA][mC][f |
| | | 24187 | | A][mG][fA][mA][fU][mA][fU][Ps][mG][Ps][fG][Ps |
| | | | | ][mU][Ps][fA][Ps][mU][Ps][mA] |
| | | | | [fU][mA][fU][mU][fC][mU][fG][mU][fU][mG][fU][ |
| | | | | Ps][mA][Ps][fA][3xGalNac] |
| 1996 | 488 | 14188_ | C5-m-38 | [5Phos][mU][Ps][fU][Ps][mU][fA][mU][fU][mU][f |
| | | 24188 | | U][mG][fU][mC][fA][mA][fU][Ps][mA][Ps][fU][Ps |
| | | | | ][mA][Ps][fU][Ps][mG][Ps][mA] |
| | | | | [fU][mU][fG][mA][fC][mA][fA][mA][fA][mU][fA][P |
| | | | | s][mA][Ps][fA][3xGalNac] |
| 1997 | 489 | 14189_ | C5-m-39 | [5Phos][mU][Ps][fA][Ps][mG][fG][mC][fU][mU][f |
| | | 24189 | | C][mA][fG][mG][fA][mA][fA][Ps][mA][Ps][fG][Ps |
| | | | | ][mA][Ps][fG][Ps][mG][Ps][mU] |
| | | | | [fU][mU][fC][mC][fU][mG][fA][mA][fG][mC][fC][ |
| | | | | Ps][mU][Ps][fA][3xGalNac] |
| 1998 | 490 | 14190_ | C5-m-40 | [5Phos][mU][Ps][fG][Ps][mG][fA][mA][fA][mA][f |
| | | 24190 | | G][mA][fG][mA][fU][mA][fA][Ps][mU][Ps][fU][Ps |
| | | | | ][mC][Ps][fC][Ps][mA][Ps][mU] |
| | | | | [fU][mA][fU][mC][fU][mC][fU][mU][fU][mU][fC][ |
| | | | | Ps][mC][Ps][fA][3xGalNac] |
| 1999 | 491 | 14191_ | C5-m-41 | [5Phos][mU][Ps][fG][Ps][mU][fU][mA][fC][mA][f |
| | | 24191 | | G][mC][fA][mA][fU][mA][fU][Ps][mA][Ps][fA][Ps] |
| | | | | [mA][Ps][fG][Ps][mG][Ps][mA] |
| | | | | [fU][mA][fU][mU][fG][mC][fU][mG][fU][mA][fA][ |
| | | | | Ps][mC][Ps][fA][3xGalNac] |
| 2000 | 492 | 14192_ | C5-m-42 | [5Phos][mU][Ps][fA][Ps][mU][fA][mA][fG][mC][f |
| | | 24192 | | A][mU][fA][mU][fG][mC][fA][Ps][mA][Ps][fU][Ps] |
| | | | | [mC][Ps][fU][Ps][mC][Ps][mU] |
| | | | | [fG][mC][fA][mU][fA][mU][fG][mC][fU][mU][fA][ |
| | | | | Ps][mU][Ps][fA][3xGalNac] |
| 2001 | 493 | 14193_ | C5-m-43 | [5Phos][mU][Ps][fA][Ps][mU][fA][mU][fU][mC][f |
| | | 24193 | | U][mU][fU][mA][fA][mC][fU][Ps][mU][Ps][fC][Ps |
| | | | | ][mA][Ps][fA][Ps][mA][Ps][mA] |
| | | | | [fG][mU][fU][mA][fA][mA][fG][mA][fA][mU][fA][P |
| | | | | s][mU][Ps][fA][3xGalNac] |
| 2002 | 494 | 14194_ | C5-m-44 | [5Phos][mU][Ps][fA][Ps][mG][fA][mC][fA][mU][f |
| | | 24194 | | C][mU][fU][mU][fG][mA][fA][Ps][mC][Ps][fA][Ps |
| | | | | ][mC][Ps][fC][Ps][mU][Ps][mU] |
| | | | | [fU][mC][fA][mA][fA][mG][fA][mU][fG][mU][fC][ |
| | | | | Ps][mU][Ps][fA][3xGalNac] |
| 2003 | 495 | 14195_ | C5-m-45 | [5Phos][mU][Ps][fC][Ps][mA][fG][mG][fA][mA][f |
| | | 24195 | | G][mA][fC][mA][fU][mC][fU][Ps][mU][Ps][fU][Ps |
| | | | | ][mG][Ps][fA][Ps][mA][Ps][mA] |
| | | | | [fG][mA][fU][mG][fU][mC][fU][mU][fC][mC][fU][ |
| | | | | Ps][mG][Ps][fA][3xGalNac] |
| 2004 | 496 | 14196_ | C5-m-46 | [5Phos][mU][Ps][fA][Ps][mC][fA][mG][fC][mA][f |
| | | 24196 | | A][mU][fA][mU][fA][mA][fA][Ps][mG][Ps][fG][Ps] |
| | | | | [mU][Ps][fA][Ps][mC][Ps][mU] |
| | | | | [fU][mU][fA][mU][fA][mU][fU][mG][fC][mU][fG][ |
| | | | | Ps][mU][Ps][fA][3xGalNac] |
| 2005 | 497 | 14197_ | C5-m-47 | [5Phos][mU][Ps][fA][Ps][mU][fU][mG][fU][mC][f |
| | | 24197 | | A][mU][fA][mG][fG][mU][fU][Ps][mA][Ps][fU][Ps |
| | | | | ][mU][Ps][fG][Ps][mG][Ps][mA] |
| | | | | [fA][mC][fC][mU][fA][mU][fG][mA][fC][mA][fA][P |
| | | | | s][mU][Ps][fA][3xGalNac] |
| 2006 | 498 | 14198_ | C5-m-48 | [5Phos][mU][Ps][fG][Ps][mA][fG][mU][fU][mA][f |
| | | 24198 | | U][mU][fU][mU][fG][mU][fC][Ps][mA][Ps][fA][Ps |
| | | | | ][mU][Ps][fA][Ps][mU][Ps][mG] |
| | | | | [fA][mC][fA][mA][fA][mA][fU][mA][fA][mC][fU][P |
| | | | | s][mC][Ps]fA][3xGalNac] |
| 2007 | 499 | 14199_ | C5-m-49 | [5Phos][mU][Ps][fG][Ps][mU][fG][mA][fG][mU][f |
| | | 24199 | | U][mA][fU][mU][fU][mU][fG][Ps][mU][Ps][fC][Ps |
| | | | | ][mA][Ps][fA][Ps][mU][Ps][mC] |
| | | | | [fA][mA][fA][mA][fU][mA][fA][mC][fU][mC][fA][P |
| | | | | s][mC][Ps][fA][3xGalNac] |
| 2008 | 500 | 14200_ | C5-m-50 | [5Phos][mU][Ps][fU][Ps][mG][fA][mG][fU][mU][f |
| | | 24200 | | A][mU][fU][mU][fU][mG][fU][Ps][mC][Ps][fA][Ps |
| | | | | ][mA][Ps][fU][Ps][mA][Ps][mA] |
| | | | | [fC][mA][fA][mA][fA][mU][fA][mA][fC][mU][fC][P |
| | | | | s][mA][Ps][fA][3xGalNac] |
| 2009 | 501 | 14201_ | C5-m-51 | [5Phos][mU][Ps][fA][Ps][mA][fU][mU][fU][mU][f |
| | | 24201 | | C][mC][fU][mU][fG][mA][fA][Ps][mA][Ps][fG][Ps |
| | | | | ][mA][Ps][fU][Ps][mC][Ps][mU] |
| | | | | [fU][mC][fA][mA][fG][mG][fA][mA][fA][mA][fU][P |
| | | | | s][mU][Ps][fA][3xGalNac] |
| 2010 | 502 | 14202_ | C5-m-52 | [5Phos][mU][Ps][fC][Ps][mU][fG][mU][fU][mA][f |
| | | 24202 | | C][mA][fG][mC][fA][mA][fU][Ps][mA][Ps][fU][Ps] |
| | | | | [mA][Ps][fA][Ps][mA][Ps][mA] |
| | | | | [fU][mU][fG][mC][fU][mG][fU][mA][fA][mC][fA][ |
| | | | | Ps][mG][Ps][fA][3xGalNac] |
| 2011 | 503 | 14203_ | C5-m-53 | [5Phos][mU][Ps][fA][Ps][mA][fU][mC][fC][mA][f |
| | | 24203 | | U][mU][fG][mU][fC][mA][fU][Ps][mA][Ps][fG][Ps |
| | | | | ][mG][Ps][fU][Ps][mU][Ps][mA] |
| | | | | [fU][mG][fA][mC][fA][mA][fU][mG][fG][mA][fU][ |
| | | | | Ps][mU][Ps][fA][3xGalNac] |
| 2012 | 504 | 14204_ | C5-m-54 | [5Phos][mU][Ps][fA][Ps][mU][fC][mC][fA][mU][f |
| | | 24204 | | U][mG][fU][mC][fA][mU][fA][Ps][mG][Ps][fG][Ps |
| | | | | ][mU][Ps][fU][Ps][mA][Ps][mU] |
| | | | | [fA][mU][fG][mA][fC][mA][fA][mU][fG][mG][fA][ |
| | | | | Ps][mU][Ps][fA][3xGalNac] |
| 2013 | 505 | 14205_ | C5-m-55 | [5Phos][mU][Ps][fA][Ps][mG][fA][mA][fA][mU][f |
| | | 24205 | | C][mC][fA][mU][fU][mG][fU][Ps][mC][Ps][fA][Ps |
| | | | | ][mU][Ps][fA][Ps][mG][Ps][mA] |
| | | | | [fC][mA][fA][mU][fG][mG][fA][mU][fU][mU][fC][ |
| | | | | Ps][mU][Ps][fA][3xGalNac] |
| 2014 | 506 | 14206_ | C5-m-56 | [5Phos][mU][Ps][fA][Ps][mG][fA][mC][fA][mU][f |
| | | 24206 | | A][mU][fU][mC][fU][mU][fU][Ps][mA][Ps][fA][Ps] |
| | | | | [mC][Ps][fU][Ps][mU][Ps][mA] |
| | | | | [fA][mA][fG][mA][fA][mU][fA][mU][fG][mU][fC][P |
| | | | | s][mU][Ps][fA][3xGalNac] |
| 2015 | 507 | 14207_ | C5-m-57 | [5Phos][mU][Ps][fA][Ps][mG][fU][mG][fC][mA][f |
| | | 24207 | | G][mA][fU][mU][fC][mC][fC][Ps][mU][Ps][fC][Ps |
| | | | | ][mC][Ps][fA][Ps][mC][Ps][mG] |
| | | | | [fG][mG][fA][mA][fU][mC][fU][mG][fC][mA][fC][ |
| | | | | Ps][mU][Ps][fA][3xGalNac] |
| 2016 | 508 | 14208_ | C5-m-58 | [5Phos][mU][Ps][fU][Ps][mC][fC][mA][fU][mU][f |
| | | 24208 | | G][mU][fC][mA][fU][mA][fG][Ps][mG][Ps][fU][Ps |
| | | | | ][mU][Ps][fA][Ps][mU][Ps][mC] |
| | | | | [fU][mA][fU][mG][fA][mC][fA][mA][fU][mG][fG][ |
| | | | | Ps][mA][Ps][fA][3xGalNac] |
| 2017 | 509 | 14209_ | C5-m-59 | [5Phos][mU][Ps][fG][Ps][mA][fC][mA][fU][mC][f |
| | | 24209 | | U][mU][fU][mG][fA][mA][fC][Ps][mA][Ps][fC][Ps |
| | | | | ][mC][Ps][fU][Ps][mU][Ps][mG] |
| | | | | [fU][mU][fC][mA][fA][mA][fG][mA][fU][mG][fU][ |
| | | | | Ps][mC][Ps][fA][3xGalNac] |
| 2018 | 510 | 14210_ | C5-m-60 | [5Phos][mU][Ps][fC][Ps][mA][fU][mU][fG][mU][f |
| | | 24210 | | C][mA][fU][mA][fG][mG][fU][Ps][mU][Ps][fA][Ps |
| | | | | ][mU][Ps][fU][Ps][mG][Ps][mA] |
| | | | | [fC][mC][fU][mA][fU][G][fA][mC][fA][mA][fU][P |
| | | | | s][mG][Ps][fA][3xGalNac] |
| 2019 | 511 | 14211_ | C5-m-61 | [5Phos][mU][Ps][fG][Ps][mA][fA][mG][fA][mG][f |
| | | 24211 | | A][mA][fA][mU][fC][mC][fA][Ps][mU][Ps][fU][Ps] |
| | | | | [mG][Ps][fU][Ps][mC][Ps][mA] |
| | | | | [fG][mG][fA][mU][fU][mU][fC][mU][fC][mU][fU][ |
| | | | | Ps][mC][Ps][fA][3xGalNac] |
| 2020 | 512 | 14212_ | C5-m-62 | [5Phos][mU][Ps][fA][Ps][mC][fA][mU][fA][mU][f |
| | | 24212 | | U][mC][fU][mU][fU][mA][fA][Ps][mC][Ps][fU][Ps |
| | | | | ][mU][Ps][fC][Ps][mA][Ps][mU] |
| | | | | [fU][mA][fA][mA][fG][mA][fA][mU][fA][mU][fG][P |
| | | | | s][mU][Ps][fA][3xGalNac] |
| 2021 | 513 | 14213_ | C5-m-63 | [5Phos][mU][Ps][fA][Ps][mG][fU][mC][fC][mU][f |
| | | 24213 | | U][mU][fU][mA][fC][mA][fC][Ps][mU][Ps][fC][Ps |
| | | | | ][mA][Ps][fA][Ps][mA][Ps][mG] |
| | | | | [fU][mG][fU][mA][fA][mA][fA][mG][fG][mA][C][ |
| | | | | Ps][mU][Ps][fA][3xGalNac] |
| 2022 | 514 | 14214_ | C5-m-64 | [5Phos][mU][Ps][fA][Ps][mU][fU][mU][fU][mC][f |
| | | 24214 | | C][mU][fU][mG][fA][mA][fA][Ps][mG][Ps][fA][Ps |
| | | | | ][mU][Ps][fC][Ps][mC][Ps][mU] |
| | | | | [fU][mU][fC][mA][fA][mG][fG][mA][fA][mA][fA][P |
| | | | | s][mU][Ps][fA][3xGalNac] |
| 2023 | 515 | 14215_ | C5-m-65 | [5Phos][mU][Ps][fG][Ps][mA][fA][mA][fU][mU][f |
| | | 24215 | | G][mU][fA][mU][fU][mU][fU][Ps][mA][Ps][fU][Ps |
| | | | | ][mC][Ps][fU][Ps][mG][Ps][mA] |
| | | | | [fA][mA][fA][mU][fA][mC][fA][mA][fU][mU][fU][P |
| | | | | s][mC][Ps][fA][3xGalNac] |
| 2024 | 516 | 14216_ | C5-m-66 | [5Phos][mU][Ps][fG][Ps][mU][fA][mA][fU][mU][f |
| | | 24216 | | U][mC][fA][mA][fA][mA][fU][Ps][mU][Ps][fC][Ps] |
| | | | | [mU][Ps][fU][Ps][mA][Ps][mA] |
| | | | | [fU][mU][fU][mU][fG][mA][fA][mA][fU][mU][fA][P |
| | | | | s][mC][Ps][fA][3xGalNac] |
| 2025 | 517 | 14217_ | C5-m-67 | [5Phos][mU][Ps][fA][Ps][mA][fA][mA][fU][mU][f |
| | | 24217 | | C][mU][fU][mA][fA][mA][fG][Ps][mU][Ps][fU][Ps |
| | | | | ][mC][Ps][fU][Ps][mU][Ps][mC] |
| | | | | [fU][mU][fU][mA][fA][mG][fA][mA][fU][mU][fU][P |
| | | | | s][mU][Ps][fA][3xGalNac] |
| 2026 | 518 | 14218_ | C5-m-68 | [5Phos][mU][Ps][fG][Ps][mA][fA][mU][fU][mU][f |
| | | 24218 | | U][mG][fG][mU][fU][mC][fU][Ps][mG][Ps][fC][P |
| | | | | s][mU][Ps][fC][Ps][mU][Ps][mA] |
| | | | | [fG][mA][fA][mC][fC][mA][fA][mA][fA][mU][fU][P |
| | | | | s][mC][Ps][fA][3xGalNac] |
| 2027 | 519 | 14219_ | C5-m-69 | [5Phos][mU][Ps][fG][Ps][mU][fC][mA][fU][mU][f |
| | | 24219 | | U][mU][fA][mU][fA][mA][fU][Ps][mU][Ps][fA][Ps] |
| | | | | [mU][Ps][fG][Ps][mU][Ps][mA] |
| | | | | [fU][mU][fA][mU][fA][mA][fA][mA][fU][mG][fA][P |
| | | | | s][mC][Ps][fA]3xGalNac] |
| 2028 | 520 | 14220_ | C5-m-70 | [5Phos][mU][Ps][fC][Ps][mA][fA][mA][fU][mC][f |
| | | 24220 | | C][mU][fG][mU][fA][mC][fU][Ps][mG][Ps][fA][Ps |
| | | | | ][mC][Ps][fA][Ps][mA][Ps][mA] |
| | | | | [fG][mU][fA][mC][fA][mG][fG][mA][fU][mU][fU][ |
| | | | | Ps][mG][Ps][fA][3xGalNac] |
| 2029 | 521 | 14221_ | C5-m-71 | [5Phos][mU][Ps][fG][Ps][mA][fU][mA][fA][mC][f |
| | | 24221 | | U][mU][fU][mU][fA][mA][fU][Ps][mA][Ps][fG][Ps |
| | | | | ][mA][Ps][fG][Ps][mA][Ps][mA] |
| | | | | [fU][mU][fA][mA][fA][mA][fG][mU][fU][mA][fU][P |
| | | | | s][mC][Ps][A]\|3xGalNac] |
| 2030 | 522 | 14222_ | C5-m-72 | [5Phos][mU][Ps][fU][Ps][mU][fA][mA][fG][mU][f |
| | | 24222 | | C][mU][fU][mC][fU][mC][fU][Ps][mU][Ps][fA][Ps |
| | | | | ][mU][Ps][fU][Ps][mC][Ps][mA] |
| | | | | [fG][mA][fG][mA][fA][mG][fA][mC][fU][mU][fA][ |
| | | | | Ps][mA][Ps][A][3xGalNac] |
| 2031 | 523 | 14223_ | C5-m-73 | [5Phos][mU][Ps][fA][Ps][mU][fA][mA][fU][mU][f |
| | | 24223 | | C][mC][fA][mA][fU][mA][fU][Ps][mG][Ps][fA][Ps] |
| | | | | [mU][Ps][fC][Ps][mA][Ps][mA] |
| | | | | [fU][mA][fU][mU][fG][mG][fA][mA][fU][mU][fA][ |
| | | | | Ps][mU][Ps][fA][3xGalNac] |
| 2032 | 524 | 14224_ | C5-m-74 | [5Phos][mU][Ps][fG][Ps][mA][fU][mA][fA][mA][f |
| | | 24224 | | U][mG][fA][mA][fC][mA][fU][Ps][mG][Ps][fG][Ps |
| | | | | ][mC][Ps][fC][Ps][mU][Ps][mA] |
| | | | | [fU][mG][fU][mU][fC][mA][fU][mU][fU][mA][fU][ |
| | | | | Ps][mC][Ps][fA][3xGalNac] |
| 2033 | 525 | 14225_ | C5-m-75 | [5Phos][mU][Ps][fA][Ps][mA][fG][mG][fU][mU][f |
| | | 24225 | | C][mA][fU][mC][fA][mU][fU][Ps][mU][Ps][fU][Ps |
| | | | | ][mC][Ps][fU][Ps][mU][Ps][mA] |
| | | | | [fA][mU][fG][mA][fU][mG][fA][mA][fC][mC][fU][ |
| | | | | Ps][mU][Ps][fA][3xGalNac] |
| 2034 | 526 | 14226_ | C5-m-76 | [5Phos][mU][Ps][fG][Ps][mG][fA][mA][fG][mU][f |
| | | 24226 | | G][mC][fU][mA][fU][mA][fA][Ps][mA][Ps][fA][Ps] |
| | | | | [mC][Ps][fA][Ps][mU][Ps][mU] |
| | | | | [fU][mA][fU][mA][fG][mC][fA][mC][fU][mU][fC][ |
| | | | | Ps][mC][Ps][fA][3xGalNac] |
| 2035 | 527 | 14227_ | C5-m-77 | [5Phos][mU][Ps][fC][Ps][mA][fA][mG][fU][mA][f |
| | | 24227 | | C][mU][fC][mU][fU][mA][fA][Ps][mA][Ps][fG][Ps |
| | | | | ][mC][Ps][fA][Ps][mA][Ps][mU] |
| | | | | [fU][mA][fA][mG][fA][mG][fU][mA][fC][mU][fU][ |
| | | | | Ps][mG][Ps][fA][3xGalNac] |
| 2036 | 528 | 14228_ | C5-m-78 | [5Phos][mU][Ps][fC][Ps][mC][fA][mA][fU][mG][f |
| | | 24228 | | A][mU][fU][mU][fC][mC][fU][Ps][mG][Ps][fU][Ps |
| | | | | ][mU][Ps][fU][Ps][mC][Ps][mA] |
| | | | | [fG][mG][fA][mA][fA][mU][fC][mA][fU][mU][fG][ |
| | | | | Ps][mG][Ps][fA][3xGalNac] |
| 2037 | 529 | 14229_ | C5-m-79 | [5Phos][mU][Ps][fA][Ps][mU][fG][G][fU][mA][f |
| | | 24229 | | U][mA][fU][mU][fC][mA][fU][Ps][mU][Ps][fU][Ps |
| | | | | ][mC][Ps][fC][Ps][mA][Ps][mA] |
| | | | | [fU][mG][fA][mA][fU][mA][fU][mA][fC][mC][fA][P |
| | | | | s][mU][Ps][fA][3xGalNac] |
| 2038 | 530 | 14230_ | C5-m-80 | [5Phos][mU][Ps][fA][Ps][mA][fC][mA][fA][mG][f |
| | | 24230 | | A][mU][fG][mA][fA][mC][fU][Ps][mU][Ps][fC][Ps |
| | | | | ][mC][Ps][fC][Ps][mA][Ps][mA] |
| | | | | [fG][mU][fU][mC][fA][mU][fC][mU][fU][mG][fU][ |
| | | | | Ps][mU][Ps][fA][3xGalNac] |
| 2039 | 531 | 14231_ | C5-m-81 | [5Phos][mU][Ps][fG][Ps][mA][fA][mC][fU][mU][f |
| | | 24231 | | C][mA][fG][mG][fA][mA][fU][Ps][mU][Ps][fU][Ps |
| | | | | ][mU][Ps][fA][Ps][mG][Ps][mA] |
| | | | | [fU][mU][fC][mC][fU][mG][fA][mA][G][mU][fU][ |
| | | | | Ps][mC][Ps][fA][3xGalNac] |
| 2040 | 532 | 14232_ | C5-m-82 | [5Phos][mU][Ps][fA][Ps][mG][fU][mC][fU][mU][f |
| | | 24232 | | C][mU][fC][mU][fU][mA][fU][Ps][mU][Ps][fC][Ps |
| | | | | ][mC][Ps][fA][Ps][mA][Ps][mA] |
| | | | | [fU][mA][fA][mG][fA][mG][fA][mA][fG][mA][fC][P |
| | | | | s][mU][Ps][fA][3xGalNac] |
| 2041 | 533 | 14233_ | C5-m-83 | [5Phos][mU][Ps][fA][Ps][mA][fU][mG][fU][mU][f |
| | | 24233 | | U][mA][fU][mA][fC][mU][fU][Ps][mU][Ps][fG][Ps |
| | | | | ][mA][Ps][fU][Ps][mA][Ps][mA] |
| | | | | [fA][mG][fU][mA][fU][mA][fA][mA][fC][mA][fU][P |
| | | | | s][mU][Ps][fA][3xGalNac] |
| 2042 | 534 | 14234_ | C5-m-84 | [5Phos][mU][Ps][fC][Ps][mG][fG][mA][fA][mU][f |
| | | 24234 | | C][mG][fU][mA][fC][mA][fC][Ps][mA][Ps][fA][Ps] |
| | | | | [mA][Ps][fG][Ps][mG][Ps][mG] |
| | | | | [fU][mG][fU][mA][fC][mG][fA][mU][fU][mC][fC][ |
| | | | | Ps][mG][Ps][fA][3xGalNac] |
| 2043 | 535 | 14235_ | C5-m-85 | [5Phos][mU][Ps][fA][Ps][mU][fA][mC][fC][mU][f |
| | | 24235 | | C][mU][fG][mC][fU][mC][fU][Ps][mU][Ps][fC][Ps |
| | | | | ][mU][Ps][fG][Ps][mA][Ps][mA] |
| | | | | [fG][mA][fG][mC][fA][mG][fA][mG][fG][mU][fA][ |
| | | | | Ps][mU][Ps][fA][3xGalNac] |
| 2044 | 536 | 14236_ | C5-m-86 | [5Phos][mU][Ps][fA][Ps][mU][fC][mA][fA][mU][f |
| | | 24236 | | U][mU][fC][mU][fU][mC][fU][Ps][mA][Ps][fC][Ps |
| | | | | ][mC][Ps][fA][Ps][mU][Ps][mA] |
| | | | | [fG][mA][fA][mG][fA][mA][fA][mU][fU][mG][fA][P |
| | | | | s][mU][Ps][fA][3xGalNac] |
| 2045 | 537 | 14237_ | C5-m-87 | [5Phos][mU][Ps][fA][Ps][mA][fC][mA][fU][mU][f |
| | | 24237 | | G][mU][fG][mU][fU][mU][fU][Ps][mG][Ps][fC][P |
| | | | | s][mA][Ps][fU][Ps][mU][Ps][mA] |
| | | | | [fA][mA][fA][mC][fA][mC][fA][mA][fU][mG][fU][P |
| | | | | s][mU][Ps][fA][3xGalNac] |
| 2046 | 538 | 14238_ | C5-m-88 | [5Phos][mU][Ps][fA][Ps][mA][fC][mU][fU][mU][f |
| | | 24238 | | A][mU][fA][mA][fG][mC][fA][Ps][mU][Ps][fA][Ps] |
| | | | | [mU][Ps][fG][Ps][mC][Ps][mU] |
| | | | | [fG][mC][fU][mU][fA][mU][fA][mA][fA][mG][fU][ |
| | | | | Ps][mU][Ps][fA][3xGalNac] |
| 2047 | 539 | 14239_ | C5-m-89 | [5Phos][mU][Ps][fA][Ps][mG][fG][mA][fU][mA][f |
| | | 24239 | | A][mC][fU][mU][fU][mU][fA][Ps][mA][Ps][fU][Ps] |
| | | | | [mA][Ps][fG][Ps][mA][Ps][mU] |
| | | | | [fA][mA][fA][mA][fG][mU][fU][mA][fU][mC][fC][P |
| | | | | s][mU][Ps][fA][3xGalNac] |
| 2048 | 540 | 14240_ | C5-m-90 | [5Phos][mU][Ps][fU][Ps][mU][fU][mA][fU][mU][f |
| | | 24240 | | G][mG][fU][mU][fG][mA][fU][Ps][mA][Ps][fC][Ps |
| | | | | ][mU][Ps][fG][Ps][mU][Ps][mA] |
| | | | | [fU][mC][fA][mA][fC][mC][fA][mA][fU][mA][fA][P |
| | | | | s][mA][Ps][fA]3xGalNac] |
| 2049 | 541 | 14241_ | C5-m-91 | [5Phos][mU][Ps][fG][Ps][mC][fA][mA][fC][mU][f |
| | | 24241 | | G][mU][fU][mU][fU][mC][fU][Ps][mU][Ps][fC][Ps |
| | | | | [mU][Ps][fG][Ps][mG][Ps][mA] |
| | | | | [fG][mA][fA][mA][fA][mC][fA][mG][fU][mU][fG][ |
| | | | | Ps][mC][Ps][fA]3xGalNac] |
| 2050 | 542 | 14242_ | C5-m-92 | [5Phos][mU][Ps][fG][Ps][mC][fU][mU][fU][mG][f |
| | | 24242 | | A][mU][fA][mC][fA][mA][fC][Ps][mU][Ps][fU][Ps] |
| | | | | [mC][Ps][fC][Ps][mA][Ps][mG] |
| | | | | [fU][mU][fG][mU][fA][mU][fC][mA][fA][mA][fG][ |
| | | | | Ps][mC][Ps][fA][3xGalNac] |
| 2051 | 543 | 14243_ | C5-m-93 | [5Phos][mU][Ps][fC][Ps][mA][fA][mA][fG][mC][f |
| | | 24243 | | U][mU][fC][mU][fC][mU][fC][Ps][mU][Ps][fU][Ps |
| | | | | ][mC][Ps][fA][Ps][mA][Ps][mG] |
| | | | | [fA][mG][fA][mG][fA][mA][fG][mC][fU][mU][fU][ |
| | | | | Ps][mG][Ps][fA][3xGalNac] |
| 2052 | 544 | 14244_ | C5-m-94 | [5Phos][mU][Ps][fG][Ps][mG][fA][mA][fC][mU][f |
| | | 24244 | | C][mC][fU][mU][fU][mC][fG][Ps][mU][Ps][fC][Ps |
| | | | | ][mU][Ps][fG][Ps][mC][Ps][mC] |
| | | | | [fG][mA][fA][mA][fG][mG][fA][mG][fU][mU][fC][ |
| | | | | Ps][mC][Ps][fA]3xGalNac] |
| 2053 | 545 | 14245_ | C5-m-95 | [5Phos][mU][Ps][fA][Ps][mU][fG][mA][fC][mA][f |
| | | 24245 | | G][mU][fU][mC][fU][mU][fU][Ps][mG][Ps][fA][Ps |
| | | | | ][mC][Ps][fU][Ps][mG][Ps][mA] |
| | | | | [fA][mA][fG][mA][fA][mC][fU][mG][fU][mC][fA][P |
| | | | | s][mU][Ps][A][3xGalNac] |
| 2054 | 546 | 14246_ | C5-m-96 | [5Phos][mU][Ps][fU][Ps][mG][fC][mA][fG][mA][f |
| | | 24246 | | A][mU][fA][mA][fC][mA][fU][Ps][mG][Ps][fU][Ps] |
| | | | | [mC][Ps][fC][Ps][mA][Ps][mA] |
| | | | | [fU][mG][fU][mU][fA][mU][fU][mC][fU][mG][fC][ |
| | | | | Ps][mA][Ps]\|fA][3xGalNac] |
| 2055 | 547 | 14247_ | C5-m-97 | [5Phos][mU][Ps][fA][Ps][mG][fA][mA][fG][mU][f |
| | | 24247 | | C][mC][fU][mA][fU][mA][fG][Ps][mU][Ps][fU][Ps |
| | | | | ][mG][Ps][fU][Ps][mA][Ps][mC] |
| | | | | [fU][mA][fU][mA][fG][mG][fA][mC][fU][mU][fC][ |
| | | | | Ps][mU][Ps][fA][3xGalNac] |
| 2056 | 548 | 14248_ | C5-m-98 | [5Phos][mU][Ps][fA][Ps][mU][fA][mA][fC][mU][f |
| | | 24248 | | U][mU][fU][mA][fA][mU][fA][Ps][mG][Ps][fA][Ps] |
| | | | | [mG][Ps][fA][Ps][mU][Ps][mU] |
| | | | | [fA][mU][fU][mA][fA][mA][fA][mG][fU][mU][fA][P |
| | | | | s][mU][Ps][fA][3xGalNac] |
| 2057 | 549 | 14249_ | C5-m-99 | [5Phos][mU][Ps][fC][Ps][mU][fA][mA][fG][mA][f |
| | | 24249 | | U][mU][fU][mC][fU][mU][fU][Ps][mU][Ps][fC][Ps |
| | | | | ][mC][Ps][fA][Ps][mA][Ps][mA] |
| | | | | [fA][mA][fG][mA][fA][mA][fU][mC][fU][mU][fA][P |
| | | | | s][mG][Ps][fA]\|3xGalNac] |
| 2058 | 550 | 14250_ | C5-m- | [5Phos][mU][Ps][fA][Ps][mU][fA][mA][fA][mU][f |
| | | 24250 | 100 | G][mA][fA][mC][fA][mU][fG][Ps][mG][Ps][C][Ps |
| | | | | ][mC][Ps][fU][Ps][mG][Ps][mC] |
| | | | | [fA][mU][fG][mU][fU][mC][fA][mU][fU][mU][fA][ |
| | | | | Ps][mU][Ps][fA][3xGalNac] |

Note =
The first nucleobase on the terminal 5' position (the sequences in the table are presented from a 5′(left) to a 3′(right direction) can be freely selected from U, A, G and C instead of the nucleobase disclosed in the table. Each of the above constructs may or may not have a phosphate modification at the 5′ end group.

TABLE 4a
Unmodified CFB-C5 muRNA constructs

| SEQ | Construct | Unmodified Combination |
|---|---|---|
| ID No. | ID No. | CFB-C5 muRNA Sequences |
| 2059 | B106-C5-30 | UUGAAUGAAACGACUUCUCCUG |
| | | UUUACAACUAUC |
| 2060 | | GAUAGUUGUAAACAGUUCCAGU |
| | | CGUUUCAUUCAA |
| 2061 | B106-C5-37 | UUGAAUGAAACGACUUCUCCAU |
| | | AUUCUGUUGUAA |
| 2062 | | UUACAACAGAAUAUGGUAUAGU |
| | | CGUUUCAUUCAA |
| 2063 | B13-C5-30 | UUGCCACAGACUCAGAGAGCUG |
| | | UUUACAACUAUC |
| 2064 | | GAUAGUUGUAAACAGUUCCCUG |
| | | AGUCUGUGGCAA |
| 2065 | B13-C5-37 | UUGCCACAGACUCAGAGAGCAU |
| | | AUUCUGUUGUAA |
| 2066 | | UUACAACAGAAUAUGGUAUCUG |
| | | AGUCUGUGGCAA |

Note =
The first nucleobase on the terminal 5′ position (the sequences in the table are presented from a 5′ (left) to a 3′ (right direction) can be freely selected from U, A, G and C instead of the nucleobase disclosed in the table).

TABLE 4b
Modified CFB-C5 muRNA constructs

Construct ID B106-C5-30
SEQ ID NO: 2067:
5′[phos] mU# fU# mG fA mA fu mG fA mA fA mC fG
mA fC mU# fU# mC# fU# rC fC# mU# fG mU fU mU fA
mC fA mA fC mU mA# mU# mC# [3XGalNAC]
SEQ ID NO: 2068
5′[phos] mG# fA# mU fA mG fU mU fG mU fA mA fA
mC fA mG# fU# fU# fC# rC fA# mG# fU mC fG mU fU
mU fC mA fU mU mC# mA# mA# [3-GalNac]
Construct ID B106-C5--37
SEQ ID NO: 2069:
5′[phos] mU# fU# mG fA mA fu mG fA mA fA mC fG
mA fC mU# fU# mC# fU# rC fC# mA# fU mA fU mU fC
mU fG mU fU mG mU# mA# mA# 3GalNAc
SEQ ID NO: 2070:
5′[phos] mU# fU# mA fC mA fA mC fA mG fA mA fU
mA fU mG# fG# mU# fA# rU fA# mG# fU mC fG mU fU
mU fC mA fU mU mC# mA# mA# [3-GalNac]
Construct ID B13-C5--30
SEQ ID NO: 2071:
5′[phos] mU# fU# mG fC mC fA mC fA mG fA mC fU
mC fA mG fA# mG# mA# rG fC# mU# fG mU fU mU fA
mC fA mA fC mU mA# mU# mC# 3GalNAc
SEQ ID NO: 2072:
5′[phos] mG# fA# mU fA mG fU mU fG mU fA mA fA
mC fA mG# fU# fU# fC# rC fC mU fG mA fG mU fC
mU fG mU fG mG mC# mA# mA# [3-GalNac]
Construct ID B13-C5-37
SEQ ID NO: 2073:
5′[phos] mU# fU# mG fC mC fA mC fA mG fA mC fU
mC fA mG fA# mG# mA# rG fC# mA# fU mA fU mU fC
mU fG mU fU mG mU# mA# mA# 3GalNAc
SEQ ID NO: 2074:
5′[phos] mU# fU# mA fC mA fA mC fA mG fA mA fU
mA fU mG# fG# mU# fA# rU fC mU fG mA fG mU fC
mU fG mU fG mG mC# mA# mA# [3-GalNac]
Note =
The first nucleobase on the terminal 5′ position (the sequences in the table are presented from a 5′ (left) to a 3′ (right direction) can be freely selected from U, A, G and C instead of the nucleobase disclosed in the table). Each of the above constructs may or may not have a phosphate modification at the 5′ end group.

Specific notes about the nomenclature in Tables 3a to 4b:
fN: 2′-Fluoro residues
rN unmodified nucleotide
mN: 2′-O-methyl residues
Ps or #: phosphorothioate
p, Phos: phosphate
(GalNAc): Sirnaomics mono-GalNAc building block

It should also be noted that the scope of the disclosed embodiments extends to sequences that correspond to those in the Tables above, and where the 5′ terminal nucleoside of the antisense (guide) strand (first region as defined in the claims herein) can include any nucleobase that can be present in an RNA molecule, in other words can be any of adenine (A), uracil (U), guanine (G) or cytosine (C). Additionally, the scope of the disclosed embodiments extends to sequences that correspond to those in the Tables above, and where the 3′ terminal nucleoside of the sense (passenger) strand (second region as defined in the claims herein) can include any nucleobase that can be present in an RNA molecule, in other words can be any of adenine (A), uracil (U), guanine (G) or cytosine (C), advantageously however a nucleobase that is complementary to the 5′ nucleobase of the antisense (guide) strand (first region as defined in the claims herein).

While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form yet additional examples.

It will be understood that the above description of a advantageous embodiment is given by way of example only and that various modifications may be made by those skilled in the art. What has been described above includes examples of one or more embodiments. It is, of course, not possible to describe every conceivable modification and alteration of the above compounds, compositions or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif or modification patterns provides reasonable support for additional oligonucleotides having the same or similar motif or modification patterns.

The syntheses of the RNAi constructs according to the disclosed embodiments and disclosed herein have been carried out using synthesis methods known to the person skilled in the art, such as synthesis methods disclosed in https://en.wikipedia.org/wiki/Oligonucleotide_synthesis {retrieved on 16 Feb. 2022}, where the methods disclosed on this website are incorporated by reference herein in their entirety. The only difference to the synthesis method disclosed in this reference is that GalNAc phosphoramidite immobilized on a support is used in the synthesis method during the first synthesis step.

### Example 1

### Materials and Methods

### Cell Culture:

HepG2 (ATCC cat. 85011430) cells were maintained by biweekly passing in EMEM supplemented with 10% FBS, 20 mM L-glutamine, 10 mM HEPES pH 7.2, 1 mM sodium pyruvate, 1×MEM non-essential amino acids, and 1× Pen/Strep (EMEM complete).

### CFB Target Identification and Duplex Preparation:

Oligomeric compounds targeting CFB were identified by bioinformatic analysis on human CFB mRNA sequence as given in RefSeq sequence ID NM_001710.5. 250 compounds were selected for synthesis as asymmetric duplexes. Compounds were dissolved to 50 uM in molecular biology grade water and annealed by heating at 95° C. for 5 minutes followed by gradual cooling to room temperature.

### CFB—Primary Screen:

On the day of transfection, HepG2 cells were collected by trypsinization, counted, and seeded in 96 well tissue culture treated plates at 10,000 cells per well in 50 uL complete EMEM with 20% FBS. Cells were allowed to rest for 4 hours before transfection with 2 pmoles of each respective CFB-targeting oligomeric compound in triplicate via RNAiMax (ThermoFisher). In brief, 8 pmoles of each compound were diluted in 100 uL OptiMEM and mixed gently with 0.8 uL of RNAiMax in 100 uL OptiMEM to make 200 uL total complex. 50 uL of each RNAiMax complexed compound was added to each respective triplicate well of HepG2 cells for a final mixture of 20 nM compound in a volume of 100 uL, 50/50 EMEM/OptiMEM at 10% FBS.

72 hours post transfection, cells were harvested and RNA isolated using the PureLink Pro 96 total RNA Purification Kit (ThermoFisher, 12173011A) according to the manufacturer protocol. Harvested RNA was assayed for CFB expression via Taqman qPCR using the Luna Universal Probe One-Step RT-qPCR Kit (NEB, E3006). Two separate qPCR assays were performed for each sample using two separate CFB Taqman probe sets multiplexed with a common GAPDH VIC probe (ThermoFisher, 4326317E). Thermocycling and data acquisition was performed with an Applied Biosystems QuantStudio 3 Real-Time PCR System.

### CFB—Secondary Screen:

Based on data from the primary screen, a yet narrower set of the best performing 30 CFB-targeting constructs were tested in dose curves. As before, HepG2 cells were collected by trypsinization and seeded in 96 well tissue culture plates at 10,000 cells per well in 50 uL complete EMEM with 20% FBS and allowed to rest for 4 hours. Transfection complexes were formed by gently mixing 36 pmoles of each compound in 180 uL OptiMEM with 2.16 uL RNAiMax in 180 uL OptiMEM to make 360 uL total complex. A two fold dilution series was then performed with basal OptiMEM. 50 uL of each dilution was added to respective triplicates of HepG2 cells to make a final dilution series of 50 nM down to 0.32 nM in a volume of 100 uL, 50/50 EMEM/OptiMEM at 10% FBS.

72 hours post transfection, cells were harvested and RNA isolated using the PureLink Pro 96 total RNA Purification Kit (ThermoFisher, 12173011A) according to the manufacturer protocol. Harvested RNA was assayed for CFB expression via Taqman qPCR using the Luna Universal Probe One-Step RT-qPCR Kit (NEB, E3006). A single qPCR assay was performed for each sample using CFB Taqman probe set multiplexed with a common GAPDH VIC probe (ThermoFisher, 4326317E). Thermocycling and data acquisition was performed with an Applied Biosystems QuantStudio 3 Real-Time PCR System.

### Example 2

### Results

Table 5 below shows CFB IC50 values (in nM) for 30 advantageous constructs selected in accordance with the examples. Max % KD indicates the maximally achieved knock-down with 0% being no knock-down and 100% full knock-down.

| SEQ ID No. | Construct ID | IC₅₀ | Max % KD |
|---|---|---|---|
| 94 and 346 | CFB94 | 4.36 | 83 |
| 247 and 499 | CFB247 | 4.42 | 84 |
| 13 and 265 | CFB13 | 5.72 | 93 |
| 106 and 358 | CFB106 | 5.84 | 88 |
| 28 and 280 | CFB28 | 6.24 | 84 |
| 135 and 387 | CFB135 | 6.91 | 89 |
| 132 and 384 | CFB132 | 6.92 | 87 |
| 32 and 284 | CFB32 | 6.96 | 81 |
| 83 and 335 | CFB83 | 7.17 | 88 |
| 102 and 354 | CFB102 | 7.39 | 83 |
| 62 and 314 | CFB62 | 7.61 | 90 |
| 241 and 493 | CFB241 | 7.84 | 82 |
| 9 and 261 | CFB09 | 8.02 | 92 |
| 54 and 306 | CFB54 | 8.24 | 82 |
| 143 and 395 | CFB143 | 8.81 | 82 |
| 103 and 355 | CFB103 | 9.16 | 80 |
| 128 and 380 | CFB128 | 9.22 | 83 |
| 53 and 305 | CFB53 | 9.52 | 83 |
| 150 and 402 | CFB150 | 10.33 | 83 |
| 82 and 334 | CFB82 | 10.34 | 88 |
| 36 and 288 | CFB36 | 10.64 | 79 |
| 25 and 277 | CFB25 | 10.88 | 81 |
| 71 and 323 | CFB71 | 11.47 | 84 |
| 17 and 269 | CFB17 | 11.60 | 77 |
| 5 and 257 | CFB05 | 12.67 | 75 |
| 141 and 393 | CFB141 | 13.37 | 77 |
| 90 and 342 | CFB90 | 13.78 | 85 |
| 127 and 379 | CFB127 | 14.04 | 73 |
| 75 and 327 | CFB75 | 16.45 | 69 |
| 95 and 347 | CFB95 | 19.25 | 72 |

The IC₅₀ data in the single- to low double-digit nanomolar range demonstrate outstanding performance of numerous constructs of the disclosed embodiments.

### Example 3

### Materials and Methods

### Cell Culture:

Human primary hepatocytes (5 donor pooled—Sekisui XenoTech, HPCH05+) were thawed immediately prior to experimentation and cultured in 1× complete Williams medium (Gibco, A1217601) supplemented with Hepatocytes plating supplement pack (Gibco, CM3000). FEBS concentration was modified from manufacture recipe to a final 2.5% (as opposed to 5%) for compound stability.

1× Complete WEM: 2.5% FEBS, 1 NM Dexamethasone, Pen/Strep (100 U/mL/100 Ng/mL), 4 μg/ml Human Insulin, 2 mM GlutaMAX, 15 mM HEPES, pH 7.4.

Hepatocytes were plated on Collagen I (rat tail) coated 96 well tissue culture plates (Gibco, A1142803).

### CFB Compound Preparation:

Compounds were dissolved to 200 μM in water and annealed by heating at 95° C. for 5 minutes followed by rapid cooling on ice.

### CFB Compound Transfections:

On the day of transfection, primary human hepatocytes were thawed in 45 mL of human OptiThaw (Sekisui XenoTech, K8000) and centrifuged down at 200 g for 5 minutes. Cells were resuspended in 2× complete WEM and counted. Cell were then plated in 50 μL of 2× complete WEM at 25,000 cells per well on 96 well type 1 rat tail Collagen plates and allowed to rest and attach for four hours before transfection.

Compounds were diluted further to 2 μM in basal WEM. A seven step, fivefold dilution series was prepared in basal WEM from 2 μM to 0.000128 μM. 50 μL of each dilution was added to respective triplicates of the plated hepatocytes for a final dilution series of 1 μM down to 0.000064 μM in a volume of 100 uL 1× complete WEM.

72 hours post transfection, cells were harvested and RNA isolated using the PureLink Pro 96 total RNA Purification Kit (ThermoFisher, 12173011A) according to the manufacturer protocol. Harvested RNA was assayed for CFB expression via TaqMan qPCR using the Luna Universal Probe One-Step RT-qPCR Kit (NEB, E3006). A single qPCR assay was performed for each sample using an CFB TaqMan probe set (Hs01011282_g1-FAM) multiplexed with a common GAPDH VIC probe (ThermoFisher, 4326317E). Thermocycling and data acquisition was performed with an Applied Biosystems QuantStudio 3/5 Real-Time PCR System.

### Results

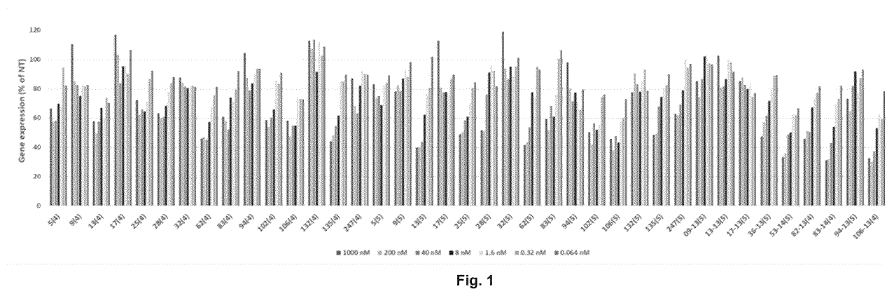

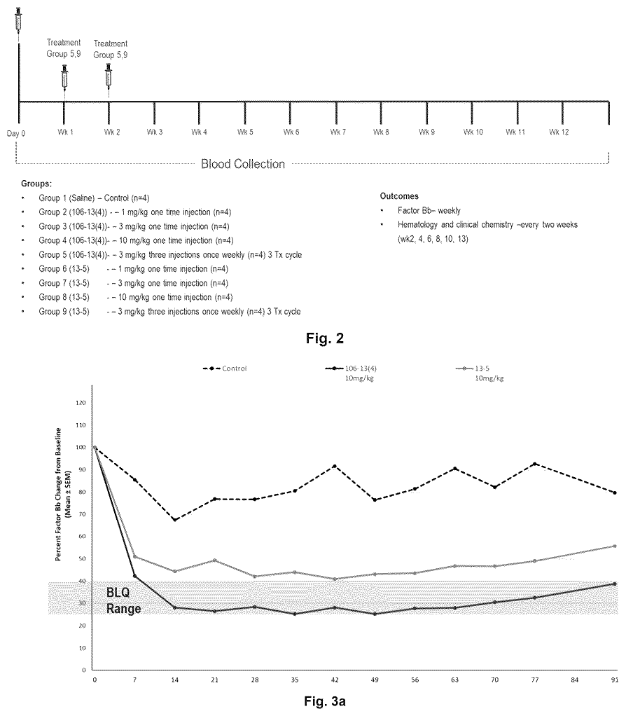

The results of the in vitro studies are shown in FIG. 1.

### Example 4

Pharmacodynamics Study of STP144G (constructs 13(5) and 106-13(4), SEQ ID Nos. 1757-1758 in Table 3b) Following Single/Repeat Subcutaneous Injection to Non-Naïve Cynomolgus Monkeys.

### Study Protocol

The following study protocol has been drafted before the animal experiments and studies have been completed and therefore uses the future tense. However, as the study has already been completely carried out, each usage of “future tense” shall be considered as the “past tense” in the following description of the study protocol.

TABLE 6
Study Design

| | | | | | TOTAL | Target | |
|---|---|---|---|---|---|---|---|
| | | | | Target | Target Dose | Dose | |
| | | CFB Test | SEQ | Dose Level | Volume | Concentration | Dose |
| Group | # of Males | Article | ID No. | (mg/animal) | (mL) | (mg/mL) | Route |
| 1 | 4 non- | saline | — | — | 3.0 mL | — | SC |
| | naïve | | | | | | |
| 2 | 4 non- | STP144G(106- | 1758 | 2.5 (Target | 3.0 mL | 1 | SC |
| | naïve | 13(4)) | | as 1 mg/kg) | | | |
| 3 | 4 non- | STP144G | 1758 | 7.5 (Target | 3.0 mL | 3 | SC |
| | naïve | (106-13(4)) | | as 3 mg/kg) | | | |
| 4 | 4 non- | STP144G | 1758 | 25 (Target | 3.0 mL | 10 | SC |
| | naïve | (106-13(4)) | | as 10 mg/kg) | | | |
| 5 | 4 non- | STP144G | 1758 | 7.5 (Target | 3.0 mL | 3 | SC |
| | naïve | (106-13(4)) | | as 3 mg/kg) | | | |
| 6 | 4 non- | STP144G | 1757 | 2.5 (Target | 3.0 mL | 1 | SC |
| | naïve | (13(5)) | | as 1 mg/kg) | | | |
| 7 | 4 non- | STP144G | 1757 | 7.5 (Target | 3.0 mL | 3 | SC |
| | naïve | (13(5)) | | as 3 mg/kg) | | | |
| 8 | 4 non- | STP144G(13(5)) | 1757 | 25 (Target | 3.0 mL | 10 | SC |
| | naïve | | | as 10 mg/kg) | | | |
| 9 | 4 non- | STP144G | 1757 | 7.5 (Target | 3.0 mL | 3 | SC |
| | naïve | (13(5)) | | as 3 mg/kg) | | | |

Note:
1. Test article storage: at 4° C., protected from light (DO NOT FREEZE the test article)
2. For all groups, animals will be fed on daily diet.
3. For all groups, saline will be used for vehicles

TABLE 7
Dose and Sample Collections

| | | Day | Day | Wk | Wk | Wk | Wk | Wk | Wk | Wk | Wk | Wk | Wk | Wk | Wk | Wk |
|---|---|---|---|---|---|---|---|---|---|---|---|---|---|---|---|---|
| | Group | −7 | 0 | 1 | 2 | 3 | 4 | 5 | 6 | 7 | 8 | 9 | 10 | 11 | 12 | 13 |
| Dose | 1 | | QD | | | | | | | | | | | | | |
| | 2 | | QD | | | | | | | | | | | | | |
| | 3 | | QD | | | | | | | | | | | | | |
| | 4 | | QD | | | | | | | | | | | | | |
| | 5 | | QD | QD | QD | | | | | | | | | | | |
| | 6 | | QD | | | | | | | | | | | | | |
| | 7 | | QD | | | | | | | | | | | | | |
| | 8 | | QD | | | | | | | | | | | | | |
| | 9 | | QD | QD | QD | | | | | | | | | | | |
| Clinical | 1-9 | BI, | | BI, | BI, | BI, | BI, | | BI, | | BI, | | BI, | | | BI, |
| Pathology ^{a} | | Se | | Se | Se | Se | Se | | Se | | Se | | Se | | | Se |
| Pharma | 1-9 | P, | P, | P, | P, | P, | P, | P, | P, | P, | P, | P, | P, | P, | P, | P, |
| co- | | Se | Se | Se | Se | Se | Se | Se | Se | Se | Se | Se | Se | Se | Se | Se |
| dynamics ^{b} | | | | | | | | | | | | | | | | |

1. Wk 1 point should be 7 days after Day 0; Wk 2 point should be 14 days after Day 0.
2. Single dose with single compound, subcutaneous injection. For Day 0, Wk 1, and Wk 2, animals will be dosed after all sample collections.

### Study Information

### 4.1.1 Study Objective

The objective of this study is to determine the pharmacodynamics (PD) of STP144G following a single/repeat subcutaneous (SC) administration in male cynomolgus monkeys.

### 4.1.2 Test Article and Vehicle Information

TABLE 8
Test Articles

| | | | Molecular | | | | | |
|---|---|---|---|---|---|---|---|---|
| | SEQ | | Weight of | Molecular | | | | |
| Test | ID | Lot/Batch | Free | Weight of | Salt | Chemical | Purity | |
| Article | No. | Number | Base | Salt | Factor | Formula | (%) | Storage |
| STP144G | 1758 | K1 | Refer to | Refer to | Refer to | Refer to | 83% | 4 C |
| (106-13(4) | | | product | product | product | product | | |
| as + s) | | | CoA | CoA | CoA | CoA | | |
| STP144G | 1757 | K1 | Refer to | Refer to | Refer to | Refer to | 83% | 4 C |
| (13(5) | | | product | product | product | product | | |
| as + s) | | | CoA | CoA | CoA | CoA | | |

*Free base
NA = Not applicable

### 4.1.3 Blood Collection for Clinical Pathology

All blood samples will be collected from a peripheral vessel from restrained, non-sedated animals.

TABLE 9
Clinical Pathology Schedule

| | | | | | | Sample |
|---|---|---|---|---|---|---|
| | | | | | | Volume |
| | | Sampling | | Tube Type/Size | | approximately |
| Groups | Sample | Schedule^{a} | Evaluations | Information | | (minimum) |
| 1-9 | Blood | Once during | Hematology | K₂EDTA | 2 mL | 2.0 mL (1.0 mL) |
| | | pre-study | Clinical | Plain with | 1.4 mL | 0.7 mL (0.6 mL) |
| | | (Day −16); | Chemistry | separating | | |
| | | Weeks 1, 2, 3, | | gel | | |
| | | 4, 6, 8, 10, 13. | | | | |

^{a}Blood sample may be collected from animals subjected to unscheduled euthanasia. Animals may not be fasted under that circumstance.

(1) Blood Collection for Hematology: Whole blood (at least 1.0 mL) will be collected from the animals into commercially available tubes with Potassium (K2) EDTA at room temperature (RT). The blood samples will be sent to clinical pathology lab in RT and tested for hematology parameters listed in the Table 8.

Erythrocyte count (RBC), Red cell distribution width (RDW), Hematocrit (HCT), Platelet count (PLT), Hemoglobin (HGB), Mean platelet volume (MPV), Mean corpuscular volume (MCV), Leukocyte counts (WBC), and Differential (absolute and percent), Mean corpuscular hemoglobin (MCH), Blood smear for possible cytology, Mean corpuscular hemoglobin concentration (MCHC), Absolute reticulocyte count (Retic), Hemoglobin Concentration Distribution Width (HDW) and Platelet Distribution Width (PDW)

A blood smear will be prepared from each hematology sample. Blood smears will be labeled, stained, and stored. Blood smears may be read to investigate results of the hematology analyses. If additional examination of blood smears is deemed necessary, the smears may be evaluated subsequently and this evaluation will be described in a study plan amendment.

(2) Blood Collection for Clinical Chemistry: Whole blood samples (approximately 1.4 mL) without anticoagulant will be collected into commercially available plain tubes with separating gel, held at RT up-right for at least 30 minutes, and sent to clinical pathology lab. The samples will be processed to serum, which will be examined for the parameters listed in Table 8.

Alkaline Phosphatase (ALP), Total Protein (TP), Alanine Aminotransferase (ALT), Albumin (ALB), Aspartate Aminotransferase (AST), g-glutamyltransferase (GGT), Bilirubin, total (TBIL), Globulin (GLB), Phosphorus (P), Albumin/Globulin Ratio Creatinine (CRE), Sodium (Na), Glucose (GLU), Chloride (Cl), Calcium (Ca), Triglycerides (TG), Total Cholesterol (TCHO), Urea (UREA), Potassium (K), Creatine Kinase (CK), Lactate Dehydrogenase (LDH), Glutamate dehydrogenase (GLDH)

### 4.1.4 Blood Collection for Pharmacodynamics (PD)

Blood: All blood samples will be collected from a peripheral vessel from restrained, non-sedated animals.

### Animals: All Available, all Groups

TABLE 10
Pharmacodynamics Schedule

| | | | | | | Sample Volume |
|---|---|---|---|---|---|---|
| | | Sampling | | Tube Type/Size | | approximately |
| Groups | Sample | Schedule^{a} | Evaluations | Information | | (minimum) |
| 1-9 | Blood | Once during | CFB ELISA | K₂EDTA | 2 mL | 1.5 mL (1.0 mL) |
| | | pre-study | | | | |
| | | (Day −16); | | | | |
| | | Day 1, and | Serum | Plain with | 3 mL | 1.5 mL (1.3 mL) |
| | | weekly | | separating | | |
| | | thereafter. | | gel | | |

1. ^{a}Blood sample may be collected from animals subjected to unscheduled euthanasia. Animals may not be fasted under that circumstance.

### Post-Dose

Blood volume: Approximately 5.0 mL total
Frequency: Refer to Table 8. Actual sample collection times will be recorded in the study records. For samples collected within the first hour of dosing, a ±1 minute is acceptable. For the remaining time points, samples that are taken within 5% of the scheduled time are acceptable and will not be considered as protocol deviation.
Sample Processing: For CFB ELISA: 2 mL Blood will be collected into a tube (Purchased from sponsor's required company) containing K2EDTA on wet ice. Then all the blood will be mixed upside down 4 times. Samples will be centrifuged (1000 g for 20 minutes at 4° C.) and approximately 1 mL plasma will be transferred into two tubes quickly (approximately 0.50 mL per tube). All tubes should be flash frozen, and then stored at −80° C.
For Serum: Whole blood samples (approximately 3.0 mL) without anticoagulant will be collected in serum separator tubes. Invert the tubes gently 4 times. Then held at RT and up-right for 30 minutes, to allow clotting, and then samples will be centrifuged (3200 g for 10 minutes at 4° C.) and approximately 1.5 mL serum will be transferred into three tubes quickly (approximately 0.50 mL per tube).

### Results

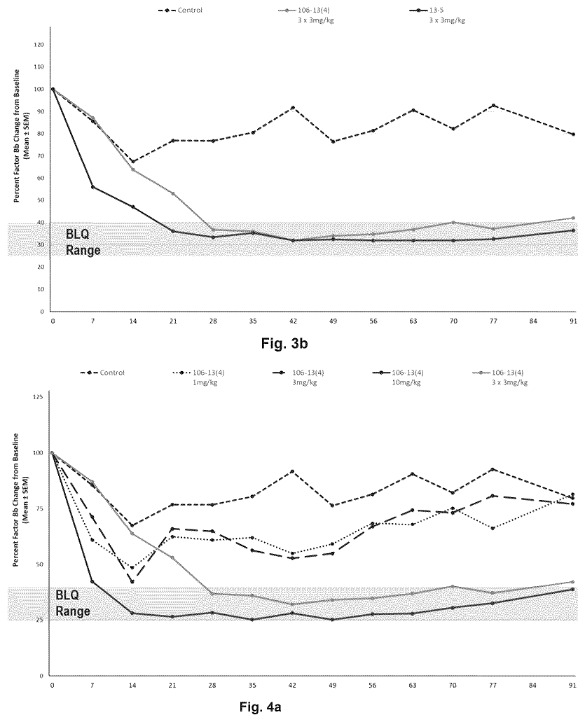

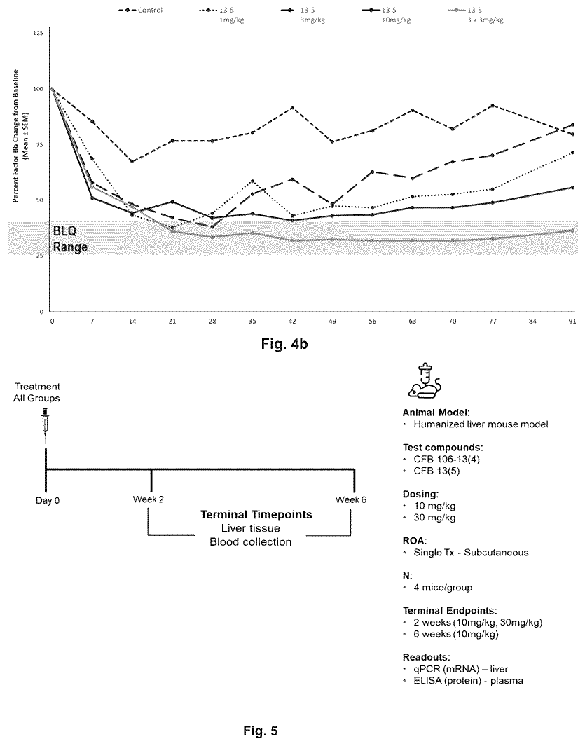

Max reduction of Factor Bb and duration of response (see FIG. 3a):
106-13(4) (SEQ ID No. 1758)
   Max suppression of 74% at week 5
   >60% reduction from week 2 to week 13
   Mean BLQ from week 2 to week 10
13(5) (SEQ ID No. 1757)
   Max suppression of 59% at week 6
   >50% reduction from week 2 to week 13
   No Mean BLQ for any of the timepoints

Max reduction of Factor Bb and duration of response (see FIG. 3b):
106-13(4) (SEQ ID No. 1758)
   Max suppression of 68% at week 6
   >50% reduction from week 4 to week 13
   Mean BLQ at week 6
13(5) (SEQ ID No. 1757)
   Max suppression of 68% at week 6
   >50% reduction from week 2 to week 13
   Mean BLQ from week 6 to week 11

### Example 5

### Dose Response and Duration Response In Vivo in a Humanized Liver Mouse Model

The purpose of this study is to evaluate a dose- and duration-response effect of selected candidate leads for GalNAc-siRNA constructs targeting Complement Factor B (CFB) in a humanized liver UPA-SCID mouse model. Test articles will be administered via subcutaneous administration and evaluated at 14 and 42 days post-dose. Endpoints will include the collection of liver punch biopsies, and the collection of serum and plasma samples for the evaluation of Factor Bb and CFB activity in the Factor Bb ELISA and hemolytic assay, respectively.

### Materials and Methods

### Test and Control Articles

Vehicle: Phosphate Buffered Saline (PBS)

| | |
|---|---|
| Identification | PBS |
| Description | Vehicle |
| Appearance | Clear solution |
| CAS Number | N/A |
| Manufacturer/ | Will be documented in study records and final report |
| Supplier | |
| Storage Conditions | Ambient |
| Catalog Number | Will be documented in study records and final report |
| Lot/Batch Number | Will be documented in study records and final report |
| Expiration/Retest | Will be documented in study records and final report |
| Date | |
| Sterility | Sterile |
| Purity | Will be documented in study records and final report |
| pH | Will be documented in study records and final report |
| Dose Concentration | 0 mg/kg |
| Dose Volume | 200 μL fixed volume |
| Route | Subcutaneous injection |
| Dosing Frequency | Once on Day 0 |
| Dose Preparation | N/A |

Test Article: CFB mxRNA #13(5)

| Identification | CFB13(5) (SEQ ID No. 1757) | |
|---|---|---|
| Description | GalNAc-siRNA targeting Complement Factor B | |
| Appearance | Clear liquid | |
| CAS Number | N/A | |
| Storage Conditions | 4° | C. |
| Lot/Batch Number | N/A | |
| Expiration/Retest | N/A | |
| Date | | |
| Sterility | <1 | EU/mg |

| | |
|---|---|
| Purity | >80% |
| pH | N/A |
| Dose Levels | 10 and 30 mg/kg (approximated - fixed |
| | volumed will be administered) |
| Dose Volume | 200 μL fixed volume |
| Dose Concentrations | N/A |
| Route | Subcutaneous injection in scruff |
| Dosing Frequency | Once on Day 0 |
| Dose Preparation | Ready to inject solutions will be provided |

Test Article: CFB mxRNA #106-13(4)

| Identification | CFB - 106-13(4) (SEQ ID No. 1758) | |
|---|---|---|
| Description | GalNAc-siRNA targeting Complement Factor B | |
| Appearance | Clear liquid | |
| CAS Number | N/A | |
| Storage Conditions | 4° | C. |
| Lot/Batch Number | N/A | |
| Expiration/Retest | N/A | |
| Date | | |
| Sterility | <1 | EU/mg |

| | |
|---|---|
| Purity | >80% |
| pH | N/A |
| Dose Levels | 10 and 30 mg/kg (approximated - fixed |
| | volumed will be administered) |
| Dose Volume | 200 μL fixed volume |
| Dose Concentrations | N/A |
| Route | Subcutaneous injection in scruff |
| Dosing Frequency | Once on Day 0 |
| Dose Preparation | Ready to inject solutions will be provided |

### Dose Formulation

No preparation is required for the test material as it will be received as ready-to-dose formulations.

### Identification of Test and Control Articles

All test article, positive control, and vehicle control storage containers will be labelled at a minimum with identification (including lot/batch number, if available), storage conditions, and expiration/retest date, if available.

### Test System

### Justification of Test System

Humanized liver uPA-SCID mice are reported to have up to 95% human hepatocyte engraftment; normal human liver histology and function; human-specific metabolism and excretion pathways; expression of human genes, mRNA, and proteins; human-like lipid profiles, production of human albumin and human-like biliary excretion, and a wide range of research applications. Thus, humanized liver uPA-SCID mice are an ideal test system for the evaluation of therapeutics that involve CFB targets as CFB is produced in the liver.

### Animals

Female humanized liver uPA-SCID mice (approximately 22-24 weeks old) were acclimated at least 7 days prior to use. Only animals in good health prior to dosing will be assigned to the study. The animals will be monitored daily for the appearance of local or systemic toxicity. All animals will be housed in clean room and animal handling will be performed in a sterilized biological safety cabinet by trained personnel wearing appropriately disinfected personal protective equipment.

### Methodology

### Study Outline

On Day 0, all study animals will be dosed by subcutaneous injection according to the Study Outline below. Clinical observations will be recorded daily. The study design will require 32 female mice (4 mice per treatment group, plus 2 extra mice). On Day 14, 4 vehicle animals (Group 1A), 8 CFB mxRNA #13(5) animals (Groups 2A and 2B), and 8 CFB mxRNA #106-13(4) animals (Groups 3A and 3B) will be euthanized. On Day 42, 4 CFB mxRNA #13(5) animals (Group 2C), and 4 CFB mxRNA #106-13(4) animals (Group 3C) will be euthanized. At each time point, terminal blood collections for serum and plasma, liver punch biopsies, and the collection of remaining liver tissue will be performed.

TABLE 11
Study Schedule
Study Outline¹

| Terminal | | | CFB mxRNA |
|---|---|---|---|
| Time | Control | CFB mxRNA #13(5) | #106-13(4) SEQ |
| point | (PBS) | SEQ ID No. 1757 | ID No. 1758 |
| Day 14 | Group 1A | Group 2A | Group 3A |
| | | (10 mg/kg) | (10 mg/kg) |
| | | Group 2B | Group 3B |
| | | (30 mg/kg) | (30 mg/kg) |
| Day 42 | Group 1B | Group 2C | Group 3C |
| (Week 6) | | (10 mg/kg) | (10 mg/kg) |

¹n = 4/group. Two extra mice will serve as replacements due to health conditions or body weight outliers.

### Body Weights

Body weights will be collected at receipt (for general health assessment), on Day −1, and at terminal time points prior to euthanasia.

### Dosing (Day 0)

On Day 0, all mice will be injected subcutaneously with vehicle or test article per the Study Outline Table. Each animal will be injected subcutaneously in scruff with an injection volume of 200 uL.

### Daily Observations

All animals will be observed at least once daily for clinical signs. As humanized mice are predisposed to opportunistic infection due to compromised immune function. Staff will monitor mice for clinical signs that may indicate infection, including ruffled fur and hunched posture that become more pronounced beyond the slightly ruffled fur and hunched posture that exists at baseline, and decreased activity. Common infections to be aware of in these mice include: infected skin wounds, cellulitis, abscesses (skin and internal organs), otitis media, conjunctivitis, panophthalmitis, and localized and widespread infections involving liver, heart, lungs, uterus, accessory sex glands. While not indicative of infection, abdominal distension (related to liver tissue engraftment) may be observed and if observed, will be noted.

### Unscheduled Deaths and Moribund Animals

Moribund animals displaying severe effects will be discussed with the veterinarian or euthanized at the veterinarian's and Study Director's recommendation. Animals will be monitored with an increased frequency (up to twice daily; at least 5 hours between observations) if adverse clinical signs are observed, including mortality of other animals on study. Abnormal findings will be recorded as they are observed.

Samples will be collected from animals found dead and carcasses will be discarded without further evaluation.

### Terminal Procedures (Day 14 and Day 42)

All mice remaining at the scheduled intervals (Days 14 and 42) will be euthanized by asphyxiation with CO2, and terminal blood collections will be collected via cardiac stick or the inferior vena cava (maximum volume, collection site to be documented in the study records) for processing to serum and plasma. Terminal body weights will be collected prior to euthanasia.

### Plasma and Serum Processing

Following terminal collections on Days 14 and 42, respectively, blood will be processed to plasma for Factor Bb analysis and to serum for the hemolytic assay. Blood sample volumes will be divided evenly for plasma and serum processing.

### Plasma

Blood samples will be placed into K2 EDTA blood collection tubes and inverted 8-10 times to ensure adequate mixing. Samples will be maintained cold, on ice, and centrifuged in an instrument set to 2-4° C. and ≤1300×g for 10 minutes, within 60 minutes of sample collection. Each plasma sample will be aliquoted into two fresh, labelled collection tubes and stored frozen at <−70° C. until analysis is performed.

### Serum

Blood samples will be placed into blood collection tubes without anti-coagulant and allowed to clot for 30-60 minutes. Samples will then be processed to serum following centrifugation at 2500×g for 5 minutes in an instrument set to room temperature. Each serum sample will be aliquoted into a fresh, labeled collection tube and snap-frozen with dry ice immediately following collection. Sample tubes will be labeled with the study number and sample type, and stored at <−70° C. until analysis is performed.

### Organ Processing

Three (3) liver punch biopsies (2 mm) will be obtained from each collected liver (taken from the left, middle, and right lobes, respectively) and placed into separate, labeled 2 mL tubes containing RNALater. Liver punch biopsies will be allowed to soak in the RNALater for 15 minutes, then will be flash-frozen and stored at <−70° C.

### Results

Mice with humanized liver cells (and retaining about 20 to 25% murine liver cells) have been used to study performance of two particularly advantageous compounds of the disclosed embodiments.

FIG. 5 shows an overview of the study protocol.

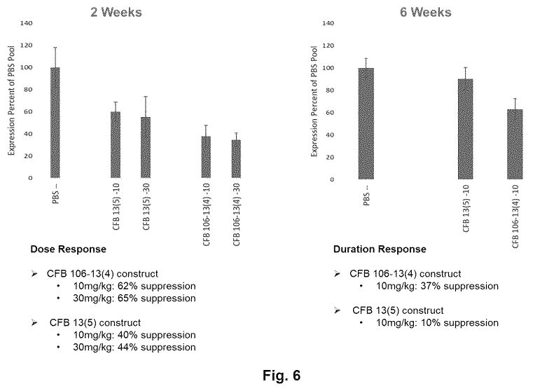

FIG. 6 shows knock-down at the mRNA level by two compounds of the disclosed embodiments as compared to negative control after 2 and 6 weeks.

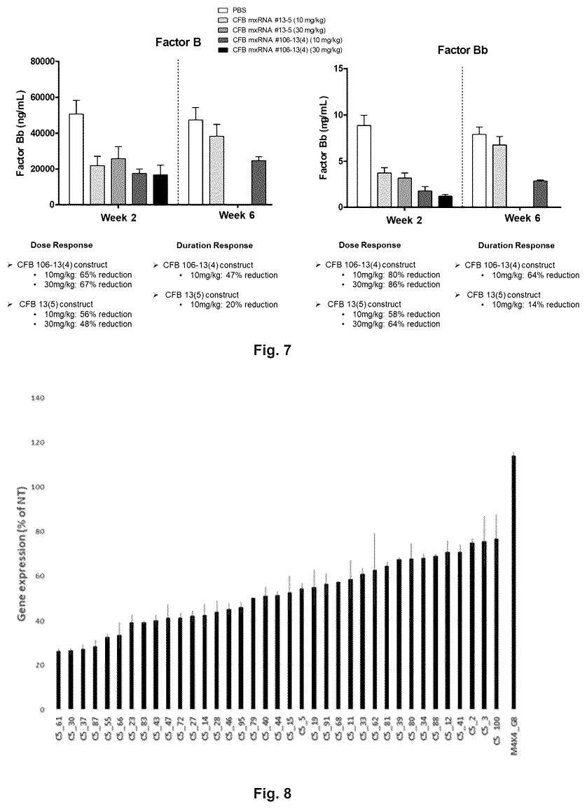

FIG. 7 shows amounts of CFB (“Factor B”) as well as of Factor Bb (as further read-out for CFB and complement pathway down-regulation) in plasma as compared to negative control after 2 and 6 weeks.

The data demonstrate significant knock-down at both mRNA and protein level for both compounds, where 106-13(4) outperforms 13(5). A certain rebound after six weeks, to a lesser extent for the former compound, can be seen.

As regards protein levels, and including component Bb of the complement pathway, an even more persistent knock-down is observed. In particular, compound 106-13(4) shows Bb knock-down in plasma at a level of 64% reduction still after 6 weeks. Such findings are indicative of a favourable dosage regimen requiring administration in large intervals such as every 2, 3, 4, 5, 6, 7, 8, 9 or 10 weeks.

### Example 6

### Materials and Methods

### Cell Culture:

Human primary hepatocytes (5 donor pooled—Sekisui XenoTech, HPCH05+) were thawed immediately prior to experimentation and cultured in 1× complete Williams medium (Gibco, A1217601) supplemented with Hepatocytes plating supplement pack (Gibco, CM3000). FBS concentration was modified from manufacture recipe to a final 2.5% (as opposed to 5%) for compound stability.

1× Complete WEM: 2.5% FBS, 1 μM Dexamethasone, Pen/Strep (100 U/mL/100 μg/mL), 4 μg/ml Human Insulin, 2 mM GlutaMAX, 15 mM HEPES, pH 7.4.

### C5 Target Identification and Duplex Preparation:

Oligomeric compounds targeting C5 were identified by bioinformatic analysis on human C5 mRNA sequence as given in RefSeq sequence ID NM_001735.2. 100 compounds were selected for synthesis as mxRNA hairpins. Compounds were dissolved to 50 uM in molecular biology grade water. Duplexes were annealed by heating at 95° C. for 5 minutes followed by gradual cooling to room temperature. mxRNAs were annealed by heating at 95° C. for 5 minutes followed by rapid cooling on ice.

### C5—Primary Screen:

On the day of transfection, primary human hepatocytes were thawed in 45 mL of human OptiThaw (Sekisui XenoTech, K8000) and centrifuged down at 200 g for 5 minutes. Cells were resuspended in 2× complete WEM and counted. Cells were then plated in 50 μL of 2× complete WEM at 25,000 cells per well on 96 well type 1 rat tail Collagen plates and allowed to rest and attach for four hours before transfection. After rest, the compounds were diluted further to 2 μM in basal WEM. 50 μL of each 2 μM compound was added to respective triplicates of the plated hepatocytes for a final concentration of 1 μM in a volume of 100 uL 1× complete WEM.

72 hours post transfection, cells were harvested and RNA isolated using the PureLink Pro 96 total RNA Purification Kit (ThermoFisher, 12173011A) according to the manufacturer protocol. Harvested RNA was assayed for C5 expression via Taqman qPCR using the Luna Universal Probe One-Step RT-qPCR Kit (NEB, E3006). A qPCR assay was performed for each sample using a C5 TaqMan probe set (Hs01004342_m1-FAM) multiplexed with a common GAPDH VIC probe (ThermoFisher, 4326317E). Thermocycling and data acquisition was performed with an Applied Biosystems QuantStudio 3/5 Real-Time PCR System.

### C5—Secondary Screen:

Based on data from the primary screen, a narrower set of the best performing 25 C5-targeting mxRNA constructs were tested in dose curves (See Table 12). Compounds were diluted further to 2 μM in basal WEM. A seven step, five fold dilution series was prepared in basal WEM from 2 μM to 0.000128 μM. 50 μL of each dilution was added to respective triplicates of the plated hepatocytes for a final dilution series of 1 μM down to 0.000064 μM in a volume of 100 uL 1× complete WEM.

72 hours post transfection, cells were harvested and RNA isolated using the PureLink Pro 96 total RNA Purification Kit (ThermoFisher, 12173011A) according to the manufacturer protocol. Harvested RNA was assayed for C5 expression via Taqman qPCR using the Luna Universal Probe One-Step RT-qPCR Kit (NEB, E3006). A qPCR assay was performed for each sample using a C5 TaqMan probe set (Hs01004342_m1-FAM) multiplexed with a common GAPDH VIC probe (ThermoFisher, 4326317E). Thermocycling and data acquisition was performed with an Applied Biosystems QuantStudio 3/5 Real-Time PCR System.

### Example 7

### Results

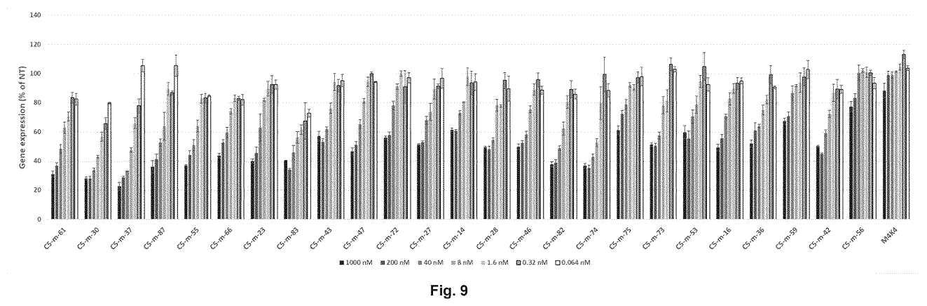

FIG. 8 shows results of the primary screening of selected compounds according to the disclosed embodiments and their activity in inhibiting C5 expression.

Table 12 below shows IC50 values (in nM) for 25 advantageous constructs selected in accordance with the examples. Max % KD indicates the maximally achieved knock-down at 1000 nM with 0% being no knock-down and 100% full knock-down. M4K4 was used as reference.

| SEQ ID No. | Construct ID | Max % KD | IC₅₀ |
|---|---|---|---|
| 1988 | C5-m-30 | 71.88574381 | 4.943 |
| 1995 | C5-m-37 | 77.48131233 | 11.25 |
| 2041 | C5-m-83 | 59.57363723 | 21.99 |
| 2019 | C5-m-61 | 68.93837532 | 40.04 |
| 2032 | C5-m-74 | 63.30023809 | 41.25 |
| 2040 | C5-m-82 | 62.26208156 | 68.84 |
| 2045 | C5-m-87 | 63.87542555 | 89.55 |
| 2013 | C5-m-55 | 63.0068919 | 90.72 |
| 1981 | C5-m-23 | 60.00147173 | 213.2 |
| 1986 | C5-m-28 | 50.59311869 | 295.4 |
| 2000 | C5-m-42 | 49.72928101 | 302.6 |
| 2013 | C5-m-73 | 48.77874599 | 351.7 |
| 2024 | C5-m-66 | 56.30345942 | 351.9 |
| 2005 | C5-m-47 | 53.46576404 | 367.2 |
| 2004 | C5-m-46 | 50.25328616 | 416.7 |
| 1985 | C5-m-27 | 48.71998765 | 583.3 |
| 1974 | C5-m-16 | 50.77488697 | 629.7 |
| 2001 | C5-m-43 | 43.01211645 | 779.6 |
| 1994 | C5-m-36 | 48.1079184 | 894.2 |
| 2030 | C5-m-72 | 44.02312363 | 1090 |
| 2011 | C5-m-53 | 40.3528029 | 1167 |
| 1972 | C5-m-14 | 38.57830035 | 2282 |
| 2033 | C5-m-75 | 38.99786713 | 3485 |
| 2017 | C5-m-59 | 32.76891127 | 5167 |
| 2014 | C5-m-56 | 22.82809496 | 6117 |
| Reference | M4K4 | 11.680749 | 3810 |

The IC50 data in the single- to low double-digit nanomolar range demonstrate outstanding performance of numerous constructs of the disclosed embodiments. Furthermore, no obvious toxicity was observed.

Further results of the screening and the outstanding performance of the above disclosed constructs in Table 12 data are shown in FIG. 9.

Table 13 below shows IC50 values (in nM) for 6 advantageous C5 constructs selected in accordance with the examples. Max % KD indicates the maximally achieved knock-down at 1000 nM with 0% being no knock-down and 100%, full knock-down.

| SEQ ID No. | Construct ID | KD % at 1000 nM | IC₅₀ (nM) |
|---|---|---|---|
| 1988 | C5-m-30 | 85.136214 | 2.939 |
| 1995 | C5-m-37 | 85.717658 | 5.605 |
| 2041 | C5-m-83 | 71.592597 | 37.45 |

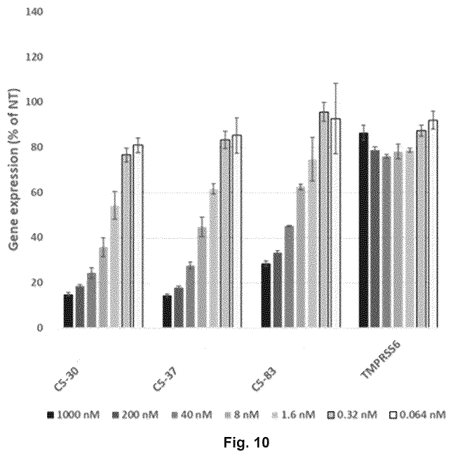

Further results of the constructs in Table 13 above with different concentrations are shown in FIG. 10.

Table 13 and FIG. 10 show C5 large scale preparations mirrored the screening synthesis very closely. C5-m-30 and C5-m-37 had 85% knockdowns and C5-m-83 had a 72% knockdown at the highest dose.

### Example 8

Complement Component C5 Targeting mxRNA Leads for Candidate Dose and Duration Response Study in Humanized Liver-uPA-SCID Mice Model, Non-GLP

### 1. Study Objective(S)

The objective of this non-GLP study is to evaluate the dose and duration response of GalNAc conjugated complement component C5 targeting mxRNA constructs in humanized liver-uPA-SCID. The compound(s) will be administered subcutaneously, and the mice will be survived for up to 42 days.

Prior to necropsy, plasma and serum will be collected. At necropsy, 3 liver biopsies (2 mm) per animal will be preserved in separate vials in RNAlater, flash frozen, and stored at −80° C. Three more liver biopsies (2 mm) will be taken, flash frozen in the same vial, and stored at −80° C.

### 2. Regulatory Compliance

This non-GLP study will not be conducted in accordance with the Food and Drug Administration's Good Laboratory Practice (GLP) regulations (21 CFR Part 58).

### 3. Animal Welfare Compliance

This protocol has been reviewed and approved by the Test Facility IACUC Committee.

### 4. Test System Information

4.1. Animal Test
   4.1.1. Common Name: Mouse
   4.1.2. Breed/Class: Rodent—humanized liver-uPA-SCID mice model
   4.1.3. Number of Animals (by gender): 44 Male all naïve
4.2. Acclimation Period:

### 4.2.1. Duration:

All animals will be acclimated for a minimum period of five (5) days prior to release by the attending veterinarian, at which time the overall health of the animals will be evaluated.

4.2.2. Required Medication and/or Vaccination:
All rodents received will come from a vendor that is certified to be free of any lethal parasites that may affect the facility's total colony.
All rodents must be accompanied by a sentinel report including statistical analysis.
Each shipment of rodents must be housed separately from others in the facility.

### 4.3. Animal Identification Method and Location:

Animals will be assigned sequential numbers. The animals will be ear notched by the vendor prior to shipment to permanently identify each animal. Animals may have color markings to distinguish between similar ear notches. A cage card will also be affixed to each animal cage denoting the animal number, gender, vendor, strain, study director, and study number.

### 5. STUDY DESIGN

### 5.1. Design Details

This study will have one type of mice, N=44. Animals will be grouped by treatment type, dosage, and survival period. Each animal will be treated by subcutaneous injection of test material. See study table 1 for details.

At necropsy, three 2 mm biopsy punches will be taken from the left, middle and right liver lobes, placed in separate vials, soaked in RNAlater for 15 minutes, flash frozen and stored at −80° C. Another three 2 mm liver biopsies from the left, middle and right liver lobes will be placed into one vial, flash frozen and stored at −80° C. The rest of the liver will be flash frozen and stored in 10 mL conical tubes at −80° C.

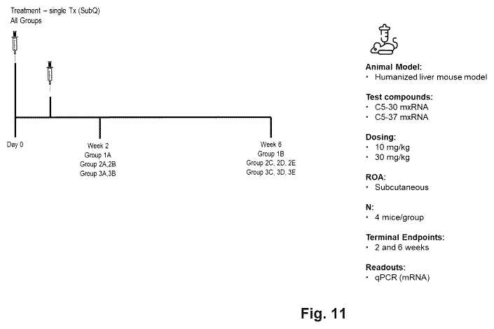

The study schedule is also shown in FIG. 11.

TABLE 14
Dose information

| Terminal | Control | C5-m-30 | C5-m-37 |
|---|---|---|---|
| Timepoint | (PBS) | SEQ ID No. 1988 | SEQ ID No. 1995 |
| Week 2 | Group | Group 2A | Group 3A |
| | 1A | (10 mg/kg) | (10 mg/kg) |
| | (N = 4) | (N = 4) | (N = 4) |
| | | Group 2B | Group 3B |
| | | (30 mg/kg) | (30 mg/kg) |
| | | (N = 4) | (N = 4) |
| Week 6 | Group | Group 2C | Group 3C |
| | 1B | (10 mg/kg) | (10 mg/kg) |
| | (N = 4) | (N = 4) | (N = 4) |
| | | Group 2D | Group 3D |
| | | (30 mg/kg) | (30 mg/kg) |
| | | (N = 3) | (N = 3) |
| | | Group 2E | Group 3E |
| | | 2x (10 mg/kg) | 2x (10 mg/kg) |
| | | (N = 3) | (N = 3) |

TABLE 15
Study table

| | Number | Treatment - | | | |
|---|---|---|---|---|---|
| | of | Subcutaneous | TX | Survival | Pre-Euthanasia and |
| Group | Animals | Injection | Days | Days | Necropsy |
| 1A | 4 | Control (PBS) | 0 | 14 | Pre-Euthanasia: |
| 1B | 4 | Control (PBS) | 0 | 42 | Plasma and serum |
| 2A | 4 | C5-m-30 | 0 | 14 | collection. |
| | | (10 mg/kg) | | | Necropsy: |
| 2B | 4 | C5-m-30 | 0 | 14 | 2 mm biopsy of left, |
| | | (30 m/kg) | | | middle and right liver |
| 2C | 4 | C5-m-30 | 0 | 42 | lobes in separate vials, |
| | | (10 m/kg) | | | in RNAlater for 15 min, |
| 2D | 3 | C5-m-30 | 0 | 42 | flash freeze then stored |
| | | (30 m/kg) | | | at −80° C. |
| 2E | 3 | C5-m-30 | 0, 7 | 42 | 2 mm biopsy of left, |
| | | (10 m/kg) | | | middle and right liver all |
| 3A | 4 | C5-m-37 (10 | 0 | 14 | in one vial, flash freeze |
| | | mg/kg) | | | then stored at −80° C. |
| 3B | 4 | C5-m-37 (30 | 0 | 14 | Rest of liver, flash freeze |
| | | mg/kg) | | | then stored at −80° C. |
| 3C | 4 | C5-m-37 (10 | 0 | 42 | |
| | | mg/kg) | | | |
| 3D | 3 | C5-m-37 (30 | 0 | 42 | |
| | | mg/kg) | | | |
| 3E | 3 | C5-m-37 (10 | 0, 7 | 42 | |
| | | mg/kg) | | | |
| Spares | 0 | | | | |
| Total | 44 | | | | |

### 6. Test Article and Ancillary Material Information

6.1. Test Drug 1:
   6.1.1. Identification: C5-m-30 (SEQ ID No. 1988)
   6.1.2. Manufacturer: Sirnaomics
   6.1.3. Description: GalNAc conjugated human Complement component C5 targeting mxRNA
   6.1.4. Lot/Batch Number: Will be recorded on study materials form.
   6.1.5. Expiration Date: Will be recorded on study materials form.
   6.1.6. Storage Temperature: 4° C.
   6.1.7. Bio-Hazard Status: None
   6.1.8. MSDS*: TBD
   6.1.9. Appearance: Clear Liquid
   6.1.10. Dose Information: See Table 14
   6.1.11. Residual Test Article Storage: None
6.2. Test Drug 2:
   6.2.1. Identification: C5-m-37 (SEQ ID No. 1995)
   6.2.2. Manufacturer: Sirnaomics
   6.2.3. Description: GalNAc conjugated human Complement component C5 targeting mxRNA
   6.2.4. Lot/Batch Number: Will be recorded on study materials form.
   6.2.5. Expiration Date: Will be recorded on study materials form.
   6.2.6. Storage Temperature: 4° C.
   6.2.7. Bio-Hazard Status: None
   6.2.8. MSDS*: TBD
   6.2.9. Appearance: Clear Liquid
   6.2.10. Dose Information: See Table 1
   6.2.11. Residual Test Article Storage: None

### Results

Results of the C5 gene knock down by constructs C5-m-30 and C5-m-37 in humanized liver-uPA-SCID mice are shown in the Tables below.

TABLE 16a
Results of C5 gene knockdown for construct C5-m-30 (see Table
3e for structure) at several time points using different doses.

| C5-m-30 | Knockdown values in liver |
|---|---|
| (SEQ ID No. 1988) | tissue |
| Single Tx - 10 mg/kg | 51% KD at 2 weeks |
| | 10% KD at 6 weeks |
| Single Tx - 30 mg/kg | 61% KD at 2 weeks |
| | 36% KD at 6 weeks |
| Repeat 2x Tx - 10 mg/kg | 17% KD at 6 weeks |

TABLE 16b
Results of C5 gene knockdown for construct C5-m-37 (see Table
3e for structure) at several time points using different doses.

| C5-m-37 | Knockdown values in liver |
|---|---|
| (SEQ ID No. 1995) | tissue |
| Single Tx - 10 mg/kg | 50% KD at 2 weeks |
| | 27% KD at 6 weeks |
| Single Tx - 30 mg/kg | 54% KD at 2 weeks |
| | 47% KD at 6 weeks |
| Repeat 2x Tx - 10 mg/kg | 40% KD at 6 weeks |

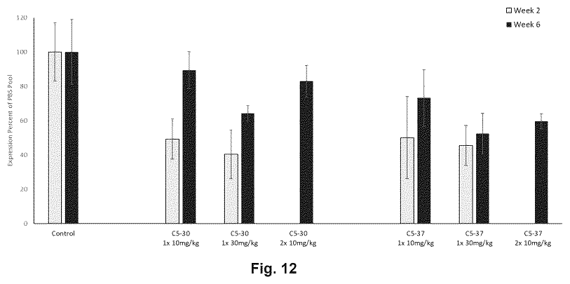

The results of the mouse study are also shown in FIG. 12.

### Example 9

### Cell Culture:

Human primary hepatocytes (5 donor pooled—Sekisui XenoTech, HPCH05+) were thawed immediately prior to experimentation and cultured in 1× complete Williams medium (Gibco, A1217601) supplemented with Hepatocytes plating supplement pack (Gibco, CM3000). FBS concentration was modified from manufacture recipe to a final 2.5% (as opposed to 5%) for compound stability.

1× Complete WEM: 2.5% FBS, 1 μM Dexamethasone, Pen/Strep (100 U/mL/100 μg/mL), 4 μg/ml Human Insulin, 2 mM GlutaMAX, 15 mM HEPES, pH 7.4.

Hepatocytes were plated on Collagen I (rat tail) coated 96 well tissue culture plates (Gibco, A1142803).

### CFB-C5 Combo Screen:

On the day of transfection, primary human hepatocytes were thawed in 45 mL of human OptiThaw (Sekisui XenoTech, K8000) and centrifuged down at 200 g for 5 minutes. Cells were resuspended in 2× complete WEM and counted. Cell were then plated in 50 μL of 2× complete WEM at 25,000 cells per well on 96 well type 1 rat tail Collagen plates and allowed to rest and attach for four hours before transfection.

Compounds were diluted further to 2 μM in basal WEM. A seven step, fivefold dilution series was prepared in basal WEM from 2 μM to 0.000128 μM. 50 μL of each dilution was added to respective triplicates of the plated hepatocytes for a final dilution series of 1 μM down to 0.000064 μM in a volume of 100 uL 1× complete WEM.

72 hours post transfection, cells were harvested and RNA isolated using the PureLink Pro 96 total RNA Purification Kit (ThermoFisher, 12173011 A) according to the manufacturer protocol. Harvested RNA was assayed for CFB and C5 expression via TaqMan qPCR using the Luna Universal Probe One-Step RT-qPCR Kit (NEB, E3006). A single qPCR assay was performed for each sample using an CFB and C5 multiplexed with a common GAPDH VIC probe (ThermoFisher, 4326317E). Thermocycling and data acquisition was performed with an Applied Biosystems QuantStudio 3/5 Real-Time PCR System.

Table 17a below shows IC50 values (in nM) for 4 advantageous muRNA constructs (see Table 4b) and gene knock down for CFB gene. Max % KD indicates the maximally achieved knock-down at 1000 nM with 0% being no knock-down and 100% full knock-down.

| | | Max KD % at | |
|---|---|---|---|
| SEQ ID No. | Construct ID | 1000 nM | IC₅₀ (nM) |
| 2067-2068 | B106-C5-30 | 82.808135 | 2.008 |
| 2069-2070 | B106-C5-37 | 84.403628 | 3.914 |
| 2071-2072 | B13-C5-30 | 6.4534394 | 7029 |
| 2073-2074 | B13-C5-37 | −0.04102 | 4.362E−08 |

Table 17b below shows IC50 values (in nM) for 4 advantageous muRNA constructs (see Table 4b) and gene knock down for C5 gene. Max % KD indicates the maximally achieved knock-down at 1000 nM with 0% being no knock-down and 100% full knock-down.

| | | Max KD % at | |
|---|---|---|---|
| SEQ ID No. | Construct ID | 1000 nM | IC₅₀ (nM) |
| 2067-2068 | B106-C5-30 | 74.461352 | 21.66 |
| 2069-2070 | B106-C5-37 | 73.32972 | 16.58 |
| 2071-2072 | B13-C5-30 | 56.448925 | 228.5 |
| 2073-2074 | B13-C5-37 | 70.985602 | 18.22 |

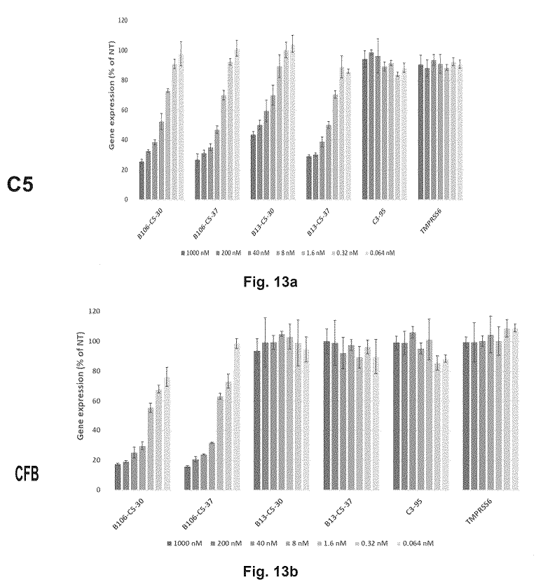

The results are also shown in FIGS. 13a and 13b.

### Example 10

Dose Response Study Evaluating Human Complement Combination (C5 and CFB; muRNA) Targeting Leads for Candidate in Humanized Liver-uPA-SCID Mice Model, Non-GLP

### 1. Study Objective(S)

The objective of this non-GLP study is to evaluate, in humanized liver-uPA-SCID mice, the dose response of GalNAc conjugated human dual targeting (C5 and CFB) muRNA constructs. The compound(s) will be administered subcutaneously, and the mice will be survived for up to 14 days.

Prior to necropsy, blood will be collected for plasma samples. At necropsy, 3 liver biopsies (2 mm) per animal will be preserved in separate vials in RNAlater, flash frozen, and stored at −80° C. Three more liver biopsies (2 mm) will be taken, flash frozen in the same vial, and stored at −80° C. The remaining liver will be flash frozen and stored at −80° C.

### 2. Regulatory Compliance

This non-GLP study will not be conducted in accordance with the Food and Drug Administration's Good Laboratory Practice (GLP) regulations (21 CFR Part 58).

### 3. Animal Welfare Compliance

This protocol has been reviewed and approved by the Test Facility IACUC Committee.

### 4. Test System Information

4.1. Animal Test
   4.1.1. Common Name: Mouse
   4.1.2. Breed/Class: Rodent—Mouse humanized liver-uPA-SCID
   4.1.3. Number of Animals (by gender): 32 Male PXB all naïve
4.2. Acclimation Period:
   4.2.1. Duration:

All animals will be acclimated for a minimum period of five (5) days prior to release by the Attending veterinarian, at which time the overall health of the animals will be evaluated. Animals, which are not released from acclimation will be treated accordingly and further evaluation will be performed prior to release. All records from the acclimation period will remain in the study file.

4.2.2. Required Medication and/or Vaccination:
All rodents received will come from a vendor that is certified to be free of any lethal parasites that may affect the facility's total colony.
All rodents must be accompanied by a sentinel report including statistical analysis.
Each shipment of rodents must be housed separately from others in the facility.

### 4.3. Animal Identification Method and Location:

Animals will be assigned sequential numbers. The animals will be ear notched to permanently identify each animal. This method involves punching holes or notches in the ear pinna while anesthetized. Alternatively, the animals may have a tattoo placed on their tail. A cage card will also be affixed to each animal cage denoting the animal number, gender, vendor, strain, study director, and study number.

### 5. Study Design

### 5.1. Design Details

This study will have one type of mice, N=32. Animals will be grouped by treatment type, dosage, and survival period. Each animal will be treated by subcutaneous injection of test material. Animals will be survived for 14 days. See study table 1 for details.

At necropsy, three 2 mm biopsy punches will be taken from the left, middle and right liver lobes, placed in separate vials, soaked in RNAlater for 15 minutes, flash frozen and stored at −80° C. Another three 2 mm liver biopsies from the left, middle and right liver lobes will be placed into one vial, flash frozen and stored at −80° C. The rest of the liver will be flash frozen and stored in 10 mL conical tubes at −80° C.

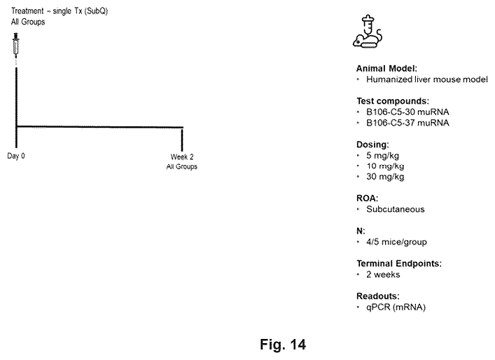

The schedule is also shown in FIG. 14.

TABLE 18
Study table

| | | Treatment | | |
|---|---|---|---|---|
| | Number | Subcutaneous | | |
| | of | Injection | Survival | Pre-Euthanasia and |
| Group | Animals | Day 0 | Days | Necropsy |
| 1A | 4 | Control (PBS) | 14 | Pre-Euthanasia: |
| 2A | 4 | B106-C5-30 5 mg/kg | 14 | Plasma and collection. |
| 2B | 5 | B106-C5-30 (10 | 14 | Necropsy: |
| | | mg/kg) | | 2 mm biopsy of left, |
| 2C | 5 | B106-C5-30 (30 | 14 | middle and right liver |
| | | mg/kg) | | lobes in separate vials, |
| 3A | 4 | B106-C5-37 (5 mg/kg) | 14 | in RNAlater for 15 min, |
| 3B | 5 | B106-C5-37 (10 | 14 | flash freeze then stored |
| | | mg/kg) | | at −80° C. |
| 3C | 5 | B106-C5-37 (30 | 14 | 2 mm biopsy of left, |
| | | mg/kg) | | middle and right liver all |
| Spares | 0 | | | in one vial, flash freeze |
| Total | 32 | | | then stored at −80° C. |
| | | | | Rest of liver, flash freeze |
| | | | | then stored at −80° C. |

### 6. Test Article and Ancillary Material Information

6.1. Test Drug 1:
   6.1.1. Identification: B106-C5-30 (STP247G, see Table 4b for structure, SEQ ID Nos. 2067-2068)
   6.1.2. Manufacturer: Sirnaomics
   6.1.3. Description: GalNAc-muRNA targeting human CFB and Complement C5 mRNA
   6.1.4. Lot/Batch Number: Will be recorded on study materials form.
   6.1.5. Expiration Date: Will be recorded on study materials form.
   6.1.6. Storage Temperature: 4° C.
   6.1.7. Bio-Hazard Status: None
   6.1.8. MSDS*: TBD
   6.1.9. Appearance: Clear Liquid
   6.1.10. Dose Information: See Table 1
   6.1.11. Residual Test Article Storage: None
6.2. Test Drug 2:
   6.2.1. Identification: B106-C5-37 (see Table 4b for structure, SEQ ID Nos. 2069-2070)
   6.2.2. Manufacturer: Sirnaomics
   6.2.3. Description: GalNAc-muRNA targeting human CFB and Complement C5 mRNA
   6.2.4. Lot/Batch Number: Will be recorded on study materials form.
   6.2.5. Expiration Date: Will be recorded on study materials form.
   6.2.6. Storage Temperature: 4° C.
   6.2.7. Bio-Hazard Status: None
   6.2.8. MSDS*: TBD
   6.2.9. Appearance: Clear Liquid
   6.2.10. Dose Information: See Table 18
   6.2.11. Residual Test Article Storage: None

### Results

Table 19a below shows results of CFB gene knockdown at 2 weeks for muRNA constructs B106-C5-30 and B106-C5-37 (see Table 4b for structure) for different doses.

% CFB mRNA KD in liver tissues

| | B106-C5-30 | B106-C5-37 |
|---|---|---|
| | (SEQ ID Nos. | (SEQ ID Nos. |
| Dosing | 2067-2068) | 2069-2070) |
| 10 mg/kg | 42% | 36% |
| 30 mg/kg | 73% | 65% |

Table 19b below shows results of C5 gene knockdown at 2 weeks for muRNA constructs B106-C5-30 and B06-C5-37 (see Table 4b for structure) for different doses.

% C5 mRNA KD in liver tissues

| | B106-C5-30 | B106-C5-37 |
|---|---|---|
| | (SEQ ID Nos. | (SEQ ID Nos. |
| Dosing | 2067-2068) | 2069-2070) |
| 10 mg/kg | 28% | 26% |
| 30 mg/kg | 50% | 58% |

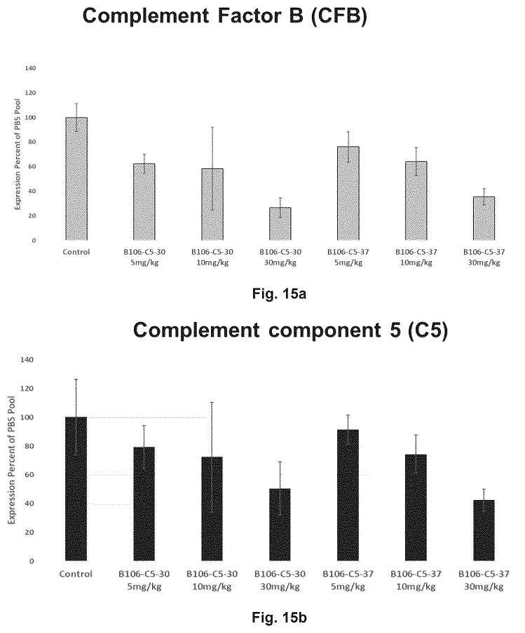

The results of the dose response study evaluating human complement combination (C5 and CFB; muRNA) targeting Leads for Candidate in humanized liver-uPA-SCID mice model are also shown in FIGS. 15a and 15b.

### Example 11

### Evaluation of Duration Effect of STP247G (Complement C5Complement C5/Factor B Dual Targeting mxRNAmuRNA), in the Humanized Liver-uPA-SCID Mice (PXB) Model, Non-GLP

### Materials and Methods

### 1. Study Number

The objective of this non-GLP study is to evaluate, in humanized liver-uPA-SCID (PXB) mice, the duration effect of STP247G, construct B106-C5-30 (SEQ ID Nos. 2067-2068), combination Complement C5/Factor B targeting muRNA The compound(s) will be administered subcutaneously, and the mice will be kept alive for up to 84 days.

### 2. Test System Information

2.1. Animal Test
   2.1.1. Common Name: Mouse
   2.1.2. Breed/Class: Rodent—Mouse PXB
   2.1.3. Number of Animals (by gender): 40 Male PXB all naïve
   2.1.4. Age Range: 14-19 weeks for PXB mice,
   2.1.5. Weight Range: Approx. 20 grams for all mice
2.2. Acclimation Period:
   2.2.1. Duration:

All animals will be acclimated for a minimum period of seven (7) days prior to release by the Attending veterinarian, at which time the overall health of the animals will be evaluated. Animals which are not released from acclimation will be treated accordingly and further evaluation will be performed prior to release. All records from the acclimation period will remain in the study file.
2.2.2. Required Medication and/or Vaccination:
   All rodents received will come from a vendor that is certified to be free of any lethal parasites that may affect the facility's total colony.
   All rodents must be accompanied by a sentinel report including statistical analysis.
   Each shipment of rodents must be housed separately from others in the facility.

### 3. Study Design

### 3.1. Design Details

This study will have one type of mice, 40 PXB. Animals will be grouped by treatment type, dosage, and survival period. Each animal will be treated by subcutaneous injection of test material.
Group 1A, 1B, 1C, and 1 D will have five animals and receive a single control dose of PBS.
Group 2A, 2B 2C, and 2D will have five animals and receive a single dose of STP247G at 50 mg/kg.

Animals will be survived for 14, 28, 56, and 84 days. See Table 20 for details.
Prior to necropsy, the animals will be deeply anesthetized, and a terminal blood draw will be performed through the vena cava. Blood volume will be collected in a plasma separation tube.
At necropsy, three 2 mm biopsy punches will be taken from the left, middle and right liver lobes, placed in separate vials, soaked in RNAlater for 15 minutes, flash frozen and stored at −80° C. Another three 2 mm liver biopsies from the left, middle and right liver lobes will be placed into one vial, flash frozen and stored at −80° C. The rest of the liver will be flash frozen and stored in 10 mL conical tubes at −80° C.

TABLE 20
Study Table

| | | | Treatment | | | |
|---|---|---|---|---|---|---|
| | Number | | Subcutaneous | | | |
| | of | Mouse | Injection | Survival | | Pre-Euthanasia and |
| Group | Animals | Type | Day 0 | Days | Blood | Necropsy |
| 1A | 5 | PXB | Control (PBS) | 14 | Blood | Pre-Euthanasia: |
| 1B | 5 | PXB | Control (PBS) | 28 | collected for | Plasma and collection. |
| 1C | 5 | PXB | Control (PBS) | 56 | plasma. | Necropsy: |
| 1D | 5 | PXB | Control (PBS) | 84 | Plasma will | 2 mm biopsy of left, |
| 3A | 5 | PXB | STP247G 50 | 14 | be evenly | middle and right liver |
| | | | mg/kg | | separated | lobes in separate |
| 3B | 5 | PXB | STP247G 50 | 28 | into two | vials, in RNAlater for |
| | | | mg/kg | | labeled vials. | 15 min, flash freeze |
| 3C | 5 | PXB | STP247G 50 | 56 | | then stored at −80° C. |
| | | | mg/kg | | | 2 mm biopsy of left, |
| 3D | 5 | PXB | STP247G 50 | 84 | | middle and right liver |
| | | | mg/kg | | | all in one vial, flash |
| Spares | 0 | PXB | | | | freeze then stored |
| Total | 40 | PXB | | | | at −80° C. |
| | | | | | | Rest of liver, flash |
| | | | | | | freeze then stored |
| | | | | | | at −80° C. |

### 4. Test Article and Ancillary Material Information

4.1. Test Drug 1:
   4.1.1. Identification: STP247G, construct B106-C5-30 (SEQ ID Nos. 2067-2068)
   4.1.2. Manufacturer: Sirnaomics
   4.1.3. Description: GalNAc-muRNA targeting human Complement C5/Factor B mRNAs
   4.1.4. Lot/Batch Number: Will be recorded on study materials form.
   4.1.5. Expiration Date: Will be recorded on study materials form.
   4.1.6. Storage Temperature: 4° C.
   4.1.7. Bio-Hazard Status: None
   4.1.8. MSDS*: TBD
   4.1.9. Appearance: Clear Liquid
   4.1.10. Dose Information: See Table 1
   4.1.11. Residual Test Article Storage: None

### 5. Technical and Analytical Procedures

### Treatment Procedure:

Procedure Description: Each animal will be injected subcutaneously in scruff with an injection volume of 200 uL according to study table 1. (Note: that the injection must be given subcutaneously. The test articles will not be functional if the subcutaneous site is missed, and injection is given within the muscular region or test articles are injected into the vein/bloodstream).

### Animal Euthanasia and Gross Necropsy

Blood Collection Prior to Necropsy:
   Prior to necropsy, the animals will be deeply anesthetized, and a terminal blood draw will be performed through the vena cava. Blood volume will be collected in a plasma separation tube. After separation the plasma sample will be split evenly into two labeled vials, flash frozen, and stored at −80° C.
Necropsy and Explant procedure:
   A 2 mm biopsy punch will be taken from the left, middle and right liver lobes. Place biopsy samples into separate 2 ml Eppendorf tubes, with 1.5 ml RNAlater and let soak for 15 minutes, flash freeze then store at −80° C. Three more 2 mm biopsy samples will be taken of the left, middle and right liver lobes all placed together into one 2 ml Eppendorf tubes, flash freeze then store at −80° C. Remaining liver will be flash frozen and stored in 10 mL conical tubes at −80° C.

### Results

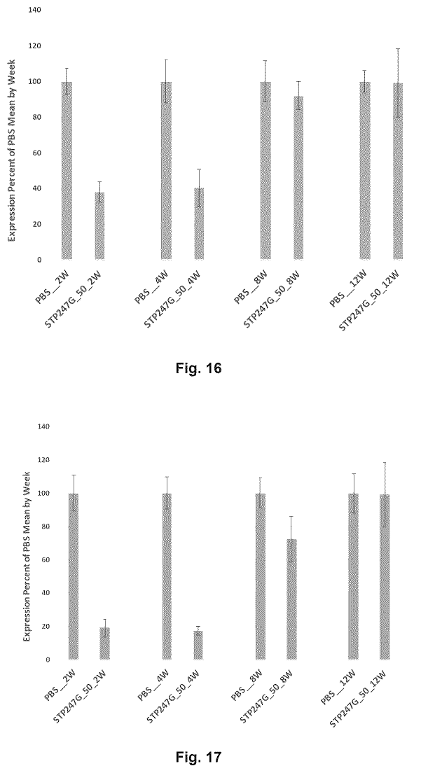

Results are shown in FIGS. 16 and 17. Knockdown of the two targets (C5 and CFB) by the construct (B106-C5-30) have been determined 2, 4, 8 and 12 weeks after a single administration of 50 mg/kg of the indicated compound. Reported values are normalized to the mean of control mice (PBS).

Significant knockdown of the mRNA of either target could be demonstrated across several weeks.

1. A nucleic acid construct comprising at least:
(a) a first nucleic acid sequence that is complementary to a first portion of an RNA which is transcribed from a targeted CFB gene;
(b) a second nucleic acid sequence that is complementary to a second portion of an RNA which is transcribed from a targeted C5 gene;
(c) a third nucleic acid sequence that is at least partially complementary to said first nucleic acid portion of (a), so as to form a first nucleic acid duplex region therewith;
(d) a fourth nucleic acid sequence that is at least partially complementary to said second nucleic acid portion of (b), so as to form a second nucleic acid duplex region therewith,
wherein the first nucleic acid sequence of (a) is directly linked to the fourth nucleic acid sequence of (d) and the second nucleic acid of (b) is directly linked to the third nucleic acid sequence of (c);
wherein the construct contains labile sites such that subsequent to in vivo administration the construct is cleaved at said labile sites to yield at least first and second discrete nucleic acid targeting molecules that respectively target the RNA portions transcribed from the targeted genes of (a) and (b);
wherein the
wherein (i) the first nucleic acid targeting molecule modulates expression of the target gene of (a), and comprises, or is derived from, the first nucleic acid portion of (a), and (ii) the second nucleic acid targeting molecule modulates expression of the targeted gene of (b), and comprises, or is derived from, the second nucleic acid portion of (b).

2-4. (canceled)

5. The construct according to claim 1, wherein said labile sites comprise one or more unmodified nucleotides.

6-8. (canceled)

9. The construct according to claim 1, wherein
(a) said first nucleic acid sequence is selected from the group consisting of SEQ ID Nos. 1-252;
(b) said second nucleic acid sequence is selected from the group consisting of SEQ ID Nos. 504-754;
(c) said third nucleic acid sequence is selected from the group consisting of SEQ ID Nos 253-504; and/or
(d) said fourth nucleic acid sequence is selected from the group consisting of SEQ ID Nos. 755-1004,
wherein said third and fourth nucleobase sequences, to the extent they have a length of 15 nucleobases, may be shorter by one, two, three or four nucleobases, and to the extent they have a length of 14 nucleobases, by one, two or three nucleobases, wherein advantageously the 5′-terminal nucleobase(s) is/are absent.

10-12. (canceled)

13. The construct according to claim 1, wherein said second and third nucleic acid sequences have are selected from the group consisting of: SEQ ID NOs: 534 and 265; 534 and 358; 541 and 265; 541 and 358; and wherein said sequences of SEQ ID NOs: 265 and 358: may be shorter by one, two, three or four nucleobases, wherein optionally the 5′-terminal nucleobase(s) is/are absent.

14-21. (canceled)

22. The construct according to claim, wherein said first and second nucleic acid sequences each independently have a length of 18 to 21, 18 to 20, or 19 nucleotides.

23-27. (canceled)

28. The construct according to claim 1, which further comprises one or more ligands.

29-36. (canceled)

37. The construct according to claim 1, which comprises one, two, or three N-Acetyl-Galactosamine moieties.

38-39. (canceled)

40. The construct according to claim 37, wherein said ligand has the following structure:

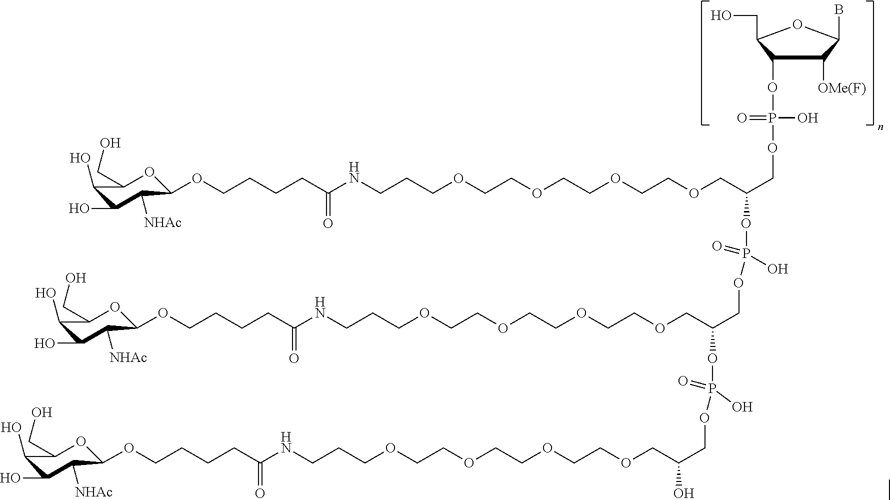

41. (canceled)

42. The construct according to claim 1, which comprises 1 to 15 phosphorothioate or phosphorodithioate internucleotide linkages.

43-46. (canceled)

47. The construct according to claim 1, wherein at least one nucleotide is 2′ modified.

48-61. (canceled)

62. The construct according to claim 47, wherein said 2′ modified sugar is a 2-O-methyl modified sugar of a 2′-F modified sugar.

63-69. (canceled)

70. The construct according to claim 5, wherein all remaining nucleotides other than the labile sites contain either 2′-O-methyl modifications or 2′-F modifications in ribose moieties.

71-72. (canceled)

73. The construct of claim 1, wherein
(a) said first nucleic acid portion has a sequence selected from the first 19 nucleotides of an oligonucleotide selected from the group consisting of SEQ ID Nos. 1505-1758, or is represented by a nucleic acid sequence: 5′[phos] mU #fU #mG fA mA fU mG fA mA fA mC fG mA fC mU #fU #mC #fU #rC (SEQ ID No. 2075); or
5′[phos] mU #fU #mG fC mC fA mC fA mG fA mC fU mC fA mG fA #mG #mA #rG (SEQ ID No. 2076);
(b) said second nucleic acid portion is selected from the group consisting of SEQ ID Nos. 1759-1858, or is represented by a nucleic acid sequence 5′[phos] mG #fA #mU fA mG fU mU fG mU fA mA fA mC fA mG #fU #fU #fC #rC (SEQ ID No. 2077); or
5′[phos] mU #fU #mA fC mA fA mC fA mG fA mA fU mA fU mG #fG #mU #fA #rU (SEQ ID No. 2078);
(c) said fourth nucleic acid portion is selected from the group consisting of SEQ ID Nos.1859-1958, or is represented by a nucleic acid sequence: fC #mU #fG mU fU mU fA mC fA mA fC mU mA #mU #mC #[3XGalNAc](SEQ ID No. 2079); or
fC #mA #fU mA fU mU fC mU fG mU fU mG mU #mA #mA #[3XGalNAC](SEQ ID No. 2090); and/or
(d) said third nucleic acid portion is selected from the group consisting of (i) the last 15 nucleotides of each of SEQ ID Nos. 1505-1756, (ii) a last 14 nucleotides of SEQ ID No. 1757, and (iii) a last 11 nucleotides of SEQ ID No. 1758, or is represented by a nucleic acid sequence: fC mU fG mA fG mU fC mU fG mU fG mG mC #mA #mA #[3XGalNAc](SEQ ID No. 2080); or
fC mU fG mA fG mU fC mU fG mU fG mG mC #mA #mA #(SEQ ID No. 2081).

74. The construct of claim 1 wherein said construct comprises a first strand selected from the group consisting of SEQ ID Nos. 2059-2066, and a second strand selected from the group consisting of SEQ ID Nos. 2067-2074.

75. The construct of claim 74, wherein the first strand has a sequence:
(SEQ ID No. 2082)
5′[phos] mU# fU# mG fA mA fU mG fA mA fA mC
fG mA fC mU# fU# mC# fU# rC fC# mU# fG mU
fU mU fA mC fA mA fC mU mA# mU# mC#
[3XGalNAC];
(SEQ ID No. 2083)
5′[phos] mU# fU# mG fA mA fU mG fA mA fA mC
fG mA fC mU# fU# mC# fU# rC fC# mA# fU mA
fU mU fC mU fG mU fU mG mU# mA# mA#
[3XGalNAc];
(SEQ ID No. 2084)
5′[phos] mU# fU# mG fC mC fA mC fA mG fA mC
fU mC fA mG fA# mG# mA# rG fC# mU# fG mU
fU mU fA mC fA mA fC mU mA# mU# mC#
[3XGalNAc];
or
(SEQ ID No. 2085)
5′[phos] mU# fU# mG fC mC fA mC fA mG fA mC
fU mC fA mG fA# mG# mA# rG fC# mA# fU mA
fU mU fC mU fG mU fU mG mU# mA# mA#
[3XGalNAc];
and/or wherein the second strand has a sequence:
(SEQ ID No. 2086)
5′[phos] mG# fA# mU fA mG fU mU fG mU fA mA
fA mC fA mG# fU# fU# fC# rC fA# mG# fU mC
fG mU fU mU fC mA fU mU mC# mA# mA#
[3XGalNAc];
(SEQ ID No. 2087)
5′[phos] mU# fU# mA fC mA fA mC fA mG fA mA
fU mA fU mG# fG# mU# fA# rU fA# mG# fU mC
fG mU fU mU fC mA fU mU mC# mA# mA#
[3XGalNAc];
(SEQ ID No. 2088)
5′[phos] mG# fA# mU fA mG fU mU fG mU fA mA
fA mC fA mG# fU# fU# fC# rC fC mU fG mA
fG mU fC mU fG mU fG mG mC# mA# mA#
[3XGalNAc];
or
(SEQ ID No. 2089)
5′[phos] mU# fU# mA fC mA fA mC fA mG fA mA
fU mA fU mG# fG# mU# fA# rU fC mU fG mA
fG mU fC mU fG mU fG mG mC# mA# mA#
[3XGalNAc],
wherein Phos being phosphate; [mN], N being any nucleoside, designates 2′-OMe; [fN], N being any nucleoside, designates: 2′-F; [rA], N being any nucleoside, designates: 2′-OH; [#] designates a phosphorothioate connecting two adjacent nucleosides; and [3XGalNAc] designates a following ligand, as shown in square brackets: 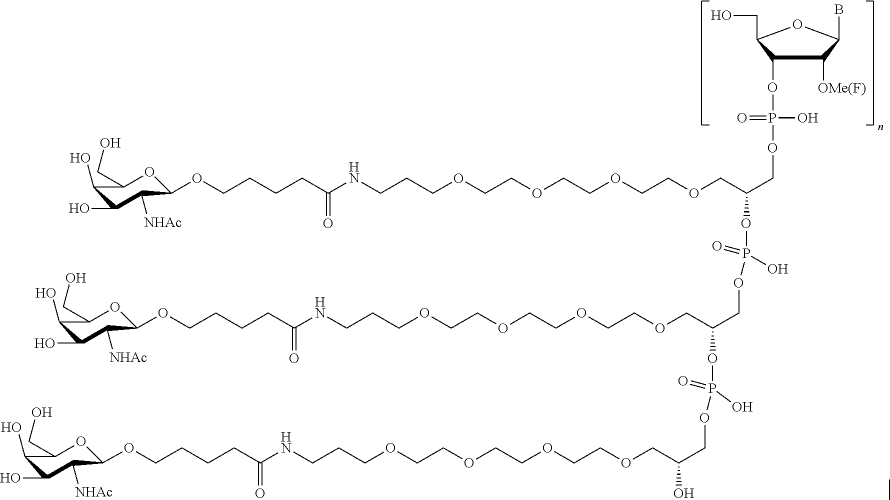
and wherein 5Phos is optional.

76. The construct according to claim 1, selected from the group consisting of SEQ ID Nos. 2059-2074.

77. The construct according to claim 1, wherein the 3′ terminal positions of said first and said third nucleic acid sequences are replaced with an unmodified nucleotide.

78-83. (canceled)

84. The construct according to claim 1, wherein the total length of the construct is 30 to 35 nucleosides, optionally 33 or 34 nucleosides.

85. (canceled)

86. A pharmaceutical composition comprising a nucleic acid construct according to claim 1 and a pharmaceutically acceptable excipient, diluent, antioxidant, and/or preservative.

87-88. (canceled)

89. The pharmaceutical composition of claim 86, wherein said pharmaceutical composition furthermore comprises one or more further pharmaceutically active agents selected from the group consisting of: an agent which modulates the innate and/or the adaptive immune system; an oligomeric compound directed to an immune system target; compounds targeting the proximal complement or Lectin pathway; MASP-2 targeting compounds; C3-targeting compounds; Sutimlimab; Narsoplimab; Pegcetacoplan AMY-102; IONIS-FB-LRx-LPN023; Lapalizumab; Mini-FH/AMY-201 MicroCept; GLG561; and combinations thereof.

90-95. (canceled)

96. A method of treating a disease or disorder comprising administration of a nucleic acid construct according to claim 1 to an individual in need of treatment of a disease or disorder requiring reduction of CFB and/or C5 expression.

94-99. (canceled)
